(19) United States
(12) Patent Application Publication
(10) Pub. No.: US 2018/0214395 A1
(43) Pub. Date: Aug. 2, 2018

(54) Compounds for Treatment or Prevention of Disorders of the Nervous System and Symptoms and Manifestations Thereof, and for Cyto-Protection Against Diseases and Aging of Cells, and Symptoms and Manifestations Thereof

(71) Applicants: Paolo L. Manfredi, New York, NY (US); Charles E. Inturrisi, New York, NY (US)

(72) Inventors: Paolo L. Manfredi, New York, NY (US); Charles E. Inturrisi, New York, NY (US)

(21) Appl. No.: 15/884,915

(22) Filed: Jan. 31, 2018

Related U.S. Application Data
(60) Provisional application No. 62/551,948, filed on Aug. 30, 2017.
(60) Provisional application No. 62/452,453, filed on Jan. 31, 2017.

(51) Int. Cl. A61K 31/137; A61K 45/06; A61K 9/00; A61K 31/485; A61P 25/00

(52) U.S. Cl. CPC A61K 31/137; A61K 45/06; A61P 25/00; A61K 31/485; A61K 9/0019

(57) ABSTRACT

The present invention relates to a method of treating or preventing cellular dysfunction and death caused by genetic, degenerative, toxic, traumatic, ischemic, infectious, neoplastic and inflammatory diseases and aging—and their neurological symptoms and manifestations, which includes administering d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, d-isomethadone, normethadone, N-methyl-methadone, N-methyl-d-methadone, N-methyl-l-methadone, l-moramide, levopropoxyphene, pharmaceutically acceptable salts, or mixtures thereof, including deuterated and tritium analogues, whether isolated from its enantiomer or synthesized de novo.

### CROSS-REFERENCE TO RELATED APPLICATIONS

This application claims the benefit of the filing date of U.S. Patent Application Ser. No. 62/452,453, entitled “d-Methadone for the Treatment of Disorders of the Nervous System and their Neurological Symptoms and Manifestations,” filed on Jan. 31, 2017, and claims the benefit of the filing date of U.S. Patent Application Ser. No. 62/551,948, entitled “Dextromethadone (d-methadone) for Cyto-Protection against Genetic, Degenerative, Toxic, Traumatic, Ischemic, Infectious and Inflammatory Diseases of Cells and Prevention and Treatment of their Symptoms,” filed on Aug. 30, 2017, the disclosures of which are incorporated by reference herein in their entireties.

### FIELD OF THE INVENTION

The present invention relates to the treatment and/or prevention of disorders of the nervous system, and their symptoms and manifestations, and to cyto-protection against various diseases, aging of cells, and processes caused by treatment of diseases, and to compounds and/or compositions for such treatment and/or prevention.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Many nervous system (NS) disorders cause, or are associated with, neurological symptoms and manifestations that are severe and debilitating, can interfere with the activities of daily living, and/or may contribute to co-morbidities in affected individuals. Some examples of such NS disorders include Alzheimer's disease; presenile dementia; senile dementia; vascular dementia; Lewy body dementia; cognitive impairment [including mild cognitive impairment (MCI) associated with aging and with chronic disease and its treatment], Parkinson's disease and Parkinsonian related disorders, including but not limited to Parkinson dementia; disorders associated with accumulation of beta amyloid protein (including but not limited to cerebrovascular amyloid angiopathy, posterior cortical atrophy); disorders associated with accumulation or disruption of tau protein and its metabolites including but not limited to frontotemporal dementia and its variants, frontal variant, primary progressive aphasias (semantic dementia and progressive non fluent aphasia), corticobasal degeneration, supranuclear palsy; epilepsy; NS trauma; NS infections; NS inflammation [including inflammation from autoimmune disorders (such as NMDAR encephalitis), and cytopathology from toxins (including microbial toxins, heavy metals, pesticides, etc.)]; stroke; multiple sclerosis; Huntington's disease; mitochondrial disorders; Fragile X syndrome; Angelman syndrome; hereditary ataxias; neuro-otological and eye movement disorders; neurodegenerative diseases of the retina like glaucoma, diabetic retinopathy, and age-related macular degeneration; amyotrophic lateral sclerosis; tardive dyskinesias; hyperkinetic disorders; attention deficit hyperactivity disorder (“ADHD”) and attention deficit disorders; restless leg syndrome; Tourette's syndrome; schizophrenia; autism spectrum disorders; tuberous sclerosis; Rett syndrome; Prader Willi syndrome; cerebral palsy; disorders of the reward system including but not limited to eating disorders [including anorexia nervosa (“AN”), bulimia nervosa (“BN”), and binge eating disorder (“BED”)], trichotillomania; dermotillomania; nail biting; substance and alcohol abuse and dependence; migraine; fibromyalgia; and peripheral neuropathy of any etiology.

Some examples of neurological symptoms and manifestations associated with these and other NS disorders may include: (1) a decline, impairment, or abnormality in cognitive abilities including executive function, attention, cognitive speed, memory, language functions (speech, comprehension, reading and writing), orientation in space and time, praxis, ability to perform actions, ability to recognize faces or objects, concentration, and alertness; (2) abnormal movements, including akathisia, bradykinesia, tics, myoclonus, dyskinesias (including dyskinesias relate to Huntington's disease, levodopa-induced dyskinesias and neuroleptic-induced dyskinesias), dystonias, tremors (including essential tremor), and restless leg syndrome; (3) parasomnias, insomnia, and disturbed sleep pattern; (4) psychosis; (5) delirium; (6) agitation; (7) headache; (8) motor weakness; spasticity; impaired physical endurance; (9) sensory impairment (including impairment and loss of vision and visual field defects, impairment and loss of sense of smell, taste and hearing) and dysesthesias; (10) dysautonomia; and/or (11) ataxia, impairment of balance or coordination, tinnitus, and neuro-otological and eye movement impairments.

In addition to any neurological symptoms or manifestations, any cognitive dysfunction in an individual may be secondary to a neurodevelopmental or neurodegenerative disease such as Alzheimer's disease or Parkinson's disease and Parkinsonian related disorders including but not limited to Parkinson dementia; disorders associated with accumulation of beta amyloid protein (including but not limited to cerebrovascular amyloid angiopathy, posterior cortical atrophy); disorders associated with accumulation or disruption of tau protein and its metabolites including but not limited to frontotemporal dementia and its variants, frontal variant, primary progressive aphasias (semantic dementia and progressive non fluent aphasia), corticobasal degeneration, supranuclear palsy, or may be caused by diseases where the cognitive decline is multifactorial and in part related to treatment of another disease, such as may be seen in cancer, renal failure, epilepsy, HIV, use of therapeutic and recreational drugs, and aging/senescence of cells. Brain radiation therapy and electroconvulsive treatment are examples of therapies potentially associated with cognitive dysfunction.

Due to the numerous NS disorders, and the multiplicity of symptoms and manifestations associated therewith, substances to treat NS disorders (and their symptoms and manifestations) are an area of major unmet medical need. One target that has been a focus of such substances includes N-methyl-d-aspartate (“NMDA”) receptors.

The NMDA receptor is a glutamate receptor. As is known to those skilled in the art, glutamic acid is one of the 20-22 proteinogenic amino acids, and the carboxylate anions and salts of glutamic acid are known as glutamates. In neuroscience, glutamate is an important neurotransmitter. Nerve impulses trigger release of glutamate from the pre-synaptic cell. And in the opposing post-synaptic cell, glutamate receptors, such as the NMDA receptor, bind glutamate and are activated.

The accumulation of glutamate in the synaptic cleft triggers excessive activation of the NMDA receptor with influx of extracellular calcium, aside from sodium ions. Calcium binds to calmodulin and this complex activates several protein kinases, including calcium calmodulin dependent protein kinase, which increases the permeability of α-amino-3-hydroxy-5-methyl-4-isoxazolepropionic acid (“AMPA”) receptors in the dendritic spine, and also promotes the movement of additional AMPA receptors from cytoplasmic stores into the synaptic membrane. Calcium may also stimulate nitric oxide (“NO”) release, which triggers more glutamate release from the presynaptic cell. After NMDA receptor activation, more AMPA receptors will therefore be expressed on post-synaptic membranes—and another stimulus will then result in an enhanced response (enhanced synapsis) with a potential for excitotoxicity (the pathological process by which neurons are damaged and/or killed, due to overactivation of glutamate receptors).

Another consequence of the rapid increase of Ca²⁺ in the cytoplasm is activation of Ca²⁺ channels on the mitochondrial membrane with resulting calcium flux into the mitochondrial matrix. Mitochondrial Ca²⁺ overload might trigger the activation of the mitochondrial permeability transmission pore, which in turn releases apoptotic and necrotic signal factors, leading to cell death [Fraysse et al., Ca²⁺overload and mitochondrial permeability transition pore activation in living delta sarcoglycan-deficient cardiomyocites. Am J Physiol 2010; 299 (3): 1158-1166]. And, neuronal energy supplies are entirely based on mitochondrial oxidative phosphorylation, making neurons especially vulnerable to mitochondrial dysfunction [Dunchen, M. R., Mitochondria, calcium-dependent neuronal death and neurodegenerative disease. Pflugers Arch. 2012: 464 (1): 111-121].

The NMDA receptor complex has important roles in numerous other NS processes, including neuronal plasticity (e.g., the production of neurons from neural progenitor cells, the growth of axons and dendrites, and the formation and reorganization of synapses), synaptic strength (long term potentiation) underlying memory formation, regulation of neuronal degeneration and apoptosis, and protection against excitotoxic injury (including neuronal protection). Disturbances in mitochondrial functions and signaling may play roles in impaired neuroplasticity and neuronal degeneration in Alzheimer's disease, Parkinson disease and Parkinsonian related disorders including but not limited to Parkinson dementia; disorders associated with accumulation of beta amyloid protein (including but not limited to cerebrovascular amyloid angiopathy, posterior cortical atrophy); disorders associated with accumulation or disruption of tau protein and its metabolites including but not limited to frontotemporal dementia and its variants, frontal variant, primary progressive aphasias (semantic dementia and progressive non fluent aphasia), corticobasal degeneration, supranuclear palsy; infection, inflammation and stroke [Cheng et al., Mitochondria and neuroplasticity. ASN Neuro. 2010 Oct. 4; 2(5)]. The NMDA receptor is the predominant molecular device for controlling synaptic plasticity and memory function and allows for the transfer of electrical signals between neurons in the brain and in the spinal column. For these electrical signals to pass, the NMDA receptor must be open. To remain open (activated), glutamate and glycine must bind to the NMDA receptor.

Several lines of evidence from studies suggest that dysfunction of the glutamatergic system may play an important role in the pathophysiology of many NS disorders, such as those listed above. For example, abnormalities in the glutamatergic system/NMDA receptor have been implicated in the development of ADHD [Bauer et al., Hyperactivity and impulsivity in adult attention-deficit/hyperactivity disorder is related to glutamatergic dysfunction in the anterior cingulate cortex. World J Biol Psychiatry. 2016 Dec. 15:1-9; Riva et al., 2 GRIN2B predicts attention problems among disadvantaged children. Eur Child Adolesc Psychiatry. 2015 July; 24(7):827-36].

As a result, NMDA receptor antagonists (chemicals that antagonize, inhibit or modulate the activity of the NMDA receptor) have been viewed as potential therapeutic agents for the treatment of excitatory neurotoxicity in the context of many NS disorders, and their symptoms and manifestations. As such, NMDA receptor antagonists have received attention from scientists and industry because of their effects on crucial neuronal circuits in chronic pain, depression, and NS disorders.

As is known to those of skilled in the art, NMDA receptor antagonists fall into four categories based on their mechanism of action at the NMDA receptor: (1) competitive antagonists, which bind to and block the binding site of the neurotransmitter glutamate; (2) glycine antagonists, which bind to and block the glycine site; (3) non-competitive antagonists, which inhibit NMDA receptors by binding to allosteric sites; and (4) uncompetitive antagonists, which block the ion channel by binding to a site within it.

Unfortunately, available treatments for NS disorders and their neurological symptoms and manifestations—including the use of NMDA receptor antagonists—are few and ineffective, are not tolerated in a high percentage of patients, or have negative side effects. For example, dextromethorphan has a very short half-life and may be ineffective for many disorders. However, dextromethorphan can be combined with quinidine to circumvent the very short half-life of dextromethorphan alone (Ahmed, A. et al., Pseudobulbar affect: prevalence and management. Therapeutics and Clinical Risk Management 2013; 9:483-489). And so, the US Food and Drug Administration (FDA) has approved dextromethorphan HBr and quinidine sulfate 20 mg/10 mg capsules (Nuedexta®; Avanir Pharmaceuticals, Inc) as the first treatment for pseudobulbar affect (PBA). Unfortunately, quinidine carries potentially fatal risks of arrhythmias and thrombocytopenia rendering Nuedexta® a weak candidate for further development for treatment of other disorders. In addition, dextromethorphan has an active metabolite and is subject to a CYP2D6 genetic polymorphism that results in variable pharmacokinetics and response in the population, a clear disadvantage compared to d-methadone (Zhou S F. Polymorphism of human cytochrome P450 2D6 and its clinical significance: part II. Clin Pharmacokinet. 48:761-804, 2009). Further, designer high affinity drugs such as MK-801 are not safe. Ketamine causes hallucinations and other psychotomimetic effects. Memantine, (approved by the FDA for Alzheimer's disease), has a very long half-life, which depends heavily on renal excretion. And, the effects of dextromethorphan and memantine may be too weak or unbalanced to offer a useful drug for many patients with NS disorders.

Other drugs (with an affinity for the NMDA receptor) have either not been considered or not used to treat NS disorders (or their symptoms or manifestations) due to perceived negative connotations of use or negative side effects. For example, methadone, in its racemic form of l- and d-methadone, is a synthetic opioid that acts by binding to opioid receptors, but also has affinity for the NMDA receptor. It is used medically as an analgesic and as a maintenance anti-addictive and reductive preparation in patients with opioid dependency. Methadone is also used in managing severe chronic pain in addition to opioid addiction owing to its long duration of action, extremely powerful effects, and very low cost. Because it is an acyclic analog of morphine, methadone acts on the same opioid receptors as morphine and thus has many of the same effects as morphine, including opioid side effects.

While the use of methadone in patients with addiction and in patients with pain has been associated with both cognitive impairment and cognitive improvement, these effects have been attributed to the opioid actions of methadone (cognitive impairment) and abstinence from illicit drugs or prescription opioids (cognitive improvement). And, the majority of research suggests that Methadone Maintenance Therapy (MMT) and opioids in general are associated with impaired cognitive function and that deficits extended across a range of domains. Further, patients suffering from conditions such as ADHD are more likely to develop dependence on illicit drugs [Biederman et al., Young adult outcome of attention deficit hyperactivity disorder: a controlled 10-year follow-up study. Psychological Medicine. 2006, 36(167-179)], and methadone maintenance patients have a higher prevalence of ADHD compared to the general population.

And so, to date, those of ordinary skill in the art have not considered NMDA receptor antagonists such as methadone and/or its isomers (d-methadone and l-methadone) to be candidate compounds for treatment of NS disorders for many reasons. These reasons include (but are not limited to) the 1) perceived opioid and psychotomimetic effects attributed to methadone and its isomers, rendering them very poor candidates for improving the cognitive function of patients and 2) the negative connotation of methadone [Bruce, R. D., The marketing of methadone: how an effective medication became unpopular. Int J Drug Policy. 2013 November; 24(6):e89-90]. Also, methadone is a strong opioid, with well-known side effects and risks. Further, any cognitive improvement seen in patients switched from other opioids to methadone has been attributed to a lower opioid dose and thus to less opioidergic side effects, and never to a direct positive effect of methadone on cognition. Methadone, like other strong opioids has many risks and side effects, including opioid related effects on cognition, which have made it very difficult, even for those skilled in the art, to recognize any positive effects on cognition related to the other actions of methadone such as those on the NMDA receptor complex or from other mechanisms.

Furthermore, there has been a chronic lack of understanding about the NMDA-activity of racemic methadone, l-methadone, and d-methadone. Due to this chronic lack of understanding to date, any positive effects of such substances on cognitive function have remained counterintuitive. Furthermore, these compounds are expected by those skilled in the art to exert psychotomimetic side effects and opioid side effects.

Aside from the misperception about the potential psychotomimetic and opioid effects of d-methadone, yet another drawback for d-methadone has been the perceived cardiovascular risk associated with d-methadone related compounds, such as racemic methadone and l-alpha-acetylmethadol (“LAAM”), both of which carry a black box warning for QT prolongation and risk of life-threatening arrhythmias. In vitro studies have shown that d-methadone has similar potential for slowing K gated ion channels and therefore for prolonging the QT interval on the electrocardiogram and thus, possibly, for increasing the risk for arrhythmias.

While the in vitro potential for influencing cardiac human ether-a-go-go-related gene K⁺ currents provides a plausible mechanism for arrhythmias in patients receiving methadone like drugs [Katchman A N et al., Influence of opioid agonists on cardiac human ether-a-go-go-related gene K⁽⁺⁾ currents. J Pharmacol Exp Ther. 2002 November; 303(2):688-94], the clinical significance of this action on humans depends on many other factors.

Some of the factors that can influence the clinical outcomes of patients may depend upon d-methadone's effects on other ion channels (aside from K⁺ channels), such as Na or Ca channels, or may depend on pharmacokinetic properties that decrease the likelihood of toxicity, or there could be alternative explanations for adverse cardiovascular outcomes described in patients and attributed to methadone (and thus to its isomers): (1) the influence on Na⁺ currents might oppose the effect on K⁺ currents; methadone and its isomers block voltage dependent K⁺, Ca²⁺ and Na⁺ currents [Horrigan F T and Gilly W F: Methadone block of K⁺current in squid giant fiber lobe neurons. J Gen Physiol. 1996 Feb. 1; 107(2): 243-260]; (2) the influence of NMDAR block on cardiac cells might be cardio-protective [Gill S S. and Pulido O M. Glutamate Receptors in Peripheral Tissues: Current Knowledge, Future Research and Implications for Toxicology. Toxicologic Pathology 2001: 29 (2) 208-223]; (3) d-methadone is 80% protein bound and this might increase the clinically safe dose of d-methadone by 5-fold by reducing the availability of circulating free d-methadone; (4) as detailed in the Examples section, d-methadone is readily transported across the blood brain barrier achieving brain levels 3-4 fold higher compared to serum levels; these novel findings presented by the inventors suggest that d-methadone might be effective at doses lower than expected based solely on serum pharmacokinetics, thus lowering the dose dependent toxicity towards organs outside of the CNS, including cardiac tissue; 5) the arrhythmogenic effects of intravenous methadone in patients might have been caused not by methadone but by the preservative chlorbutanol contained in the intravenous solution [Kornick C A et al., QTc interval prolongation associated with intravenous methadone. Pain. 2003 October; 105(3):499-506], as suggested by the observation that a switch oral doses of methadone was associated with a normalization of the QTc. In other isolated case reports of arrhythmias associated with methadone, concomitant prescription drugs or concomitant illicit drugs may have been the culprit instead of methadone. A recent scientific publication provides support for the cardiac safety of racemic methadone [Bart G et al., Methadone and the QTc Interval: Paucity of Clinically Significant Factors in a Retrospective Cohort. Journal of Addiction Medicine 2017. 11(6):489-493], and another study suggests cardio-protective effects of d-methadone for cardiac ischemic morbidity [Marmor M et al., Coronary artery disease and opioid use. Am J Cardiol. 2004 May 15; 93(10):1295-7], underscoring how clinical data are needed in order to translate in vitro studies and QTc prolongation into clinical contexts. As serum levels of methadone isomers, including d-methadone, are present and measurable in the serum of patients treated with racemic methadone, the results of these observational studies suggest that the effects of racemic methadone and its isomers including d-methadone on voltage dependent K⁺ channels and QT prolongation may not result in cardiac morbidity.

Furthermore, the blood pressure lowering effects of d-methadone observed by the inventors and detailed in the Examples section, and the demonstrated presence of NMDA receptors on extra-neural tissues, including the heart and its conduction system [Gill S S. and Pulido O M. Glutamate Receptors in Peripheral Tissues: Current Knowledge, Future Research and Implications for Toxicology. Toxicologic Pathology 2001: 29 (2) 208-223], suggest that d-methadone may be cardio-protective against arrhythmias and against ischemic heart disease. Ranolazine, a drug approved for the treatment of angina, inhibits persistent or late inward sodium current in heart muscle voltage-gated sodium channels, thereby reducing intracellular calcium level; d-methadone has similar regulatory activity on ionic currents of cells, not only on squid neurons, but also on chick myoblasts [Horrigan F T and Gilly W F: Methadone block of K⁺current in squid giant fiber lobe neurons. J Gen Physiol. 1996 Feb. 1; 107(2): 243-260], suggesting potential cardiac effects similar to those of ranolazine; furthermore, by regulating NMDAR, d-methadone will also result in decreased intracellular calcium overload. Ranolazine influences Na+K+ currents and while it causes prolongation of the Qtc interval, it appears to be cardio-protective rather than arrhythmogenic [Scirica B M et al., Effect of ranolazine, an antianginal agent with novel electrophysiological properties, on the incidence of arrhythmias in patients with non ST-segment elevation acute coronary syndrome: results from the Metabolic Efficiency with Ranolazine for Less Ischemia in Non ST Elevation ST Elevation Acute Coronary Syndrome Thrombolysis in Myocardial Infarction36 (MERLIN-TIMI 36) randomized controlled trial. Circulation. 2007; 116:1647-1652].

Methadone has been associated with decreased cardiovascular morbidity in experimental models [Gross E R et al., Acute methadone treatment reduces myocardial infarct size via the delta-opioid receptor in rats during reperfusion. Anesth Analg. 2009 November; 109(5): 1395-402] and epidemiological studies [Marmor M et al., Coronary artery disease and opioid use. Am J Cardiol. 2004 May 15; 93(10):1295-7]. While these effects have been attributed to opioid effects, the new joint work of the inventors suggests instead that these cardiovascular protecting effects may be intrinsic to non-opioid mechanisms, such as actions at the level of NMDAR and actions on regulation of K+, Na+, Ca currents. A drug like d-methadone, shown by the inventors to be devoid of psychotomimetic and devoid of the opioidergic effects, unlike racemic methadone and l-methadone, could therefore potentially prevent and treat cardiac ischemic disease, including patients with unstable angina, without negative cognitive side effects. The sustained blood pressure lowering effects and hypoglycemic effects also discovered by the inventors and detailed in the Examples section, could also induce cardiovascular protection. Direct vasodilation, possibly through blocking L-type calcium channels, could also signal a potential benefit for patients with cardiac ischemia [Tung K H et al. Contrasting cardiovascular properties of the μ-opioid agonists morphine and methadone in the rat. Eur J Pharmacol 2015 Sep. 5; 762:372-81]. d-Methadone could therefore prevent and treat cardiovascular disease, alone or in combination with other anti-hypertensive or ant-ischemic drugs. All of these observations are unlikely to be known, or to be taken in due consideration in their entirety, even by those skilled in the art, and therefore d-methadone is perceived as a drug with cardiac risks and thus a poor candidate for development for the multiplicity of clinical indications outlined throughout the present application, including cardiovascular indications.

Thus, while there is a great need for medications to treat NS disorders and their symptoms and manifestations, current treatments and medications for the most part have not been effective, and the use of NMDA receptor antagonists, such as methadone or its isomers, has not been considered due to a multiplicity of perceived drawbacks, as described above and, more importantly, there has been no indication of clinical efficacy for the use of d-methadone for treating or preventing nervous system disorders, symptoms and manifestations of nervous system disorders, or improving cognitive function, or treating or preventing endocrine-metabolic disorders, or high blood pressure, or ischemic heart disease, or age related disorders, or eye diseases, or skin diseases, except for the novel work presented by the inventors throughout this application. In fact, to date, no evidence, indication, or signal that a drug like d-methadone may be effective for these disorders has been discovered. Presently available medications are inadequate for the treatment of NS disorders, their symptoms, and/or their manifestations—and there has been little innovation in this area in the last decade. The need for better treatments remains.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

In view of the drawbacks listed above, safe and effective compounds, compositions, drugs, and methods that prevent and/or treat NS disorders and/or their neurological symptoms and manifestations are greatly needed. And so, the present invention relates to treating and preventing various nervous system (NS) disorders [including those of the central nervous system (CNS) and peripheral nervous system (PNS)] and their neurological symptoms and manifestations via compounds, compositions, drugs, and methods that heretofore have not been used—and indeed would not be considered by those of ordinary skill in the art, due to the many perceived drawbacks of certain substances (as described in the Background). Further, the present invention relates to treating and preventing cellular dysfunction and death caused by genetic, developmental, degenerative, toxic, traumatic, ischemic, infectious, neoplastic, and inflammatory diseases, and aging. Further, the present invention relates to treating and preventing diseases of the eye and the endocrine-metabolic system, including diseases and symptoms due to hypothalamic-pituitary axis imbalance.

To that end, apart from the NMDA receptor (discussed above), the norepinephrine transporter (“NET”) system, the serotonin transporter (“SERT”) system, neurotrophic factors such as brain derived neurotrophic factor (“BDNF”), reproductive hormones such as testosterone, and K⁺, Ca²⁺ and Na⁺ cellular currents also have important roles in numerous NS, endocrine, metabolic and trophic processes. And, in addition to abnormalities in the NMDA receptor complex, abnormalities associated with the NET system, SERT system, BDNF, K⁺, Ca²⁺ and Na⁺ cellular currents, and in the reproductive/gonadal system, have also been implicated in the pathogenesis and worsening of many NS, metabolic and trophic disorders, including those NS disorders listed in this Background section. For example, decreased levels of BDNF are associated with neurodegenerative diseases with neuronal impairment, such as Parkinson's disease, Alzheimer's disease, multiple sclerosis, and Huntington's disease [Binder, D. K. et al., Brain-derived neurotrophic factor. Growth Factors. 2004 September; 22(3):123-31]. Markedly decreased levels of BDNF and nerve growth factor (NGF) have been observed in the nigrostriatal dopamine regions of Parkinson disease patients and in the hippocampus of Alzheimer's patients.

Additionally, as described above, abnormalities in the NMDA receptor have been implicated in the development of ADHD. The BDNF gene and the NGFR (nerve growth factor receptor) gene belong to the neurotrophin family and are involved in the development, plasticity and survival of neurons and play an important role in learning and memory formation but also other cognitive functions. Aside from the glutamatergic system and NMDA receptor influence on the development of ADHD, the epigenetic regulation of the BDNF system as well as the NET system and SERT system have been recently found to be implicated in the development of ADHD [Banaschewski, T. et al., Molecular genetics of attention-deficit/hyperactivity disorder: an overview. Eur. Child Adolesc. Psychiatry 19, 237-257 (2010); Heinrich et al., Attention, cognitive control and motivation in ADHD: Linking event-related brain potentials and DNA methylation patterns in boys at early school age. Scientific Reports 7, Article number: 3823 (2017)]. Thus, again, abnormalities in the NET system and SERT system, in BDNF, and in the reproductive/gonadal system seem to negatively affect many of the same disorders as do abnormalities in the NMDA receptor.

The NET and SERT are proteins that function as plasma-membrane transporters to regulate concentrations of extracellular monoamine neurotransmitters. They are responsible for the reuptake of their associated amine neurotransmitters (norepinephrine and serotonin). Compounds that target the NET and SERT include drugs such as the tricyclic antidepressants (TCA's), and selective serotonin reuptake inhibitors (SSRIs). These reuptake inhibitors result in sustained increases in the synapse of the concentration the neurotransmitters norepinephrine and serotonin. d-Methadone can inhibit the NET and SERT [Codd et al., Serotonin and Norepinephrine activity of centrally acting analgesics: Structural determinants and role in antinociception. IPET 1995; 274 (3)1263-1269] and thus increase availability of both norepinephrine (NE) and serotonin in the CNS with potential positive effects on cognitive function. This inhibitory activity on both NE and serotonin re-uptake was confirmed and characterized with new in vitro studies presented by the inventors, as will be described in greater details below in the Examples section.

BDNF is a protein that, in humans, is encoded by the BDNF gene. BDNF is a member of the neurotrophin family of growth factors. Neurotrophic factors are found in the brain and the periphery. BDNF acts on certain neurons of the central nervous system and the peripheral nervous system, helping to support the survival of existing neurons, and encourages the growth and differentiation of new neurons and synapses. In the brain, it is active in the hippocampus, cortex, and basal forebrain—areas vital to learning, memory, and higher cognitive functions. BDNF binds to receptors (TrkA, TrkB, p75NTR) and modulates their downstream pathways. The inventors discovered that d-methadone can up-regulate BDNF serum levels in humans, as will be described in greater details below in the Examples section.

Reproductive/gonadal hormones and in particular testosterone are implicated in the pathogenesis of the metabolic syndrome, type 2 diabetes, obesity [Corona G et al., Testosterone supplementation and body composition: results from a meta-analysis of observational studies. J Endocrinol Invest. 2016 September; 39(9):967-81], and epilepsy [Taubøll E et al., Interactions between hormones and epilepsy. Seizure. 2015 May; 28:3-11; Frye C A. Effects and mechanisms of progestogens and androgens in ictal activity. Epilepsia. 2010 July; 51 Suppl 3:135-40]. Testosterone levels influence depression and cognitive functions [Yeap B B. Hormonal changes and their impact on cognition and mental health of ageing men. Maturitas. 2014 October; 79(2):227-35]. Furthermore, testosterone may be neuroprotective [Chisu V et al., Testosterone induces neuroprotection from oxidative stress. Effects on catalase activity and 3-nitro-L-tyrosine incorporation into alpha-tubulin in a mouse neuroblastoma cell line. Arch Ital Biol. 2006 May; 144(2):63-73] and thus may slow the deterioration that characterizes the aging of cells. Finally, some of the actions of testosterone may be mediated through BDNF [Rasika S et al., BDNF Mediates the Effects of Testosterone on the Survival of New Neurons in an Adult Brain. Proc Natl Acad Sci USA. 1994 Aug. 16; 91(17):7854-8]. The inventors discovered that d-methadone can up-regulate testosterone serum levels in humans, as will be described in greater detail below in the Examples. While not being bound to any theory, it is believed this effect might be mediated by NMDA antagonistic activity at the level of NMDA receptors of hyper-stimulated hypothalamic neurons and thus may represent an effect mediated via regulation of the hypothalamic-pituitary axis. The blood pressure changes, serum glucose levels, oxygen saturation changes described in the Examples section may also be mediated by the same NMDAR antagonistic actions at hypothalamic neurons.

Thus, a drug that modulates the NMDA receptor (and NET and SERT systems), and up-regulates BDNF levels and testosterone serum levels, may reduce excitotoxicity, potentially protect mitochondria from Ca²⁺ overload, and provide neuroprotection and enhance connectivity and trophic functions of neurons, including hypothalamic and retinal neurons and other cells. Additionally, if this drug shows signs of effectiveness in humans, and is found to be safe without psychotomimetic or opioid side effects, it may hold great potential for treating NS disorders and their neurological symptoms and manifestations. Furthermore, a drug that increases BDNF and testosterone serum levels in humans may also be useful for peripheral nerve disorders, such as peripheral neuropathies of different etiology, including diabetic peripheral neuropathy and metabolic disorders and disorders associated with aging of cells and their symptoms and manifestations.

Additionally, it is known that neuroplasticity is connected with the developmental stages of life; however, there is now growing evidence confirming that structural and functional reorganization occurs throughout our lifetime, and may influence the onset, clinical course, and recovery of most diseases of the CNS and PNS [Ksiazek-Winiarek et al., Neural Plasticity in Multiple Sclerosis: The Functional and Molecular Background. Neural Plast. 2015, Article ID 307175]. As described above, BDNF acts on certain neurons of the central nervous system and the peripheral nervous system, helping to support the survival of existing neurons, and encourages the growth and differentiation of new neurons and synapses. And so, drugs that up-regulate serum levels of testosterone and BDNF, by influencing neuronal function and plasticity and trophic functions of cells, are potential therapeutic targets to prevent, alter the course, and/or treat symptoms and manifestations of many disorders, including those associated with normal senescence and accelerated senescence, including senescence accelerated by diseases and their treatments, such as impaired physical endurance and other symptoms of aging.

Since BDNF appears to be involved in activity-dependent synaptic plasticity, there is great interest in its role in learning and memory [Binder D K and Scharfman H E, Brain-derived neurotrophic factor. Growth Factors. 2004 September; 22(3):123-31]. The hippocampus, which is required for many forms of long-term memory in humans and animals, appears to be an important site of BDNF action. Rapid and selective induction of BDNF expression in the hippocampus during contextual learning has been demonstrated [Hall, J. et al., Rapid and selective induction of BDNF expression in the hippocampus during contextual learning. Nat Neurosci. 2000; 3:533-535]. Another study demonstrated up-regulation of BDNF in monkey parietal cortex associated with tool-use learning [Ishibashi, H. et al., Tool-use learning induces BDNF expression in a selective portion of monkey anterior parietal cortex. Brain Res Mol Brain Res. 2002; 102:110-112]. In humans, a valine to methionine polymorphism at the 5′ pro-region of the human BDNF protein was found to be associated with poorer episodic memory; in vitro, neurons transfected with met-BDNF-GFP exhibited reduced depolarization-induced BDNF secretion [Egan, M. F. et al., The BDNF val66met polymorphism affects activity-dependent secretion of BDNF and human memory and hippocampal function. Cell. 2003; 112:257-26].

It is known that BDNF exerts trophic and protective effects on dopaminergic neurons as well as other neuronal systems. Thus, impairment of cognitive function may result from, or be exacerbated by, reduction in BDNF. It has been found that memantine (an NMDA receptor antagonist used to treat Alzheimer's disease) specifically upregulates mRNA and protein expression of BDNF in monkeys [Falko, M. et al., Memantine Upregulates BDNF and Prevents Dopamine Deficits in SIV-Infected Macaques: A Novel Pharmacological Action of Memantine. Neuropsychopharmacology (2008) 33, 2228-2236], suggesting that the protective effect of memantine on dopamine function may be mechanistically remote from NMDA receptor antagonism and may be related to BDNF. Further, Marvanova [Marvanova M. et al. The Neuroprotective Agent Memantine InducesBrain-Derived Neurotrophic Factor and trkB Receptor Expression in Rat Brain. Molecular and Cellular Neuroscience 2001; 18, 247-258] reported that memantine increased production of BDNF in rat brain. BDNF has been suggested as a possible therapeutic candidate for treatment of many NS diseases [Kandel, E. R. et al., Principles of Neural Science, Fifth Edition, 2013].

Against that backdrop, it has been reported that l-methadone (the levo-isomer of racemic methadone) decreases blood levels of BDNF in Methadone Maintenance (MMT) patients (Schuster R. et al., Elevated methylation and decreased serum concentrations of BDNF in patients in levomethadone compared to diamorphine maintenance treatment Eur Arch Psychiatry Clin Neurosci 2017; 267:33-40). However, Tsai et al. [Tsai, M. C. et al., Brain-derived neurotrophic factor (BDNF) and oxidative stress in heroin-dependent male patients undergoing methadone maintenance treatment. Psychiatry Res. 2016 Dec. 27; 249:46-50], found that racemic methadone increases BDNF levels in a similar group of heroin-dependent MMT patients. The present inventors thus reached a novel conclusion that the findings of these studies, when taken together, could indirectly support the idea that d-methadone, rather than l-methadone, is primarily responsible for increasing BDNF levels, and that d-methadone is likely more active in increasing BDNF levels than racemic methadone (which contains 50% l-methadone, which not only as described by Schuster et al., decreases BDNF levels, but also exerts powerful opioid effects, which would obscure any positive cognitive effect of d-methadone. This conclusion has not been previously reached by those skilled in the art, and—to date—there have been thought to be myriad drawbacks to the use of racemic methadone, d-methadone, and l-methadone (as described above), including drawbacks to the use of isomers produced by chiral separation or by de novo synthesis (such as without regard to impurities to a degree that doesn't counteract the benefits of the compounds—such as d-methadone—described herein). And so, the joint work of the present inventors teaches that—in at least one embodiment—d-methadone, in its known compositions, is safe and effective for a multiplicity of indications. Further, certain embodiments pertain to d-methadone produced by chiral separation or by de novo synthesis. Such production thereby allows for effective compounds or compositions, that can be prepared without more exacting and lengthy preparations used to provide compounds of heightened purity.

Further, the effect discussed in Tsai et al may be mediated via modulation at the NMDA and/or NET and/or SERT systems or via upregulation of mRNA, as suggested by Falko et al. (2008), and thus may also be inherent to d-methadone, as suggested by the effects of d-methadone on BDNF levels discovered by the inventors and detailed in the Examples section, and not only to racemic methadone. The present inventors thus reached another novel conclusion (and one heretofore not contemplated by those skilled in the art): That this mRNA-mediated increase in BDNF offers another likely explanation, in addition to the actions at the NMDA receptor, NET system, and SERT system, for the cognitive improvements from d-methadone discovered by the inventors in humans as described below. Furthermore, this signal for increase in BDNF in MMT patients reported by Tsai et al. as resulting from dosing with racemic methadone was seen at doses comparable to the safe and effective doses of d-methadone tested by the inventors.

As is known, l-methadone is principally an opioid agonist, while d-methadone is a very weak opioid agonist, and this activity at central opioid receptors was found by the inventors to be clinically negligible at doses expected by the inventors to exert clinical effects modulating actions at the NMDA receptor, NET system, and SERT system, and potentially up-regulate BDNF and testosterone serum levels in humans. And so, the present inventors have determined for the first time that a drug like d-methadone—which (1) is safe and well-tolerated, (2) is devoid of opioid activity and psychotomimetic effects at doses expected to maintain modulating actions on the NMDA receptor, NET system, and SERT system, and (3) potentially up-regulates BDNF and testosterone—can improve cognitive performance, exert neuroprotective actions and exert trophic functions on cells and regulate the metabolic endocrine axis and treat diseases of the eye without the negative opioid-like effects or psychotomimetic side effects. Thus, when methadone is substituted for other opioids such as in the studies conducted and re-analyzed by the inventors (including Santiago-Palma, J. et al., Intravenous methadone in the management of chronic cancer pain: safe and effective starting doses when substituting methadone for fentanyl. Cancer 2001; 92 (7):1919-1925), the opioidergic effects of methadone and the of prior opioid (the opioid substituted with methadone) neutralize each other, and the effects of other actions of methadone (modulation of the NMDA receptor, NET system, and SERT system and increase in BDNF and testosterone) become apparent and clinically measurable. These other actions (modulation of the NMDA receptor, NET system, and SERT system, and increase in BDNF and testosterone), as shown by the inventors, are present in the d-methadone isomer without opioid effects, while in racemic methadone and in l-methadone they remain combined with strong opioid effects (and therefore are of limited clinical use).

These NMDA, NET, SERT, BDNF, testosterone effects, and modulation of K⁺, Ca²⁺ and Na⁺ currents, might also explain why elderly frail patients with baseline cognitive impairment have better cognitive function while treated with methadone rather than other opioids, as indicated by the present inventor (Manfredi P L. Opioids versus antidepressants in postherpetic neuralgia: A randomized placebo-controlled trial. [Letter]. Neurology. Neurology. 2003 Mar. 25; 60(6):1052-3) and other authors (Vu Bach T et al., Use of Methadone as an Adjuvant Medication to Low-Dose Opioids for Neuropathic Pain in the Frail Elderly: A Case Series. J Palliat Med. 2016 December; 19 (12):1351-1355). This improvement in cognitive function has never before been attributed to a direct effect of methadone or its isomers and has been instead ascribed to lesser opioid side effects from other opioids (the opioid discontinued when methadone is introduced). Furthermore, while the use of methadone in patients with addiction has been associated with cognitive improvement, these effects have not been attributed to direct actions of d-methadone mediated by modulation at the NMDA receptor, NET system, or SERT system, or an increase in BDNF and/or testosterone, and/or a modulating effect on K⁺, Ca²⁺ and Na⁺ currents, as now taught by the present inventors.

The majority of research suggests that Methadone Maintenance Therapy (MMT) and opioids in general are associated with impaired cognitive function and that deficits extend across a range of domains. However, many studies compared cognitive impairment in patients on methadone to healthy controls. These studies overlook the fact that these are not comparable groups and patients with opioid addiction often have pre-existing cognitive impairments (high prevalence of ADHD, cognitive impairment caused by illicit substance use, and co-morbidities such as HIV and HCV that are known to impair cognition).

In fact, while many studies ascribe to methadone a negative effect on cognitive function [see Wang, G. Y. et al., Methadone maintenance treatment and cognitive function: a systematic review. Curr Drug Abuse Rev. 2013 September; 6(3): 220-30], opposite results are found when the cognitive performance of patients on methadone is compared to the cognitive performance of patients using illicit opioids. Wang et al., Soyka et al., and Gruber et al. found that cognitive function or sensory information processing in patients undertaking MMT is improved compared to those of patients using illicit opiates. [See Wang, G. Y. et al., Neuropsychological performance of methadone-maintained opiate users. J Psychopharmacol. 2014 August; 28 (8):789-99; Soyka, M. et al., Better cognitive function in patients treated with methadone than in patients treated with heroin: A comparison of cognitive function in patients under maintenance treatment with heroin, methadone, or buprenorphine and healthy controls: an open pilot study. Am J Drug Alcohol Abuse. 2011 November; 37(6):497-508; Gruber, S. A. et al., Methadone maintenance improves cognitive performance after two months of treatment. Exp Clin Psychopharmacol. 2006 May; 14 (2):157-64, and Wang, G. Y. et al., Auditory event-related potentials in methadone substituted opiate users. J Psychopharmacol. 2015 September; 29 (9):983-95]. And Grevert et al., found no effect of levo-alpha-acetylmethadol, LAAM, on memory (a strong opioid like LAAM would be expected to impair memory processing) [see Grevert, P. et al., Failure of methadone and levomethadyl acetate (levo-alpha-acetylmethadol, LAAM) maintenance to affect memory. Arch Gen Psychiatry. 1977 July; 34(7):849-53]. This unexpected finding by Grevert et al. 1977 and the improvements noted by Wang et al., 2014, Soyka et al., 2011, Gruber et al. 2006, and Wang et al., 2015, in light of joint knowledge and discoveries, signal to the inventors that d-methadone, which is devoid of opioid activity, when tested in patients (or even in subjects with no known disease or impairment), might have a direct positive effect on cognition and sensory information processing.

In light of the joint knowledge of the inventors, these unexpected findings on cognition and memory may be a direct effect of methadone on modulation of NMDA, NET, and SERT systems and/or BDNF and testosterone and therefore inherent to methadone—while not opioid related—and not due to a reduction of illicit opioid use. Thus, a drug like d-methadone might improve deficits in cognitive function and information processing and might be useful in conditions such as ADHD—which is frequent in illicit substance users—and in other conditions associated with cognitive impairment of unspecified etiology. Such a drug, as described herein, may be produced by chiral separation or de novo synthesis. And, that drug (as will be described below in greater detail, and in the Examples) may be a drug produced without regard to achieving impurity levels in the ppm range (which enhances the ease of preparation and use of the present compounds).

To that end, the inventors now provide herein new human data showing that d-methadone up-regulates BDNF and testosterone serum levels in humans. The inventors have also discovered new signals for effectiveness for improving cognitive function in several human studies, new evidence for linear pharmacokinetics, and new pharmacodynamic data that demonstrate lack of opioid cognitive side effects and psychotomimetic side effects at doses potentially therapeutic and new overall safety data (therefore confirming d-methadone's potential for improving cognitive impairment and NS disorders, as discovered by the inventors). The inventors also provide herein new data on characterization of NMDA receptor interactions for d-methadone in the micromolar range and provide new experimental data showing higher than expected CNS levels of d-methadone after systemic administration.

In testing by the inventors (described herein), d-methadone has shown great promise for the treatment or prevention of NS disorders and their symptoms or manifestations. d-Methadone so far has demonstrated an excellent safety profile in three different Phase 1 trials (described herein); furthermore, its predictable half-life and its hepatic metabolism offers clear advantages over memantine (NMDA antagonist approved for moderate and advanced dementia), especially for patients with renal impairment. Because of its favorable pharmacokinetics (uncovered by the inventors), d-methadone can be given once or twice a day without the added risks of quinidine or other drugs as is the case with dextromethorphan, another commercially available NMDA antagonist approved in combination with quindine for pseudobulbar affect (PBA) (Neudexta®). Furthermore, data from the Phase 1 studies of d-methadone (referenced above and described in greater detail in the Examples section) show that it is safe and well tolerated, without the cardiac and hematologic risks and other side effects potentially seen with Neudexta®.

Recent evidence suggests that the degree to which some NMDA antagonists produce effects within a given domain is related to the extent of the stimulation within that domain. This particular mode of action is especially important when the NMDA receptors of patients are abnormally stimulated in circumscribed NS regions, as may happen with several NS disorders. In other words, d-methadone would selectively modulate glutamergic activity where this activity is abnormally enhanced [Krystal J. H. et al. NMDA agonists and antagonists as probes of glutamatergic dysfunction and pharmacotherapies in neuropsychiatric disorders. Harv Rev Psychiatry. 1999 September-October; 7(3) 125-43] and is producing diseases and symptoms.

Altogether, the mounting evidence discovered by the present inventors suggests that d-methadone is not only a safe agent but that it may exert clinically measurable effects on cognitive function—aside from analgesic and psychiatric actions already disclosed by the inventors in distinct d-methadone patents. These new findings render d-methadone suitable for development for the treatment of all NS diseases associated with neurological impairments that can be potentially helped by NMDA antagonists and NE/SER reuptake inhibitors, and increases in BDNF and testosterone. Of note, in addition to possible benefits from the mechanisms described above, the modulating effects of d-methadone on K⁺ currents might provide additional actions for improving cognitive function [Wulff H et al., Voltage-gated potassium channels as therapeutic targets. Nat Rev Drug Discov. 2009 December; 8(12): 982-1001].

Further, the present inventors have performed a multiplicity of in vivo and clinical experiments over the last 30 years. Based on their joint knowledge and the new data presented throughout this application, including the Examples section, the present inventors uncovered the potential clinical potential usefulness of d-methadone for a multiplicity of new clinical indications. Previously, present inventor Charles Inturrisi discovered the involvement of d-methadone in the processing of nociceptive information, including the development of tolerance to the analgesic effects of opioids (see U.S. Pat. No. 6,008,258) and present inventors Paolo Manfredi and Charles Inturrisi jointly discovered the potential for d-methadone efficacy in the treatment of depression and other psychiatric symptoms (see U.S. Pat. No. 9,468,611).

The unique joint knowledge of the present inventors [Manfredi is the senior author for Kornick et al., 2003 (above), and co-author for Katchman et al. 2002 (above)] has enabled them to further pursue the question of d-methadone's cardiac safety in humans. In order to test the cardiac safety of d-methadone administration in humans the inventors provide now new prospective data on cardiac safety and on the effects of d-methadone on the QTc of healthy volunteers (see Examples section) in a multiple ascending dose study (MAD) and in a single ascending dose study (SAD). In particular, while ECGs and cardiodynamic ECG analysis performed in MAD study showed that the QTcF interval increases in a d-methadone concentration-dependent manner, these increases never reached clinical significance and no subject in the study exhibited pronounced QTcF prolongation defined as change from baseline of >60 msec or absolute QTcF >480 msec. More importantly, no subject suffered from cardiac AE during these safety studies and there were no clinically significant abnormal ECGs. These novel data from double blinded prospective studies on the cardiac safety of d-methadone are aligned with the observational findings by Bart and Marmor on racemic methadone [Bart G et al., Methadone and the QTc Interval: Paucity of Clinically Significant Factors in a Retrospective Cohort. Journal of Addiction Medicine 2017. 11(6):489-493] [Marmor M et al. Coronary artery disease and opioid use. Am J Cardiol. 2004 May 15; 93(10):1295-7] and support further development of d-methadone for the multiplicity of clinical indications outlined in the current application.

Glutamate infusions have been shown to be beneficial for patients with heart failure, and synthesis of Krebs-cycle intermediates is a major fate of the glutamate extracted by the human heart [Pietersen H G et al., Glutamate metabolism of the heart during coronary artery bypass grafting. Clin Nutr. 1998 April; 17(2):73-5]; glutamine may be cardioprotective in patients with coronary heart disease [Khogali S E et al., Is glutamine beneficial in ischemic heart disease? Nutrition. 2002 February; 18(2):123-6]. Reperfusion arrhythmias caused by glutamate may be prevented by antagonizing NMDA receptors [Sun X et al., Increasing glutamate promotes ischemia-reperfusion-induced ventricular arrhythmias in rats in vivo. Pharmacology. 2014; 93(1-2):4-9]. Glutamate release may be used as an early indicator of ongoing ischemia after cardiac arrest [Liu Z1 et al., Glutamate release predicts ongoing myocardial ischemia of rat hearts. Scand J Clin Lab Invest. 2010 Apr. 19; 70(3):217-24]. The above findings by Pietersen and Khogali on the beneficial effects of glutamine, Liu on glutamate association with ongoing ischemia, and Sun, on the effects of NMDA antagonists on reperfusion arrhythmias, taken together, suggest that glutamine in combination with an NMDA antagonist, such as d-methadone, may be synergistic for the treatment of cardiac ischemia, including unstable angina, with less risk of arrhytmias. Glutamine may be consumed by ischemic cardiac cells, while d-methadone may prevent excess calcium entry from pathologically open NMDARs.

Finally, the modulating effects of d-methadone on K⁺, Ca²⁺ and Na⁺ currents [Horrigan F T and Gilly. WF: Methadone block of K⁺current in squid giant fiber lobe neurons. J Gen Physiol. 1996 Feb. 1; 107(2): 243-260] and the modulating effects on human ether-a-go-go-related gene K⁺ currents [Katchman A N et al., Influence of opioid agonists on cardiac human ether-a-go-go-related gene K⁽⁺⁾ currents. J Pharmacol Exp Ther. 2002 November; 303(2):688-94], provide additional potential mechanisms for explaining the novel d-methadone's therapeutic actions and indications discovered by the inventors in NS diseases and their symptoms and manifestations, including cognitive improvement and therapeutic effects on schizophrenia and multiple sclerosis and muscle wasting [Wulff H et al., Voltage-gated potassium channels as therapeutic targets. Nat Rev Drug Discov. 2009 December; 8(12): 982-1001]. Furthermore, not only the actions on the K+ currents, but also the data on inhibition of Na+ and Ca+ currents, provide additional support for the multiplicity of indications presented in this application.

Another drawback for the use of opioidergic drugs, including racemic methadone, is the risk of hypogonadism [Gudin J A, Laitman A, Nalamachu S. Opioid Related Endocrinopathy. Pain Med. 2015 October; 16 Suppl 1:S9-15]. Those skilled in the art would have thought that this risk might be shared by d-methadone. As detailed in the Examples section below, in a novel clinical study presented by the inventors, d-methadone not only did not cause hypogonadism, as might be expected by those skilled in the art, but instead increased (and in some cases normalized) testosterone serum levels, signaling the unexpected lack of a known opioid side effect and thus a safer side effect profile, rendering d-methadone a better candidate for development for the multiplicity of indications presented in this application. The normalization of serum testosterone levels from d-methadone not only signals an improved side effect profile but signals additional unexpected therapeutic uses for the treatment of hypogonadism in general and also for the treatment of particular forms of hypogonadism associated neurological disorders [Alsemari A. Hypogonadism and neurological diseases. Neurol Sci. 2013 May; 34(5):629-38], such as cognitive dysfunction, epilepsy or other neurological impairments, and Prader-Willi syndrome.

Some examples of such NS disorders include Alzheimer's disease; presenile dementia; senile dementia; vascular dementia; Lewy body dementia; cognitive impairment [including mild cognitive impairment (MCI) associated with aging and with chronic disease and its treatment]; Parkinson's disease and Parkinsonian related disorders including but not limited to Parkinson dementia; disorders associated with accumulation of beta amyloid protein (including but not limited to cerebrovascular amyloid angiopathy, posterior cortical atrophy); disorders associated with accumulation or disruption of tau protein and its metabolites including but not limited to frontotemporal dementia and its variants, frontal variant, primary progressive aphasias (semantic dementia and progressive non fluent aphasia), corticobasal degeneration, supranuclear palsy; epilepsy; NS trauma; NS infections; NS inflammation [including inflammation from autoimmune disorders, including NMDAR encephalitis, and cytopathology from toxins (including microbial toxins, heavy metals, pesticides, etc.); stroke; multiple sclerosis; Huntington's disease; mitochondrial disorders; Fragile X syndrome; Angelman syndrome; hereditary ataxias; neuro-otological and eye movement disorders; neurodegenerative diseases of the retina like glaucoma, diabetic retinopathy and age-related macular degeneration; amyotrophic lateral sclerosis; tardive dyskinesias; hyperkinetic disorders; attention deficit hyperactivity disorder (“ADHD”) and attention deficit disorders; restless leg syndrome; Tourette's syndrome; schizophrenia; autism spectrum disorders; tuberous sclerosis; Rett syndrome; cerebral palsy; eating disorders [including anorexia nervosa (“AN”), bulimia nervosa (“BN”), and binge eating disorder (“BED”)]; trichotillomania; dermotillomania; nail biting; substance and alcohol abuse and dependence; migraine; fibromyalgia; and peripheral neuropathy of any etiology.

In addition to neurological diseases and their symptoms and manifestations as outlined above, the present invention relates to the treatment and/or prevention of metabolic-endocrine diseases including the metabolic syndrome and increased blood pressure, high blood sugar, excess body fat including liver fat, and abnormal cholesterol and/or triglyceride levels, type 2 diabetes and obesity, and diseases of the eye, including optic nerve diseases, retinal diseases, vitreal diseases, corneal diseases, glaucoma and dry eye syndrome.

Some examples of neurological symptoms and manifestations associated with these and other NS disorders may include: (1) a decline, impairment, or abnormality in cognitive abilities including executive function, attention, cognitive speed, memory, language functions (speech, comprehension, reading and writing), orientation in space and time, praxis, ability to perform actions, ability to recognize faces or objects, concentration, and alertness; (2) abnormal movements, including akathisia, bradykinesia, tics, myoclonus, dyskinesias (including dyskinesias relate to Huntington's disease, levodopa-induced dyskinesias and neuroleptic-induced dyskinesias), dystonias, tremors (including essential tremor), and restless leg syndrome; (3) parasomnias, insomnia, and disturbed sleep pattern; (4) psychosis; (5) delirium; (6) agitation; (7) headache; (8) motor weakness; spasticity; impaired physical endurance (9) sensory impairment (including impairment of vision and visual field defects, smell, taste and hearing) and dysesthesias; (10) dysautonomia; and/or (11) ataxia, impairment of balance or coordination, tinnitus, and neuro-otological and eye movement impairments.

In addition, the present invention relates to the treatment and/or prevention of endocrine and metabolic diseases including the metabolic syndrome (increased blood pressure, high blood sugar, excess body fat, and abnormal cholesterol or triglyceride levels), type 2 diabetes and obesity, and hyopotalamic-pitutary axis deregulation; and diseases of the eye, including retinal diseases, vitreal diseases, corneal diseases, glaucoma and dry eye syndrome.

And so, one aspect of the present invention provides a method of treating NS disorders and their neurological symptoms and manifestations, metabolic diseases, diseases of the eye and aging and its symptoms and manifestations in a subject having an NMDA receptor. The method includes administering a NMDA receptor antagonist substance (such as d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, d-isomethadone, normethadone, N-methyl-methadone, N-methyl-d-methadone, N-methyl-l-methadone, l-moramide, pharmaceutically acceptable salts thereof, or mixtures thereof) to a subject under conditions effective for the substance to bind to the NMDA receptor of the subject and thereby improve NS disorders and their neurological symptoms and manifestations, metabolic diseases, diseases of the eye and aging. The substance may be isolated from its enantiomer or synthesized de novo.

Yet another aspect of the present invention provides a method of treating NS disorders and their neurological symptoms and manifestations, endocrine-metabolic diseases, diseases of the eye and aging and its symptoms and manifestations in a subject having a NET and/or SERT. The method includes administering a substance (such as d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, d-isomethadone, normethadone, N-methyl-methadone, N-methyl-d-methadone, N-methyl-l-methadone, l-moramide, pharmaceutically acceptable salts thereof, or mixtures thereof) to a subject under conditions effective for the substance to bind to the NET and/or SERT of the subject and thereby improve NS disorders and their neurological symptoms and manifestations, metabolic diseases, diseases of the eye and aging. The substance may be isolated from its enantiomer or synthesized de novo.

Yet another aspect of the present invention provides a method of treating NS disorders and their neurological symptoms and manifestations, endocrine-metabolic diseases, diseases of the eye and aging and its symptoms and manifestations in a subject having BDNF receptors. The method includes administering a substance (such as d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, d-isomethadone, normethadone, N-methyl-methadone, N-methyl-d-methadone, N-methyl-l-methadone, l-moramide, pharmaceutically acceptable salts thereof, or mixtures thereof) to a subject under conditions effective for the substance to increase the BDNF levels of the subject and thereby improve NS disorders and their neurological symptoms and manifestations, metabolic diseases, diseases of the eye and aging. The substance may be isolated from its enantiomer or synthesized de novo.

Yet another aspect of the present invention provides a method of treating NS disorders and their neurological symptoms and manifestations, endocrine-metabolic diseases, diseases of the eye and aging and its symptoms and manifestations in a subject having testosterone receptors. The method includes administering a substance (such as d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, d-isomethadone, normethadone, N-methyl-methadone, N-methyl-d-methadone, N-methyl-l-methadone, l-moramide, pharmaceutically acceptable salts thereof, or mixtures thereof) to a subject under conditions effective for the substance to increase the testosterone levels of the subject and thereby improve NS disorders and their neurological symptoms and manifestations, metabolic diseases, diseases of the eye and aging. The substance may be isolated from its enantiomer or synthesized de novo.

Yet another aspect of the present invention provides a method of treating NS disorders and their neurological symptoms and manifestations, endocrine-metabolic diseases, diseases of the eye and aging and its symptoms and manifestations in a subject having a hypothalamic-pituitary axis. The method includes administering a substance (such as d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, d-isomethadone, normethadone, N-methyl-methadone, N-methyl-d-methadone, N-methyl-l-methadone, l-moramide, pharmaceutically acceptable salts thereof, or mixtures thereof) to a subject under conditions effective for the substance to regulate the hyopotalamic-pituitary axis of the subject and thereby improve NS disorders and their neurological symptoms and manifestations, endocrine and metabolic diseases, diseases of the eye and aging and its symptoms and manifestations. By exerting NMDAR antagonistic activity on hypothalamic neurons and thus regulating the hypothalamic-pituitary axis, d-methadone potentially influences body functions governed by all factors secreted by hypothalamic neurons (including corticotrophin-releasing hormone, dopamine, growth hormone-releasing hormone, somatostatin, gonadotrophin-releasing hormone and thyrotrophin-releasing hormone, oxytocin and vasopressin) and by consequence the factors released by the pituitary gland (including adrenocorticotropic hormone, thyroid stimulating hormone, growth hormone follicle stimulating hormone, luteinizing hormone, prolactin) and the glands, hormones and functions activated and regulated by these factors (adrenals, thyroid, gonads, sexual function, bone and muscle mass, blood pressure, glycemia, heart and kidney function, red blood cell production, immune system et cetera). The substance may be isolated from its enantiomer or synthesized de novo.

Embodiments of the various aspects of the present invention may include the use of d-methadone for the treatment of NS disorders and their symptoms such as those listed above, metabolic diseases, diseases of the eye and aging. Further, embodiments of the various aspects of the present invention may include the use of d-methadone for the treatment of neurological symptom or manifestation of NS disorders such as 1) a decline, impairment, or abnormality in cognitive abilities including executive function, attention, cognitive speed, memory, language functions (speech, comprehension, reading and writing), orientation in space and time, praxis, ability to perform actions, ability to recognize faces or objects, concentration, and alertness; (2) abnormal movements including akathisia, bradykinesia, tics, myoclonus, dyskinesias (including dyskinesias relate to Huntington's disease, levodopa induced dyskinesias and neuroleptic induced dyskinesias), dystonias, tremors (including essential tremor), and restless leg syndrome; (3) parasomnias, insomnia, disturbed sleep pattern; (4) psychosis; (5) delirium; (6) agitation; (7) headache; (8) motor weakness; spasticity; impaired physical endurance; (9) sensory impairment (including impairment of vision and visual field defects, smell, taste and hearing) and dysesthesias; (10) dysautonomia; and/or (11) ataxia, impairment of balance or coordination, tinnitus, neuro-otological and eye movement impairments.

In addition, the present invention relates to the treatment and/or prevention of metabolic diseases including the metabolic syndrome (increased blood pressure, high blood sugar, excess body fat, and abnormal cholesterol or triglyceride levels), type 2 diabetes and obesity, and diseases of the eye, including retinal diseases, vitreal diseases, corneal diseases, glaucoma and dry eye syndrome.

In another embodiment of the present invention, the method may include administering more than one substance to a subject. For example, the method may further comprise administering a drug used for treating NS disorders, endocrine-metabolic disorders and eye diseases and eye symptoms to the subject in combination with the administering of d-methadone. In various embodiments, this NS drug may be chosen from cholinesterase inhibitors; other NMDA antagonists, including memantine, dextromethorphan, and amantadine; mood stabilizers; anti-psychotics including clozapine; CNS stimulants; amphetamines; anti-depressants; anxiolytics; lithium; magnesium; zinc; analgesics, including opioids; opioid antagonists, including naltrexone, nalmefene, naloxone, 1-naltrexol, dextronaltrexone, and including Nociceptin Opioid Receptor (NOP) antagonists and selective k-opioid receptor antagonists; nicotine receptor agonists and nicotine; tauroursodeoxycholic acid (TUDCA) and other bile acids, obeticholic acid, phenylbutyric acid (PBA) and other aromatic fatty acids, calcium-channel blockers and nitric oxide synthase inhibitors, levodopa, bromocriptine and other anti-Parkinson drugs, riluzole, edavarone, antiepileptic drugs, prostaglandins, beta-blockers, alpha-adrenergic agonist, carbonic anhydrase inhibitors, parasympathomimetics, epinephrine, hyperosmotic agents, hypoglycemic agents, antihypertensive agents, anti-obesity drugs, drugs and supplements that treat nonalcoholic fatty liver disease (NAFLD) and nonalcoholic steatohepatitis (NASH).

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1 shows the structure of d-methadone [the term d-methadone indicates the dextrorotatory optical isomer salt of methadone (dextromethadone), (+)-methadone HCL].

FIG. 2 is a graph showing methadone concentrations in plasma and brain.

FIGS. 3A-3L show numeric data in table and graph form for NR1/NR2A peak current amplitude measurements based on various compounds.

FIGS. 4A-4L show numeric data in table and graph form for NR1/NR2B peak current amplitude measurements based on various compounds.

FIGS. 5A-5L show numeric data in table and graph form for NR1/NR2A steady state current amplitude measurements based on various compounds.

FIGS. 6A-6L show numeric data in table and graph form for NR1/NR2B steady state current amplitude measurements based on various compounds.

FIGS. 7A-7H are graphs, each showing PK and BDNF concentrations for one of the eight test subjects listed in Table 12 of this application. (FIG. 7A showing subject no. 1001, FIG. 7B showing subject no. 1002, FIG. 7C showing subject no. 1003, FIG. 7D showing subject no. 1004, FIG. 7E showing subject no. 1005, FIG. 7F showing subject no. 1006, FIG. 7G showing subject no. 1007, and FIG. 7H showing subject no. 1008.)

FIG. 8 is a graph showing testosterone levels for three test subjects (subject nos. 1001, 1002, and 1003).

FIG. 9 is a graph showing the effects of ketamine and d-methadone on immobility, climbing and swimming counts. Data represent mean±SEM. *p<0.05 compare to vehicle group.

FIG. 10 shows the time course of the effects of ketamine and d-methadone on locomotor activity. Data represent mean±SEM.

FIG. 11 shows the effects of ketamine and d-methadone on total distance traveled during the first 5 minutes of a forced swim test and during the whole 60 minute test period. Data represent mean±SEM.

FIG. 12 shows the time course of the effects of ketamine and d-methadone on rearing activity. Data represent mean±SEM

FIG. 13 shows the effects of ketamine and d-methadone on rearing activity during the first 5 minutes of a forced swim test and during the whole 60 minute test period. Data represent mean±SEM.

FIG. 14 shows the dosing schedule for rates subjected to the Female Urine-Sniffing Test (FUST) and/or Novelty Suppressed Feeding Test (NSFT) discussed in Example 8.

FIGS. 15A and 15B are graphs showing the results of a female urine sniffing test.

FIGS. 15C and 15D are graphs showing the results of a novelty-suppressed feeding test.

FIG. 16 is a histogram for NMDA (antagonist radioligand), showing the percentage of inhibition of control specific binding for (S)-methadone hydrochloride and (R)-methadone hydrochloride.

FIG. 17 is a histogram for δ (DOP) (h) (agonist radioligand), showing the percentage of inhibition of control specific binding for oxymorphone hydrochloride monohydrate, (S)-methadone hydrochloride, and (R)-methadone hydrochloride.

FIG. 18 is a histogram for κ (KOP) (agonist radioligand), showing the percentage of inhibition of control specific binding for oxymorphone hydrochloride monohydrate, (S)-methadone hydrochloride, and (R)-methadone hydrochloride.

FIG. 19 is a histogram for μ (MOP) (h) (agonist radioligand), showing the percentage of inhibition of control specific binding for oxymorphone hydrochloride monohydrate, (S)-methadone hydrochloride, and (R)-methadone hydrochloride.

FIG. 20 is a histogram for norepinephrine uptake, showing the percentage inhibition of control values for (S)-methadone hydrochloride, (R)-methadone hydrochloride, and tapentadol hydrochloride.

FIG. 21 is a histogram for 5-HT uptake, showing the percentage inhibition of control values for (S)-methadone hydrochloride, (R)-methadone hydrochloride, and tapentadol hydrochloride.

FIG. 22 is a histogram for δ (DOP) (h) (agonist radioligand), showing pIC₅₀ (M) for oxymorphone hydrochloride monohydrate, (S)-methadone hydrochloride, and (R)-methadone hydrochloride.

FIG. 23 is a histogram for κ (KOP) (agonist radioligand), showing pIC₅₀ (M) for oxymorphone hydrochloride monohydrate, (S)-methadone hydrochloride, and (R)-methadone hydrochloride.

FIG. 24 is a histogram for μ (MOP) (h) (agonist radioligand), showing pIC₅₀ (M) for oxymorphone hydrochloride monohydrate, (S)-methadone hydrochloride, and (R)-methadone hydrochloride.

FIG. 25 is a histogram for PCP (antagonist radioligand), showing pIC₅₀ (M) for oxymorphone hydrochloride monohydrate, (S)-methadone hydrochloride, and (R)-methadone hydrochloride.

FIG. 26 is a graph of oxymorphone hydrochloride monohydrate on δ (DOP) (h) (agonist radioligand), showing log oxymorphone hydrochloride monohydrate (M) versus the percentage inhibition of control specific binding.

FIG. 27 is a graph of (S)-methadone hydrochloride on δ (DOP) (h) (agonist radioligand), showing log (S)-methadone hydrochloride (M) versus the percentage inhibition of control specific binding.

FIG. 28 is a graph of (R)-methadone hydrochloride on δ (DOP) (h) (agonist radioligand), showing log (R)-methadone hydrochloride (M) versus the percentage inhibition of control specific binding.

FIG. 29 is a graph of oxymorphone hydrochloride monohydrate on κ (KOP) (agonist radioligand), showing log oxymorphone hydrochloride monohydrate (M) versus the percentage inhibition of control specific binding.

FIG. 30 is a graph of (S)-methadone hydrochloride on κ (KOP) (agonist radioligand), showing log (S)-methadone hydrochloride (M) versus the percentage inhibition of control specific binding.

FIG. 31 is a graph of (R)-methadone hydrochloride on κ (KOP) (agonist radioligand), showing log (R)-methadone hydrochloride (M) versus the percentage inhibition of control specific binding.

FIG. 32 is a graph of oxymorphone hydrochloride monohydrate on μ (MOP) (h) (agonist radioligand), showing log oxymorphone hydrochloride monohydrate (M) versus the percentage inhibition of control specific binding.

FIG. 33 is a graph of (S)-methadone hydrochloride on μ (MOP) (h) (agonist radioligand), showing log (S)-methadone hydrochloride (M) versus the percentage inhibition of control specific binding.

FIG. 34 is a graph of (R)-methadone hydrochloride on μ (MOP) (h) (agonist radioligand), showing log (R)-methadone hydrochloride (M) versus the percentage inhibition of control specific binding.

FIG. 35 is a graph of oxymorphone hydrochloride monohydrate on PCP (antagonist radioligand), showing log oxymorphone hydrochloride monohydrate (M) versus the percentage inhibition of control specific binding.

FIG. 36 is a graph of (S)-methadone hydrochloride on PCP (antagonist radioligand), showing log (S)-methadone hydrochloride (M) versus the percentage inhibition of control specific binding.

FIG. 37 is a graph of (R)-methadone hydrochloride on PCP (antagonist radioligand), showing log (R)-methadone hydrochloride (M) versus the percentage inhibition of control specific binding.

FIG. 38 is a histogram for norepinephrine uptake, showing pIC₅₀ (M) for tapentadol hydrochloride, (S)-methadone hydrochloride, and (R)-methadone hydrochloride.

FIG. 39 is a histogram for 5-HT uptake, showing pIC₅₀ (M) for tapentadol hydrochloride, (S)-methadone hydrochloride, and (R)-methadone hydrochloride.

FIG. 40 is a graph of tapentadol hydrochloride on norepinephrine uptake, showing log tapentadol hydrochloride (M) versus the percentage inhibition of control values.

FIG. 41 is a graph of (S)-methadone hydrochloride on norepinephrine uptake, showing log (S)-methadone hydrochloride (M) versus the percentage inhibition of control values.

FIG. 42 is a graph of (R)-methadone hydrochloride on norepinephrine uptake, showing log (R)-methadone hydrochloride (M) versus the percentage inhibition of control values.

FIG. 43 is a graph of tapentadol hydrochloride on 5-HT uptake, showing log tapentadol hydrochloride (M) versus the percentage inhibition of control values.

FIG. 44 is a graph of (S)-methadone hydrochloride on 5-HT uptake, showing log (S)-methadone hydrochloride (M) versus the percentage inhibition of control values.

FIG. 45 is a graph of (R)-methadone hydrochloride on 5-HT uptake, showing log (R)-methadone hydrochloride (M) versus the percentage inhibition of control values.

FIG. 46 includes graphs that show that d-methadone treatment decreases systolic blood pressure.

FIG. 47 includes graphs that show that d-methadone treatment decreases diastolic blood pressure.

FIG. 48 includes graphs showing the effect of d-methadone on oxygen saturation.

FIG. 49 is a chart of a linear regression analysis of BDNF and testosterone plasma levels.

FIG. 50 is a graph demonstrating a QT_{c} prolonging effect of d-methadone with a statistically significant slope for the relationship between plasma concentrations and AAQT_{c}F. In the figure, ΔΔQTcF=placebo-corrected change from baseline in QTcF interval, CI=confidence interval, log transformation model; analysis was based on the PK/QTc Population. Squares with vertical bars denote the observed mean ΔΔQTcF with 90% CI displayed at the median plasma concentration within each decile. The solid black line with gray shaded area denotes the model-predicted mean ΔΔQTcF with 90% CI. The horizontal line with notches shows the range of d-methadone concentrations divided into deciles.

FIG. 51 is a graph of d-methadone-D9 on δ (DOP) (h) (agonist radioligand), showing log d-methadone-D9 (M) versus the percentage inhibition of control specific binding.

FIG. 52 is a graph of d-methadone-D10 on δ (DOP) (h) (agonist radioligand), showing log d-methadone-D10 (M) versus the percentage inhibition of control specific binding.

FIG. 53 is a graph of d-methadone-D16 on δ (DOP) (h) (agonist radioligand), showing log d-methadone-D16 (M) versus the percentage inhibition of control specific binding.

FIG. 54 is a graph of d-methadone-D9 on κ (KOP) (agonist radioligand), showing log d-methadone-D9 (M) versus the percentage inhibition of control specific binding.

FIG. 55 is a graph of d-methadone-D10 on κ (KOP) (agonist radioligand), showing log d-methadone-D10 (M) versus the percentage inhibition of control specific binding.

FIG. 56 is a graph of d-methadone-D16 on κ (KOP) (agonist radioligand), showing log d-methadone-D16 (M) versus the percentage inhibition of control specific binding.

FIG. 57 is a graph of d-methadone-D9 on μ (MOP) (h) (agonist radioligand), showing log d-methadone-D9 (M) versus the percentage inhibition of control specific binding.

FIG. 58 is a graph of d-methadone-D10 on μ (MOP) (h) (agonist radioligand), showing log d-methadone-D10 (M) versus the percentage inhibition of control specific binding.

FIG. 59 is a graph of d-methadone-D16 on μ (MOP) (h) (agonist radioligand), showing log d-methadone-D16 (M) versus the percentage inhibition of control specific binding.

FIG. 60 is a graph of d-methadone-D9 on PCP (antagonist radioligand), showing log d-methadone-D9 (M) versus the percentage inhibition of control specific binding.

FIG. 61 is a graph of d-methadone-D10 on PCP (antagonist radioligand), showing log d-methadone-D10 (M) versus the percentage inhibition of control specific binding.

FIG. 62 is a graph of d-methadone-D16 on PCP (antagonist radioligand), showing log d-methadone-D16 (M) versus the percentage inhibition of control specific binding.

FIG. 63 is a graph of d-methadone-D9 on norepinephrine uptake, showing log d-methadone-D9 (M) versus the percentage inhibition of control values.

FIG. 64 is a graph of d-methadone-D10 on norepinephrine uptake, showing log d-methadone-D10 (M) versus the percentage inhibition of control values.

FIG. 65 is a graph of d-methadone-D16 on norepinephrine uptake, showing log d-methadone-D16 (M) versus the percentage inhibition of control values.

FIG. 66 is a graph of d-methadone-D9 on 5-HT uptake, showing log d-methadone-D9 (M) versus the percentage inhibition of control values.

FIG. 67 is a graph of d-methadone-D10 on 5-HT uptake, showing log of d-methadone-D10 (M) versus the percentage inhibition of control values.

FIG. 68 is a graph of d-methadone-D16 on 5-HT uptake, showing log d-methadone-D16 (M) versus the percentage inhibition of control values.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In view of the drawbacks listed above, safe and effective compounds, compositions, drugs, and methods, etc. that prevent and/or treat NS disorders and/or their neurological symptoms and manifestations are greatly needed. There is also a great need for safe and effective compounds, compositions, drugs, and methods, etc. that prevent and/or treat metabolic diseases and eye diseases and symptoms. And so, the present invention relates to treating and preventing various nervous system (NS) disorders [including those of the central nervous system (CNS) and peripheral nervous system (PNS)] and their neurological symptoms and manifestations, and metabolic-endocrine diseases and aging of cells and its symptoms and manifestations and eye diseases and symptoms, via compounds compositions, drugs, and methods, etc. that heretofore have not been used—and indeed would not be considered by those of ordinary skill in the art, due to the lack of the novel data here presented by the inventors and the many perceived drawbacks of certain substances (as described in the Background). Further, the present invention relates to treating and preventing cellular dysfunction and death caused by genetic, degenerative, toxic, traumatic, ischemic, infectious, neoplastic and inflammatory diseases and aging and associated diseases, symptoms and manifestations.

To that end, apart from the NMDA receptor, the NET system, the SERT system, and neurotrophic factors such as brain derived neurotrophic factor (“BDNF”) and testosterone, and Na⁺, Ca⁺, K⁺ ion channels and currents, also have important roles in numerous NS and metabolic processes and eye diseases and symptoms. And, in addition to abnormalities in the NMDA receptor complex, abnormalities associated with the NET system, SERT system, and in BDNF and testosterone, and Na⁺, Ca⁺, K⁺ ion channels and currents, have also been implicated in the pathogenesis and worsening of many disorders, including those NS disorders listed in the Background and metabolic-endocrine and eye diseases and symptoms. For example, decreased levels of BDNF are associated with neurodegenerative diseases with neuronal impairment, such as Parkinson's disease, Alzheimer's disease, multiple sclerosis, and Huntington's disease [Binder, D. K. et al., Brain-derived neurotrophic factor. Growth Factors. 2004 September; 22(3):123-31]. Markedly decreased levels of BDNF and nerve growth factor (NGF), have been observed in the nigrostriatal dopamine regions of Parkinson's disease patients and in the hippocampus of Alzheimer's patients.

Additionally, as described above, abnormalities in the NMDA receptor have been implicated in the development of ADHD. The BDNF gene and the NGFR (nerve growth factor receptor) gene belong to the neurotrophin family and are involved in the development, plasticity and survival of neurons and may play an important role regarding learning and memory but also cognitive functions. Aside from the glutamatergic system and NMDA receptor influence on the development of ADHD, the epigenetic regulation of the BDNF system as well as the NET system and SERT system have been recently found to be implicated in the development of ADHD [Banaschewski, T. et al., Molecular genetics of attention-deficit/hyperactivity disorder: an overview. Eur. Child Adolesc. Psychiatry 19, 237-257 (2010); Heinrich, H. et al., Attention, cognitive control and motivation in ADHD: Linking event-related brain potentials and DNA methylation patterns in boys at early school age. Scientific Reports 7, Article number: 3823 (2017)]. Thus, again, abnormalities in the NET system, SERT system, in BDNF, and in testosterone, seem to negatively affect many of the same NS disorders as do abnormalities in the NMDA receptor.

The NET is an extracellular monoamine transporter. Compounds that block this transporter result in sustained increases in the concentration of the neurotransmitter norepinephrine. This will generally result in a stimulation of the sympathetic nervous system and effects on mood and memory (see below).

The SERT is an extracellular monoamine transporter. Compounds that block this transporter result in sustained increases in the concentration of the neurotransmitter serotonin. The SERT is the target of many antidepressant medications of the SSRI and tricyclic antidepressant classes (see below).

NE and serotonin, aside from their known effects on mood disorders, are also involved in memory and learning (Zhang G and Stackman R S Jr. The role of serotonin 5-HT2A receptors in memory and cognition. Front. Pharmacol., October 2015 Volume 6, article 225). The in vitro receptor studies presented by the inventors (between the Examples) show unique d-methadone affinity values for inhibition of NET and SERT; the enhanced availability of these neurotransmitters in select brain areas may contribute to explain some of the cognitive improvements from d-methadone uncovered by the inventors.

BDNF is a protein that, in humans, is encoded by the BDNF gene. BDNF is a member of the neurotrophin family of growth factors. Neurotrophic factors are found in the brain and the periphery. BDNF acts on certain neurons of the central nervous system and the peripheral nervous system, helping to support the survival of existing neurons, and encourages the growth and differentiation of new neurons and synapses between neurons. In the brain, it is particularly active in the hippocampus, cortex, and basal forebrain—areas vital to learning, memory, and higher thinking. BDNF binds to receptors (TrkA, TrkB, p75NTR) that are capable of responding to this growth factor.

Testosterone is a well known hormone that plays important roles in the body. It regulate sex drive (libido), bone mass, fat distribution, muscle mass and strength, endurance, and the production of red blood cells and sperm. A small amount of circulating testosterone is converted to estradiol, a form of estrogen. Cognitive dysfunction including age related cognitive dysfunction, metabolic syndrome (increased blood pressure, high blood sugar, excess body fat, and abnormal cholesterol or triglyceride levels), type 2 diabetes, epilepsy, aging of tissues including neurons, nerves, muscles (including sarcopenia and impaired physical endurance), bone (including osteoporosis), skin, including wrinkling, gonads (including impaired sexual function and decreased sexual drive), cornea (including dry eye syndrome), retina (including degenerative diseases of the retina), age related hearing and balance impairment. All of the above conditions, including normal aging and its symptoms and manifestations, and accelerated aging caused by diseases and their treatment (e.g., therapies against cancer such as impaired physical endurance associated with chemotherapy) may be improved by up-regulating endogenous testosterone levels. Another indication is low testosterone of any cause. Furthermore iatrogenic low testosterone from opioid therapy and other drugs or medical treatments may be treated or prevented by d-methadone.

Thus, a drug that modulates the NMDA receptor, NET system, and/or SERT system, up-regulates BDNF and testosterone levels, may reduce excitotoxicity, potentially protect mitochondria from Ca²⁺ overload, and potentially improve cognition and other neurological diseases and symptoms and metabolic and eye diseases and symptoms via different mechanisms. Additionally, if this drug shows signs of effectiveness in humans and is found to be safe without psychotomimetic or opioid side effects, it may hold great potential for treating NS disorders and their neurological symptoms and manifestations and metabolic-endocrine and eye diseases and symptoms. Further, a drug that increases BDNF levels may also be useful for peripheral nerve disorders, such as peripheral neuropathies of different etiology, including diabetic peripheral neuropathy.

Additionally, it is known that neuroplasticity is connected with the developmental stages of life; however, there is now growing evidence confirming that structural and functional reorganization occurs throughout our lifetime, and may influence the onset, clinical course, and recovery of most diseases of the CNS and PNS (Ksiazek-Winiarek, D. J. et al., Neural Plasticity in Multiple Sclerosis: The Functional and Molecular Background. Neural Plast. 2015:307175). As described above, BDNF acts on certain neurons of the central nervous system and the peripheral nervous system, helping to support the survival of existing neurons, and encourages the growth and differentiation of new neurons and synapses. And so, BDNF, by influencing neuronal plasticity, is a potential therapeutic target to prevent, alter the course, and/or treat symptoms and manifestations of many NS disorders.

Since BDNF appears to be involved in activity-dependent synaptic plasticity, there is great interest in its role in learning and memory [Binder, D. K. et al., Brain-derived neurotrophic factor. Growth Factors. 2004 September; 22(3):123-31]. The hippocampus, which is required for many forms of long-term memory in humans and animals, appears to be an important site of BDNF action. Rapid and selective induction of BDNF expression in the hippocampus during contextual learning has been demonstrated (Hall, J. et al., Rapid and selective induction of BDNF expression in the hippocampus during contextual learning. Nat Neurosci. 2000; 3:533-535). Another study demonstrated upregulation of BDNF in monkey parietal cortex associated with tool-use learning (Ishibashi, H. et al., Tool-use learning induces BDNF expression in a selective portion of monkey anterior parietal cortex. Brain Res Mol Brain Res. 2002; 102:110-112). In humans, a valine to methionine polymorphism at the 5′ pro-region of the human BDNF protein was found to be associated with poorer episodic memory; in vitro, neurons transfected with met-BDNF-GFP exhibited reduced depolarization-induced BDNF secretion (Egan, M. F. et al., The BDNF val66met polymorphism affects activity-dependent secretion of BDNF and human memory and hippocampal function. Cell. 2003; 112:257-269).

It is known that BDNF exerts trophic and protective effects on dopaminergic neurons as well as other neuronal systems. Thus, impairment of cognitive function may result from, or be exacerbated by, reduction in BDNF. However, as described above, Falko et al. found that memantine (an NMDA receptor antagonist used to treat Alzheimer's disease) specifically upregulated mRNA and protein expression of BDNF in monkeys, suggesting that the protective effect of memantine on dopamine function may be mechanistically remote from NMDA receptor antagonism and may be related to BDNF. Further, Marvanova M. et al., The Neuroprotective Agent Memantine Induces Brain-Derived Neurotrophic Factor and trkB Receptor Expression in Rat Brain-Molecular and Cellular Neuroscience 2001; 18, 247-258, reported that memantine increased production of BDNF in rat brain. And so, BDNF has been suggested as a possible therapeutic candidate for treatment of many NS diseases (Kandel, E. R. et al., Principles of Neural Science, Fifth Edition, 2013).

Against that backdrop, it has been reported that l-methadone decreases blood levels of BDNF in Methadone Maintenance (MMT) patients (see Schuster R. et al., Elevated methylation and decreased serum concentrations of BDNF in patients in levomethadone compared to diamorphine maintenance treatment Eur Arch Psychiatry Clin Neurosci 2017; 267:33-40). However, as described above, Tsai et al., 2016, found that racemic methadone increases BDNF levels in a similar group of heroin-dependent MMT patients. The present inventors thus reached a novel conclusion that the findings of these studies, when taken together, could indirectly support the idea that d-methadone, rather than l-methadone, is primarily responsible for increasing BDNF levels, and that d-methadone is likely more active in increasing BDNF levels than racemic methadone (which contains 50% l-methadone, which not only as described by Schuster et al., decreases BDNF levels and could counteract the effects of d-methadone but also has strong opioid effects). This conclusion has not been previously reached by those skilled in the art, and—to date—there have been thought to be myriad drawbacks to the use of racemic methadone, d-methadone, and l-methadone.

Further, the effect discussed in Tsai et al may be mediated via modulation at the NMDA and/or NET systems or via upregulation of mRNA, as suggested by Falko et al., and thus may also be inherent to d-methadone, as suggested by the effects of d-methadone on BDNF levels discovered by the inventors and detailed in the Examples, and not only to racemic methadone. The present inventors thus reached another novel conclusion (and one heretofore not contemplated by those skilled in the art): That this mRNA-mediated increase in BDNF offers another likely explanation, in addition to the actions at the NMDA receptor, NET system, and SERT system, for the cognitive improvements from d-methadone discovered by the inventors. Furthermore, this increase in BDNF in MMT patients reported by Tsai as resulting from dosing with racemic methadone was seen at doses comparable to the safe and effective doses of d-methadone tested by the inventors.

As is known, l-methadone is principally an opioid agonist, while d-methadone is a very weak opioid agonist and this activity at opioid receptors was found by the inventors to be absent at doses expected by the inventors to exert clinical effects modulating actions at the NMDA receptor, NET system, and SERT system, and (3) potentially up-regulate BDNF. And so, the present inventors have determined for the first time that a drug like d-methadone—which (1) is safe and well-tolerated, (2) is devoid of opioid activity and psychotomimetic effects at doses expected to maintain modulating actions on the NMDA receptor, NET system, and SERT system, and (3) potentially up-regulate BDNF—can improve cognitive performance without negative opioid-like effects and without psychotomimetic effects. Thus, when methadone is substituted for other opioids such as in the studies conducted and re-analyzed by the inventors, including the Santiago-Palma et al. 2001study, the opioid effects of methadone and the prior opioid (the opioid substituted with methadone) neutralize themselves, and the effects of other actions of methadone (modulation of the NMDA receptor, NET system, and SERT system and increase in BDNF) become apparent and clinically measurable. These other actions (modulation of the NMDA receptor, NET system, and SERT system and increase in BDNF), as shown by the inventors, are present in the d-methadone isomer without opioid effects, while in racemic methadone and in l-methadone they remain combined with strong opioid effects (and therefore are of limited clinical use).

These NMDA, NET, SERT, BDNF, testosterone effects, and modulation of K⁺, Ca²⁺ and Na⁺ currents, might also explain why elderly frail patients with baseline cognitive impairment have better cognitive function while treated with methadone rather than other opioids, as indicated by the present inventor Manfredi and other authors [see Vu et al., Use of Methadone as an Adjuvant Medication to Low-Dose Opioids for Neuropathic Pain in the Frail Elderly: A Case Series. J Palliat Med. 2016 December; 19(12):1351-1355]. This improvement in cognitive function has never before been attributed to a direct effect of methadone or its isomers and has always been ascribed to the disappearance of opioid side effects from the prior opioid (the opioid discontinued when methadone is introduced). Furthermore, while the use of methadone in patients with addiction has been associated with cognitive improvement, these effects have not been attributed to direct actions of d-methadone mediated by modulation at the NMDA receptor, NET system, SERT system or an increase in BDNF, or testosterone or a modulating effect on K⁺, Ca²⁺ and Na⁺ currents, as now taught by the present inventors.

The majority of research suggests that Methadone Maintenance Therapy (MMT) and opioids in general are associated with impaired cognitive function and that deficits extended across a range of domains. However, many studies compared cognitive impairment in patients on methadone to healthy controls. These studies overlook the fact that these are not comparable groups and patients on with opioid addiction often have pre-existing cognitive impairments (high prevalence of ADHD), cognitive impairment caused by illicit substance use, and co-morbidities such as HIV and HCV that are known to impair cognition.

In fact, while many studies ascribe to methadone a negative effect on cognitive function [see Wang et al., Methadone maintenance treatment and cognitive function: a systematic review. Curr Drug Abuse Rev. 2013 September; 6(3):220-30], opposite results are found when the cognitive performance of patients on methadone is compared to the cognitive performance of patients using illicit opioids. Wang et al., Soyka et al., and Gruber et al. found that cognitive function or sensory information processing in patients undertaking MMT, is improved compared to those of patients using illicit opiates [see Wang et al., Neuropsychological performance of methadone-maintained opiate users. J Psychopharmacol. 2014 August; 28 (8):789-99; Soyka et al., Better cognitive function in patients treated with methadone than in patients treated with heroin: A comparison of cognitive function in patients under maintenance treatment with heroin, methadone, or buprenorphine and healthy controls: an open pilot study. Am J Drug Alcohol Abuse. 2011 November; 37(6):497-508; Gruber et al., Methadone maintenance improves cognitive performance after two months of treatment. Exp Clin Psychopharmacol. 2006 May; 14 (2):157-64 and Wang et al., Auditory event-related potentials in methadone substituted opiate users. J Psychopharmacol. 2015 September; 29 (9):983-95]. And Grevert et al. found no effect of levo-alpha-acetylmethadol, LAAM, on memory (a strong opioid like LAAM would be expected to impair memory processing) [see Grevert et al., Failure of methadone and levomethadyl acetate (levo-alpha-acetylmethadol, LAAM) maintenance to affect memory. Arch Gen Psychiatry. 1977 July; 34(7):849-53]. This unexpected finding by Grevert et al. 1977 and the improvements noted by Wang et al., 2014, Soyka et al., 2011, Gruber et al. 2006, and Wang et al., 2015 now signal to the inventors that d-methadone, which is devoid of opioid activity, when tested in patients, might have a positive effect on cognition and sensory information processing.

Further, as is known, patients with ADHD are more likely to develop dependence on illicit drugs (Biederman et al., Young adult outcome of attention deficit hyperactivity disorder: a controlled 10-year follow-up study. Psychological Medicine. 2006, 36(167-179), and methadone maintenance patients have a higher prevalence of ADHD compared to the general population. When compared to users of illicit drugs, patients on MMT have been found to have improved cognitive function [Wang et al., Neuropsychological performance of methadone-maintained opiate users. J Psychopharmacol. 2014 August; 28 (8):789-99; Soyka et al., Better cognitive function in patients treated with methadone than in patients treated with heroin: A comparison of cognitive function in patients under maintenance treatment with heroin, methadone, or buprenorphine and healthy controls: an open pilot study. Am J Drug Alcohol Abuse. 2011 November; 37(6):497-508; and Gruber et al., Methadone maintenance improves cognitive performance after two months of treatment. Exp Clin Psychopharmacol. 2006 May; 14 (2):157-64] and improved sensory processing [Wang et al. Auditory event-related potentials in methadone substituted opiate users. J Psychopharmacol. 2015 September; 29(9):983-95]. Memantine has been found to improve cognitive function of patients with ADHD [Mohammadi et al., Memantine versus Methylphenidate in Children and Adolescents with Attention Deficit Hyperactivity Disorder: A Double-Blind, Randomized Clinical Trial. Iran J Psychiatry. 2015 April; 10(2):106-14] and the NMDA receptor system bears a key role in learning, cognitive functions and memory (Kandel, E. R. et al., Principles of Neural Science, Fifth Edition, 2013). Opioids are well known to cause sedation and therefore it is likely that any cognitive improvement is independent of the opioid effect of methadone. On the other hand, a drug like d-methadone, devoid of opioid activity and effective on the NMDA, NET, SERT, and BDNF systems, based on the inventors' work described herein, might improve deficits in information processing and be useful in conditions such as ADHD and mild cognitive impairment of unspecified etiology, often seen in patients in MMT and in other disorders, such as HIV disease and epilepsy.

In light of the joint knowledge of the inventors, these unexpected findings on cognition and memory may be a direct effect of methadone on modulation of NMDA, NET, SERT systems, and/or BDNF and therefore inherent to methadone—while not opioid related—and not due to a reduction of illicit opioid use. Thus, a drug like d-methadone might improve deficits in information processing and might be useful in conditions such as ADHD—which is frequent in illicit substance users—and in other conditions associated with cognitive impairment of unspecified etiology. Before this discover of the present inventors, this sort of treatment, method, etc. using a drug like d-methadone had not been considered.

To that end, the inventors now provide herein new human data showing that d-methadone up-regulates BDNF and testosterone serum levels and potentially regulates blood pressure and glycemia. The inventors have also discovered new signals for effectiveness for improving cognitive function in humans in human studies, new evidence for linear pharmacokinetics, and new pharmacodynamic data that demonstrate lack of opioid cognitive side effects and psychotomimetic side effects at doses potentially therapeutic and new overall safety data (therefore confirming d-methadone's potential for improving cognitive impairment, as discovered by the inventors). The inventors also provide herein new data on characterization of NMDA receptor interactions for d-methadone in the micromolar range and provide new experimental data showing higher than expected CNS levels of d-methadone after systemic administration. The inventors also provide new in vitro data on receptor studies showing unique d-methadone affinity values for inhibition of NET and SERT.

Memantine is FDA approved for the treatment of Alzheimer's disease in the moderate to severe stages. d-Methadone however, as noted by the inventors, may have better NMDA receptor affinity over memantine to be effective for the regulation of the NMDA system disrupted in Alzheimer's disease. In addition to NMDA antagonistic activity, d-methadone inhibits NE and SER reuptake [Codd et al., Serotonin and Norepinephrine activity of centrally acting analgesics: Structural determinants and role in antinociception. IPET 1995; 274:(3)1263-1269], as confirmed by the inventors, and potentially increases BDNF levels, as shown herein for the first time by the inventors. These actions of d-methadone may also contribute to its therapeutic actions against many NS disorders, in addition to Alzheimer's disease (Kandel, E. R. et al., Principles of Neural Science, Fifth Edition, 2013). And so, d-methadone's action at the NET [Codd et al., Serotonin and Norepinephrine activity of centrally acting analgesics: Structural determinants and role in antinociception. IPET 1995; 274:(3)1263-1269] and on BDNF may offer further advantages against the symptoms of Alzheimer's disease: mounting evidence indicates that the impairment of noradrenergic innervation greatly exacerbates AD pathogenesis and progression (Gannon, M. et al., Noradrenergic dysfunction in Alzheimer's disease. Front Neurosci. 2015; 9:220).

In testing by the inventors (described herein), d-methadone has shown great promise for the treatment or prevention of NS disorders, or their symptoms or manifestations. d-Methadone so far has demonstrated an excellent safety profile in three different Phase 1 trials (described in greater detail in the Examples. Furthermore, its predictable half-life and its hepatic metabolism offers clear advantages over memantine, particularly for patients with renal impairment. Because of its favorable pharmacokinetics (uncovered by the inventors) d-methadone can be given once or twice a day without the added risks of quinidine or other drugs. Furthermore, data from the Phase 1 studies of d-methadone (referenced above) show that it is safe and well tolerated, without the cardiac and hematologic risks and other potential side effects from combination drugs such as Neudexta®.

Recent evidence suggests that the degree to which NMDA antagonists produce effects within a given domain is related to the extent of the stimulation within that domain. This particular mode of action may be important when the NMDA receptors of patients are abnormally stimulated in circumscribed regions of the human body, as may happen with several disorders, including NS disorders, endocrine-metabolic disorders and eye disorders and disorders of hypothalamic neurons and thus the hypothalamus-pituitary axis. In other words, d-methadone could selectively modulate glutamergic activity only where this activity is abnormally enhanced [Krystal J. H. et al. NMDA agonists and antagonists as probes of glutamatergic dysfunction and pharmacotherapies in neuropsychiatric disorders (Harv Rev Psychiatry. 1999 September-October; 7(3) 125-43].

Altogether, the mounting evidence discovered by the present inventors suggests that d-methadone is not only a safe agent but that it may exert clinically measurable effects on cognitive function and endocrine-metabolic and eye functions. These new findings render d-methadone suitable for development for the treatment of diseases associated with neurological, endocrine-metabolic, eye impairments that can be potentially helped by NMDA antagonists and NE reuptake inhibitors, increases in BDNF and testosterone, such as Alzheimer's disease; presenile dementia; senile dementia; vascular dementia; Lewy body dementia; cognitive impairment [including mild cognitive impairment (MCI) associated with aging and with chronic disease and its treatment]; Parkinson's disease and Parkinsonian related disorders including but not limited to Parkinson dementia; disorders associated with accumulation of beta amyloid protein (including but not limited to cerebrovascular amyloid angiopathy, posterior cortical atrophy); disorders associated with accumulation or disruption of tau protein and its metabolites including but not limited to frontotemporal dementia and its variants, frontal variant, primary progressive aphasias (semantic dementia and progressive non fluent aphasia), corticobasal degeneration, supranuclear palsy; epilepsy; NS trauma; NS infections; NS inflammation [including inflammation from autoimmune disorders, including NMDAR encephalitis, and cytopathology from toxins (including microbial toxins, heavy metals, pesticides, etc.); stroke; multiple sclerosis; Huntington's disease; mitochondrial disorders; Fragile X syndrome; Angelman syndrome; hereditary ataxias; neuro-otological and eye movement disorders; neurodegenerative diseases of the retina like glaucoma, diabetic retinopathy and age-related macular degeneration; amyotrophic lateral sclerosis; tardive dyskinesias; hyperkinetic disorders; attention deficit hyperactivity disorder (“ADHD”) and attention deficit disorders; restless leg syndrome; Tourette's syndrome; schizophrenia; autism spectrum disorders; tuberous sclerosis; Rett syndrome; cerebral palsy; eating disorders [including anorexia nervosa (“AN”) and bulimia nervosa (“BN”) and binge eating disorder (“BED”), trichotillomania, dermotillomania, nail biting, and substance and alcohol abuse and dependence;]; migraine; fibromyalgia; and peripheral neuropathy of any etiology. In addition the present invention relates to the treatment and/or prevention of endocrine metabolic diseases including the metabolic syndrome, type 2 diabetes and increased body and liver fat, hypertension, obesity, and diseases of the eye, including retinal diseases, vitreal diseases, corneal diseases, glaucoma and dry eye syndrome. And, the present inventors have discovered that even patients with very mild cognitive impairment of unspecified cause may respond to a drug like d-methadone, which combines NMDA antagonisms with inhibition of NE and serotonin re-uptake, while increasing BDNF and testosterone, alone or in combination with standard therapy.

And so, one aspect of the present invention provides a method of treating NS disorders and their neurological symptoms and manifestations, endocrine-metabolic diseases, diseases of the eye and aging and its symptoms and manifestations in a subject having an NMDA receptor. The method includes administering a NMDA receptor antagonist substance (such as d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, d-isomethadone, normethadone, N-methyl-methadone, N-methyl-d-methadone, N-methyl-l-methadone, l-moramide, pharmaceutically acceptable salts thereof, or mixtures thereof) to a subject under conditions effective for the substance to bind to the NMDA receptor of the subject and thereby improve NS disorders and their neurological symptoms and manifestations, endocrine-metabolic diseases, diseases of the eye and aging. The substance may be isolated from its enantiomer or synthesized de novo.

Yet another aspect of the present invention provides a method of treating NS disorders and their neurological symptoms and manifestations, endocrine-metabolic diseases, diseases of the eye and aging and its symptoms and manifestations in a subject having a NET and/or SERT. The method includes administering a substance (such as d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, d-isomethadone, normethadone, N-methyl-methadone, N-methyl-d-methadone, N-methyl-l-methadone, l-moramide, pharmaceutically acceptable salts thereof, or mixtures thereof) to a subject under conditions effective for the substance to bind to the NET (and/or SERT) of the subject and thereby improve NS disorders and their neurological symptoms and manifestations, metabolic diseases, diseases of the eye and aging. The substance may be isolated from its enantiomer or synthesized de novo.

Yet another aspect of the present invention provides a method of treating NS disorders and their neurological symptoms and manifestations, endocrine-metabolic diseases, diseases of the eye and aging and its symptoms and manifestations in a subject having BDNF receptors. The method includes administering a substance (such as d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, d-isomethadone, normethadone, N-methyl-methadone, N-methyl-d-methadone, N-methyl-l-methadone, l-moramide, pharmaceutically acceptable salts thereof, or mixtures thereof) to a subject under conditions effective for the substance to increase the BDNF levels of the subject and thereby improve NS disorders and their neurological symptoms and manifestations, metabolic diseases, diseases of the eye and aging. The substance may be isolated from its enantiomer or synthesized de novo.

Yet another aspect of the present invention provides a method of treating NS disorders and their neurological symptoms and manifestations, endocrine-metabolic diseases, diseases of the eye and aging and its symptoms and manifestations in a subject having testosterone receptors. The method includes administering a substance (such as d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, d-isomethadone, normethadone, N-methyl-methadone, N-methyl-d-methadone, N-methyl-l-methadone, l-moramide, pharmaceutically acceptable salts thereof, or mixtures thereof) to a subject under conditions effective for the substance to increase the testosterone levels of the subject and thereby improve NS disorders and their neurological symptoms and manifestations, metabolic diseases, diseases of the eye and aging. The substance may be isolated from its enantiomer or synthesized de novo.

Yet another aspect of the present invention provides a method of treating NS disorders and their neurological symptoms and manifestations, endocrine-metabolic diseases, diseases of the eye and aging and its symptoms and manifestations in a subject having a hypothalamic-pituitary axis. The method includes administering a substance (such as d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, d-isomethadone, normethadone, N-methyl-methadone, N-methyl-d-methadone, N-methyl-l-methadone, l-moramide, pharmaceutically acceptable salts thereof, or mixtures thereof) to a subject under conditions effective for the substance to increase the tregulate the hyopotalamic-pituitary axis of the subject and thereby improve NS disorders and their neurological symptoms and manifestations, endocrine and metabolic diseases, diseases of the eye and aging. The substance may be isolated from its enantiomer or synthesized de novo.

Embodiments of the various aspects of the present invention may include the use of d-methadone for the treatment of NS disorders such as those listed above. Further, embodiments of the various aspects of the present invention, in addition the treatment and/or prevention of endocrine-metabolic diseases including the metabolic syndrome, type 2 diabetes and increased body and liver fat, hypertension, obesity, and diseases of the eye, including retinal diseases, vitreal diseases, corneal diseases, glaucoma and dry eye syndrome, may include the use of d-methadone for the treatment of neurological symptom or manifestation of NS disorders such as (1) a decline, impairment, or abnormality in cognitive abilities including executive function, attention, cognitive speed, memory, language functions (speech, comprehension, reading and writing), orientation in space and time, praxis, ability to perform actions, ability to recognize faces or objects, concentration, and alertness; (2) abnormal movements, including akathisia, bradykinesia, tics, myoclonus, dyskinesias (including dyskinesias relate to Huntington's disease, levodopa-induced dyskinesias and neuroleptic-induced dyskinesias), dystonias, tremors (including essential tremor), and restless leg syndrome; (3) parasomnias, insomnia, and disturbed sleep pattern; (4) psychosis; (5) delirium; (6) agitation; (7) headache; (8) motor weakness; spasticity; impaired physical endurance (9) sensory impairment (including impairment of vision and visual fields, smell, taste and hearing) and dysesthesias; (10) dysautonomia; and/or (11) ataxia, impairment of balance or coordination, tinnitus, and neuro-otological and eye movement impairments.

In various embodiments, d-methadone may be used alone for the treatment of the subject's NS disorders and their symptoms and manifestations, metabolic diseases and diseases of the eye, or in combination with other drugs potentially useful to treat the disorders listed above and or other NMDA antagonists. And so, in another embodiment of the present invention, the method may include administering more than one substance to a subject. For example, the method may further comprise administering a drug used for treating NS disorders to the subject in combination with the administering of d-methadone. In various embodiments, this NS drug may be chosen from cholinesterase inhibitors; other NMDA antagonists, including memantine, dextromethorphan, and amantadine; mood stabilizers; anti-psychotics including clozapine; CNS stimulants; amphetamines; anti-depressants; anxiolytics; lithium; magnesium; zinc; analgesics, including opioids; opioid antagonists, including naltrexone, nalmefene, naloxone, 1-naltrexol, dextronaltrexone, and including NOP antagonists and selective k opioid receptor antagonists; nicotine receptor antagonists and nicotine; tauroursodeoxycholic acid (TUDCA) and other bile acids, obethicolic acid, idebenone, phenylbutyric acid (PBA) and other aromatic fatty acids, calcium-channel blockers and nitric oxide synthase inhibitors, levodopa, bromocriptine and other anti-Parkinson drugs, riluzole, edavarone, antiepileptic drugs, prostaglandins, beta-blockers, alpha-adrenergic agonist, carbonic anhydrase inhibitors, parasympathomimetics, epinephrine, hyperosmotic agents.

Further, the actions of d-methadone for all of the above indications may be enhanced by a combination with other drugs. NMDA antagonists have been used for the treatment of Alzheimer's disease (memantine) and Parkinson disease (amantadine). Magnesium is a NMDAR blocker and magnesium supplementation has been shown to the potential of improving hypertension, insulin sensitivity, hyperglycemia, diabetes mellitus, left ventricular hypertrophy, and dyslipidemia; in addition it can treat certain types of seizures, e.g., those occurring as part of eclampsia (Euser A G. Cipolla M J. Magnesium sulfate for the treatment of eclampsia: a brief review. Stroke. 2009 April; 40(4):1169-75) and can be used for arrhythmias such as torsades de pointes. [Houston M. The role of magnesium in hypertension and cardiovascular disease. J Clin Hypertens (Greenwich). 2011 November; 13(11):843-7]; [Rosanoff A. Magnesium and hypertension. Clin Calcium. 2005 February; 15(2):255-60]. Magnesium has also been implicated in the pathogenesis or treatment of headaches, CNS trauma, Parkinson disease and Alzheimer disease (Vink R1. Magnesium in the CNS: recent advances and developments. Magnes Res. 2016 Mar. 1; 29(3):95-101).

Drugs that may enhance the actions of d-methadone and or reduce its side effects include cholinesterase inhibitors; other NMDA antagonists, including memantine, dextromethorphan, and amantadine; mood stabilizers; anti-psychotics including clozapine; CNS stimulants; amphetamines; anti-depressants; anxiolytics; lithium; magnesium; zinc; analgesics, including opioids; opioid antagonists, including naltrexone, nalmefene, naloxone, 1-naltrexol, dextronaltrexone, and including NOP antagonists and selective k opioid receptor antagonists; nicotine receptor antagonists and nicotine; tauroursodeoxycholic acid (TUDCA) and other bile acids, obethicolic acid, idebenone, phenylbutyric acid (PBA) and other aromatic fatty acids, calcium-channel blockers and nitric oxide synthase inhibitors, levodopa, bromocriptine and other anti-Parkinson drugs, riluzole, edavarone, antiepileptic drugs, prostaglandins, beta-blockers, alpha-adrenergic agonist, carbonic anhydrase inhibitors, parasympathomimetics, epinephrine, hyperosmotic agents.

Opioid antagonists such as naltrexone, may have activity against psychiatric syndromes, such as depersonalization disorder, depression, and anxiety, and may enhance the effects of other anti-depressants and improve depression (Mischoulon D et al., Randomized, proof-of-concept trial of low dose naltrexone for patients with breakthrough symptoms of major depressive disorder on antidepressants. J Affect Disord. 2017 Jan. 15; 208:6-14). and are used for the treatment of addiction, including behavioral addiction, obesity, and are used off label (use not FDA or EMEA approved) for fibromyalgia, impaired physical endurance, and multiple sclerosis. In particular, a combination of d-methadone with an opioid antagonist such as naltrexone may be synergistic and reduce side effects and risks when administered for the treatment of chronic pain, including neuropathic pain, fibromyalgia, migraine and other headaches; may be synergistic and have reduced side effects when administered for the treatment of psychiatric symptoms and diseases, including depression, anxiety, obsessive compulsive disorder, self-injurious behaviors like trichotillomania, dermotillomania, nail biting, pseudobulbar affect, depersonalization disorder, addiction to various substances including alcohol, opioids, nicotine, benzodiazepines, stimulants and other recreational drugs, behavioral addictions and may be synergistic and have reduced side effects when administered for all of and all of the indications (diseases and symptoms) listed with the present application and obesity and cough.

Selective k opioid receptor antagonists have been used and are under investigation for the treatment of psychiatric disease (Carroll F I and Carlezon W A. Development of Kappa Opioid Receptor Antagonists. Journal of medicinal chemistry. 2013; 56(6):2178-2195.); a combination of a selective k-antagonist with d-methadone might be synergistic for the treatment of depression and other psychiatric conditions, including addiction to drugs and pathological behaviors, and the conditions listed below. Disease and conditions possibly improved by a combination of d-methadone with an opioid antagonist include: Alzheimer's disease; presenile dementia; senile dementia; vascular dementia; Lewy body dementia; cognitive impairment [including mild cognitive impairment (MCI) associated with aging and with chronic disease and its treatment]; Parkinson's disease and Parkinsonian related disorders including but not limited to Parkinson dementia; disorders associated with accumulation of beta amyloid protein (including but not limited to cerebrovascular amyloid angiopathy, posterior cortical atrophy); disorders associated with accumulation or disruption of tau protein and its metabolites including but not limited to frontotemporal dementia and its variants, frontal variant, primary progressive aphasias (semantic dementia and progressive non fluent aphasia), corticobasal degeneration, supranuclear palsy; epilepsy; NS trauma; NS infections; NS inflammation [including inflammation from autoimmune disorders, including NMDAR encephalitis, and cytopathology from toxins (including microbial toxins, heavy metals, pesticides, etc.); stroke; multiple sclerosis; Huntington's disease; mitochondrial disorders; Fragile X syndrome; Angelman syndrome; hereditary ataxias; neuro-otological and eye movement disorders; neurodegenerative diseases of the retina like glaucoma, diabetic retinopathy and age-related macular degeneration; amyotrophic lateral sclerosis; tardive dyskinesias; hyperkinetic disorders; attention deficit hyperactivity disorder (“ADHD”) and attention deficit disorders; restless leg syndrome; Tourette's syndrome; schizophrenia; autism spectrum disorders; tuberous sclerosis; Rett syndrome; cerebral palsy; eating disorders [including anorexia nervosa (“AN”) and bulimia nervosa (“BN”) and binge eating disorder (“BED”), trichotillomania, dermotillomania, nail biting, and substance and alcohol abuse and dependence;]; migraine; fibromyalgia; and peripheral neuropathy of any etiology, metabolic diseases and diseases of the eye.

Some examples of neurological symptoms and manifestations associated with these and other NS disorders and possibly improved by a combination of d-methadone with an opioid antagonist may include: (1) a decline, impairment, or abnormality in cognitive abilities including executive function, attention, cognitive speed, memory, language functions (speech, comprehension, reading and writing), orientation in space and time, praxis, ability to perform actions, ability to recognize faces or objects, concentration, and alertness; (2) abnormal movements, including akathisia, bradykinesia, tics, myoclonus, dyskinesias (including dyskinesias relate to Huntington's disease, levodopa-induced dyskinesias and neuroleptic-induced dyskinesias), dystonias, tremors (including essential tremor), and restless leg syndrome; (3) parasomnias, insomnia, and disturbed sleep pattern; (4) psychosis; (5) delirium; (6) agitation; (7) headache; (8) motor weakness; spasticity; impaired physical endurance (9) sensory impairment (including impairment of vision and visual fields, smell, taste and hearing) and dysesthesias; (10) dysautonomia; and/or (11) ataxia, impairment of balance or coordination, tinnitus, and neuro-otological and eye movement impairments. Some examples of metabolic diseases and eye diseases include the metabolic syndrome, type 2 diabetes and increased body and liver fat, hypertension, obesity, and retinal diseases, vitreal diseases, corneal diseases, glaucoma and dry eye syndrome and midriasis.

Cough might also be alleviated by a combination of d-methadone (or other opioids—e.g., codeine —, opioid isomers and opioid congeners and metabolites—e.g., dextromethorphan, racemorphan, dextrorphan, 3-methoxymorphinan to 3-hydroxymorphinan) with an opioid antagonist. The combination between an opioid and an opioid antagonist will retain the non-opioid actions, such as actions on the NMDA, NA/SERT, BDNF, mTOR systems, testosterone levels, while reducing or abolishing the unwanted opioid side effects and risks (these combinations will also become abuse deterrent formulation of opioid drugs and congeners of opioid drugs, defined as drugs that bind to opioid receptors and their isomers with little or no opioid activity). This opioid agonist/antagonist combination would have the advantage of nonopioid effects listed above in the absence of opioid effects with an added opioid deterrent feature; in particular, the combination drug might be more effective or equally effective for the intended indications but will have greatly reduced or no opioid effects (e.g., sedative effects) and risks (e.g., risk of misuse and addiction) and will deter from the use of other opioids.

As an example, a cough syrup combining codeine and/or d-methadone and/or dextromethorphan with naltrexone might be equally effective against cough with less sedation and addiction potential compared to the currently marketed products (Benylin®, Robitussin®, among others) that do not include an opioid antagonist such as naltrexone in their formulations and therefore carry a risk for abuse, addiction and other opioid side effects. The combination of naltrexone with an opioid drug will render the opioid not only free of side effects but an opioid abuse deterrent drug. This combination might also allow a change in the FDA and DEA schedule of an opioid or an opioid combination, for example when used as a cough remedy. To date, it has been counterintuitive to those of ordinary skill in the art to combine an opioid with an opioid antagonist, such as naltrexone, at doses sufficient to counteract all or most of the effects mediated by the opioid receptor agonistic actions. The work of the present inventors described herein, however, has now revealed how there are several actions of certain opioids other than those exerted at the opioid receptors that can be useful for the treatment or prevention of a variety of diseases, symptoms and conditions.

Racemic methadone has been used for the treatment of cough (Molassiotis et al., Clinical expert guidelines for the management of cough in lung cancer: report of a UK task group on cough. Cough. 2010 Oct. 6; 6:9) and intractable hiccups. A novel drug like d-methadone, which combines NMDA antagonistic activity and NE re-uptake inhibition and potentially increases BDNF levels, but is devoid of opioid activity, and is safe and well tolerated, may offer unique advantages for the treatment of these intractable symptoms and clinically more useful than racemic methadone, alone or in combination with naltrexone.

Examples of possible combinations of d-methadone with naltrexone include d-methadone at doses of 1-5000 mg and naltrexone at doses of 1-5000 mg (e.g., d-methadone 1-250 mg combined with naltrexone 1-50 mg) for (1) cyto-protection against genetic, degenerative, toxic, traumatic, ischemic, infectious, neoplastic and inflammatory diseases of cells and prevention and treatment of their symptoms, (2) treatment of pain and opioid tolerance, (3) treatment of psychiatric diseases and symptoms, including addiction to drugs, alcohol, nicotine, and behavioral addictions, (4) cough, (5) obesity (6) metabolic diseases and aging and its symptoms and manifestations (7) eye disease (8) diseases of the NS and their symptoms and manifestations. The d-methadone/naltrexone combination might also prevent misuse of d-methadone and abolish or curtail even mild opioid effects which in some patients could potentially be caused by the higher doses of d-methadone, such as decreased alertness, decreased concentration, decreased short-term memory and attention span, lethargy, somnolence, respiratory depression, nausea and vomiting, constipation, dizziness and vertigo, itching, nasal stuffiness and congestion, worsening of asthma, cough suppression, physical dependence, addiction, miosis.

Because of the curtailing of the listed possible opioid related side effects, a combination of naltrexone or nalmefene may offer synergy and reduced side effects when used with any opioid with actions at the NMDAR catecholaminergic or serotoninerg systems or BDNF or testosterone systems, such as with methadone like drugs (d-methadone, l-methadone, methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, isomethadone, l-isomethadone, d-isomethadone, normethadone and N-methyl-methadone, N-methyl-d-methadone, N-methyl-l-methadone); phenaxodone, l-phenaxodone, d-phenaxodone; diampromide, l-diampromide and d-diampromide; moramide, d-moramide and l-moramide; and also when used with racemorphan like drugs (dextromethorphan, racemorphan, dextrorphan, 3-methoxymorphinan, 3-hydroxymorphinan, levorphanol, levallorphan) or other opioids like buprenorphine, tramadol, and meperidine (pethidine), its metabolite normeperidine (norpethidine), and propoxyphene, its metabolite norpropoxyphene, dextropropoxyphene, levopropoxyphene, fentanyl, its metabolite norfentanyl, morphine, oxycodone, hydromorphone and their metabolites and deuterated and tritium analogues for all listed drugs. In summary, this naltrexone/opioid combination, by blocking opioid effects and therefore allowing other effects (NMDA, NET, SERT, BDNF, testosterone mediated effects) to exert clinically useful actions (in the absence of opioidergic actions), may be useful for 1) cyto-protection against genetic, degenerative, toxic, traumatic, ischemic, infectious, neoplastic and inflammatory diseases and aging of cells and prevention and treatment of their symptoms 2) treatment of pain 3) treatment of psychiatric diseases and symptoms. (4) cough, (5) obesity (6) endocrine and metabolic diseases and aging and its symptoms and manifestations (7) eye diseases (8) diseases of the NS and their symptoms and manifestations.

Another aspect of the present invention includes the use of d-methadone for the treatment of cognitive symptoms associated with chronic pain and its treatment, including cancer pain.

Another aspect of the present invention includes the use of d-methadone to treat d-methadone for the treatment of cognitive symptoms associated with cancer and its treatments, including chemotherapy, radioisotopes, immunotherapy and radiation therapy, including brain radiotherapy.

Another aspect of the present invention includes the use of d-methadone to treat cognitive symptoms associated with opioid therapy.

Another aspect of the present invention includes the use of d-methadone to treat or prevent NS impairment after the occurrence of a stroke and after the occurrence of other NS disorders and/or to treat or prevent the associate cognitive symptoms. Through NMDAR antagonism and the other mechanisms outlined in this application, d-methadone has the potential for providing neuroprotection after acute NS injury, including stroke, and thus limit NS impairment.

As described above, aspects of the present invention are directed to administering substances to a subject to affect the presence of neurotransmitters (by blocking receptors and/or reuptake of neurotransmitters or by increasing BDNF or testosterone). Thus, the NMDA receptor is capable of biological action, and the administering of the substance in the present invention is effective to block the biological action of the NMDA receptor. The NMDA receptor may be located in the nervous system of the subject.

Alternatively, or additionally, the subject may have an NET and/or SERT that is capable of biological action, and the administering of the substance in the present invention is effective to inhibit the NE reuptake at the NET and/or serotonin uptake at the SERT. The NET and/or the SERT may be located in the nervous system of the subject.

Alternatively, or additionally, the subject may have a BDNF receptor that is capable of biological action, and the administering of the substance in the present invention is effective to increase BDNF at the BDNF receptor. The BDNF receptor may be located in the nervous system of the subject.

Alternatively, or additionally, the subject may have a testosterone receptor that is capable of biological action, and the administering of the substance in the present invention is effective in increasing testosterone at the testosterone receptor. The testosterone receptor may be located in the nervous system of the subject or in other organs.

In various aspects and embodiments of the present invention, the administering of the NS drug and the d-methadone is performed orally, buccally, sublingualy, rectally, vaginally, nasally, via aereosol, trans-dermally, parenterally (e.g., intravenous, intradermal, subcutaneous, and intramuscular injection), epidurally, intrathecally, intraocularly, intra-auricularly, including implanted depot formulations, or topically, including eye drops. Further, the subject may be a mammal, such as a human.

In various aspects and embodiments, the present invention may further comprise administering at least one d-isomer of an analog of d-methadone in combination with the administering of d-methadone.

In one particular embodiment, the substance administered may be d-methadone. And the d-methadone may be in the form of a pharmaceutically acceptable salt. Further, the d-methadone may be delivered at a total daily dosage of about 0.01 mg to about 5,000 mg.

Another aspect of the present invention may include administering another drug to the subject in combination with the administering of d-methadone. In various embodiments, the drug may be chosen from cholinesterase inhibitors; other NMDA antagonists, including memantine, dextromethorphan, and amantadine; mood stabilizers; anti-psychotics including clozapine; CNS stimulants; amphetamines; anti-depressants; anxiolytics; lithium; magnesium; zinc; analgesics, including opioids; opioid antagonists, including naltrexone, nalmefene, naloxone, 1-naltrexol, dextronaltrexone, and including NOP antagonists and selective k opioid receptor antagonists; nicotine receptor agonists and nicotine; tauroursodeoxycholic acid (TUDCA) and other bile acids, obethicolic acid, idebenone, phenylbutyric acid (PBA) and other aromatic fatty acids, calcium-channel blockers and nitric oxide synthase inhibitors, levodopa, bromocriptine and other anti-Parkinson drugs, riluzole, edavarone, antiepileptic drugs, prostaglandins, beta-blockers, alpha-adrenergic agonist, carbonic anhydrase inhibitors, parasympathomimetics, epinephrine, hyperosmotic agents.

Turning now to the inventors' discovery of the use of a substance such as d-methadone for the treatment or prevention of NS disorders (and/or their symptoms and manifestations): Researchers at Memorial Sloan Kettering conducted a clinical study of d-methadone (designed by the present inventors) to establish its safety and analgesic potential. The results of this trial were published in 2016 (Moryl, N. et al., A phase I study of d-methadone in patients with chronic pain. Journal of Opioid Management 2016: 12:1; 47-55, incorporated by reference herein in its entirety). This phase I-2a study studied the effects of d-methadone administered to patients with chronic cancer pain at a dose of 40 mg every 12 hours for 12 days. Upon a novel analysis of the data from this study, the inventors discovered that patients taking d-methadone experienced improvement in their Modified Mini Mental State (3MS) scores on day 12 of treatment with d-methadone compared with baseline pre-treatment scores. (As is known to those skilled in the art, the Modified Mini Mental State (3MS) is designed to assess the person's attention, concentration, orientation to time and place, long-term and short-term memory, language ability, constructional praxis, abstract thinking, and list-generating fluency.)

In particular, five out of six evaluable patients improved by at least one point, with one patient improving by as many as 6 points (mean improvement 1.8). Only one patient worsened on day 12 compared to pre-treatment with d-methadone; this patient worsened by 2 points. These were all patients with a high baseline 3MS score (mean 96.7), and so the inventors determined that (1) d-methadone may potentially be beneficial for patients with even mild neurological impairment, as opposed, for example, to memantine (which is FDA approved only for patients with moderate or severe dementia) and (2) the data suggested possible benefits from d-methadone in NS disorders where abnormalities in the NMDA, NET, and/or SERT systems, BDNF or testosterone levels could be modulated by a drug like d-methadone (such as the NS disorders recited above).

Of note, at the time of the study, the investigators simply concluded that d-methadone was free of cognitive side effects, overlooking any possible direct therapeutic benefit. An excerpt from the study protocol indicates that the investigators had hypothesized possible cognitive benefits only in the event of an opioid reduction, and not from a direct effect of the drug, stating: “Other NMDA antagonists have been shown to cause cognitive side effects (23, 24, 30). It is unclear if d-methadone will have such effects or if, by decreasing opioid requirements, it will instead improve cognitive function” (see p. 15 of Moryl, N. et al., A Phase I/II Study of D-Methadone in Patients with Chronic Pain—THERAPEUTIC/DIAGNOSTIC PROTOCOL, Memorial Sloan-Kettering Cancer Center (2008) IRB#: 01-017A(12):1-28).

In fact, throughout the discussion/conclusion of the Moryl 2016 study, notably there is no mention about the possible direct benefits of d-methadone on the cognitive function. Rather, in the study, the investigators state that numerous clinical reports have underscored the superior analgesic efficacy of methadone compared to other opioids and less dose escalation with methadone compared with morphine, implying less tolerance to analgesic effects of methadone. And so, the investigators state that these unique advantages of methadone (such as effectiveness of methadone in difficult to control pain and less tolerance to methadone) are usually attributed to NMDA antagonism of the d-methadone isomer. The investigators further concluded that their study demonstrated that d-methadone appears to be safe and well tolerated in patients with chronic pain at the dose of 80 mg a day given in two divided doses.

The new observation of the inventors, based on data from the only prospective human trial with d-methadone in patients with cancer related pain, is that d-methadone is not only safe, as concluded by the 2016 Moryl paper, but may have a direct effect on cognitive abilities. The inventors' discovery is corroborated by the known effects of other NMDA antagonists, NE and SER reuptake inhibitors, and BDNF and testosterone on the cognitive system, and particularly on learning, memory, and neuronal plasticity. The cognitive improvement described in these patients, signal possible therapeutic benefits of d-methadone in many NS disorders, particularly in light of new actions of d-methadone discovered by the inventors, particularly in regards to newly discovered up-regulation of BDNF and testosterone.

This new finding, that d-methadone may directly improve cognition, is also shown by a second line of evidence discovered by the inventors, and also based on their joint knowledge about methadone and d-methadone: Manfredi (one of the present inventors), and other authors, experts in the use of methadone for pain, over the years have published studies and case series showing that the administration of racemic methadone improves analgesia and is associated with less opioid cognitive side effects compared to other opioids [Morley, J. S. et al., Methadone in pain uncontrolled by morphine. Lancet. 1993 Nov. 13; 342(8881):1243; Manfredi, P. L. et al., Intravenous methadone for cancer pain unrelieved by morphine and hydromorphone. Pain 1997; 70: 99-101; De Conno, F. et al. Clinical experience with oral methadone administration in the treatment of pain in 196 advanced cancer patients. C. J Clin Oncol. 1996 October; 14 (10): 2836-42; Santiago-Palma, J. et al., Intravenous methadone in the management of chronic cancer pain: safe and effective starting doses when substituting methadone for fentanyl. Cancer 2001; 92 (7):1919-1925; Moryl, N. et al. Pitfalls of opioid rotation: substituting another opioid for methadone in the treatment of cancer pain. Pain 2002; 96(3):325-328]. These authors, including the present inventor Manfredi, have previously always attributed the improvement in cognition and alertness that followed a switch from another opioid to methadone, to decreased opioid tolerance—and thus to a lowering in equivalent opioid dose and less opioid side effects. This was the conventional wisdom of those of ordinary skill in the art. Those skilled in the art never contemplated a direct positive effect of methadone on cognition and alertness, and thus never considered the possible therapeutic implications for d-methadone in diseases of the NS.

In particular, in the 2001 prospective clinical study by Santiago-Palma et al., for which Manfredi (a present inventor) is the senior corresponding author (incorporated by reference herein in its entirety), 18 patients were switched from fentanyl to methadone because of sedation or confusion. In these patients, sedation decreased from 1.5 to 0.16 (P=0.001). Out of the 18 patients, 6 were confused immediately prior to the switch; following the switch, 5 of these 6 patients improved subjectively (feeling clear minded and not feeling confused) and objectively (testing of orientation, simple calculations and short term memory). The present inventors, after reviewing the data from this and other similar studies were able to newly conclude that the cognitive improvement and the resolution of sedation and confusion seen in these patients is possibly determined by a direct effect of racemic methadone on the NMDA, NET, and SERT systems, and/or BDNF levels and/or testosterone levels, and not, as previously assumed, by a the sudden lack of fentanyl's opioid side effects. Therefore d-methadone, shown by the inventors to be devoid of opioid activity and psychotomimetic effects, may have an effect at the NMDA, NET, and SERT systems, and BDNF and testosterone levels, that will benefit patients with cognitive impairment from different causes.

In Moryl N, Santiago-Palma J, Kornick C, Derby S, Fischberg D, Payne R, Manfredi P. Pitfalls of opioid rotation: substituting another opioid for methadone in patients with cancer pain. Pain 96 (2002) 325-328—for which Manfredi is the senior corresponding author-incorporated by reference herein in its entirety, 13 patients were prospectively switched from methadone to a different opioid. 12 of these 13 patients had to be switched back to methadone because of side effects such as confusion (4 patients), sedation (3 patients), dysphoria (4 patients) and myoclonus (1 patient). The inventors, after reviewing the data from this and other similar studies are now able to conclude that the cognitive worsening seen in these patients when methadone was discontinued, is possibly determined by a sudden lack of a direct effect of racemic methadone on the NMDA, NET, and SERT systems and/or BDNF levels and/or testosterone levels and not caused by a toxic effect of the second opioid, as previously assumed. Therefore, the sudden appearance of cognitive symptoms after discontinuation of methadone represent indirect but strong evidence that d-methadone may have an effect at the NMDA, NET, and SERT systems and/or BDNF levels and/or testosterone levels that directly benefits patients with cognitive impairment, without the side effects and risk of opioids, including racemic methadone and l-methadone (opioid side effects include worsening of cognitive functions), as shown by the inventors (as will be demonstrated in the studies of the Examples, below).

Furthermore, the clinical work performed over the years by present inventor Manfredi with the use of opioids, in particular racemic methadone, for the treatment of pain in patients with cognitive impairment ranging from mild to extremely severe [Manfredi, P. L. et al., Opioid Treatment for Agitation in Patients with Advanced Dementia. Int J Ger Psy 2003; 18:694-699; Manfredi, P. L. et al., Pain Assessment in Elderly Patients with Severe Dementia. J Pain Sympt Manag 2003; 25(1):48-52; Manfredi, P. L., Opioids versus antidepressants in postherpetic neuralgia: A randomized placebo-controlled trial. [Letter]. Neurology. Neurology. 2003 Mar. 25; 60(6):1052-3] has suggested improved cognitive performance in patients treated with racemic methadone compared to patients treated with other opioids. This finding had also been previously always attributed to decreased opioid tolerance and NMDA effects on pain—and thus lowering in equivalent opioid dose. The collaboration of the present inventors has allowed them to jointly discover that the improved cognitive and functional abilities in patients treated with racemic methadone instead of other opioids, including patients with baseline cognitive impairment unrelated to opioids, might be indicative of a direct therapeutic role of the NMDA antagonistic activity and/or NE or serotonin reuptake inhibition and or related to the increase in BDNF and/or related to the increase in testosterone and thus directly induced by d-methadone in these patients, and not, as previously believed, an indirect effect from decreased opioid tolerance and a lowering of the equivalent opioid dose and decreased opioid side effects.

The important implication of this discovery is that a drug like d-methadone is potentially effective for many NS disorders and their symptoms and manifestations. As observed by the present inventors: (1) Patients treated with methadone were less likely to suffer cognitive side effects than patients treated with other opioids [Santiago-Palma, J. et al., Intravenous methadone in the management of chronic cancer pain: safe and effective starting doses when substituting methadone for fentanyl. Cancer 2001; 92 (7):1919-1925; Moryl, N. et al., Pitfalls of opioid rotation: substituting another opioid for methadone in the treatment of cancer pain. Pain 2002; 96(3):325-328]; (2) Patients switched from other opioids to methadone had rapid improvement in cognitive impairment with resolution of confusion (Santiago-Palma, J. et al., Intravenous methadone in the management of chronic cancer pain: safe and effective starting doses when substituting methadone for fentanyl. Cancer 2001); (3) Elderly patients with cognitive impairment caused by CNS disorders have better cognitive function on methadone compared to other opioids [Manfredi, P. L., Opioids versus antidepressants in postherpetic neuralgia: A randomized placebo-controlled trial. [Letter]. Neurology. Neurology. 2003 Mar. 25; 60(6):1052-3]; (4) Agitated and restless patients achieved relief from their restlessness immediately after a switch to methadone from another opioid; in these patients, abnormal movement such as myoclonus also improved [Santiago-Palma, J. et al., Intravenous methadone in the management of chronic cancer pain: safe and effective starting doses when substituting methadone for fentanyl. Cancer 2001]; (5) Patients treated with methadone had improved sleep [this finding was also noted and published by De Conno, F. et al., Clinical experience with oral methadone administration in the treatment of pain in 196 advanced cancer patients. C. J Clin Oncol. 1996 October; 14 (10): 2836-42]; and (6) Patients on methadone switched to another opioid developed confusion, sedation, restlessness, myoclonus [Moryl, N. et al. Pitfalls of opioid rotation: substituting another opioid for methadone in the treatment of cancer pain. Pain 2002; 96(3):325-328].

In light of their joint work, the present inventors are now able to attribute the improvement in cognition and agitation and sleep outlined in points 1-5 above to a direct effect at the NMDA receptor and NET, SERT, and/or BDNF and or testosterone, rather than to a decrease in opioid side effects, as previously thought.

Because of its direct effect on cognition, d-methadone may not benefit only patients cognitively impaired by opioids, by allowing a lowering in equivalent opioid dose. Instead, by improving cognitive function directly, independently of the opioid treatment, it will have potential therapeutic indications for patients with cognitive impairment from any CNS condition susceptible of improvement by modulation of the NMDA, NET, and/or SERT systems, and/or by increasing BDNF levels and/or testosterone levels.

The collaboration between the inventors led to the discovery that d-methadone may have a measurable direct therapeutic effect on CNS symptoms, rather than simply decrease the side effects of other opioids, as hitherto accepted by experts. Based on this discovery, d-methadone will not only benefit patients in need of analgesia or suffering psychiatric symptoms, but also patients suffering from NS diseases and their symptoms and manifestations. Further, as discovered by the inventors after review of the data from the 2016 Moryl phase I study, and the review of their own d-methadone and racemic methadone studies, d-methadone may also have a direct effect on neurological symptoms and manifestation and not simply decrease the side effect of other opioids, as previously assumed.

While the patients' improvement in 3MS scores and other cognitive improvements (described in studies performed by Manfredi and other authors) were overlooked and mis-interpreted even by those experienced in the art, from the unique joint perspective of the inventors, based on decades of experimental and clinical studies on d-methadone and methadone, the cognitive improvements seen in patients treated with d-methadone and racemic methadone signal a probable direct beneficial effect of d-methadone for patients suffering from CNS disorders and their neurological symptoms and manifestations, including patients with minimal or mild cognitive impairment from other drugs or other diseases. Memory and learning abnormalities and other cognitive impairments secondary to recreational drugs, including opioid, cannabinoids, cocaine, LSD, amphetamines, and others such as 3,4-Methylenedioxymethamphetamine (MDMA) may also be improved by d-methadone treatment.

Below are some examples of diseases and conditions that are candidates for treatment(s) as described herein.

Alzheimer's Disease and Parkinson's Disease

Alzheimer's disease is a progressive, neurodegenerative disorder resulting in impairment of memory, executive function, visuospatial functions, and language, and behavioral changes. Affected neurons, which produce neurotransmitters such as acetylcholine, break connections with other nerve cells and ultimately die. For example, short-term memory fails when Alzheimer's disease first destroys nerve cells in the hippocampus, and language skills and judgment decline when neurons die in the cerebral cortex. Alzheimer's disease is the most common cause of dementia, or loss of intellectual function, among people aged 65 and older.

Parkinson's disease (PD) is a multifaceted neurodegenerative disorder characterized by both motor (bradykinesia, resting tremor, rigidity, and postural instability) and non-motor symptoms (REM behavior disorder [RBD], hyposmia, constipation, depression and, cognitive impairment). Even in early stages of PD, cognition is commonly impacted on a range of subdomains, including problems with executive function, attention/working memory, and visuospatial function. Wang reports a significant correlation between subdomains of cognitive impairment and motor dysfunction; notably, executive function and attention was significantly associated with bradykinesia and rigidity, while visuospatial function was associated with bradykinesia and tremor (Wang Y et al. Associations between cognitive impairment and motor dysfunction in Parkinson's disease. Brain and Behavior. 2017; 7(6)). The association between motor dysfunction and cognitive decline in PD might highlight deficits represented by a shared neurochemical pathway. This shared neurochemical pathway can potentially be targeted with d-methadone.

Dysfunction of central nervous system NMDA receptors by the excitatory amino acid glutamate contributes to the symptomatology of Alzheimer's disease and other CNS disorders, including Parkinson's disease and related disorders (Paoletti P et al., NMDA receptor subunit diversity: impact on receptor properties, synaptic plasticity and disease. Nature Reviews Neuroscience 14, 383-400 (2013) such as Parkinsonian related disorders including but not limited to Parkinson dementia; disorders associated with accumulation of beta amyloid protein (including but not limited to cerebrovascular amyloid angiopathy, posterior cortical atrophy); disorders associated with accumulation or disruption of tau protein and its metabolites including but not limited to frontotemporal dementia and its variants, frontal variant, primary progressive aphasias (semantic dementia and progressive non fluent aphasia), corticobasal degeneration, supranuclear palsy.

Further, the brain noradrenergic system supplies the neurotransmitter NE (norepinephrine) throughout the brain via widespread efferent projections, and plays a pivotal role in modulating cognitive activities in the cortex. Profound noradrenergic degeneration in Alzheimer's disease (AD) patients has been observed for decades, with recent research suggesting that the locus coeruleus (where noradrenergic neurons are mainly located) is a predominant site where AD-related pathology begins. Mounting evidence indicates that the loss of noradrenergic innervation greatly exacerbates AD pathogenesis and progression (Gannon, M. et al., Noradrenergic dysfunction in Alzheimer's disease. Front Neurosci. 2015; 9: 220). Of note cognitive deterioration and Alzheimer's have been associated with a decline in reproductive hormones including testosterone (Gregory C W and Bowen R L. Novel therapeutic strategies for Alzheimer's disease based on the forgotten reproductive hormones. Cell Mol Life Sci. 2005 February; 62(3):313-9).

Presently, there are limited treatment options for Alzheimer's disease (Eleti S. Drugs in Alzheimer's disease Dementia: An overview of current pharmacological management and future directions. Psychiatr Danub. 2016 September; 28(Suppl-1):136-140). There are only five FDA approved drugs for Alzheimer's disease and only one of these drugs, memantine (also shown to have beneficial effects in Parkinson's disease), is an NMDA antagonist. As has been described above, NMDA (N-methyl-D-aspartate) receptor antagonists regulate the activity of glutamate, an important neurotransmitter in the brain involved in learning and memory. Attachment of glutamate to cell surface “docking sites” called NMDA receptors permits calcium to enter the cell. This process is important for cell signaling, as well as learning and memory.

In Alzheimer's disease, excess glutamate can be released from damaged cells, leading to chronic overexposure to calcium, which can accelerate cell damage. NMDA antagonists such as memantine, may help prevent this destructive chain of events by partially blocking NMDA receptors. More specifically, memantine is postulated to exert its therapeutic effect through its action as a low to moderate affinity uncompetitive (open-channel) NMDA receptor antagonist, which binds preferentially to the NMDA receptor-operated cation channels. In clinical trials, the glutamatergic modulator memantine was found to offer improvement over placebo for patients suffering from moderate to severe Alzheimer's disease, improving functional and cognitive abilities. However, many patients do not respond or respond poorly to memantine and some suffer side effects that stop them from using the drug. Memantine is eliminated by the kidneys and renal impairment causes accumulation and side effects.

NS disorders and their neurological symptoms and manifestations unresponsive to memantine, may instead respond to a drug like d-methadone, which combines NMDA antagonisms with inhibition of NET and SERT and serotonin and up-regulation of BDNF and testosterone, alone or in combination with standard therapy. As described above, in addition to NMDA antagonistic activity, d-methadone is an inhibitor of NE and serotonin reuptake [Codd, E. E. et al., Serotonin and Norepinephrine activity of centrally acting analgesics: Structural determinants and role in antinociception. IPET 1995; 274 (3)1263-1269] and this combined modulating activity may uniquely contribute to alleviate cognitive symptoms of neurodegenerative disorders, particularly for patients with Alzheimer's disease.

And so, a drug like d-methadone which combines NMDA antagonistic activity and NE and serotonin re-uptake inhibition, and potentially increases BDNF and testosterone levels, may therefore offer unique advantages for the treatment of Alzheimer's disease and Parkinson's disease and other CNS diseases, and their symptoms and manifestations. The discovery by the present inventors, that d-methadone improves cognitive function and that racemic methadone—despite its strong opioid effects—can in some patients reduce sedation, confusion, and agitation, suggests that d-methadone, which, as shown by the inventors, is devoid of opioid effects and psychotomimetic side effects and improves cognitive function at potentially therapeutic doses, may be effective for the management of many CNS disorders, including Alzheimer's disease and Parkinson's disease.

Schizophrenia Including Neurological Side Effects from its Treatment

Disruption at the NMDA [Coyle, J. T., NMDA Receptor and Schizophrenia: A Brief History. Schizophrenia Bulletin vol. 38 no. 5 pp. 920-926, 2012; Paoletti, P. et al., NMDA receptor subunit diversity: impact on receptor properties, synaptic plasticity and disease. Nature Reviews Neuroscience 14, 383-400 (2013)] and NE (Shafti S S et al., Amelioration of deficit syndrome of schizophrenia by norepinephrine reuptake inhibitor. Ther Adv Psychopharmacol 2015, Vol. 5(5) 263-270.) systems have been implicated in the pathophysiology of schizophrenia and its manifestations.

Memantine, an NMDA antagonist with affinities in the micromolar range similarly to d-methadone as shown by the inventors in the Examples, significantly improved the positive and negative symptoms in patients maintained on olanzapine after six weeks compared to olanzapine alone (P<0.001) [Fakhri, A. et al. Memantine Enhances the Effect of Olanzapine in Patients With Schizophrenia: A Randomized, Placebo-Controlled Study. Acta Med Iran. 2016 November; 54(11):696-703]. In another study by Mazinani (Mazinani R et al., Effects of memantine added to risperidone on the symptoms of schizophrenia: A randomized double-blind, placebo-controlled clinical trial. Psychiatry Res. 2017 January; 247:291-295) memantine treatment failed to show improvement on positive and general psychopathologic symptoms; negative symptoms, however, improved significantly in the intervention group. Cognitive function was also significantly improved in the intervention group.

There are several reports of amelioration of symptoms in schizophrenic patients from methadone [Brizer, D. A. et al., Effect of methadone plus neuroleptics on treatment-resistant chronic paranoid schizophrenia. Am J Psychiatry. 1985 September; 142(9):1106-7]. In the 2001 prospective study by Santiago Palma et al., discussed in more detail above, 5 of 6 delirious patients improved within two days of starting methadone.

However, there are several reports of acute psychosis following the discontinuation of methadone [Berken, G H et al., Methadone in schizophrenic rage: a case study. Am J Psychiatry. 1978 February; 135(2):248-9; Judd, L. L. et al., Behavioral effects of methadone in schizophrenic patients. Am J Psychiatry. 1981 February; 138(2):243-5; Levinson, I. et al., Methadone withdrawal psychosis. J Clin Psychiatry. 1995 February; 56(2):73-6; Sutter, M. et al. Psychosis after Switch in Opioid Maintenance Agonist and Risperidone-Induced Pisa Syndrome: Two Critical Incidents in the Treatment of a Patient with Dual Diagnosis. J Dual Diagn. 2016 Dec. 9:0]. In the study from Willi et al. 2016, increased severity of positive psychotic symptoms was significantly related to methadone-abstinence (Willi T S et al., Factors affecting severity of positive and negative symptoms of psychosis in a polysubstance using population with psychostimulant dependence. Psychiatry Res. 2016 Jun. 30; 240:336-42). And, one of the inventors, Manfredi, observed severe dysphoria, agitation and paranoid ideation in patients with pain after discontinuation of methadone (Moryl N et al., Pitfalls of opioid rotation: substituting another opioid for methadone in patients with cancer pain. Pain 96 (2002) 325-328).

The above publications and observations, after a careful review in light of the joint work of the present inventors (described in further detail in the Examples section, below), suggest a therapeutic role for d-methadone in the management of schizophrenia and its symptoms. A drug like d-methadone may help both positive and negative symptoms of schizophrenia and associated cognitive deficits by modulating the NMDA, NET, and/or SERT systems, and/or potentially increase BDNF levels and/or testosterone levels. Of note, in addition to possible benefits from the mechanisms described above, the modulating effects of d-methadone on K⁺ currents might provide additional actions for improving schizophrenia and its symptoms [Wulff H et al., Voltage-gated potassium channels as therapeutic targets. Nat Rev Drug Discov. 2009 December; 8(12): 982-1001].

The absence of opioid effects and psychotomimetic effects in d-methadone uncovered by the inventors is crucial in order to avoid risks associated with opioids side effects, including addiction and cognitive side effects that limit the clinical usefulness of racemic methadone.

Autism Spectrum Disorders and Impairment of Social Interactions

Autism spectrum disorder (ASD) is characterized by difficulty with social communication and restricted, repetitive patterns of behavior, interest, or activities. The Diagnostic and Statistical Manual of Mental Disorders, 5th ed., created an umbrella diagnosis that includes several previously separate conditions: autistic disorder, Asperger syndrome, childhood disintegrative disorder, and pervasive developmental disorder not otherwise specified [Sanchack, K. E. et al., Autism Spectrum Disorder: Primary Care Principles. Am Fam Physician. 2016 Dec. 15; 94(12):972-979].

There are overlapping impairments in Autism Spectrum Disorder (ASD) and Schizophrenia (SCZ) (Morrison K E et al., Distinct profiles of social skill in adults with autism spectrum disorder and schizophrenia. Autism Res. 2017 May; 10(5):878-887). A drug like d-methadone may therefore also be useful for patients with ASD, in addition to its potential to treat patients with SCZ, alone or as an adjunct to standard therapy.

By modulating the NMDA and NET systems and potentially increasing BDNF levels, d-methadone is potentially useful for ASD. Its effects on improving cognitive function, as discovered by the present inventors, is also suggestive of potential usefulness for patients with ASD. The absence of clinically significant opioid side effects and psychotomimetic effects shown by the inventors for d-methadone as detailed in the examples section, is crucial in order to avoid risks associated with opioids side effects, including addiction and cognitive side effects that would limit clinical usefulness. Opioid receptors have been implicated in ASD and impairment of social interactions (Pellissier L P et al., p opioid receptor, social behaviour and autism spectrum disorder: reward matters. Br J Pharmacol. 2017 Apr. 3 doi: 10.1111/bph. 13808. [Epub ahead of print]. Furthermore, family relations among MMT patients were consistently improved over time after MMT. Only 37.9% of drug users reported having a good relationship with their family before receiving MMT interventions; however, this rate increased significantly to 59.6% after 6 months, 75.0% after 12 months and 83.2% after >12 months of treatment [Sun H M et al. Methadone maintenance treatment program reduces criminal activity and improves social well-being of drug users in China: a systematic review and meta-analysis. BMJ Open. 2015 Jan. 8; 5(1)]. While this improvement has been attributed to abstinence from illicit drugs and actions at the opioid receptors, based on their joint work, the inventors suggest a possible beneficial effect at the neuronal level not mediated by stereochemically specific methadone actions (opioidergic actions), but mediated by non stereospecific effects on NMDARs, SERT, NET, and K, Na and Ca ion channels and effects on BDNF, all effects not limited to racemic methadone but shared by d-methadone. Clinical trials in specific patient populations with d-methadone, without the opioid effects of racemic methadone and without the confounding effects of the psychiatric co-morbidities of opioid addiction, will allow a better understanding of the specific neuropsychiatric indications for d-methadone, including ASD and its related impairment of social skills. Therefore a drug like d-methadone could improve ASD and individuals with impairment of social interactions and skills through a multiplicity of mechanisms, including low affinity interactions with opioid receptors, modulating the NMDA, NET, and/or SERT systems, the KNa and Ca ion channels and/or potentially regulate BDNF levels and/or gonadal hormonal levels.

Dysfunctional mTOR signaling may represent a molecular abnormality present in several well-characterized syndromes with high prevalence of ASD. ASD may be part of the clinical presentation of well-characterized genetic syndromes, such as tuberous sclerosis complex, fragile X syndrome, Rett syndrome, Angelman syndrome, phosphatase and tensin homolog (PTEN)-related syndromes, neurofibromatosis type 1, Timothy syndrome, 22q13.3 deletion syndrome, among others. These ASD-related syndromes, although representing only 5%-10% of all ASD cases, have contributed greatly to our understanding of ASD pathogenesis (Magdalon J et al. “Dysfunctional mTORC1 Signaling: A Convergent Mechanism between Syndromic and Nonsyndromic Forms of Autism Spectrum Disorder?” Ed. Merlin G. Butler. International Journal of Molecular Sciences 18.3 (2017): 659. PMC. Web. 21 Aug. 2017). BDNF exerts some of its actions by activating the Mammalian Target of Rapamycin (mTOR) (Smith D E et al., Rapamycin and Interleukin-1β Impair Brain-derived Neurotrophic Factor-dependent Neuron Survival by Modulating Autophagy. Jul. 25, 2014 The Journal of Biological Chemistry 289, 20615-20629). The activation of mTOR can be induced by BDNF in neuronal dendrites, thus, certain kinds of synaptic plasticity induced by BDNF might be mediated by mTOR-dependent, regulated local translation in neuronal dendrites (Takei N et al., Brain-Derived Neurotrophic Factor Induces Mammalian Target of Rapamycin-Dependent Local Activation of Translation Machinery and Protein Synthesis in Neuronal Dendrites. The Journal of Neuroscience, Nov. 3, 2004⋅24(44):9760-9769). The researchers demonstrated that BDNF in neuronal dendrites activates mTOR and 4EBP phosphorylation, which are key steps for cap-dependent translation. This is the molecular basis for mTOR-dependent local activation of the translation machinery, and this activation leads to local protein synthesis in the dendrites of cortical neurons after exposure to BDNF. Thus, according to the research of Takei et al., certain kinds of synaptic plasticity induced by BDNF might be mediated by mTOR-dependent, regulated local translation in neuronal dendrites. Drugs that increase BDNF, like d-methadone, may therefore exert neuroprotection by regulating dysfunctional mTOR signaling and might potentially offer new treatments approaches for disorders of the NS and their symptoms and manifestations.

Tuberous Sclerosis

Tuberous sclerosis complex (TSC) is a rare multisystem genetic disease that causes benign tumors to grow in the brain and on other vital organs such as the kidneys, heart, liver, eyes, lungs, and skin. A combination of symptoms may include seizures, intellectual disability, developmental delay, behavioral problems, skin abnormalities, and lung and kidney disease. TSC is caused by a mutation of either of two genes, TSC1 and TSC2, which code for the proteins hamartin and tuberin, respectively. These proteins act as tumor growth suppressors, agents that regulate cell proliferation and differentiation. The quality of life for those afflicted by Tuberous Sclerosis Complex (TSC) is affected by intellectual and neurological disabilities mediated in part by excessive glutamatergic activity in the brain. Interestingly, the severity of the intellectual disability in Tuberous Sclerosis Complex may relate more to metabolic disturbance (such as excessive glutamatergic activity, overactivity of mTOR signaling and lowered BDNF levels) than the density of cortical tubers (Burket J A et al., (2015). NMDA receptor activation regulates sociability by its effect on mTOR signaling activity. Progress in Neuro-Psychopharmacology & Biological Psychiatry, 60, 60-65).

A drug like d-methadone by blocking the NMDAR and NET systems and potentially increasing BDNF levels and thus modulating mTOR signaling, is potentially useful for improving the quality of life, sociability and cognitive function in patients with tuberous sclerosis.

Rett Syndrome

Rett syndrome, including its variants, is an important cause of disability in women. Onset of symptoms occurs between 6 and 18 months with developmental regression of language and motor milestones, purposeful hand use is lost, and acquired deceleration in the rate of head growth (resulting in microcephaly in some) is seen. Hand stereotypes are typical, and breathing irregularities such as hyperventilation and breath-holding spells are often seen. Autistic behavior is also seen. While the cause is genetic, various abnormalities in neurotransmitters, receptors, and neurotrophic factors have been observed in these patients. Classic Rett syndrome is due to a de novo mutation in an X-linked gene (MECP2) that encodes for a chromatin protein (MeCP2) that regulates gene expression.

Brain levels of norepinephrine are lowered in patients with Rett syndrome [Zoghbi H Y et al., Cerebrospinal fluid biogenic amines and biopterin in Rett syndrome. Annals of Neurology. 25 (1): 56-60]. Researchers found increased spinal fluid levels of glutamate and increased NMDA receptors in the brain of patients with Rett syndrome [Blue M E et al., Development of amino acid receptors in frontal cortex from girls with Rett syndrome. Annals of Neurology 1999; 45 (4): 541-5]. In experimental studies, chronic administration of ketamine has been shown to improve Rett syndrome phenotype in MecP2-null mice (Patrizi A et al., Chronic Administration of the N-Methyl-D-Aspartate Receptor Antagonist Ketamine Improves Rett Syndrome Phenotype. Biol Psychiatry. 2016 May 1; 79(9):755-64). Patients with Rett syndrome have been treated with dextromethorphan and ketamine with some success.

d-Methadone might have clinical effects as powerful or more powerful than ketamine, based on the new data from the forced swim test (FST), the female urine smelling test (FUST) and the novelty food suppression test (NSFT) described in greater detail below in the Examples section; in all of these tests, d-methadone at doses comparable to the effective ketamine doses used by Patrizi in the Rett mouse model, [Patrizi A et al., Chronic Administration of the N-Methyl-D-Aspartate Receptor Antagonist Ketamine Improves Rett Syndrome Phenotype. Biol Psychiatry. 2016 May 1; 79(9):755-64] exerted strong behavioral responses, comparable to those exerted by ketamine; additionally, d-methadone is devoid of psychotomimetic effects typical of ketamine, as demonstrated by the novel phase I data provided by the inventors in the Examples section. Also, the PK data for d-methadone are shown by the inventors (in the Examples) to be compatible with once a day administration, unlike dextrometorphan which requires the addition of quinidine, a potentially arrhytmogenic drug, in order to achieve satisfactory blood levels. In addition, dextromethorphan has an active metabolite and is subject to a CYP2D6 genetic polymorphism that results in variable pharmacokinetics and response in the population, a clear disadvantage compared to d-methadone [Zhou S F. Polymorphism of human cytochrome P450 2D6 and its clinical significance: part II. Clin Pharmacokinet. 48:761-804, 2009].

BDNF is deregulated in Rett syndrome suggesting that therapeutic interventions based on improving BDNF function may be effective in treating or alleviating the symptoms and signs of this disease (Li W. and Pozzo-Miller L. BDNF deregulation in Rett syndrome. Neuropharmacology 2014:76). A drug like d-methadone, by modulating the NMDA and NET systems and by up-regulating BDNF levels as revealed by the inventors in the Examples section, holds therapeutic potential for alleviating symptoms and signs of Rett syndrome, including respiratory abnormalities. The strong potential for improving Rett phenotype by administering d-methadone is signaled by the ketamine-like behavioral effects of d-methadone on experimental models, FST, FUST, NSFT, as outlined and the experimental section.

Eating Disorders

Eating disorders, which include anorexia nervosa (“AN”) and bulimia nervosa (“BN”), and Binge Eating Disorder (“BED”), are disorders characterized by abnormal patterns of weight regulation and eating behaviors, and by disturbances in attitudes and perceptions toward weight and body shape.

Brain-derived neurotrophic factor (BDNF) plays a critical role in regulating neural survival, development, function, and plasticity in the brain. Recent findings using heterozygous BDNF (+/−) knock-out (reduced BDNF levels) mice have provided evidence that BDNF plays a role in regulating eating behaviors. Hashimoto et al., 2005, found that serum levels of BDNF in patients with eating disorders are significantly decreased compared with normal controls; in addition, an association between the BDNF gene polymorphism and eating disorders has been demonstrated; furthermore, Hashimoto reviewed the role of BDNF in the pathophysiology of eating disorders and the BDNF gene as a susceptibility gene for eating disorders; Providing confirmation that the BDNF gene is the true susceptibility gene for eating disorders could lead to rapid therapeutic progress in treating these disorders. In addition, a more complete understanding of the signal transduction pathway via the p75 neurotrophin receptor (p75NTR) and TrkB receptors would provide new perspectives for treating eating disorders (Hashimoto K et al. Role of brain-derived neurotrophic factor in eating disorders: recent findings and its pathophysiological implications. Prog Neuropsychopharmacol Biol Psychiatry. 2005 May; 29(4):499-504).

A novel drug like d-methadone shown by the inventors to have NMDA receptor affinities in the micromolar range similar to memantine, to exert behavioral effects in rats more potently than ketamine (while devoid of psychotomimetic side effects) and, perhaps more importantly, to potentially increase serum BDNF levels, could be useful for the treatment of eating disorders including AN, BN and BED.

Human Obesity and Rare Syndromes and Common Variants of the Brain-Derived Neurotrophic Factor Gene and the Metabolic Syndrome.

Rare genetic disorders that cause BDNF haploinsufficiency, such as WAGR syndrome, 11p deletion, and 11p inversion, serve as models for understanding the role of BDNF in human energy balance and neurocognition. Patients with BDNF haploinsufficiency or inactivating mutations of the BDNF receptor exhibit hyperphagia, childhood-onset obesity, intellectual disability, and impaired nociception. Prader-Willi, Smith-Magenis, and ROHHAD syndromes are separate genetic disorders that do not directly affect the BDNF locus but share many similar clinical features with BDNF haploinsufficiency, and BDNF insufficiency is believed to possibly contribute to the pathophysiology of each of these conditions. In the general population, common variants of BDNF that affect BDNF gene expression or BDNF protein processing have also been associated with modest alterations in energy balance and cognitive functioning. Thus, variable degrees of BDNF insufficiency appear to contribute to a spectrum of excess weight gain and cognitive impairment that ranges in phenotypic severity (Han J C. Rare Syndromes and Common Variants of the Brain-Derived Neurotrophic Factor Gene in Human Obesity. Prog Mol Biol Transl Sci. 2016). Furthermore, as detailed by the inventors in Example 8 in the Examples section, administration of d-methadone results in a dose dependent decreased weight gain in rats, signaling a possible effect on weight regulation.

A novel drug like d-methadone, found by the inventors to improve cognitive performance and to enhance BDNF levels and up-regulate testosterone could be useful for treating obesity and neurodevelopmental disorders including BDNF insufficiency, including WAGR syndrome, 11p deletion, and 11p inversion, and Prader-Willi (decreased weight gain and regulation of serum glucose and blood pressure from d-methadone described in the Examples section can also contribute to ameliorating symptoms in Prader-Willi syndrome), Smith-Magenis, and ROHHAD syndromes and hypothalamic-pituitary axis disorders.

The regulation of appetite involves hypothalamic circuits which include the arcuate nucleus. In the event of excess glutamate, arcuate nucleus neurons may be vulnerable to excitotoxity. There is clinical evidence that memantine, an NMDAR antagonists may decrease appetite and suppress binge-eating in obese patients [Hermanussen, M. et al., A new anti-obesity drug treatment: first clinical evidence that, antagonising glutamate-gated Ca²⁺ ion channels with memantine normalizes binge-eating disorders. Econ Hum Biol. 2005 July; 3(2):329-37; Brennan, B. P. et al., Memantine in the treatment of binge eating disorder: an open-label, prospective trial. Int J Eat Disord. 2008 41(6):520-6].

Methadone has been found to act as a hypoglycemic, and hypoglycemia caused by methadone has been described in the literature. In a study by Flory, J. H. et al. [Methadone Use and the Risk of Hypoglycemia for Inpatients with Cancer Pain. Journal of pain and symptom management. 2016; 51(1):79-87], linear multivariable regression showed methadone to be significantly associated with reduced average minimum daily blood sugar by −5.7 mg/dl (95% CI −7.3, −4.1, equivalent to mmol/I 0.31), with increasing doses associated with greater effect. The study warns against the risks of hypoglycemia from methadone but does not suggest its use as a hypoglycemic drug, because methadone is a strong opioid with known risks that limit its clinical use.

A recent study by Bathina S et al., [Bathina S et al., BDNF protects pancreatic β cells (RIN5F) against cytotoxic action of alloxan, streptozotocin, doxorubicin and benzo(a)pyrene in vitro. Metabolism. 2016 May; 65(5):667-84], suggests that BDNF has potent cytoprotective actions, restores anti-oxidant defenses to normal and thus, prevents apoptosis and preserves insulin secreting capacity of pancreatic 3 cells. In addition, BDNF enhanced viability of RIN 5F in vitro. Thus, BDNF not only has anti-diabetic actions but also preserves pancreatic β cells integrity and enhances their viability. These results imply that BDNF functions as an endogenous cytoprotective molecule that may explain its beneficial actions in some neurological conditions as well.

Furthermore, the metabolic syndrome and its individual features (increased blood pressure, high blood sugar, excess body fat, and abnormal cholesterol or triglyceride levels) may also be treated by a drug like d-methadone which can up regulate testosterone and BDNF. Testosterone, aside from the known effects on sexual drive and function, has been shown to reverse the main features of the metabolic syndrome. With a quarter of the American adult population affected, the metabolic syndrome and type 2 diabetes mellitus have been referred to as the most significant public health threats of the 21st century. The risk benefit of testosterone supplementation is not clearly established (Kovac J R et al., Testosterone supplementation therapy in the treatment of patients with metabolic syndrome. Postgrad Med. 2014 November; 126(7):149-56). A recent meta-analysis supports the view of a positive effect of testosterone on body composition and on glucose and lipid metabolism. In addition, a significant effect on body composition was observed, suggesting a role for testosterone supplementation in the treatment and prevention of obesity (Corona G et al. Testosterone supplementation and body composition: results from a meta-analysis of observational studies. J Endocrinol Invest. 2016 September; 39(9):967-81). Aside the metabolic syndrome, the up-regulation of testosterone/BDNF from d-methadone may also improve other medical complications of aging and its symptoms and manifestations, such as sarcopenia, osteoporosis, impaired physical endurance and anemia. Sarcopenia is clinically defined as a loss of muscle mass coupled with functional deterioration (either walking speed or distance or grip strength). As sarcopenia is a major predictor of frailty, hip fracture, disability, and mortality in older persons, the development of drugs to prevent it and treat it is eagerly awaited (Morley J E. Pharmacologic Options for the Treatment of Sarcopenia. Calcif Tissue Int. 2016 April; 98(4):319-3). Of note, in addition to possible benefits from testosterone and BDNF up-regulation, the modulating effects of d-methadone on K⁺ currents might provide therapeutic actions for improving muscle wasting [Wulff H et al., Voltage-gated potassium channels as therapeutic targets. Nat Rev Drug Discov. 2009 December; 8(12): 982-1001]. Osteoporosis and the metabolic syndrome may also be treated by a drug like d-methadone which up regulates testosterone and BDNF. As exogenous testosterone replacement therapy carries potential risks (Gabrielsen J S et al., Trends in Testosterone Prescription and Public Health Concerns. Urol Clin North Am. 2016 May; 43(2):261-71), a drug like d-methadone that up-regulates levels of endogenous testosterone and BDNF is likely to be beneficial without the side effects and risks of exogenous testosterone.

Restless Leg Syndrome

Restless leg syndrome (RLS) is a rest-induced, movement-responsive, mostly nocturnal, urge to move the legs commonly associated with periodic leg movements during sleep. Sleep disruption is the primary factor producing most of the morbidity of moderate to severe RLS. While the dopaminergic system has been primarily implicated in the pathophysiology of this syndrome, abnormalities in the glutaminergic system have also been implicated (Allen, R. P. et al., Thalamic glutamate/glutamine in restless legs syndrome. Neurology 2013; 80:2028-2034).

In a study by Rottach, K. G. et al., [Restless legs syndrome as side effect of second generation antidepressants. J Psychiatr Res. 2008 November; 43(1):70-5], on the role of second generation antidepressants (fluoxetine, paroxetine, citalopram, sertraline, escitalopram, venlafaxine, duloxetine, reboxetine, and mirtazapine), only reboxetine, a selective NE reuptake inhibitor, did not trigger or worsen RSL.

Interestingly, methadone is a second line, off label, non-FDA approved, treatment for restless leg syndrome (Ondo W G1. Methadone for refractory restless legs syndrome. Mov Disord. 2005 March; 20(3):345-8. Trenkwalder, C. et al., Treatment of restless legs syndrome: an evidence-based review and implications for clinical practice. Mov Disord. 2008 Dec. 15; 23(16):2267-302). d-Methadone, which combines modulating activity at the NMDA and NET and SERT systems and potentially increase BDNF levels but is devoid of opioid activity may be as effective or more effective than methadone, without the opioid risks and side effects, as shown by the inventors in two novel phase 1 trials detailed in the Examples section.

Insomnia, Sleep, Arousal Sleep Disturbances-Parasomnias

Memantine was recently found to improve sleep in patients with Alzheimer's disease [Ishikawa, I. et al., The effect of memantine on sleep architecture and psychiatric symptoms in patients with Alzheimer's disease. Acta Neuropsychiatr. 2016 June; 28(3):157-64]. Further, substance abuse is associated with sleep disorders. Methadone is a strong opioid used to treat patients with opioid use disorder. Compared to patients treated with opium, patients treated with methadone were found to have improved sleep, suggesting a role of methadone in mitigating sleep problems [Khazaie, H. et al. Sleep Disorders in Methadone Maintenance Treatment Volunteers and Opium-dependent Patients. 2016 April; 8(2):84-89]; and other researchers also found improved sleep in patients switched to methadone from other opioids [DeConno F et al., Clinical experience with oral methadone administration in the treatment of pain in 196 advanced cancer patients. C. J Clin Oncol. 1996 October; 14 (10):2836-42].

Based on their own experimental and clinical research, the inventors postulate that this favorable activity of racemic methadone on sleep disorders may not be inherent to methadone (opioid use is in fact known to be associated with sleep disturbances) but might instead apply to d-methadone. While methadone, because of its known opioid effects, which may include sleep disruption, should not be used for sleep disorders, a drug like d-methadone, which retains NMDA and NE modulating activities and, as detailed by the inventors in the Examples, increases BDNF levels but is devoid of opioid activity, may be useful for sleep disorders. And so, the sleep improvement effect, attributed by De Conno et al. to the opioid effects of methadone, according to the work by the inventors is instead potentially due to the NMDA and NE balancing activities inherent to d-methadone, shown by the Inventors to be free of opioid effects. NMDA and NET systems and BDNF all potentially play a role in the pathophysiology of sleep disorders.

Stroke, and Traumatic and Inflammatory Brain Injury, Including Infectious and Autoimmune Brain Injury

It is known that excessive activation of NMDA glutamate receptors contributes to neuronal death after acute injury of different etiology, including infection, trauma and stroke. (Wang Y et al., Network-Based Approach to Identify Potential Targets and Drugs that Promote Neuroprotection and Neurorepair in Acute Ischemic Stroke, Nature Scientific Reports, January 2017; Martin, H. G. S., et al., Blocking the Deadly Effects of the NMDA Receptor in Stroke. Cell 140, Jan. 22, 2010). Memantine has been reported to enhance recovery from stroke [López-Valdés, H. E. et al. Memantine enhances recovery from stroke. Stroke. 2014 July; 45(7):2093-2100].

Further, BDNF plays important roles in brain plasticity and repair, and it influences stroke outcomes in animal models. Circulating BDNF concentrations are lowered in patients with traumatic brain injury, and low BDNF predicts poor recovery after this injury. Circulating concentrations of BDNF protein are lowered in the acute phase of ischemic stroke, and low levels are associated with poor long-term functional outcome [Stanne, T. M. et al., Low Circulating Acute Brain-Derived Neurotrophic Factor Levels Are Associated With Poor Long-Term Functional Outcome After Ischemic Stroke. Stroke. 2016 July; 47(7):1943-5].

Thus, d-methadone, by reducing excitotoxic damage and increasing BDNF levels, as discovered by the inventors, may help not only for recovering from the cognitive impairment that often follows one or more strokes and traumatic and inflammatory brain injury but it may also curtail neuronal damage during acute stroke and traumatic and inflammatory brain injury.

(NMDAR) Encephalitis

Memantine has been found to hasten recovery from anti-N-methyl-D-aspartate receptor (NMDAR) encephalitis. This rare encephalitis is caused by anti-NMDAR autoantibodies. Excitotoxicity and NMDAR dysfunction play the central roles of anti-NMDAR encephalitis, causing symptoms that range from psychosis to involuntary movements, consciousness disturbance, and dysautonomia. A drug like d-methadone, which combines modulating activity at the NMDA and NET, and potentially increase BDNF levels, but is devoid of opioid activity, may be as effective or more effective than memantine.

Seizures, Epilepsy and Developmental Disorders

A substantial amount of research has shown that NMDA receptors may play a key role in the pathophysiology of several neurological diseases, including epilepsy of different etiology. Animal models of epilepsy and clinical studies demonstrate that NMDA receptor activity and expression can be altered in association with epilepsy and particularly in some specific seizure types. Mutations in the NMDA receptors have been associated with several childhood-onset epilepsy syndromes/developmental disorders including those within the epilepsy-aphasia spectrum. These syndromes include benign epilepsy with centro-temporal spikes (BECTS), Landau-Kleffner syndrome (LKS), and epileptic encephalopathy with continuous-spike- and waves-during-slow-wave-sleep (CSWSS). Furthermore, other mutations extend the range of phenotypes beyond disorders in the epilepsy-aphasia spectrum to include early-onset epileptic encephalopathy, which is characterized by severe infantile-onset epilepsy and lack of development. Rare epilepsies and developmental disorders, including those associated with Dravet Syndrome, Lennox-Gastaut Syndrome, Tuberous Sclerosis Complex, and those within the epilepsy-aphasia spectrum might be helped by NMDA receptor antagonists [Hani, A. J. et al. Genetics of pediatric epilepsy. Pediatr Clin North Am. 2015 June; 62(3):703-22; Tyler, M. P. et al., GRIN2A mutation and early-onset epileptic encephalopathy: personalized therapy with memantine. Annals of Clinical and Translational Neurology 2014; 1(3):190-198], in particular memantine, as it has been shown to improve seizure control by Tyler et al., 2014.

NMDA receptor antagonists have been shown to have antiepileptic effects in both clinical and preclinical studies [Ghasemi, M. et al., The NMDA receptor complex as a therapeutic target in epilepsy: a review. Epilepsy Behav. 2011 December; 22(4): 617-40]. An experimental model has shown that memantine can prevent cognitive impairment after status epilepticus (Kalemenev S V et al., Memantine attenuates cognitive impairments after status epilepticus induced in a lithium-pilocarpine model. Dokl Biol Sci. 2016 September; 470(1):224-227). Berman, E. F. et al., [Opioids reduce tonic component of seizures, not naloxone dependent mechanism: The anticonvulsant effect of opioids and opioid peptides against maximal electroshock seizures in rats. Neuropharmacology. 1984 March; 23(3):367-71], observed that methadone, among other opioids, not only influences the threshold to a seizure [Cowan, A. et al., Differential effects of opioids on flurothyl seizure thresholds in rats. NIDA Res Monogr 1979; 27:198-204] but decrease the tonic component of seizures. Of note, memantine significantly improved cognitive impairment in epilepsy patients [Marimuthu, P. et al., Evaluating the efficacy of memantine on improving cognitive functions in epileptic patients receiving anti-epileptic drugs: A double-blind placebo-controlled clinical trial (Phase IIIb pilot study). Ann Indian Acad Neurol. 2016 July-September; 19(3): 344-50].

Testosterone can have anti-seizure activity and testosterone-derived 3alpha-androstanediol has been shown to be an endogenous protective neurosteroid in the brain [Reddy D S. Anticonvulsant activity of the testosterone-derived neurosteroid 3alpha-androstanediol. Neuroreport. 2004 Mar. 1; 15(3):515-8]. Testosterone may reduce seizures in men with epilepsy [Herzog A G. Psychoneuroendocrine aspects of temporolimbic epilepsy. Part II: Epilepsy and reproductive steroids. Psychosomatics. 1999 March-April; 40(2): 102-8]. Up-regulation of testosterone may decrease seizure frequency in epileptic patients [Taubøll E et al., Interactions between hormones and epilepsy. Seizure. 2015 May; 28:3-11. Frye C A. Effects and mechanisms of progestogens and androgens in ictal activity. Epilepsia. 2010 July; 51 Suppl 3:135-40].

The inventors studied the in vitro effects d-methadone in comparison to memantine in screen patch assays, which will be described in greater detail below in the Examples. The antagonistic effects of d-methadone on the electrophysiological response of human cloned NMDA NR1/NR2 A and NR1/NR2 B receptors expressed in HEK293 cells were proven to be in in the low μM range, and therefore potentially exert clinical effects and possibly neuroprotection in humans.

This study, presented by the inventors in the Examples section, confirms the potential of d-methadone for the treatment of seizures and epilepsy, including developmental and seizure disorders that are associated with mutations of genes coding for subunits of NMDA receptors.

Thus, a drug like d-methadone, which combines modulating activity at the NMDA and NET, and potentially increase BDNF and testosterone levels, and regulates K⁺, Ca⁺ and Na⁺ cellular currents but is devoid of opioid activity, may be as effective or more effective than memantine or methadone in preventing or shortening seizures of different etiologies, including seizures of epileptic syndromes. Finally, as described throughout the application, d-methadone could be useful in preventing or treating cognitive impairment, including therefore cognitive impairment caused by repeated or prolonged seizures (including seizure mediated excitotoxicity), and cognitive impairment associated with seizure disorders and their treatment, alone or with other anti-epileptics or other NMDA antagonists, without opioid risks and side effects or ketamine-like psychotomimetic effects.

Tourette's Disorder and Obsessive Compulsive Disorder and Self-Injurious Behaviors

There are indications that the NMDA receptor system and the NET may be implicated in the pathogenesis of Tourette's syndrome (TS) and obsessive-compulsive disorder (OCD) and OCD related disorders such as self-injurious behaviors like trichotillomania, dermotillomania, nail biting. A study by Liu, S. et al, [Do obsessive-compulsive disorder and Tourette syndrome share a common susceptibility gene? An association study of the BDNF Val66Met polymorphism in the Chinese Han population. World J Biol Psychiatry. 2015; 16(8):602-9], supports the involvement of the BDNF Val66Met polymorphism as a common genetic susceptibility for OCD and Tourette's syndrome. There have been reports about the use of atypical opioids including methadone, for the treatment of these disorders [Meuldijk, R. et al., Methadone treatment of Tourette's disorder. Am J Psychiatry. 1992 January; 149(1):139-40; Rojas-Corrales, M. O. et al., Role of atypical opiates in OCD. Experimental approach through the study of 5-HT(2A/C) receptor-mediated behavior. Psychopharmacology (Berl). 2007 February; 190(2):221-31]. Aside from TS and OCD, NMDAR antagonists may be useful for the treatment of self-injurious behaviors including trichotillomania, dermotillomania, excoriation disorder and nail biting [Grados, M et al., A selective review of glutamate pharmacological therapy in obsessive—compulsive and related disorders. Psychol Res Behav Manag. 2015; 8: 115-131; Muehlmann A M, Devine D P. Glutamate-mediated neuroplasticity in an animal model of self-injurious behaviour. Behav Brain Res. 2008 May 16; 189(1):32-40]. Self-injurious behaviors may occur as isolated manifestations but also occur as part of syndromes and diseases such as Lesch-Nyhan, Prader-Willi and Rett syndromes, which also could be improved by a drug like d-methadone, as detailed in different sections of this application.

However, opioids have well known risks and side effects and therefore are unlikely candidates for the treatment of these disorders. Furthermore, the opioid activity may itself be detrimental to these disorders. Thus, a drug like d-methadone, which combines NMDA antagonistic activity and NE and serotonin re-uptake inhibition and potentially increases BDNF levels, but is devoid of opioid activity, and is safe and well tolerated, may offer unique advantages for the treatment of these NS disorders and their symptoms.

Multiple Sclerosis

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and psychiatric problems. Specific symptoms include double vision, blindness, imbalance, muscle weakness, impaired sensation and coordination. Between attacks, symptoms may disappear completely, however, permanent neurological problems often remain, especially as the disease advances [Compston, A. et al., “Multiple sclerosis”. (April 2002) Lancet. 359(9313):1221-31].

BDNF may improve axonal and oligodendroglial deficits that occur as a result of demyelinating lesions in Multiple Sclerosis [Huang, Y. et al., The role of growth factors as a therapeutic approach to demyelinating disease. Exp Neurol. 2016 September; 283(Pt B):531-40]. Cognitive dysfunction has been associated with decreased BDNF in patients with MS [Prokopova, B. et al., Early cognitive impairment along with decreased stress-induced BDNF in male and female patients with newly diagnosed multiple sclerosis. J Neuroimmunol. 2017 Jan. 15; 302:34-40].

Thus, a drug like d-methadone, which combines NMDA antagonistic activity and NE and serotonin re-uptake inhibition and potentially increase BDNF levels, but is devoid of opioid activity, and is safe and well tolerated, may offer unique advantages for the treatment and MS and its neurological symptoms and manifestations and diseases such as acute encephalitis, encephalomyelitis, optic neuritis, neuromyelitis optica spectrum disorders and transverse myelitis. Of note in addition to possible benefits from the mechanisms described above, the modulating effects of d-methadone on K⁺ currents might provide additional actions for improving multiple sclerosis (Wulff H et al., Voltage-gated potassium channels as therapeutic targets. Nat Rev Drug Discov. 2009 December; 8(12): 982-1001).

Amyotrophic Lateral Sclerosis

Amyotrophic lateral sclerosis (ALS) is a devastating neurodegenerative disease that results in progressive loss of motoneurons, motor weakness and death usually within 3-5 years after disease onset. Therapeutic options remain limited. Thus far, only two drugs are FDA approves for the treatment of ALS. The first drug, riluzole, a drug that preferentially blocks TTX-sensitive sodium channels, possibly preventing excitotoxicity by different postulated mechanisms [Doble. The pharmacology and mechanism of action of riluzole. Neurology. 1996 December; 47(6 Suppl 4):S233-41]. The second drug, edavarone, is a free radical scavenger and was shown to play a role in the treatment of ALS (Abe, Koji et al. Confirmatory Double-Blind, Parallel-Group, Placebo-Controlled Study of Efficacy and Safety of Edaravone (MCI-186) in Amyotrophic Lateral Sclerosis Patients.” Amyotrophic Lateral Sclerosis & Frontotemporal Degeneration 15.7-8 (2014): 610-617). Edaravone was approved by the FDA in May 2017, 22 years after the approval of riluzole (Traynor K. FDA approves edaravone for amyotrophic lateral sclerosis. Am J Health Syst Pharm. 2017 Jun. 15; 74(12):868). Both approve drugs have shown only a modest disease-modifying efficacy. Treatments with better efficacy are needed. Neurotrophic growth factors are known to promote the survival of neurons and foster regeneration in the central nervous system and there is renewed hope for their efficacy for ALS (Henriques, A. et al. Neurotrophic growth factors for the treatment of amyotrophic lateral sclerosis: where do we stand? Frontiers in Neuroscience, June 2010 Vol 4 Art 32). There is also some evidence to support the hypothesis that β2-agonists may be efficacious in ALS [Bartus, R. T. et al., β2-Adrenoceptor agonists as novel, safe and potentially effective therapies for Amyotrophic lateral sclerosis (ALS) Neurobiology of Disease 85 (2016) 11-24]. More importantly, glutamate-induced excitotoxicity has lain at the core of theories behind the spiraling events, including mitochondrial dysfunction, oxidative stress, and protein aggregation, that lead to neurodegenerative cell death in ALS (Blasco H et al., The glutamate hypothesis in ALS: pathophysiology and drug development. Curr Med Chem. 2014; 21(31):3551-75).

A novel drug like d-methadone, which combines NMDA antagonistic activity thus regulating the glutamate pathways, potentially preventing excitotoxicity while increasing BDNF levels, and regulating NE re-uptake and is safe and well tolerated, as shown by the inventors in the Examples section, may offer unique advantages for the treatment of ALS. d-Methadone might show effectiveness for ALS either alone or in combination with riluzole or edavarone.

Huntington's Disease

Huntington's disease (HD) is a fatal progressive neurodegenerative disorder with autosomal dominant inheritance. In humans mutated huntingtin (htt) induces a preferential loss of medium spiny neurons (MSN) of the striatum and causes motor, cognitive and emotional deficits. One of the proposed cellular mechanisms underlying medium spiny neurons degeneration is excitotoxic pathways mediated by glutamate receptors (Anitha M et al., Targeting glutamate mediated excitotoxicity in Huntington's disease: neural progenitors and partial glutamate antagonist—memantine. Med Hypotheses. 2011 January; 76(1):138-40). A drug like d-methadone that blocks the hyperactive NMDA open ion channels has the potential to prevent excess calcium influx into the neurons and decrease the vulnerability of medium spiny neurons to glutamate mediated excitotoxicity. Further, neurotrophic growth factors are known to promote the survival of neurons and foster regeneration in the central nervous system.

Thus, a drug like d-methadone, which combines NMDA antagonistic activity thus regulating the glutamate pathways, and NE re-uptake inhibition and potentially increases BDNF levels, but is devoid of opioid activity, and is safe and well tolerated, may offer unique advantages for the treatment of Huntington's disease and its manifestations.

Mitochondrial Disorders

The NS is often affected in mitochondrial disorders, particularly in respiratory chain diseases (RCDs). NS manifestations of RCDs comprise stroke-like episodes, epilepsy, migraine, ataxia, spasticity, movement disorders, neuropathy, psychiatric disorders, cognitive decline, pathology of the retina, and even dementia (mitochondrial dementia). In particular, mitochondrial dementia has been reported in MELAS, MERRF, LHON, CPEO, KSS, MNGIE, NARP, Leigh syndrome, and Alpers-Huttenlocher disease. Friedreich's ataxia is an autosomal recessive disorder that occurs when the FXN gene contains amplified intronic GAA resulting in a deficiency in the protein frataxin and mitochondrial dysfunction. Therapy of mitochondrial diseases is limited to symptom management and prevention of further mitochondrial malfunction.

Furthermore, disruptions in mitochondrial function may play a critical role in pathophysiology of CNS illness: NMDA driven behavioral, synaptic, and brain oscillatory functions were found to be impaired in UCP2 knockout mice [Hermes, G. et al., Role of mitochondrial uncoupling protein-2 (UCP2) in higher brain functions, neuronal plasticity and network oscillation. Mol Metab. 2016 Apr. 9; 5(6):415-21]. Chronic NMDA administration causes mitochondrial dysfunction in rats [Kim, H. K. et al., Mitochondrial dysfunction and lipid peroxidation in rat frontal cortex by chronic NMDA administration can be partially prevented by lithium treatment. J Psychiatr Res. 2016 May; 76:59-65]. Excessive extracellular glutamate leads to uncontrolled continuous depolarization of neurons, a toxic process known as excitotoxicity. In regard to excitotoxicity, NMDARs play the most important role as larger quantities of Ca²⁺ ions can be moved through them. This abnormally elevated Ca²⁺ intracellular concentration causes mitochondrial dysfunction [Kritis, A. A. et al., Researching glutamate-induced cytotoxicity in different cell lines: a comparative/collective analysis/study. Front Cell Neurosci. 2015 Mar. 17; 9:91; Prentice, H. et al., Mechanisms of Neuronal Protection against Excitotoxicity, Endoplasmic Reticulum Stress, and Mitochondrial Dysfunction in Stroke and Neurodegenerative Diseases. Oxid Med Cell Longev. 2015; Dunchen, M. R., Mitochondria, calcium-dependent neuronal death and neurodegenerative disease. Pflugers Arch. 2012: 464(1):111-121]. Direct exposure to N-methyl-d-aspartate alters mitochondrial function [Korde, A. S. et al., Direct exposure to N-methyl-d-aspartate alters mitochondrial function. Neurosci Lett. 2016 Jun. 3; 623:47-51].

Mitochondrial diseases may become clinically apparent once the number of affected mitochondria reaches a certain level; this phenomenon is called “threshold expression”. Mitochondrial Ca²⁺ accumulation leading to mitochondrial malfunction is a key event in glutamate excitotoxicity. Cells maintained by glycolysis in the absence of a mitochondrial membrane potential are highly resistant to glutamate excitotoxicity because they do not take up Ca2⁺ into mitochondria [Nicholls, D. G. et al., Neuronal excitotoxicity: the role of mitochondria. Biofactors. 1998; 8(3-4):287-99]. Excitotoxic injury has been postulated as a concurrent pathogenic factor in Leber Hereditary Optic Neuropathy (Howell N. Leber hereditary optic neuropathy: respiratory chain dysfunction and degeneration of the optic nerve. 1988 Vis Res 38:1495-1504) and in Leigh disease (Lake N J et al., Leigh syndrome: neuropathology and pathogenesis. J Neuropathol Exp Neurol. 2015 June; 74(6):482-92).

Cognitive impairment is also a feature of Duchenne muscular dystrophy. While not a primarily mitochondrial disease, mitochondria are affected in Duchenne muscular dystrophy and, as described throughout this section, d-methadone potentially prevents mitochondrial dysfunction and thus could ameliorate signs and symptoms of this disease.

Safe and effective treatments for mitochondrial diseases are lacking. Only single patients benefit from cholinesterase inhibitors or memantine, antioxidants, vitamins, coenzyme-Q, or other substitutes [Finsterer, J., Mitochondrial disorders, cognitive impairment and dementia. J Neurol Sci. 2009 Aug. 15; 283(1-2):143-8].

A novel drug like d-methadone, which combines NMDA antagonistic activity thus regulating the glutamate pathways and potentially protecting mitochondria from excitotoxicity, and NE and serotonin re-uptake inhibition and potentially increases BDNF levels, and regulates K+, Ca⁺ and Na cellular currents but is devoid of clinically significant opioid activity and psychotomimetic side effects, and is safe and well tolerated, may offer unique advantages that affect mitochondria and for their symptoms and manifestations and may slow their progression, alone or in combination with cholinesterase inhibitors, antioxidants, vitamins, idebenone, coenzyme-Q or other substitutes, memantine or other NMDAR blockers.

Fragile X Syndrome and Fragile X-Associated Tremor/Ataxia Syndrome (FXTAS)

Cellular neuropathological studies have demonstrated abnormal neuronal response to glutamate in the fragile X gene (FMR1) premutation. In human induced pluripotent stem cell (iPSC)-derived neurons carrying the premutation, Liu and colleagues documented an increased response to glutamate, and higher amplitude and more frequent calcium spiking activity [Liu, J. et al., Signaling Defects in iPSC-Derived Fragile X Premutation Neurons. Hum Mol Genet (2012) 21, 3795-3805].

Memantine was found to benefit attentional processes that represent fundamental components of executive function/dysfunction, thought to comprise the core cognitive deficit in Fragile X-associated tremor/ataxia syndrome (FXTAS) [Yang, J. C. et al., Memantine Improves Attentional Processes in Fragile X-Associated Tremor/Ataxia Syndrome: Electrophysiological Evidence from a Randomized Controlled Trial. Sci Rep. 2016; 6: 217-19]. The FMRP is implicated in glutamergic pathways that control neural plasticity, including the mechanisms of learning and memory (McLennan Y et al., Fragile X Syndrome. Curr Genomics. 2011 May; 12(3): 216-224). A drug like d-methadone, now shown by the inventors to improve cognitive function without psychotomimetic or opioid effects and to have NMDAR affinities in the micro molar range similar to memantine, and to exert behavioral actions similar to ketamine in experiments presented in the Examples section of this application, and to potentially increase serum BDNF levels, thereby influencing neural plasticity, is likely to prevent the worsening of many neurological conditions where glutamate excitotoxicity plays a role including neurodevelopmental disorders, including fragile X syndrome, Rett syndrome, Prader Willi syndrome, Angelman syndrome and their neurological symptoms and manifestations, including obesity.

Interestingly, while FMRP deficiency is the cause of Fragile X syndrome, one report shows a deficiency of FMRP in the brains of individuals with neuropsychiatric disorders that do not have an FMR1 mutation. Post-mortem brain tissue from the lateral cerebella of controls compared to subjects with psychiatric disorders revealed FMRP was reduced by 78% in the brains of those with schizophrenia as compared to control brains, suggesting further evidence for d-methadone effectiveness for this indication. (Napoli I. et al., The fragile X syndrome protein represses activity-dependent translation through CYFIP1, a new 4E-BP. Cell, 2008, 134 (6), 1042-1054).

Angelman Syndrome

Angelman syndrome is a neurogenetic disorder characterized by developmental delay, severe intellectual disability, absent speech, exuberant behavior with happy demeanor, motor impairment, and epilepsy, due to deficient UBE3A gene expression that may be caused by various abnormalities of chromosome 15. The NMDA mediated synaptic transmission appears to be altered in Angelman syndrome and this abnormality is likely to contribute to the symptoms of this syndrome (Dan B. Angelman syndrome: Current understanding and research prospects. Epilepsia, 2009 50: 2331-2339.). Some or all of its symptoms may be ameliorated by a drug like d-methadone, now shown by the inventors to improve cognitive function without psychotomimetic or opioid effects and to have NMDAR affinities in the micro molar range similar to memantine, and to potentially increase serum BDNF levels; d-methadone is likely to prevent the worsening of many neurological conditions where glutamate excitotoxicity plays a role, including Angelman syndrome its neurological symptoms and manifestations.

The Hereditary Ataxias, Including Friedreich's Ataxia, Olivopontocerebellar Atrophies and their Neurological Symptoms and Manifestations, and Vestibular Disorders and Nystagmus. Stiff Person Syndrome.

Friedreich's ataxia is an autosomal recessive disorder that occurs when the FXN gene contains amplified intronic GAA resulting in a deficiency in the protein frataxin and mitochondrial dysfunction. Memantine was found to be a potential treatment for acute optic nerve atrophy in Friedreich's ataxia [Peter, S. et al., Memantine for optic nerve atrophy in Friedreich's Ataxia. Article in German. Ophthalmologe. 2016 August; 113(8):704-7]. lizuka, A. et al., [Long-term oral administration of the NMDA receptor antagonist memantine extends life span in spinocerebellar ataxia type 1 knock-in mice. Neurosci Lett. 2015 Apr. 10; 592:37-41] describe the contribution of aberrant activation of extrasynaptic NMDARs to neuronal cell death in spinocerebellar ataxia type 1SCA1 KI mice. In KI mice, the exon in the ataxin 1 gene is replaced with abnormally expanded 154CAG repeats. Memantine was administered orally to the SCA1 KI mice from 4 weeks of age until death. The treatment significantly attenuated body-weight loss and prolonged the life span of SCA1 KI mice. Furthermore, memantine significantly suppressed the loss of Purkinje cells in the cerebellum and motor neurons in the dorsal motor nucleus of the vagus, which are critical for motor function and parasympathetic function, respectively.

These results suggest that memantine may also have therapeutic benefits in human SCA1 patients. According to Rosini, F. et al., [Ocular-motor profile and effects of memantine in a familial form of adult cerebellar ataxia with slow saccades and square wave saccadic intrusions]. PLoS One. 2013 Jul. 22; 8(7)], memantine was found to decrease macrosaccadic oscillations (MSO) and improve fixation in patients with spinocerebellar ataxia with saccadic intrusions (SCASI) and other forms of hereditary ataxias: memantine may have some general suppressive effect on saccadic intrusions, including both square wave intrusions (SWI) and MSO, thereby restoring the capacity of reading and visual attention in these and in other recessive forms of ataxia, including Friedreich's, in which saccadic intrusions are prominent.

Spinocerebellar ataxia type 2 (SCA2) and type 3 (SCA3) are autosomal-dominant neurodegenerative disorders. SCA2 primarily affects cerebellar Purkinje neurons. SCA3 primarily affects dentate and pontine nuclei and substantia nigra. Both disorders belong to a class of polyglutamine (polyQ) expansion disorders. SCA2 is caused by a polyQ expansion in the amino-terminal region of a cytosolic protein ataxin-2 (Atxn2). SCA3 is caused by a polyQ expansion in the carboxy-terminal portion of a cytosolic protein ataxin-3 (Atxn3). Both disorders are found worldwide and no effective treatments exist for SCA2, SCA3 or any other polyQ-expansion disorder.

Recent preclinical studies in SCA2 and SCA3 genetic mouse model suggested that abnormal neuronal calcium (Ca²⁺) signaling may play an important role in SCA2 and SCA3 pathology. These studies also suggested Ca²⁺ signaling inhibitors and stabilizers like memantine, and thus potentially d-methadone, may have a therapeutic value for treatment of SCA2 and SCA3 (Bezprozvanny I and Klockgether T. Therapeutic prospects for spinocerebellar ataxia type 2 and 3. Drugs Future. 2009 December; 34(12). Botez et al., 1996, describe the rationale of amantadine and memantine use in olivopontocerebellar atrophy and other heredodegenerative ataxias by direct involvement of N-methyl-D-aspartate (NMDA) in glutamate mediated neurotoxicity in cerebellar granular cells (Botez M I et al., Amantadine hydrochloride treatment in heredodegenerative ataxias: a double blind study. J Neurol Neurosurg Psychiatry. 1996 September; 61(3):259-64).

Antibodies directed against glutamic acid decarboxylase (GAD) are present in many patients with stiff person syndrome and are also increasingly found in patients with other symptoms indicative of central nervous system (CNS) dysfunction, such as ataxia, progressive encephalomyelitis with rigidity and myoclonus (PERM), limbic encephalitis, and even epilepsy. It is presumed that antibodies directed against GAD impair GABA production, but the precise pathogenic mechanism of GAD-antibody-related neurologic disorders is uncertain [Dayalu P and Teener J W. Stiff Person syndrome and other anti-GAD-associated neurologic disorders. Semin Neurol. 2012 November; 32(5):544-9]. Excessive or unbalanced glutamate stimulation could also contribute to these disorders. Few patients respond to treatment with immunomodulating therapy and symptomatic agents that enhance GABA activity, such as benzodiazepines and baclofen, provide some help.

Additionally, NMDA antagonists and memantine may improve vestibular disorders and nystagmus including pendular and infantile nystagmus, Meniére's disease, vestibular paroxysmia, vestibular migraine [Strupp, M. et al., Pharmacotherapy of vestibular disorders and nystagmus. Semin Neurol. 2013 July; 33(3):286-96].

A novel drug like d-methadone, now shown by the inventors to improve cognitive function without psychotomimetic or opioid effects and to have NMDAR affinities in the micromolar range similar to memantine, and to potentially increase serum BDNF levels, is likely to prevent the worsening of many neurological conditions where glutamate excitotoxicity plays a role, including the hereditary ataxias, including Friedreich's ataxia, olivopontocerebellar atrophies and their neurological symptoms and manifestations, acute optic nerve atrophy and vestibular disorders and nystagmus including pendular and infantile nystagmus, Meniére's disease, vestibular paroxysmia, vestibular migraine, and stiff person syndrome and other neurological disorders associated with GAD antibodies.

Neurodegenerative, Neurodevelopmental and Inflammatory Diseases of the Retina Like Glaucoma, Diabetic Retinopathy, Age-Related Macular Degeneration, Retinitis Pigmentosa, Optic Neuritis and LHON. Diseases and Symptoms of the Anterior Segment of the Eye, Including Dry Eye Syndrome.

In diseases of the retina such as glaucoma, diabetic retinopathy and age-related macular degeneration, during metabolic stress, glutamate is released, initiating dysfunction and death of neurons containing ionotropic NMDA receptors, such retinal ganglion cells and a specific type of amacrine cells. The major causes for cell death following activation of NMDA receptors is the influx of calcium into cells, the generation of free radicals linked to the formation of advanced glycation endproducts (AGEs) and/or advanced lipoxidation endproducts (ALEs), as well as defects in the mitochondrial respiratory chain. Macular edema represents the end-stage of multiple pathophysiological pathways in a multitude of vascular, inflammatory, metabolic and other diseases; novel treatments, such as neuroprotective agents, like nerve growth factors and NMDA antagonists, may inhibit neuronal cell death in the retina [Wolfensberger T J. Macular Edema—Rationale for Therapy. Dev Ophthalmol. 2017; 58:74-86]. NMDA induced nerve cell damage can occur in glaucoma and optic neuritis. Memantine, an NMDA antagonist shown by the inventors to have affinity for NMDAR blockage in the micromolar range similarly to d-methadone, has been found to potentially benefit glaucoma in experimental studies [Celiker H et al., Neuroprotective Effects of Memantine in the Retina of Glaucomatous Rats: An Electron Microscopic Study. J Ophthalmic Vis Res. 2016 April-June; 11(2):174-82]; the authors concluded that when started in the early phase of glaucomatous process, memantine may help to preserve the retinal ultrastructure and thus prevent neuronal injury in experimentally induced glaucoma. Memantine was also found to be effective in reduction of retinal nerve fiber layer (RNFL) thinning in patients with optic neuritis (Esfahani M R et al., Memantine for axonal loss of optic neuritis. Graefes Arch Clin Exp Ophthalmol. 2012 June; 250(6):863-9), although it did not improve vision.

Substances preventing excitocytotoxic events are considered to be potentially neuroprotective. Experimental studies demonstrate that several drugs reduce or prevent the death of retinal neurons deficient of nutrients. These agents generally block NMDA receptors to prevent excessive action of glutamate and halt the subsequent pathophysiologic cycle resulting in cell death [Schmidt K G et al., Neurodegenerative diseases of the retina and potential for protection and recovery. Curr Neuropharmacol. 2008 June; 6(2):164-78]. Glutamate induced optic atrophy has also been found to be associated with alterations in BDNF expression [Ito Y et al., Degenerative alterations in the visual pathway after NMDA-induced retinal damage in mice. Brain Res. 2008 May 30; 1212:89-101]. Excitotoxic injury has been postulated as a concurrent pathogenic factor in Leber Hereditary Optic Neuropathy [Howell N. Leber hereditary optic neuropathy: respiratory chain dysfunction and degeneration of the optic nerve. 1988 Vis Res 38:1495-1504; Sala G. Antioxidants Partially Restore Glutamate Transport Defect in Leber Hereditary Optic Neuropathy Cybrids. Journal of Neuroscience Research 2008 86:3331-3337]. Alterations in glutamate metabolism have been described in different models of retinitis pigmentosa; glutamate-mediated excitotoxic mechanisms were found to contribute to rod photoreceptor death in the retinal degeneration mouse model (Delyfer M N et al., Evidence for glutamate-mediated excitotoxic mechanisms during photoreceptor degeneration in the rd1 mouse retina. Mol Vis. 2005 Sep. 1; 11:688-96).

A novel drug like d-methadone, now shown by the inventors to be devoid of psychotomimetic or opioid effects and to have NMDAR affinities in the micromolar range similar to memantine, and to potentially increase serum BDNF and testosterone levels and regulate metabolic parameters, is likely to treat and prevent conditions where glutamate excitotoxicity plays a role and BDNF regulates neuronal plasticity, including diseases of the retinal ganglion cells including fotoreceptors, bipolar, ganglion, horizontal and amacrine and Muller cells and optic nerve, whether administered systemically, topically, including via eye drops or ointments, and/or intra-ocularly, including intravitreal injections, including depot formulations and via iontophoresis. As detailed in the Examples section, d-methadone increases BDNF levels. The effects of BDNF on cells of the eye, including retinal cells and corneal cells, may prevent or treat neurodegenerative, toxic, metabolic, and inflammatory diseases of the retina and the eye, in association or independently from the actions on NMDAR, including the retina and including the cornea. Furthermore, one of the major factors in progression of glaucoma and its complications is increased intraocular pressure (IOP). Opioids have been found to decrease IOP by acting on intraocular (peripheral) opioid receptors [Drago F et al., Effects of opiates and opioids on intraocular pressure of rabbits and humans. 1985 Clin Exp Pharmacol Physiol. 1985 March-April; 12(2):107-13]. While opioid agonists such as morphine have known side effects and risks, even when administered topically (up to 50% of a drug administered via eye drops is potentially absorbed intra-nasally, with rapid systemic effects, and in the case of opioidergic drugs, such as morphine, racemic methadone, l-methadone, opioid related effects), a drug like d-methadone, found by the inventors to be free of central cognitive opioid side effects and free of psychotomimetic effects, may be potentially useful to lower IOP, topically or systemically, alone or in combination with other drugs that lower IOP including prostaglandins, beta-blockers, alpha-adrenergic agonist, carbonic anhydrase inhibitors, parasympathomimetics, epinephrine, hyperosmotic agents. Dextromethorphan an opioid with NMDA antagonistic activity similar to d-methadone may also exert similar actions. However, dextromethorphan has many drawbacks, including a very short half life and an active metabolite and is subject to a CYP2D6 genetic polymorphism that results in variable pharmacokinetics and response in the population, (Zhou S F. Polymorphism of human cytochrome P450 2D6 and its clinical significance: part II. Clin Pharmacokinet. 48:761-804, 2009) clear disadvantages compared to d-methadone

In a study detailed in the Examples section, the inventors analyzed the effects of d-methadone 25 mg, 50 mg and 75 mg administered orally once a day for ten days to healthy volunteers on pupillary constriction. Overall, the mean pupillary constriction (MPC) values during the dosing period from Day 1 to Day 10 were smallest in magnitude (least constriction) for the placebo group, intermediary for the 25 mg and 50 mg d-methadone groups, and largest in magnitude (most constriction) for the 75 mg d-methadone group. The 75 mg d-methadone group exhibited the greatest mean pupil constriction at the earliest time point in the dosing period: mean (SD) MPC for the 25 mg group was −1.32 (0.553) mm on Day 9, for the 50 mg group was −1.43 (0.175) mm on Day 6, and for the 75 mg group was −2.24 (0.619) mm on Day 5. The lack of cognitive central opioid side effects at doses that cause pupillary constriction indirectly confirms that peripheral opioid receptors in the eye may be activated by d-methadone administered orally, without the central side effects of opioids; oral or topical d-methadone could therefore also be useful when pupillary constriction is advantageous without systemic opioid effects of opiodergic drugs, e.g., for glaucoma and after pupillary dilatation for eye examination purposes. D-Methadone induced miosis from oral administration, described in the phase 1 MAD study by the inventors and described in the Examples, could potentially also intervene when the drug is administered topically via eye drops, not from systemic absorption and central effects, but also from activity on peripheral opioid receptors.

Disease of the anterior segment of the eye including dry eye syndrome are increasingly becoming a generalized health concern, affecting as many 40-70% of the elderly population, with increasing prevalence with aging and in populations living in polluted urban areas. While experimental studies have found that the opioid antagonist naltrexone facilitates reepithelialization of the cornea by blocking endogenous opioids [Zagon I S et al., Naltrexone, an opioid antagonist, facilitates reepithelialization of the cornea in diabetic rat. Invest Ophthalmol Vis Sci. 2000 January; 41(1):73-81], the administration of topical morphine was found to provide analgesia without interfering with corneal healing [Peyman G A et al. Effects of morphine on corneal sensitivity and epithelial wound healing: implications for topical ophthalmic analgesia. Br J Ophthalmol. 1994 February; 78(2): 138-141].

d-Methadone, aside from preventing cellular damage from an excessive presence of glutamate (non-competitive NMDA open channel blocker), was found by the authors to increase BDNF and testosterone serum levels. The cornea has a very high density of nerve terminals, up to 7000 per square millimeter; nerve-secreted factors, such as BDNF, are crucial for epithelial regeneration [Bikbova G et al., Neuronal Changes in the Diabetic Cornea: Perspectives for Neuroprotection. Biomed Res Int. 2016; Article ID:5140823]. Loss of nerve fibers in the cornea is a major complication of diabetes and dry eye syndrome, with severe complications ranging from corneal ulceration to impairment of vision and blindness. The increase in BDNF induced by d-methadone may prevent and treat corneal denervation induced by various factors, including diabetes and dry eye syndrome. d-Methadone's effect on up-regulation of testosterone, also discovered by the inventors, may further improve the course of dry eye syndrome [Sullivan D A et al., Androgen deficiency, Meibomian gland dysfunction, and evaporative dry eye. Ann N Y Acad Sci. 2002 June; 966:211-22] and exert trophic effects on the cornea in synergy with BDNF. Furthermore, the weak activity of d-methadone on peripheral opioid receptors, aside from decreasing IOP, may provide alleviation of symptoms such as neurogenic pruritus, discomfort and local inflammation, and hyper-sensitivity, all symptoms known to be a significant burden for patients with dry eye syndrome. d-Methadone inhibition of NE and serotonin re-uptake could also improve local symptoms of dry eye syndrome and its effects on mood could ameliorate the perception of discomfort.

In summary, due to the various effects outlined above, including those on NMDAR, BDNF, testosterone, peripheral opioid receptors, IOP, d-methadone could be potentially therapeutic in many diseases of the eye and it could be administered topically, including in the form of eye drops or ointments, and via iontophoresis to increase vitreal penetration, or via intraocular injection, including as an intravitreal depot form, or it could be administered systemically for all of eye diseases and indications described above.

We have initiated formulation of an ophthalmic solution of d-methadone and we are planning studies with eye drops for ascertaining the effects of d-methadone administered topically for the relief of symptoms and manifestations of diseases of the eye.

Dermatologic Diseases and Symptoms

Through a plurality of modes of action, d-methadone has the potential for relieving skin inflammation and itching in many dermatologic diseases and conditions, such as psoriasis [Brunoni A R et al., Decreased brain-derived neurotrophic factor plasma levels in psoriasis patients. Braz J Med Biol Res. 2015 August; 48(8):711-4], vitiligo [Kuala M et al., Reduced serum brain-derived neurotrophic factor in patients with first onset vitiligo. Neuropsychiatr Dis Treat. 2014 Dec. 12; 10:2361-7] and could therefore also exert skin anti-aging and regenerating effects when administered systemically or even topically onto the skin in the form of creams, lotions, gels and ointments. Aside for its regulatory action on BDNF, d-methadone could relieve skin inflammation seen in many dermatologic diseases via opioid receptors present on keratinocytes [Slominski A T. On the Role of the Endogenous Opioid System in Regulating Epidermal Homeostasis. Journal of Investigative Dermatology. 2015; 135, 333-334] and by blocking peripheral NMDAR [Fuziwara S et al., NMDA-type glutamate receptor is associated with cutaneous barrier homeostasis. J Invest Dermatol. 2003 June; 120(6):1023-9]. Through the mechanisms outlined above, aging of the skin and cutaneous adnexa, including hair, accelerated skin aging from cancer treatment, including external radiation therapy, could also be treated by systemic or topical d-methadone.

Itching is a common symptom of skin diseases and in some circumstances can also contribute to sustain the disease process itself. d-Methadone, through its central and peripheral NMDA blocking action [Haddadi N S et al., Peripheral NMDA Receptor/NO System Blockage Inhibits Itch Responses Induced by Chloroquine in Mice. Acta Derm Venereol. 2017 May 8; 97(5):571-577] and via peripheral opioid receptor binding when administer topically (Iwaszkiewicz K S et al., Targeting peripheral opioid receptors to promote analgesic and anti-inflammatory actions. Front Pharmacol 2013; 4: 132-137), could provide relief for skin inflammation, itching and related skin pathology. Eczema and cutaneous manifestations of autoimmune disorders could thus also be improved by d-methadone administered topically or systemically.

Dyskinesias

Dyskinesias are involuntary muscle movements that occur spontaneously in Huntington's disease (HD) and after long-term treatments for Parkinson's disease (levodopa-induced dyskinesia; LID) or for schizophrenia (tardive dyskinesia, TD). Tardive dyskinesia is a syndrome of abnormal, involuntary movements, which occurs as a complication of long-term neuroleptic therapy. While the pathophysiology of dyskinesias is still incompletely elucidated, alterations in striatal enkephalinergic neurons due to excessive glutamatergic activity may be implicated.

According to a recent study (Konitsiotis S et al., Effects of N-methyl-D-aspartate receptor antagonism on neuroleptic-induced orofacial dyskinesias. Psychopharmacology (Berl). 2006 April; 185(3):369-77), NMDA receptor blockers, especially those showing selectivity for NMDA receptors containing NR2B subunit, may be particularly effective for the treatment of tardive dyskinesias.

In a study, Andreassen, O. A. et al., [Inhibition by memantine of the development of persistent oral dyskinesias induced by long-term haloperidol treatment of rats. British Journal of Phamacology. 1996; 119, 751-757] found that that long-lasting tardive dyskinesia analogue—vacuous chewing movements (VCM)-induced by haloperidol are prevented by memantine. This finding supports the theory that excessive NMDA receptor stimulation may be a mechanism underlying the development of persistent VCM in rats and therefore also TD in human subjects.

In another study [Andreassen, O. A. et al., Memantine attenuates the increase in striatal preproenkephalin mRNA expression and development of haloperidol-induced persistent oral dyskinesias in rats. Brain Res. 2003; 24; 994(2):188-92], memantine inhibited the development of haloperidol-induced persistent vacuous chewing movements (VCM) that were induced by 20 weeks of haloperidol administration.

Naidu, P. S. I. et al., [Excitatory mechanisms in neuroleptic-induced vacuous chewing movements (VCMs): possible involvement of calcium and nitric oxide. Behav. Pharmacol. 2001 June; 12(3):209-16], implicated NMDA receptor involvement in haloperidol-induced VCMs, and also suggested the possibility of targeting calcium and nitric oxide pathways, which are also regulated by NMDA antagonists.

d-Methadone, as shown by the inventors, can block hyperactive NMDA receptors and potentially prevent excess calcium influx into the neurons, mitochondrial toxicity and NO production, decreasing the vulnerability of neurons to glutamate mediated excitotoxicity and inducing BDNF production. Neurotrophic growth factors are known to promote the survival of neurons and foster regeneration in the central nervous system. A novel drug like d-methadone, which combines NMDA antagonistic activity thus regulating the glutamate pathways, and NE re-uptake inhibition and potentially increases BDNF levels, but is devoid of opioid activity, and is safe and well tolerated, may offer unique advantages for the treatment of dyskinesias and dystonias of different etiology, including dyskinesias associated with Huntington's disease, treatment of PD and schizophrenia.

Essential Tremor

Essential tremor (ET) is one of the most common movement disorders among adults and may be disabling. While the disease course is benign, its amelioration by alcohol intake may cause complications related to ethanol abuse in some patients. The drug treatment of ET remains unsatisfactory. Additional therapies are required for patients with inadequate response or intolerable side effects from currently approved treatments.

Memantine was shown to exert neuroprotective effects on cerebellar and inferior olivary neurons and have anti-tremor in an animal model (Iseri P K et al., The effect of memantine in harmaline-induced tremor and neurodegeneration. Neuropharmacology. 2011 September; 61(4):715-23).

A novel drug like d-methadone, which combines NMDA antagonistic activity thus regulating the glutamate pathways, and NE re-uptake inhibition and potentially increases BDNF levels, but is devoid of opioid activity, and is safe and well tolerated, may offer unique advantages for the treatment of essential tremor and other tremors and movement disorders.

Hearing Impairment

Sensory-neural hearing impairment is associated with the impairment of spiral ganglion neurons (SGNs). SGNs are bipolar neurons that transmit auditory information from the ear to the brain. SGNs are indispensable for the preservation of normal hearing and their survival depends mainly on genetic and environmental interactions. Noise-induced, toxic, infectious, inflammatory, and neurodegenenerative diseases involving the SGNs are possible causes of sensory-neural hearing impairment. Aside from noise exposure, other factors, genetic and environmental, such as ototoxic medication, other toxins, overuse of cellular/smart phones and genetic factors, can potentially lead to the loss of SGNs and therefore result in sensorineural hearing impairment.

One possible mechanism of damage is thought to involve glutamate excitotoxicity. NMDAR antagonists may be useful for post-exposure treatment and prevention of further damage [Imam, L. et al., Noise-induced hearing loss: a modern epidemic? Br J Hosp Med (Lond). 2017 May 2; 78(5):286-290]. It is widely accepted that glutamate is an important excitatory neurotransmitter in mammalian brains, but excessive amount of glutamate can cause “excitotoxicity” and lead to neuronal death in some injuries and diseases, such as cerebral ischemia, traumatic brain disorder, HIV, and neurodegenerative disorders. Exposure to excessive glutamate in rats results in high-frequency hearing loss. And there was a dramatic and selective reduction of neurons in the basal, high-frequency-related portion of the spiral ganglion, but no loss of hair cells was discovered. Traumatic sound exposure, aminoglycoside antibiotics, cochlea ischemia, or traumatic/infection, autoimmune diseases, all lead to an excessive release of glutamate from inner hair cells into the synaptic cleft. Glutamate excitotoxicity causes neuronal cell death primarily through the excessive activation of glutamate receptors which triggers massive Ca²⁺ influx into neurons. Ca²⁺-loaded mitochondria generate reactive oxygen species (ROS), which comprises superoxide and nitric oxide [Bai, X. I. et al., Protective Effect of Edaravone on Glutamate-Induced Neurotoxicity in Spiral Ganglion Neurons. Neural Plast 2016; 2016:4034218].

A novel drug like d-methadone shown by the inventors to have NMDAR affinities in the micromolar range similar to memantine, and to potentially increase serum BDNF levels, is likely to prevent the worsening of many neurological conditions where glutamate excitotoxicity plays a role, including prevention, treatment or attenuation of sensory-neural hearing loss. Further, d-methadone may also be useful in tinnitus, which has been found to be associated with low BDNF levels [Coskunoglu, A. et al., Evidence of associations between brain-derived neurotrophic factor (BDNF) serum levels and gene polymorphisms with tinnitus. Noise Health. 2017 May-June; 19(88):140-148].

Impaired Sense of Smell and Taste

The sense of smell (and consequently the sense of taste) can be impaired because of genetic, degenerative, toxic, infectious, neoplastic inflammatory and traumatic causes. Adult neurogenesis results from proliferation and differentiation of neural stem cells. The olfactory epithelium has the capability to continuously regenerate olfactory receptor neurons throughout life. Frontera, J. L. et al., [Brain-derived neurotrophic factor (BDNF) expression in normal and regenerating olfactory epithelium of Xenopus laevis. Ann Anat. 2015 March; 198:41-8], confirmed the expression and presence of BDNF in the olfactory epithelium and bulb: in normal physiological conditions glial cells and stem cells express BDNF in the olfactory epithelium as well as the granular cells in the olfactory bulb. Moreover in the same article, during massive regeneration, Frontera et al., also demonstrated a drastic increase in basal cells expressing BDNF as well as an increase in BDNF in the olfactory bulb and nerve. Together, these results suggest an important role of BDNF in the maintenance and regeneration of the olfactory system.

The results of the study by McDole, B. et al., [BDNF over-expression increases olfactory bulb granule cell dendritic spine density in vivo. Neuroscience. 2015 Sep. 24; 304:146-60] indicate that increased levels of endogenous BDNF can promote the maturation and/or maintenance of dendritic spines on olfactory bulb granule cells. Amnestic Mild Cognitive Impairment (AMCI) often progresses to Alzheimer's disease. In the study by Turana, Y. et al. [Combination of Olfactory Test, Pupillary Response Test, BDNF Plasma Level, and APOE Genotype. Int J Alzheimers Dis. 2014; 2014:912586], low BDNF plasma level was related significantly with olfactory deficits and aMCI (P<0.05). Brain-derived neurotrophic factor (BDNF) is linked to neurodegenerative diseases (e.g., Alzheimer's disease and Parkinson's disease) that are often characterized by olfactory impairment.

A specific single nucleotide polymorphism of the BDNF gene, the Val66Met, which intracellular trafficking and activity-dependent secretion of BDNF protein was found by Tonacci, A. et al., to be associated with olfactory impairment underscoring the neuroprotective effect of BDNF on olfactory functions [Tonacci et al., Brain-derived neurotrophic factor (Val66Met) polymorphism and olfactory ability in young adults. J Biomed Sci. 2013 Aug. 7; 20:57].

A recent study (Uranagase A et al., BDNF expression in olfactory bulb and epithelium during regeneration of olfactory epithelium. Neurosci Lett. 2012 May 10; 516(1):45-9) suggests that BDNF in olfactory epithelium contributes to the early stage of regeneration, and BDNF in olfactory bulb has its role in the late stage of regeneration of olfactory receptor neurons. The 2017 study by Ortiz-Lopez, L. et al. [Human neural stem/progenitor cells derived from the olfactory epithelium express the TrkB receptor and migrate in response to BDNF. Neuroscience. 2017 Jul. 4; 355:84-100], shows that human neural stem/progenitor cells derived from the olfactory epithelium express the TrkB receptor and migrate in response to BDNF.

Smell dysfunction significantly influences physical well-being, quality of life, nutritional status as well as everyday safety and is associated with increased mortality (Attems J et al., Olfaction and Aging: A Mini-Review. Gerontology. 2015; 61(6):485-90). A drug like d-methadone which can increase BDNF levels might be able to slow progression, prevent and reverse impaired sense of smell, including hyposmia and dysosmia, caused by different etiologies, diseases, and their treatment, including cancer treatment.

Taste dysfunction can also significantly influence physical well-being, quality of life, nutritional status as well as everyday safety. Gustatory neurons are dependent on BDNF for survival; 50% of these neurons die in Bdnf(−/−) mice (Patel A V et al., Lingual and palatal gustatory afferents each depend on both BDNF and NT-4, but the dependence is greater for lingual than palatal afferents (J Comp Neurol. 2010 Aug. 15; 518(16):3290-301). A drug like d-methadone which can increase BDNF levels might be able to slow progression, prevent and reverse impaired sense of taste including hypogeusia a dysgeusia caused by different etiologies, diseases and their treatment, including cancer treatment.

Migraine, Cluster Headache and Other Headaches

There are indications that the NMDA receptor system and the NET may be implicated in the pathogenesis migraine, cluster headache and other headaches [Nicolodi, M. et al., Exploration of NMDA receptors in migraine: therapeutic and theoretic implications. Int J Clin Pharmacol Res. 1995; 15(5-6):181-9; Nicolodi, M. et al., Modulation of excitatory amino acids pathway: a possible therapeutic approach to chronic daily headache associated with analgesic drugs abuse. Int J Clin Pharmacol Res. 1997; 17(2-3):97-100; Roffey, P. et al., NMDA receptor blockade prevents nitroglycerin-induced headaches. Headache. 2001 July-August; 41(7):733; Farinelli, I. et al., Future drugs for migraine. Intern Emerg Med. 2009 October; 4(5):367-73]. Memantine, an NMDA antagonist, has been used successfully for the treatment and prevention of headaches [Lindelof, K. I. et al., Memantine for prophylaxis of chronic tension-type headache—a double-blind, randomized, crossover clinical trial. Cephalalgia. 2009 March; 29(3):314-21; Huang, L. et al., Memantine for the prevention of primary headache disorders. Ann Pharmacother. 2014 November; 48(11):1507-11; Noruzzadeh R et al., Memantine for Prophylactic Treatment of Migraine Without Aura: A Randomized Double-Blind Placebo-Controlled Study. Headache. 2016 January; 56(1):95-103).

Patients with refractory and recurring headaches including migraine, atypical headache syndromes, daily headaches, cluster headaches, have been successfully treated with l-methadone [Sprenger, T. et al., Successful prophylactic treatment of chronic cluster headache with low-dose levomethadone. J Neurol. 2008 November; 255(11):1832-3] and racemic methadone (Ribeiro, S. et al., Opioids for treating nonmalignant chronic pain: the role of methadone. Rev Bras Anestesiol. 2002 September; 52(5):644-51].

In a recent study [Glue, P. et al., Switching Opioid-Dependent Patients From Methadone to Morphine: Safety, Tolerability, and Methadone Pharmacokinetics. Clin Pharmacol. 2016 August; 56(8):960-5] of patients switched from methadone to morphine, the most frequent side effects were headache, nausea and neck pain, suggesting the sudden lack of a protective action of methadone against these symptoms, which are typical of migraine. A recent meta-analysis suggests that BDNF rs6265 and rs2049046 polymorphism are associated with common migraine [Cai, X. et al., The association between brain-derived neurotrophic factor gene polymorphism and migraine: a meta-analysis. J Headache Pain. 2017 18(1):13]. Patients with chronic migraine were found to have lower levels of BDNF [Martins, L. B. et al., Migraine is associated with altered levels of neurotrophins. Neurosci Lett. 2015 Feb. 5; 587:6-10]. Low testosterone has been implicated in migraine and cluster headache (Glaser R, et al., Testosterone pellet implants and migraine headaches: a pilot study. Maturitas. 2012 April; 71(4):385-8. Stillman M J. Testosterone replacement therapy for treatment refractory cluster headache. Headache. 2006 June; 46(6):925-33).

A novel drug like d-methadone, which combines NMDA antagonistic activity and NE re-uptake inhibition and potentially increases BDNF levels, and up-regulate testosterone levels while devoid of opioid activity, and is safe and well tolerated, may offer unique advantages for the treatment and prevention of migraine and other headaches.

Neurological Symptoms Caused by Acute Alcohol Withdrawal

Accumulation of the excitatory neurotransmitters may partly mediate the variety of neurological symptoms which are seen in alcohol withdrawal, such as delirium tremens, headache, sweating, delirium, tremors, seizures and hallucinations. Testosterone and BDNF decreased significantly during acute alcohol withdrawal (p<0.001) (A. Heberlein et al. Association of testosterone and BDNF serum levels with craving during alcohol withdrawal. Alcohol 54 (2016) 67e72). The above findings suggest a role for d-methadone which has NMDA antagonistic actions and has now been shown by the inventors to increase testosterone and BDNF levels, in the treatment of acute neurological symptoms and signs of alcohol withdrawal, such as headache, delirium, tremors, seizures and hallucinations. High blood pressure as a consequence of ETOH withdrawal and possibly mediated by excitotoxicity might also be treated by d-methadone as shown in the Examples and in the blood pressure section below.

Fibromyalgia

There are indications that the NMDA receptor system and the NET and abnormal levels of BDNF may be implicated in the pathogenesis of fibromyalgia. Memantine has been used successfully for fibromyalgia [Olivan-Blázquez, B. et al., Efficacy of memantine in the treatment of fibromyalgia: A double-blind, randomised, controlled trial with 6-month follow-up. Pain. 2014 December; 155(12):2517-25]. Methadone has been reportedly used for fibromyalgia with success [Ribeiro, S. et al., Opioids for treating nonmalignant chronic pain: the role of methadone. Rev Bras Anestesiol. 2002 September; 52(5):644-51].

Based on the inventors' body of work, the long-lasting body pains observed in a subset of patients treated with methadone for opioid addiction and or pain when tapering methadone, may not be a symptom of prolonged withdrawal, as previously assumed, but might represent the uncovering of latent fibromyalgia. Furthermore, low testosterone levels are implicated in the development of fibromyalgia (White H D et al., Treatment of pain in fibromyalgia patients with testosterone gel: Pharmacokinetics and clinical response. Int Immunopharmacol. 2015 August; 27(2):249-56.

A novel drug like d-methadone, which combines NMDA antagonistic activity and NE re-uptake inhibition and potentially increases BDNF levels and testosterone levels, and potentially modulates extra-neural glutamate receptors, while devoid of opioid activity and psychotomimetic effects and is safe and well tolerated, may offer unique advantages for the treatment and prevention of fibromyalgia.

Diseases of the Peripheral Nervous System (PNS) and Dysautonomia

BDNF is the sole neurotrophin upregulated in sensory neurons after peripheral nerve injury; BDNF was found to induce the cell body response in injured sensory neurons and increase their ability to extend neurites (Geremia N M et al., Endogenous BDNF regulates induction of intrinsic neuronal growth programs in injured sensory neurons. Exp Neurol. 2010 May; 223(1): 128-42.). Higher levels of BDNF were found to be related to lower scores on the Neuropathy rank-sum score (NRSS) [Andreassen, C. S. I. et al., Expression of neurotrophic factors in diabetic muscle—relation to neuropathy and muscle strength. Brain. 2009 October; 132(Pt 10):2724-33]. Researchers found that BDNF stimulates faster peripheral nerve regeneration (Vögelin E et al., Effects of local continuous release of brain derived neurotrophic factor (BDNF) on peripheral nerve regeneration in a rat model. Exp Neurol. 2006 June; 199(2): 348-53).

A novel drug like d-methadone, which combines NMDA antagonistic activity and NE re-uptake inhibition and potentially increase BDNF levels, but is devoid of opioid activity, and is safe and well tolerated, may offer unique advantages for the treatment of peripheral neuropathies of different etiology and diabetes mellitus, including its CNS and PNS neurological symptoms and manifestations. Peripheral neuropathies may be caused by metabolic disorders including diabetes and the metabolic syndrome, inflammatory and autoimmune diseases, infections, vascular disease, trauma and neurotoxins, including drugs, radiation therapy, and genetic diseases, including hereditary sensory and autonomic neuropathies. Peripheral neuropathies, aside from sensory and motor deficits may also cause dysautonomia. Aside from dysautonomia caused by PNS dysfunction, dysautonomia can also be caused by CNS dysfunction (including Parkinson disease and multisystem atrophy) or by both CNS and CNS dysfunction as in familial dysautonomia (Axelrod F B. Familial dysautonomia. Muscle & Nerve 2004; 29 (3):352-363).

Endocrine and Metabolic Disorders and Disorders of the Hypothalamic-Pituitary Axis

As detailed in the Examples, the inventors discovered that d-methadone up-regulates the serum levels of testosterone. Of note two of the three tested patients had low testosterone levels at baseline (defined as serum testosterone <7.6 nMol/L) and all three patients according to expert guidelines could be candidates for testosterone supplementation in the presence of specific symptoms and signs (Isidori A M, Balercia G, Calogero A E, Corona G, Ferlin A, Francavilla S, Santi D, Maggi M. Outcomes of androgen replacement therapy in adult male hypogonadism: recommendations from the Italian society of endocrinology. J Endocrinol Invest. 2015 January; 38(1):103-12).

This low testosterone level at baseline is particularly important because it suggests that the tested subjects may have had an abnormality in the hypothalamo-pituitary-gonadal axis (HPG axis) resulting in low testosterone levels.

As indicated in several sections of this application, d-methadone is a non-competitive low affinity open channel NMDAR antagonist with the potential of reaching the CNS in higher than expected concentrations and thus reach hypothalamic neurons and exert its actions selectively on pathologically open NMDARs on such neurons. While the discovery that d-methadone up-regulates testosterone serum levels in humans is based on a small number of subjects, in the 3/3 subjects tested, the results also correlate with BDNF levels in the same patients, reaching statistical significance for the correlkation. These results would be unexpected by those skilled in the art especially in light of the known testosterone lowering effect of opioids (Vuong C et al., The effects of opioids and opioid analogs on animal and human endocrine systems. Endocr Rev. 2010 February; 31(1):98-132). While unexpected, these results are indirectly supported by experimental work in vitro (Mahachoklertwattana P et al., N-methyl-D-aspartate (NMDA) receptors mediate the release of gonadotropin-releasing hormone (GnRH) by NMDA in a hypothalamic GnRH neuronal cell line (GT1-1). Endocrinology. 1994 March; 134(3):1023-30); and in vivo (Estienne M J1, Barb C R. Modulation of growth hormone, luteinizing hormone, and testosterone secretion by excitatory amino acids in boars. Reprod Biol. 2002 March; 2(1):13-24), showing that ketamine, an NMDA antagonists acting at the same site of the open NMDAR as d-methadone, has the potential to increase testosterone levels in boars.

While we have shown that testosterone and BDNF are potentially up-regulated by d-methadone and we postulate this up-regulation is mediated by NMDAR antagonism at dysfunctional hypothalamic neurons, we also postulate that this same mechanism could involve all of the main axes of the hypothalamus and pituitary which are similarly regulated, including the hypothalamo-pituitary-adrenal axis (HPA axis) the hypothalamo-pituitary-thyroid axis (HPT) and the hypothalamo-pituitary-gonadal axis (HPG) and the oxytocin and vasopressin secretion by the posterior pituitary, which all could therefore be potentially regulated by a drug like d-methadone. This mechanism of action on hypothalamic neurons has profound implications on regulation of many body functions that may be affected by abnormally functioning hypothalamic neurons secondary to NMDAR mediated excitotoxicity. Therefore the actions of d-methadone on pathologically open NMDAR of hypothalamic neurons might not only affect testosterone/BDNF, as shown in the subjects of the study presented in this application, but has also the potential to regulate body functions governed by all other factors secreted by hypothalamic neurons (including corticotrophin-releasing hormone, dopamine, growth hormone-releasing hormone, somatostatin, gonadotrophin-releasing hormone and thyrotrophin-releasing hormone, oxytocin and vasopressin) and by consequence the factors released by the pituitary gland (including adrenocorticotrophic hormone, thyroid stimulating hormone, growth hormone follicle stimulating hormone, luteinizing hormone, prolactin) and the glands, hormones and functions activated and regulated by these factors (adrenals, thyroid, gonads, sexual function, bone and muscle mass, blood pressure, glycemia, heart and kidney function, red blood cell production, immune system et cetera).

Finally, while targeting the cause of excitotoxicity in the CNS and hypothalamus might be a logic therapeutic strategy, in many instances this strategy turns out to be impractical or impossible, and regulation of abnormally functioning NMDAR by a drug like d-methadone might then become a potential therapeutic target not only for NS diseases, but also for endocrine-metabolic dysfunction and diseases, including those listed in this application.

To summarize, deregulation of the hypothalamic neurons caused by hyperactive NMDAR can be reset by a drug like d-methadone with the potential to block NMDARs only where they are pathologically hyper-stimulated, for example by excessive amounts of a neurotransmitters, such as glutamate.

d-Methadone therefore has the potential for becoming a therapeutic target in many diseases and conditions where hyperactivity of NMDAR on hypothalamic neurons is a contributing factor.

Eating disorders might also be successfully treated by a drug like d-methadone that can potentially regulate NMDARs at hypothalamic neurons (Stanley B G et al., Lateral hypothalamic NMDA receptors and glutamate as physiological mediators of eating and weight control. Am J Physiol. 1996 February; 270(2 Pt 2):R443-9).

Aside from well known metabolic effects and effects on sexual drive and function, testosterone appears to induce neuroprotection from oxidative stress (Chisu V, Manca P, Lepore G, Gadau S, Zedda M, Farina V. Testosterone induces neuroprotection from oxidative stress. Effects on catalase activity and 3-nitro-L-tyrosine incorporation into alpha-tubulin in a mouse neuroblastoma cell line. Arch Ital Biol. 2006 May; 144(2):63-73). The results from this study suggest a potential role of testosterone in preventing or reversing oxidative damage caused by normal aging and accelerated aging caused by diseases and their treatment.

Experimental results demonstrate that at least some of the effects of testosterone on neuronal plasticity and neuronal replacement are mediated through BDNF (Rasika S, Alvarez-Buylla A, Nottebohm F. BDNF Mediates the Effects of Testosterone on the Survival of New Neurons in an Adult Brain. Proc Natl Acad Sci USA. 1994 Aug. 16; 91(17):7854-8). This suggested mechanism correlates with the increase BDNF and testosterone seen in our human subjects treated with d-methadone 25 mg per day; the combined up-regulation of testosterone and BDNF offers further support to the effectiveness of d-methadone for all of the neurological diseases and other conditions claimed in this application, in addition to the prevention of neurological deterioration caused by normal and accelerated aging, diseases of the eye and obesity and the metabolic syndrome indications, including increased blood pressure, high blood sugar, excess body fat, including liver fat, and abnormal cholesterol or triglyceride levels. A significant positive association was found by Wickramatilake C M et al., between testosterone and HDL-Cholesterol (r=0.623, P=0.001), whereas a negative association was found with LDL-Cholesterol (r=−0.579, P=0.001). This observed association between testosterone and HDL-Cholesterol suggests a protective effect of the hormone for cardiovascular disease (Wickramatilake C M et al., Association of serum testosterone with lipid abnormalities in patients with angiographically proven coronary artery disease. Indian J Endocrinol Metab. 2013 November-December; 17(6): 1061-1065). Low testosterone appears to have adverse effects on the lipid profile and thus represent a risk factor for hypercholesterolemia, hypertriglyceridemia, high LDL-C, and low HDL-C, supporting the importance of maintaining an appropriate testosterone levels in men. (Zhang N et al., The relationship between endogenous testosterone and lipid profile in middle-aged and elderly Chinese men. European Journal of Endocrinology. (2014) 170, 487-494.) Finally, testosterone replacement therapy in hypogonadal and elderly men may have a beneficial effect on lipid metabolism through decreasing total cholesterol and atherogenic fraction of LDL-cholesterol without significant alterations in HDL-cholesterol levels or its subfractions HDL2-C and HDL3-C. (Zgliczynski S et al., Effect of testosterone replacement therapy on lipids and lipoproteins in hypogonadal and elderly men. Atherosclerosis. 1996 March; 121(1):35-43).

The above effects on lipid metabolism may also improve liver alcoholic and non-alchoholic fatty liver disease (NAFLD) and alcoholic and nonalcoholic steatohepatitis (NASH). NAFLD and NASH are associated with the metabolic syndrome (den Boer M et al., Hepatic steatosis: a mediator of the metabolic syndrome. Lessons from animal models. Arterioscler Thromb Vasc Biol. 2004 April; 24(4):644-9. Epub 2004) and an altered lipid profile similar to that seen in low testosterone states. On statistical analysis increasing grades of steatosis were significantly associated with increasing values of total cholesterol (P value—0.001), LDL (P value—0.000) and VLDL (P value—0.003) and decreasing HDL (P value—0.000) (Mahaling D U et al., Comparison of lipid profile in different grades of non-alcoholic fatty liver disease diagnosed on ultrasound. Asian Pac J Trop Biomed. 2013 November; 3(11): 907-912).

In summary, a drug like d-methadone which is safe and well-tolerated, is devoid of opioid activity and psychotomimetic effects at doses expected to maintain modulating actions on the NMDA receptor, NET system, and SERT system, and potentially up-regulates BDNF and testosterone might be useful for treating for one or more of the abnormalities associated with the metabolic syndrome, such as high blood pressure, high serum glucose levels, lipid profile abnormalities, increased body fat and increased fat in the liver, such as nonalcoholic fatty liver disease (NAFLD) and nonalcoholic steatohepatitis (NASH). These actions of d-methadone may also prevent the onset and progression of cardiovascular disease, including coronary artery disease, cerebrovascular disease and peripheral vascular disease. Of note, cognitive deterioration and Alzheimer's have been associated with a decline in reproductive hormones, including testosterone (Gregory C W and Bowen R L. Novel therapeutic strategies for Alzheimer's disease based on the forgotten reproductive hormones. Cell Mol Life Sci. 2005 February; 62(3):313-9).

While the risk benefit of supplemental testosterone in aging men is controversial, there is a clear association of decreased testosterone levels and declining cognitive function (Yeap B B. Hormonal changes and their impact on cognition and mental health of ageing men. Maturitas. 2014 October; 79(2):227-35).

Aside from neurological diseases and age related cognitive decline, the up-regulation of testosterone/BDNF from d-methadone may also improve other medical complications of aging such as sarcopenia. Sarcopenia is clinically defined as a loss of muscle mass coupled with functional deterioration (either walking speed or distance or grip strength). As sarcopenia is a major predictor of frailty, hip fracture, disability, and mortality in older persons, the development of drugs to prevent it and treat it is eagerly awaited (Morley J E. Pharmacologic Options for the Treatment of Sarcopenia. Calcif Tissue Int. 2016 April; 98(4):319-3). By preventing muscle mass loss and reducing body fat d-methadone is likely to prevent the progressive loss of strength and endurance seen with aging.

Osteoporosis and the metabolic syndrome may also be treated by a drug like d-methadone that up regulates testosterone and BDNF.

Testosterone, aside from the known effects on sexual drive and function, and overall energy levels, has been shown to reverse the main features of the metabolic syndrome. With a quarter of the American adult population affected, the metabolic syndrome and type 2 diabetes mellitus have been referred to as the most significant public health threats of the 21st century. The risk benefit of exogenous testosterone supplementation is not clearly established (Kovac J R, Pastuszak A W, Lamb D J, Lipshultz L I. Testosterone supplementation therapy in the treatment of patients with metabolic syndrome. Postgrad Med. 2014 November; 126(7):149-56). A recent meta-analysis supports the view of a positive effect of testosterone on body composition and on glucose and lipid metabolism. In addition, a significant effect on body composition was observed, suggesting a role for testosterone supplementation in the treatment and prevention of obesity (Corona G, Giagulli V A, Maseroli E, Vignozzi L, Aversa A, Zitzmann M, Saad F, Mannucci E, Maggi M. Testosterone supplementation and body composition: results from a meta-analysis of observational studies. J Endocrinol Invest. 2016 September; 39(9):967-81).

Epilepsy and Testosterone

Testosterone can have anti-seizure activity and testosterone-derived 3alpha-androstanediol has been shown to be an endogenous protective neurosteroid in the brain (Reddy D S. Anticonvulsant activity of the testosterone-derived neurosteroid 3alpha-androstanediol. Neuroreport. 2004 Mar. 1; 15(3):515-8). Testosterone may reduce seizures in men with epilepsy. Herzog A G. Psychoneuroendocrine aspects of temporolimbic epilepsy. Part II: Epilepsy and reproductive steroids. Herzog A G1. Psychosomatics. 1999 March-April; 40(2): 102-8. Up-regulation of testosterone may decrease seizure frequency in epileptic patients (Taubøll E et al., Interactions between hormones and epilepsy. Seizure. 2015 May; 28:3-11. Frye C A. Effects and mechanisms of progestogens and androgens in ictal activity. Epilepsia. 2010 July; 51 Suppl 3:135-40). Hypogonadism and low testosterone or estrogen levels are also remarkably associated with many neurological disorders such as epilepsy, ataxia, dysmyelination, nerve muscle disease, movement disorders, mental retardation and deafness, suggesting a possible causal or con-causal relationship. (Alsemari A. Hypogonadism and neurological diseases. Neurol Sci. 2013 May; 34(5):629-38). As exogenous testosterone replacement therapy carries potential risks (Gabrielsen J S, Najari B B, Alukal J P, Eisenberg M L. Trends in Testosterone Prescription and Public Health Concerns. Urol Clin North Am. 2016 May; 43(2):261-71), a drug like d-methadone that up-regulates levels of endogenous testosterone and BDNF by potentially acting on abnormally functioning NMDAR of hypothalamic neurons is likely to be beneficial without the side effects and risks of exogenous testosterone.

Hypogonadism is a side effect of opioid therapy and other drugs. Millions of patients continue to require opioid analgesics for control of moderate to severe chronic pain. A consequence of opioid treatment is opioid induced androgen deficiency (OPIAD). Chronic opioid use may predispose to hypogonadism through alteration of the hypothalamic-pituitary-gonadal axis as well as the hypothalamic-pituitary-adrenal-axis. The resulting hypogonadism and hypotestosteronism may contribute to impaired sexual function, decreased libido, infertility, and osteoporosis (Gudin J A, Laitman A, Nalamachu S. Opioid Related Endocrinopathy. Pain Med. 2015 October; 16 Suppl 1:S9-15). All of these symptoms and conditions and the risk of metabolic syndrome and hypertension may be prevented by a drug like d-methadone that up-regulates testosterone production.

In light of its effect on up-regulating testosterone and BDNF levels, d-methadone may be indicated for patients with: cognitive dysfunction, including age related cognitive dysfunction and Alzheimer's disease; metabolic syndrome; hypertension; endocrine diseases and diseases from deregulation of the hypothalamic-pituitary axis; epilepsy; aging of tissues including neurons, nerves, muscles (including sarcopenia), bone (including osteoporosis), skin, gonads (including impaired sexual function and decreased sexual drive), cornea (including dry eye syndrome), retina (including degenerative diseases of the retina), age related hearing and balance impairment. All of the above conditions, including normal aging and its symptoms and manifestations and accelerated aging caused by diseases and their treatment (e.g., therapies against cancer) may be improved by up-regulating endogenous testosterone levels and BDNF and reducing excitotoxicity.

Another indication is low testosterone of any cause including low testosterone caused by psychological distress, such as depression and anxiety or concomitant diseases and their treatment. Furthermore, iatrogenic low testosterone from opioid therapy and other drugs or medical treatments may be treated or prevented by d-methadone.

Effects of d-Methadone on Blood Pressure

Hypertension is a major risk factor for cardiovascular and cerebrovascular diseases. While numerous classes of drugs have antihypertensive actions, there are several drawbacks to existing therapies and new drugs with an improved side effect profile are needed.

To better understand the effects of d-methadone on blood pressure we analyzed the data from the phase 1 multiple ascending dose d-methadone double blind trial. The results of this analysis are presented in the Examples section of this application. The inventors noted a statistically significant lowering of blood pressure in the d-methadone treated subject. This blood pressure lowering effect was accompanied by an increase in oxygen saturation.

While this decrease in mean systolic and diastolic blood pressure remained within the parameters of safety, it signals regulating effects that are potentially useful for treatment of hypertension and the metabolic syndrome. The lowering of blood pressure seen in these subjects might be mediated by NMDA antagonistic effects at hypothalamic neurons with regulation of the hypothalamic-pituitary axis (Gören M Z et al., F. Cardiovascular responses to NMDA injected into nuclei of hypothalamus or amygdala in conscious rats. Pharmacology. 2000 November; 61(4):257-62): the study by Goren provides strong evidence for the tonic glutamatergic influence on blood pressure and heart rate via NMDA receptors located within the dorsomedial nucleus and to a lesser extent via those located within the paraventricular nucleus of the hypothalamus. Another study by Glass M J et al., (Glass M J et al., NMDA Receptor Plasticity in the Hypothalamic Paraventricular Nucleus Contributes to the Elevated Blood Pressure Produced by Angiotensin II. Journal of Neuroscience, 2015, 35 (26) 9558-9567) indicates that NMDA receptor plasticity in PVN neurons significantly contributes to the elevated blood pressure mediated by angiotensin II. This potential mechanism of action of d-methadone suggests that it might have many advantages as a novel antihypertensive because by regulating dysfunctional hypothalamic neurons it is not expected to have the side effects seen with commonly used antihypertensive drugs. Other possible mechanisms for the observed effect of lowering blood pressure include direct vasodilation, possibly through blocking L-type calcium channels [Tung K H et al. Contrasting cardiovascular properties of the μ-opioid agonists morphine and methadone in the rat. Eur J Pharmacol 2015 Sep. 5; 762:372-81]. As many patients suffering from hypertension require more than one drug for successful blood pressure control, d-methadone could also be a very useful add on therapy.

Finally, a drug like d-methadone which influences the catecholamine reuptake and serotonin reuptake, exerts NMDAR antagonism and up-regulates BDNF and testosterone levels and decreases blood pressure, aside from its activity on CNS and PNS NMDA receptors at peripheral nerves, and thus improving neurogenic dysfunction, (developmental or degenerative or toxic) and excitotoxic dysfunction of the gastrointestinal, cardiovascular, respiratory and renal systems, has also the potential of reducing excitotoxicity in non-neuronal cells with NMDARs. For example, non-neuronal cells in the gastrointestinal (including pancreatic cells and thus exerting metabolic effects such as glucose regulation; excitotoxicity of GI cells may also cause GI symptoms such as nausea), cardiovascular (thus influencing cardiac pathology including antiarrhythmic effects and anti-ischemic effects), respiratory (influencing asthma and other respiratory symptoms), reproductive and renal and skin systems [Gill S S. and Pulido O M. Glutamate Receptors in Peripheral Tissues: Current Knowledge, Future Research and Implications for Toxicology. Toxicologic Pathology 2001: 29 (2) 208-223]. These NMDAR blocking effects on peripheral cells may be particularly important in the treatment of acute and chronic exposure to toxins that can contaminate foods, such as domoic acid and food additives or enhancers (glutamate and aspartate like products). Furthermore, in addition to potentially act at the level of CNS NMDA receptors and at peripheral NMDA receptors at both neuronal and non-neuronal cells, as outlined above, d-methadone may also exert its pharmacological actions by regulating NMDA receptors at the level of hypothalamic neurons and therefore d-methadone can potentially regulate the hypothalamic-pituitary axis and influence all organs under its influence, as exemplified by the effect of d-methadone on up-regulating testosterone and lowering blood pressure, as detailed by the inventors in the sections above and in the Examples section.

Stereochemical Specificity of Methadone Analogues and Other Opioids

Among methadone analogues and other drugs classified as opioids there are a handful for which the stereochemical affinity for the opioid receptor is similar to the stereochemical affinity shown by methadone and its isomers: one of the isomers has much lower affinity for opioid receptors than the racemate or its chiral counterpart. These isomers with clinically negligible opioidergic effects are likely to instead have clinically significant non-stereospecific actions at other systems, such as the NMDAR, SERT, NET or actions at K, Na, Ca channels, as described for methadone. In the absence of opioidergic effects, the non-opioid effects of these opioid drug isomers could be potentially therapeutic for the same diseases and conditions and their symptoms and manifestations outlined in this application for d-methadone and, in particular for d-isomethadone and for l-moramide, these drugs could also be indicated for the treatment of pain and for the treatment of psychiatric symptoms, including depression. Some examples of these compounds therefore include:

1) Isomethadone and its isomers, d-isomethadone and l-isomethadone: d-isomethadone is 50 times less potent compared to l-isomethadone;

2) Moramide and its isomers, d-moramide and l-moramide: d-moramide is a schedule I drug in the USA because its high opioidergic potency, its high abuse potential and its highly euphoric effects; d-moramide is however in clinical use in certain European countries as an analgesic; l-moramide has instead negligible opioid binding activity (d-moramide is 700 times more potent than l-moramide in the mouse hot plate test); l-moramide could therefore have clinically significant actions at other systems, such as the NMDA receptor system, SERT, NET or actions at K, Na, Ca channels, as outlined above, without interfering opioidergic effects; furthermore the highly euphoric effects of d-moramide could be due to opioid effects combined with other effects that are not stereochemically specific, such as effects at the NMDAR, SERT, NET or actions at K, Na, Ca channels, or could be due exclusively to these non-opioid mechanisms, signaling added potential for l-moramide for the treatment of the same diseases and conditions and their symptoms and manifestations outlined in this application for d-methadone and in addition for the treatment of pain and for the treatment of psychiatric disorders including depression, conditions where effects on mood are particularly important and already disclosed for d-methadone but not for d-isomethadone or l-moramide. Similar differences exist for phenaxodone and its isomers and diampromide and its isomers. [The steric factor in medicinal chemistry; dissymetric probes of pharmacological receptors (Opioid ligands part 2): A. F. Casy. 503-543 pp. 1993. Plenum Press]. Propoxyphene is another such example of such opioidergic drugs: while the racemate and dextropropoxyphene have been used as analgesics for thei opidergic actions, the levo chiral counterpart, levoproxyphene, does not have clinically meaningful opioid effects (National Center for Biotechnology Information. PubChem Compound Database; CID=200742, https://pubchem.ncbi.nlm.nih.gov/compound/200742 (accessed Jan. 30, 2018) and thus may instead have clinically significant non-stereospecific actions at other systems, such as the NMDAR, SERT, NET or actions at K, Na, Ca channels which could be useful for the indications outlined in this application.

The various aspects of the present invention will be described in greater detail with respect to the following Examples.

### Examples

Based on their experimental and clinical studies and their joint experience, the inventors have discovered that a substance such as d-methadone may not only be effective for pain and psychiatric symptoms, but may also have a role in treating or preventing NS disorders and their neurological symptoms and manifestations, and a role in improving cognitive function, by modulating the NMDA, NET, and/or SERT systems, and potentially increasing BDNF levels and testosterone levels and by modulating K⁺, Ca²⁺ and Na⁺ cellular currents. Further, the inventors have discovered how these effects may be therapeutic especially if the disorder, symptom, or manifestation is associated with excitotoxicity, low BDNF levels and low testosterone levels or abnormalities in the NET and or SERT and/or cellular K⁺, Ca²⁺ and Na⁺ currents.

In order to demonstrate the clinical efficacy of d-methadone in treating or preventing NS disorders and their neurological symptoms or manifestations in humans, or in improving cognitive function, endocrine-metabolic disorders, eye diseases, disorders associated with aging, the inventors performed novel clinical and preclinical studies (described below). In summary, those studies show: (1) d-methadone is devoid of psychotomimetic effects at certain doses (e.g., at doses up to 200 mg); (2) d-methadone, at safe and potentially effective doses, is devoid of opioid effects, including cognitive side effects; (3) d-methadone follows linear pharmacokinetics (“PK”) at doses that are expected to be effective to bind to the NMDA receptor and NET of the subject and increase BDNF and testosterone levels without causing clinically significant QTc prolongation; (4) after subcutaneous administration d-methadone reaches the CNS (ng/g brain concentration) in concentrations 3.5 (10 mg/kg)-4.2 (20 mg/kg) times higher than systemic concentrations (ng/ml plasma concentration), suggesting effectiveness at doses lower (and safer) than expected; (5) the antagonistic effects of d-methadone on the electrophysiological response of human cloned NMDA NR1/NR2 A and NR1/NR2 B receptors expressed in HEK293 cells are in the low μM range, and therefore potentially exert clinical effects and possibly offer neuroprotection in humans; (6) d-methadone increases serum BDNF in humans (at a dose of 25 mg per day over ten days); (7) d-methadone increases serum testosterone in humans (at a dose of 25 mg per day over ten days); (8) a signal for improvement of cognitive function in humans due to d-methadone (from a single 5 mg d-methadone dose in humans), (9) a signal for lowering blood glucose in humans due to administration of d-methadone (via administration of 25 mg of d-methadone per day over ten days), and a signal for dose dependent decreased weight gain in rats from d-methadone, (10) d-methadone has in vivo behavioral effects that are comparable or stronger than those seen with ketamine and adequate to exert clinical effect and thus possibly neuroprotection in humans, (11) confirmation and characterization of the inhibitory activity exerted by d-methadone at NMDAR and on both NE and serotonin re-uptake and the characterization of NMDAR effects of deuterated d-methadone analogues. The studies leading to these results are described in detail below:

### Example 1: d-Methadone Exhibits No Psychotomimetic Effects, Exhibits No Opioid Effects, Exhibits No Clinically Significant Effects on the QTc Interval, Follows Linear Pharmacokinetics and has Blood Pressure Regulating Effects

The first of the study results listed above—the demonstration of the lack of psychotomimetic effects—is an important aspect, because drugs that effectively block the NMDA receptor (like ketamine and MK801) are associated with psychotomimetic effects that limit or impede their clinical use (especially their use for improving cognitive function). The second of the study results listed above—the lack of central opioid effects (and thus the lack of cognitive side effects of opioids)—is also important because opioid effects are likely to diminish and obscure any cognitive improvements mediated by non-opioid mechanisms. It would not be useful to administer a drug with potential psychotomimetic or central opioid effects for the purpose of improving cognitive function. The study results showing that d-methadone prolongs the QTc in a clinically non-significant manner is also important because drugs that exert proarrhythmic actions are poor candidates for clinical development. And the finding that d-methadone follows linear pharmacokinetics (the fourth study result listed above) is important because methadone is considered, by those skilled in the art, a drug with a long and unpredictable half-life with risk of delayed overdose, and therefore d-methadone has been expected to share the same risks.

The inventors performed experiments that were able to demonstrate that d-methadone does not to convert to l-methadone (a strong opioid with opioid related side effects) after in vivo administration to human subjects. And, the experiments demonstrated that d-methadone did not induce withdrawal upon abrupt discontinuation; thus eliminating another concern for its clinical usefulness that has, until the work of the present inventors, been present in the prior art.

In order to obtain data proving these points, the inventors performed and analyzed two novel sequential Phase 1 studies in 66 healthy volunteers and two preclinical studies. These studies were performed to characterize pharmacokinetic and pharmacodynamics parameters for d-methadone and to identify a well tolerated dose that could modulate the NMDA receptor and NET of the subject and to potentially increase BDNF levels in human subjects. The Phase I studies [a single ascending dose study (SAD) and a multiple ascending dose study (MAD)] are now described:

Single Ascending Dose (SAD) Study of d-Methadone in Healthy Volunteers (42 Subjects):

For the SAD study, subjects were assigned to the following cohorts: 5 mg, 20 mg, 60 mg, 100 mg, 150 mg, 200 mg. In each cohort (n=8), except the 200 mg cohort, subjects were randomly assigned to receive placebo (2 subjects) or d-methadone (6 subjects). The 200 mg cohort (n=2) included only sentinel subjects. Each cohort included 2 sentinel subjects, 1 who received d-methadone and 1 who received placebo. The remaining 6 subjects in the cohort, 1 who received placebo, were dosed at least 48 hours after the sentinel subjects.

Multiple Ascending Dose (MAD) Study of d-Methadone in Healthy Volunteers (24 Subjects):

The MAD study included 3 cohorts: 25 mg, 50 mg, and 75 mg. In each cohort (n=8), subjects were randomly assigned to receive placebo (2 subjects) or d-methadone (6 subjects). For 10 consecutive days, subjects received a single oral dose of d-methadone. Subjects remained in the clinic for at least 72 hours after the last dose and returned for 3 follow-up visits within 9 days after the last drug administration.

Summary and Results of the SAD and MAD Studies:

These two novel Phase 1, double-blind, randomized, placebo-controlled sequential SAD and MAD studies (carried out in sequential cohorts of healthy male and female subjects to investigate the safety, tolerability, and PK of d-methadone) confirmed that d-methadone is safe at doses that, based on the work of the inventors, are expected to be effective for the substance to bind to the NMDA receptor and NET/SERT, modulate K⁺, Ca⁺ and Na currents of the subject and increase BDNF and testosterone levels. The safety evaluation included the evaluation of treatment-emergent adverse events (TEAEs), laboratory values including testosterone levels, vital signs and cardiac monitoring, including electrocardiograms (EKGs), telemetry and Holter monitoring. Vital signs consisted of blood pressure, heart rate, respiratory rate, oxygen saturation.

Single doses up to 150 mg and multiple doses (once a day for ten days) up to 75 mg were well tolerated; none of the recorded TEAs were considered clinically meaningful. These doses (25-50 and 75 mg), based on the work of the inventors, are expected to bind to the NMDA receptor and NET/SERT and modulate K⁺, Ca⁺ and Na currents of the subject and to increase BDNF and testosterone levels. Steady state was achieved after 6-7 doses in the MAD study as expected from the elimination half-life of approximately 30 hours seen in the SAD study. The linearity of PK was demonstrated in the MAD portion of the study.

PK blood samples for the PK study were centrifuged, aliquoted, and stored at −20° C. (±5° C.) pending shipment to the bioanalytical laboratory. The plasma samples were analyzed for d-methadone and l-methadone by NWT, Inc. (Salt Lake City, Utah) using validated methods. The lower limit of quantification (LLOQ) was 5 ng/mL. The possibility of conversion of d-methadone to l-methadone in vivo was tested using a chiral bioanalytical assay: all l-methadone concentrations were below the limit of quantification for all doses, therefore, in subjects administered d-methadone, conversion to l-methadone did not occur. This finding is important because avoidance of the effects of the l-isomer (including opioid side effects on the cognitive function) is crucial in order to take full advantage of the cognitive improvement from d-methadone.

Tables 1-5 (below) show the results from these Phase 1 SAD and MAD studies.

TABLE 1
Summary of Baseline Demographics
Single Ascending Dose Study

| | Placebo | | 5 mg | | 20 mg | | 60 mg | | 100 mg | | 150 mg | | 200 mg |
|---|---|---|---|---|---|---|---|---|---|---|---|---|---|
| | (n = 11) | | (n = 6) | | (n = 6) | | (n = 6) | | (n = 6) | | (n = 6) | | (n = 1) |
| Age (years) | | | | | | | | | | | | | |
| Mean (SD) | 44 | (8.1) | 45 | (11.1) | 44 | (8.7) | 44 | (6.8) | 34 | (7.7) | 46 | (6.9) | 47 |
| Range | 31-54 | | 24-54 | | 31-54 | | 33-52 | | 24-43 | | 38-55 | | — |
| Sex, n (%) | | | | | | | | | | | | | |
| Male | 7 | (63.6) | 3 | (50.0) | 4 | (66.7) | 5 | (83.3) | 5 | (83.3) | 4 | (66.7) | 1 (100) |
| Female | 4 | (36.4) | 3 | (50.0) | 2 | (33.3) | 1 | (16.7) | 1 | (16.7) | 2 | (33.3) | 0 |
| BMI (kg/m²) | | | | | | | | | | | | | |
| Mean (SD) | 26.1 | (2.22) | 24.8 | (2.66) | 24.2 | (2.59) | 25.7 | (2.01) | 27.0 | (1.98) | 27.1 | (2.23) | 28.2 |

| | | | | | | | |
|---|---|---|---|---|---|---|---|
| Range | 22.6-29.7 | 21.2-28.9 | 21.2-27.5 | 22.2-27.7 | 23.1-28.7 | 23.1-29.2 | — |

Multiple Ascending Dose Study

| | Placebo | 25 mg | 50 mg | 75 mg |
|---|---|---|---|---|
| | (n = 6) | (n = 6) | (n = 6) | (n = 6) |
| Age (years) | | | | |

| | | | | | | | | |
|---|---|---|---|---|---|---|---|---|
| Mean (SD) | 39 | (12.2) | 39 | (7.5) | 46 | (6.2) | 43 | (7.0) |

| Range | 22-51 | 31-49 | 38-54 | 35-55 |
|---|---|---|---|---|
| Sex, n (%) | | | | |

| | | | | | | | | |
|---|---|---|---|---|---|---|---|---|
| Male | 3 | (50.0) | 3 | (50.0) | 3 | (50.0) | 3 | (50.0) |
| Female | 3 | (50.0) | 3 | (50.0) | 3 | (50.0) | 3 | (50.0) |

BMI (kg/m²)

| | | | | | | | | |
|---|---|---|---|---|---|---|---|---|
| Mean (SD) | 26.6 | (2.02) | 26.2 | (2.68) | 26.9 | (1.79) | 25.0 | (3.78) |

| | | | | |
|---|---|---|---|---|
| Range | 24.2-28.9 | 21.2-28.4 | 24.6-29.5 | 20.9-29.1 |

BMI = body mass index,
SD = standard deviation

TABLE 2
Pharmacokinetic Parameters for d-Methadone
Single Ascending Dose Study

| | | 5 mg | | 20 mg | | 60 mg | |
|---|---|---|---|---|---|---|---|
| Parameter | Statistic | (n = 6)^{a} | | (n = 6)^{a} | | (n = 6) | |
| Cₘₐₓ | n | 6 | | 6 | | 6 | |
| (ng/mL) | Mean (SD) | 53.30 | (19.042) | 163.3 | (56.302) | 403.7 | (222.46) |
| | Range | 29.6-83.9 | | 107-263 | | 186-686 | |
| Tₘₐₓ (h) | n | 6 | | 6 | | 6 | |
| | Median | 2.50 | (2.00-3.00) | 2.50 | (2.00-3.00) | 3.00 | (2.00-5.00) |
| | (Range) | | | | | | |
| AUC₀₋ₗₐₛₜ | n | 6 | | 6 | | 6 | |
| (h*ng/mL) | Mean (SD) | 1247 | (686.95) | 4616 | (2499.2) | 13,619 | (11,195) |
| | Range | 437-2188 | | 2078-7892 | | 1913-32,652 | |
| AUC₀₋₂₄ | n | 6 | | 6 | | 6 | |
| (h*ng/mL) | Mean (SD) | 674.9 | (286.74) | 2306 | (823.61) | 5539 | (3311.7) |
| | Range | 351-1153 | | 1376-3723 | | 1733-10,722 | |
| t_{1/2} (h) | n | 5 | | 4 | | 6 | |
| | Mean (SD) | 32.65 | (13.618) | 33.20 | (15.416) | 30.48 | (11.696) |

| | Range | 15.4-53.4 | 19.8-52.4 | | 9.02-43.3 | |
|---|---|---|---|---|---|---|
| AUC_{0-inf} | n | 1 | 4 | | 6 | |
| (h*ng/mL) | Mean (SD) | 1189 | 6226 | (2649.0) | 14,145 | (11,348) |
| | Range | — | 3795-8800 | | 2041-33,370 | |
| V_{d}/F | n | 1 | 4 | | 6 | |
| (L) | Mean (SD) | 178.9 | 154.0 | (25.418) | 255.0 | (107.47) |
| | Range | — | 127-184 | | 112-382 | |
| CL/F | n | 1 | 4 | | 6 | |
| (L/h) | Mean (SD) | 4.207 | 3.716 | (1.5824) | 8.979 | (10.269) |

| | | | |
|---|---|---|---|
| Range | — | 2.27-5.27 | 1.80-29.4 |

Single Ascending Dose Study

| | | 100 mg | | 150 mg | | 200 mg |
|---|---|---|---|---|---|---|
| Parameter | Statistic | (n = 6) | | (n = 6)^{a} | | (n = 1)^{a} |
| Cₘₐₓ | n | 6 | | 5 | | 1 |
| (ng/mL) | Mean (SD) | 738.7 | (225.40) | 1057 | (303.44) | 1530 |
| | Range | 583-1180 | | 804-1530 | | — |
| Tₘₐₓ (h) | n | 6 | | 5 | | 1 |
| | Median | 4.00 | (3.00-5.00) | 2.00 | (2.00-5.00) | 3.00 |
| | (Range) | | | | | |
| AUC₀₋ₗₐₛₜ | n | 6 | | 5 | | 1 |
| (h*ng/mL) | Mean (SD) | 29,690 | (10,640) | 37,184 | (15,090) | 67,673 |
| | Range | 18,781-43,449 | | 21,272-57,030 | | — |
| AUC₀₋₂₄ | n | 6 | | 5 | | 1 |
| (h*ng/mL) | Mean (SD) | 10,739 | (3432.9) | 14,599 | (3646.1) | 20,673 |
| | Range | 8097-17,376 | | 10,607-19,608 | | — |
| t_{1/2} (h) | n | 6 | | 4 | | 0 |
| | Mean (SD) | 42.31 | (8.3887) | 33.84 | (3.3830) | — |
| | Range | 32.6-53.1 | | 31.2-38.6 | | — |
| AUC_{0-inf} | n | 6 | | 4 | | 0 |
| (h*ng/mL) | Mean (SD) | 31,058 | (11,536) | 32,609 | (11,854) | — |
| | Range | 19,061-45,238 | | 21,506-49,398 | | — |
| V_{d}/F | n | 6 | | 4 | | 0 |
| (L) | Mean (SD) | 213.7 | (60.781) | 239.9 | (59.277) | — |
| | Range | 113-270 | | 169-314 | | — |
| CL/F | n | 6 | | 4 | | 0 |
| (L/h) | Mean (SD) | 3.612 | (1.2986) | 5.023 | (1.6086) | — |

| | | | |
|---|---|---|---|
| Range | 2.21-5.25 | 3.04-6.97 | — |

Multiple Ascending Dose Study

| | | | 25 mg | | 50 mg | | 75 mg | |
|---|---|---|---|---|---|---|---|---|
| Parameter | Dose | Statistic | (n = 6) | | (n = 6) | | (n = 5) | |
| Cₘₐₓ | 1 | Mean (SD) | 111.0 | (40.891) | 250.5 | (46.371) | 293.0 | (50.990) |
| (ng/mL) | | Range | 72.4-173 | | 175-293 | | 220-346 | |
| | 10 | Mean (SD) | 287.7 | (100.02) | 672.0 | (205.22) | 662.6 | (71.842) |
| | | Range | 154-423 | | 334-891 | | 559-723 | |
| Tₘₐₓ (h) | 1 | Median (Range) | 2.350 | (2.10-4.10) | 2.100 | (2.10-2.10) | 2.100 | (2.10-4.10) |
| | 10 | Median (Range) | 2.100 | (2.10-4.10) | 3.100 | (2.10-6.10) | 2.100 | (2.10-2.10) |
| AUC₀₋ₗₐₛₜ | 1 | Mean (SD) | 1367 | (436.57) | 2775 | (808.96) | 3441 | (325.73) |
| (h*ng/mL) | | Range | 819-2016 | | 1379-3771 | | 3061-3879 | |
| | 10 | Mean (SD) | 12,335 | (6863.0) | 30,204 | (18,354) | 27,953 | (6770.1) |
| | | Range | 3604-21,388 | | 8711-61,103 | | 17,859-34,964 | |
| AUCₜₐᵤ | 1 | Mean (SD) | 1404 | (449.50) | 2873 | (858.36) | 3636 | (329.17) |
| (h*ng/mL) | | Range | 834-2066 | | 1408-3888 | | 3271-4092 | |
| | 10 | Mean (SD) | 4392 | (1770.0) | 10,147 | (4317.6) | 10,537 | (1555.3) |
| | | Range | 2131-6976 | | 4108-17,272 | | 8181-12,409 | |
| t_{1/2} (h) | 10 | Mean (SD) | 36.63 | (8.9902) | 38.58 | (8.4579) | 37.07 | (7.0068) |
| | | Range | 21.6-45.0 | | 27.3-50.8 | | 29.3-46.6 | |
| AUC_{0-inf} | 10 | Mean (SD) | 12,985 | (7141.9) | 31,974 | (20,176) | 28,948 | (7203.8) |
| (h*ng/mL) | | Range | 3767-22,401 | | 8909-66,404 | | 18,443-37,026 | |
| V_{z}/F | 10 | Mean (SD) | 319.9 | (68.659) | 302.6 | (92.630) | 383.3 | (63.607) |

| | | | | |
|---|---|---|---|---|
| (L) | Range | 233-421 | 212-479 | 292-446 |

AUC₀₋₂₄ = area under the plasma concentration-time curve from time zero to 24 hours,
AUC_{0-inf} = area under the plasma concentration-time curve from time zero to infinity,
AUC₀₋ₗₐₛₜ = area under the plasma concentration-time curve from time zero until the last measurable concentration,
AUCₜₐᵤ = area under the plasma concentration-time curve for a dosing interval,
CL/F = clearance,
Cₘₐₓ = maximum observed plasma concentration,
SD = standard deviation,
t_{1/2} = apparent terminal elimination half-life,
Tₘₐₓ = time to maximum observed plasma concentration,
V_{d}/F = volume of distribution,
V_{z}/F = terminal volume of distribution
^{a}Subjects with parameters that were considered unreliable were not included in summary statistics.

TABLE 3
MAD Pharmacokinetic Steady-State Parameters

| Parameter | Statistic | 25 mg (n = 6) | | 50 mg (n = 6) | | 75 mg (n = 5) | |
|---|---|---|---|---|---|---|---|
| AUCₜₐᵤ | Mean (SD) | 4392 | (1770.0) | 10,147 | (4317.6) | 10,537 | (1555.3) |
| (h*ng/mL) | Range | 2131-6976 | | 4108-17,272 | | 8181-12,409 | |
| Cₛₛ | Mean (SD) | 142.7 | (66.087) | 326.2 | (185.29) | 338.8 | (55.585) |
| (ng/mL) | Range | 53.0-230 | | 116-641 | | 252-391 | |
| CLₛₛ/F | Mean (SD) | 6.606 | (2.9237) | 5.941 | (3.2394) | 7.255 | (1.1737) |
| (L/h) | Range | 3.58-11.7 | | 2.89-12.2 | | 6.04-9.17 | |
| Fluctuation | Mean (SD) | 81.65 | (23.718) | 97.38 | (36.275) | 84.57 | (20.585) |
| ratio (%) | Range | 59.5-116 | | 47.9-134 | | 59.3-102 | |
| R_{Cmax} | Mean (SD) | 2.617 | (0.38585) | 2.628 | (0.42457) | 2.294 | (0.29594) |
| | Range | 2.13-3.19 | | 1.91-3.18 | | 2.08-2.81 | |
| R_{AUCtau} | Mean (SD) | 3.077 | (0.48806) | 3.440 | (0.60145) | 2.910 | (0.45090) |
| | Range | 2.38-3.57 | | 2.90-4.44 | | 2.24-3.45 | |
| R_{Ctrough} | Mean (SD) | 3.405 | (0.59931) | 3.377 | (0.54377) | 2.747 | (0.31060) |

| | | | |
|---|---|---|---|
| Range | 2.32-3.88 | 2.55-4.14 | 2.23-3.00 |

AUCₜₐᵤ = area under the plasma concentration-time curve for a dosing interval,
CLₛₛ/F = steady-state clearance,
Cₛₛ = concentration at steady state,
R_{AUCtau} = accumulation ratio of AUCₜₐᵤ,
R_{Cmax} = accumulation ratio of Cₘₐₓ,
R_{Ctrough} = accumulation ratio of C_{trough},
SD = standard deviation

TABLE 4
Treatment-Emergent Adverse Events in ≥3 Subjects Overall, by MedDRA Preferred Term
Single Ascending Dose Study

| | Placebo | | 5 mg | 20 mg | | 60 mg | 100 mg | 150 mg | | 200 mg |
|---|---|---|---|---|---|---|---|---|---|---|
| | (n = 11) | | (n = 6) | (n = 6) | | (n = 6) | (n = 6) | (n = 6) | | (n = 1) |
| Adverse Event | n (%) [number of events] | | | | | | | | | |
| Any event | 7 | (63.6) [13] | 4 (66.7) [5] | 2 | (33.3) [11] | 4 (66.7) [4] | 1 (16.7) [4] | 6 | (100) [16] | 1 (100) [6] |
| Somnolence | 2 | (18.2) [2] | 1 (16.7) [1] | 2 | (33.3) [2] | 1 (16.7) [1] | 0 | 3 | (50.0) [3] | 1 (100) [1] |

| | | | | | | | | |
|---|---|---|---|---|---|---|---|---|
| Nausea | 0 | 1 (16.7) [1] | 0 | 0 | 1 (16.7) [1] | 3 | (50.0) [4] | 1 (100) [1] |
| Dizziness | 0 | 1 (16.7) [1] | 0 | 0 | 1 (16.7) [1] | 3 | (50.0) [3] | 0 |
| Vomiting | 0 | 0 | 0 | 0 | 0 | 2 | (33.3) [2] | 1 (100) [2] |

Multiple Ascending Dose Study

| | | | |
|---|---|---|---|
| Placebo | 25 mg | 50 mg | 75 mg |
| (n = 6) | (n = 6) | (n = 6) | (n = 6) |

| Adverse Event | n (%) [number of events] | | | | | | | |
|---|---|---|---|---|---|---|---|---|
| Any event | 3 | (50.0) [17] | 5 | (83.3) [16] | 5 | (83.3) [27] | 5 | (83.3) [45] |

| | | | | | | | |
|---|---|---|---|---|---|---|---|
| Somnolence | 2 | (33.3) [5] | 3 | (50.0) [3] | 0 | 4 | (66.7) [14] |

| | | | | | | |
|---|---|---|---|---|---|---|
| Nausea | 0 | 0 | 1 | (16.7) [1] | 3 | (50.0) [6] |

| | | | | | | | | |
|---|---|---|---|---|---|---|---|---|
| Dizziness | 1 | (16.7) [2] | 1 | (16.7) [1] | 1 | (16.7) [1] | 1 | (16.7) [6] |

| Skin irritation | 0 | 0 | | 3 | (50.0) [3] | 0 |
|---|---|---|---|---|---|---|
| Ventricular | 0 | 2 | (33.3) [2] | 1 | (16.7) [2] | 0 |
| extrasystoles | | | | | | |

MedDRA = Medical Dictionary for Regulatory Activities

TABLE 5
Respiratory Parameters: Maximum Mean Decrease from Baseline
Single Ascending Dose Study

| | | | | | | |
|---|---|---|---|---|---|---|
| Placebo | 5 mg | 20 mg | 60 mg | 100 mg | 150 mg | 200 mg |
| (n = 11) | (n = 6) | (n = 6) | (n = 6) | (n = 6) | (n = 6) | (n = 1) |

Mean (SD)

Respiratory Rate (breaths/min)

| | | | | | | | |
|---|---|---|---|---|---|---|---|
| Baseline | 14.2 (1.89) | 13.3 (1.03) | 15.3 (1.03) | 15.0 (2.45) | 14.3 (1.97) | 17.0 (2.76) | 16.0 |
| Maximum CFB | ^{a} | −1.0 (1.10) | −2.0 (2.53) | −1.0 (2.45) | −1.7 (1.51) | −2.0 (2.19) | −2.0 |

Oxygen Saturation (%)

| | | | | | | | |
|---|---|---|---|---|---|---|---|
| Baseline | 98.9 (0.70) | 98.7 (0.52) | 98.8 (0.75) | 98.5 (0.55) | 98.5 (0.84) | 97.8 (1.17) | 98.0 |
| Maximum CFB | −0.9 (1.45) | −1.2 (1.72) | −0.8 (1.17) | −0.8 (0.41) | −1.2 (0.75) | −0.5 (2.07) | −1.0 |

Multiple Ascending Dose Study

| | | | |
|---|---|---|---|
| Placebo | 25 mg | 50 mg | 75 mg |
| (n = 6) | (n = 6) | (n = 6) | (n = 6)^{b} |

Mean (SD)

Respiratory Rate (breaths/min)

| | | | | |
|---|---|---|---|---|
| Baseline | 14.3 (1.97) | 13.7 (1.51) | 15.3 (1.63) | 13.7 (2.66) |
| Maximum CFB | −1.0 (2.10) | −1.7 (1.51) | −2.0 (1.79) | −1.6 (3.29) |

Oxygen Saturation (%)

| | | | | |
|---|---|---|---|---|
| Baseline | 98.8 (0.41) | 98.7 (0.52) | 97.8 (1.17) | 98.2 (0.98) |
| Maximum CFB | −1.0 (1.26) | −0.5 (1.05) | −0.5 (2.35) | −1.0 (1.22) |
| | −1.0 (1.10) | −0.5 (0.55) | | −1.0 (1.87) |
| | | −0.5 (0.84) | | |

CFB = change from baseline,
SD = standard deviation
The reference range for respiratory rate was 12 to 20 breaths/min, and for oxygen saturation it was ≥95%. For the SAD study, the observation period was 72 hours post-dose. For the MAD study, the observation period for respiratory rate was 12 hours post-dose from Day 1 to Day 9 and 72 hours post-dose for Day 10; the observation period for oxygen saturation was 8 hours post-dose from Day 1 to Day 10.
^{a} No negative decrease from baseline occurred in the placebo group.
^{b}For respiratory rate, n = 5 for this cohort for Day 5 onward; for oxygen saturation, n = 5 for this cohort on Day 3 and on Day 5 onward

Vital Signs:

None of the mean values at any time point were outside the normal range for any of the vital sign parameters evaluated.

Table 6 below summarizes the mean changes from baseline in blood pressure and heart rate. All assessment time points on Day 1 and Day 10 are included; however, from Day 2 to Day 9, only the 2 hour post-dose values (ie, Tₘₐₓ) are summarized in the table. Post-dose decreases in systolic and diastolic blood pressure were observed in all treatment groups, including placebo, but the change from baseline was consistently negative for the 50 mg and 75 mg groups throughout the study and, overall, the magnitude of the change was greatest in the 75 mg d-methadone group. Minor fluctuations in heart rate occurred in all treatment group, but a similar pattern as for blood pressure was observed—overall, the 75 mg group exhibited the greatest negative changes from baseline.

TABLE 6
Summary of Mean Changes in Blood Pressure and
Pulse Rate from Baseline (Safety Population)

| Placebo | d-Methadone | | |
|---|---|---|---|
| (N = 6) | 25 mg (N = 6) | 50 mg (N = 6) | 75 mg (N = 6)^{a} |

| | |
|---|---|
| Time Point | Mean (SD) |

Systolic Blood Pressure (mmHg)

| | | | | | | | | | |
|---|---|---|---|---|---|---|---|---|---|
| Day 1 | pre-dose | 115.3 | (4.84) | 120.0 | (10.60) | 125.2 | (14.95) | 118.5 | (16.08) |
| | CFB 2 h | −2.0 | (4.29) | 0.7 | (3.39) | −9.5 | (17.07) | −6.0 | (13.62) |
| | CFB 4 h | 0.5 | (3.62) | −6.7 | (8.07) | −17.2 | (19.89) | −11.7 | (12.24) |
| | CFB 6 h | 2.5 | (6.77) | −8.3 | (4.23) | −14.0 | (16.98) | −11.2 | (16.83) |
| | CFB 8 h | 1.5 | (5.96) | −5.8 | (4.58) | −9.5 | (19.99) | −10.0 | (11.76) |
| | CFB 12 h | −0.7 | (5.61) | 2.5 | (5.05) | −7.3 | (23.94) | −11.0 | (9.36) |
| Day 2 | CFB | −4.3 | (3.78) | −0.7 | (7.63) | −6.3 | (14.50) | −18.2 | (7.57) |
| | pre-dose | | | | | | | | |
| | CFB 2 h | −1.2 | (17.68) | 0.7 | (3.98) | −10.5 | (19.33) | −10.5 | (15.27)^{b} |
| Day 3 | CFB | −1.7 | (9.83) | −1.0 | (5.97) | −8.5 | (19.43) | −10.2 | (9.17)^{b} |
| | pre-dose | | | | | | | | |
| | CFB 2 h | 1.3 | (8.07) | 0.7 | (9.00) | −5.7 | (23.87) | −7.3 | (16.16)^{b} |
| Day 4 | CFB | −4.2 | (7.88) | −4.5 | (8.02) | −13.3 | (21.27) | −6.7 | (9.61)^{b} |
| | pre-dose | | | | | | | | |
| | CFB 2 h | 0.8 | (11.65) | −2.0 | (5.55) | −8.8 | (17.23) | −8.0 | (15.36)^{b} |
| Day 5 | CFB | −0.7 | (11.59) | 1.7 | (9.07) | −8.8 | (18.13) | −9.0 | (11.73) |
| | pre-dose | | | | | | | | |
| | CFB 2 h | 3.2 | (9.64) | −2.5 | (7.97) | −4.7 | (20.94) | −8.8 | (7.63) |
| Day 6 | CFB | −0.3 | (11.02) | 0.2 | (11.79) | −9.0 | (19.09) | −9.4 | (11.82) |
| | pre-dose | | | | | | | | |
| | CFB 2 h | −0.7 | (9.24) | −3.7 | (6.62) | −8.5 | (16.72) | −8.2 | (21.25) |
| Day 7 | CFB | −5.0 | (13.11) | −6.8 | (7.57) | −9.3 | (16.02) | −11.6 | (9.91) |
| | pre-dose | | | | | | | | |
| | CFB 2 h | 3.2 | (11.79) | −5.0 | (5.55) | −10.8 | (20.07) | −13.2 | (21.28) |
| Day 8 | CFB | 4.2 | (12.54) | −10.0 | (7.16) | −11.7 | (17.92) | −8.4 | (14.22) |
| | pre-dose | | | | | | | | |
| | CFB 2 h | −4.0 | (6.51) | −3.5 | (11.11) | −12.3 | (20.08) | −11.4 | (14.89) |
| Day 9 | CFB | −1.7 | (7.23) | 1.3 | (6.65) | −10.0 | (20.33) | −11.6 | (15.19) |
| | pre-dose | | | | | | | | |
| | CFB 2 h | −3.2 | (7.11) | −8.3 | (6.59) | −11.5 | (18.80) | −5.2 | (18.13) |
| Day 10 | CFB | 0.5 | (4.64) | −5.8 | (8.84) | −11.5 | (17.51) | −10.6 | (13.96) |
| | pre-dose | | | | | | | | |
| | CFB 2 h | −1.2 | (6.31) | −6.5 | (5.32) | −8.8 | (17.98) | −11.4 | (16.15) |
| | CFB 4 h | −0.2 | (11.20) | −13.0 | (7.18) | −10.3 | (21.75) | −8.8 | (17.05) |
| | CFB 6 h | −2.2 | (8.50) | −6.7 | (8.26) | −6.5 | (27.20) | −10.4 | (16.77) |
| | CFB 8 h | 4.7 | (13.05) | −6.0 | (8.94) | −1.2 | (23.97) | −3.2 | (19.72) |
| | CFB 12 h | 1.3 | (11.15) | −2.0 | (6.45) | −4.3 | (25.30) | −3.6 | (24.14) |
| Day 11 | CFB 24 h | −2.8 | (5.12) | −7.5 | (7.82) | −6.5 | (14.49) | −8.8 | (7.60) |
| Day 12 | CFB 48 h | −4.2 | (5.00) | −9.5 | (9.59) | −4.7 | (18.07) | −14.8 | (16.99) |
| Day 13 | CFB 72 h | −2.5 | (11.33) | −7.8 | (7.81) | −5.5 | (14.31) | −11.6 | (12.66) |

Diastolic Blood Pressure (mmHg)

| | | | | | | | | | |
|---|---|---|---|---|---|---|---|---|---|
| Day 1 | pre-dose | 67.0 | (4.60) | 73.5 | (6.72) | 73.3 | (9.83) | 75.7 | (10.17) |
| | CFB 2 h | 0.7 | (4.32) | −4.0 | (3.16) | −4.0 | (7.51) | −3.7 | (7.23) |
| | CFB 4 h | −0.7 | (1.37) | −8.2 | (4.45) | −8.0 | (10.08) | −13.0 | (12.55) |
| | CFB 6 h | −1.5 | (4.68) | −8.2 | (5.56) | −9.3 | (8.21) | −7.2 | (12.97) |
| | CFB 8 h | −0.3 | (5.89) | −5.0 | (5.25) | −6.3 | (9.73) | −9.8 | (11.58) |
| | CFB 12 h | −4.5 | (6.66) | −2.7 | (5.85) | −4.2 | (11.32) | −13.0 | (11.30) |
| Day 2 | CFB | −1.0 | (5.06) | −2.8 | (5.71) | −1.8 | (10.05) | −11.2 | (6.74) |
| | pre-dose | | | | | | | | |
| | CFB 2 h | −2.7 | (6.56) | −5.0 | (4.38) | −6.0 | (8.94) | −10.7 | (14.01)^{b} |
| Day 3 | CFB | −3.3 | (7.69) | −3.8 | (5.04) | −3.2 | (10.98) | −6.5 | (5.17)^{b} |
| | pre-dose | | | | | | | | |
| | CFB 2 h | −2.0 | (6.84) | −4.2 | (6.65) | −4.3 | (9.48) | −8.5 | (9.99)^{b} |
| Day 4 | CFB | −0.5 | (5.36) | −2.0 | (3.85) | −5.8 | (10.09) | −6.7 | (6.25)^{b} |
| | pre-dose | | | | | | | | |
| | CFB 2 h | −0.3 | (8.21) | −5.8 | (5.42) | −5.7 | (8.41) | −7.5 | (10.86)^{b} |
| Day 5 | CFB | −0.7 | (5.99) | −1.7 | (5.32) | −3.0 | (10.68) | −5.8 | (6.83) |
| | pre-dose | | | | | | | | |
| | CFB 2 h | 0.2 | (3.54) | −6.5 | (8.31) | −5.5 | (9.79) | −10.0 | (8.25) |
| Day 6 | CFB | −2.3 | (6.28) | −3.3 | (7.15) | −5.5 | (9.09) | −3.6 | (11.78) |
| | pre-dose | | | | | | | | |
| | CFB 2 h | −3.0 | (5.10) | −9.0 | (5.73) | −5.3 | (9.75) | −5.0 | (13.11) |
| Day 7 | CFB | −1.5 | (6.35) | −3.2 | (7.94) | −2.7 | (8.19) | −6.0 | (10.27) |
| | pre-dose | | | | | | | | |
| | CFB 2 h | −3.3 | (8.52) | −5.0 | (5.59) | −5.0 | (9.25) | −7.0 | (10.89) |
| Day 8 | CFB | 1.0 | (6.60) | −4.8 | (7.22) | −2.2 | (10.91) | −8.0 | (9.77) |
| | pre-dose | | | | | | | | |
| | CFB 2 h | −5.2 | (4.58) | −6.3 | (6.28) | −6.5 | (8.55) | −7.4 | (11.95) |
| Day 9 | CFB | −1.7 | (6.71) | −5.7 | (8.19) | −3.2 | (10.98) | −7.2 | (10.35) |
| | pre-dose | | | | | | | | |
| | CFB 2 h | −6.0 | (6.78) | −11.5 | (7.92) | −7.0 | (8.63) | −6.2 | (11.41) |
| Day 10 | CFB | −2.5 | (3.94) | −5.2 | (7.78) | −6.0 | (10.16) | −7.2 | (12.68) |
| | pre-dose | | | | | | | | |
| | CFB 2 h | −3.3 | (2.88) | −8.0 | (6.07) | −6.5 | (7.23) | −11.2 | (11.08) |
| | CFB 4 h | −4.8 | (3.43) | −10.0 | (6.54) | −10.7 | (9.63) | −9.2 | (13.16) |
| | CFB 6 h | −4.3 | (3.83) | −9.3 | (8.80) | −9.0 | (12.81) | −10.6 | (10.16) |
| | CFB 8 h | −0.5 | (5.09) | −10.2 | (7.55) | −2.7 | (8.31) | −3.0 | (10.89) |
| | CFB 12 h | −0.2 | (6.34) | −6.8 | (7.36) | −7.7 | (14.36) | −4.4 | (13.15) |
| Day 11 | CFB 24 h | −2.2 | (2.93) | −5.8 | (8.18) | −6.2 | (9.06) | −11.8 | (11.54) |
| Day 12 | CFB 48 h | −1.7 | (4.13) | −6.7 | (8.41) | −1.7 | (12.44) | −10.0 | (11.66) |
| Day 13 | CFB 72 h | 0.0 | (4.69) | −7.3 | (6.65) | −0.5 | (9.33) | −11.2 | (9.44) |

Heart Rate (beats/min)

| | | | | | | | | | |
|---|---|---|---|---|---|---|---|---|---|
| Day 1 | pre-dose | 60.5 | (4.81) | 61.7 | (11.02) | 64.8 | (7.94) | 60.7 | (6.06) |
| | CFB 2 h | 2.8 | (6.24) | 0.0 | (9.01) | −2.3 | (9.05) | −4.8 | (10.40) |
| | CFB 4 h | 2.8 | (5.49) | −0.5 | (7.97) | −5.3 | (7.71) | −5.3 | (9.93) |
| | CFB 6 h | 4.3 | (3.67) | 3.3 | (6.95) | −1.8 | (9.28) | −1.8 | (7.83) |
| | CFB 8 h | 3.0 | (4.86) | −0.2 | (8.84) | −3.2 | (11.86) | −3.8 | (8.33) |
| | CFB 12 h | 3.5 | (3.73) | 3.5 | (6.02) | −3 3 | (10.35) | −3.2 | (9.43) |
| Day 2 | CFB | −3.5 | (5.05) | −0.2 | (8.47) | −2.5 | (9.61) | −8.3 | (6.83) |
| | pre-dose | | | | | | | | |
| | CFB 2 h | 5.3 | (4.18) | 4.2 | (12.45) | 0.3 | (12.63) | −2.7 | (14.12)^{b} |
| Day 3 | CFB | −0.3 | (3.61) | −2.8 | (6.01) | −2.8 | (9.77) | 1.3 | (12.71)^{b} |
| | pre-dose | | | | | | | | |
| | CFB 2 h | 3.2 | (3.82) | 2.0 | (9.19) | 0.2 | (10.25) | 1.3 | (12.91)^{b} |
| Day 4 | CFB | 1.0 | (4.98) | 1.2 | (7.28) | −1.0 | (9.14) | 2.8 | (10.15)^{b} |
| | pre-dose | | | | | | | | |
| | CFB 2 h | 10.3 | (4.27) | 3.5 | (6.75) | 0.3 | (9.67) | 0.8 | (13.82)^{b} |
| Day 5 | CFB | 2.0 | (1.41) | 3.7 | (9.83) | 0.5 | (10.73) | −3.2 | (4.32) |
| | pre-dose | | | | | | | | |
| | CFB 2 h | 3.7 | (9.22) | 5.7 | (9.71) | 0.5 | (11.57) | −3.0 | (13.64) |
| Day 6 | CFB | 0.5 | (3.27) | 1.0 | (8.67) | 1.0 | (6.72) | −3.4 | (6.80) |
| | pre-dose | | | | | | | | |
| | CFB 2 h | 3.8 | (6.49) | 6.0 | (11.83) | 1.5 | (10.25) | −1.8 | (15.42) |
| Day 7 | CFB | 0.3 | (6.65) | 2.7 | (8.26) | −0.3 | (8.96) | −3.0 | (7.11) |
| | pre-dose | | | | | | | | |
| | CFB 2 h | 6.7 | (6.09) | 4.8 | (12.34) | 1.5 | (12.37) | −1.0 | (14.92) |
| Day 8 | CFB | 2.2 | (7.88) | 3.8 | (8.18) | 0.5 | (10.33) | −1.8 | (7.26) |
| | pre-dose | | | | | | | | |
| | CFB 2 h | 6.8 | (6.49) | 4.5 | (10.78) | 0.8 | (12.64) | 2.4 | (14.29) |
| Day 9 | CFB | −0.7 | (6.09) | 7.2 | (7.08) | 1.0 | (10.04) | −5.6 | (6.80) |
| | pre-dose | | | | | | | | |
| | CFB 2 h | 7.7 | (9.40) | 7.3 | (6.80) | 3.3 | (9.24) | −3.2 | (12.87) |
| Day 10 | CFB | 1.8 | (6.91) | 3.5 | (6.19) | 3.7 | (8.14) | −2.4 | (10.09) |
| | pre-dose | | | | | | | | |
| | CFB 2 h | 5.8 | (9.13) | 3.5 | (7.50) | 3.3 | (11.76) | −3.2 | (15.02) |
| | CFB 4 h | 9.7 | (7.37) | 2.2 | (7.11) | 6.7 | (12.42) | −6.4 | (9.48) |
| | CFB 6 h | 12.5 | (6.53) | 8.2 | (8.04) | 3.7 | (11.76) | −3.2 | (6.76) |
| | CFB 8 h | 7.2 | (7.47) | 5.0 | (6.57) | 3.8 | (13.64) | −5.2 | (7.05) |
| | CFB 12 h | 8.2 | (6.82) | 2.7 | (5.68) | 2.7 | (12.79) | −1.0 | (10.77) |
| Day 11 | CFB 24 h | 1.8 | (7.63) | 3.5 | (7.37) | 0.5 | (12.14) | 3.6 | (13.39) |
| Day 12 | CFB 48 h | 0.7 | (7.03) | 3.3 | (7.09) | 2.2 | (9.02) | 2.6 | (13.78) |
| Day 13 | CFB 72 h | 5.5 | (6.47) | 6.3 | (5.05) | 5.8 | (7.14) | 7.8 | (10.55) |

CFB = change from baseline,
SD = standard deviation
^{a}N = 5 for this cohort from Day 5 pre-dose onward.
^{b}Mean values include Subject 9018's vital signs during monitoring from Day 2 to Day 4 for an adverse event; however, the subject was not dosed on Day 2.
Baseline is defined as pre-dose on Day 1.

All mean respiratory rate and oxygen saturation values were normal at all time points during the study. There was little variation in respiratory rate or oxygen saturation during the course of the study. Mean change from baseline data are summarized in Table 7. Overall, most changes in respiratory rate were positive, and there was no dose-response relationship. For oxygen saturation, all changes from baseline were small in magnitude (ie, ≤1%), and the placebo group exhibited the most negative changes during the course of the study. No subject had a respiratory rate or an oxygen saturation level that was below the reference range.

TABLE 7
Summary of Mean Change from Baseline for Respiratory
Rate and Oxygen Saturation (Safety Population)

| | d-Methadone | | |
|---|---|---|---|
| Placebo (N = 6) | 25 mg (N = 6) | 50 mg (N = 6) | 75 mg (N = 6)^{a} |

| | |
|---|---|
| Time Point | Mean (SD) |

Respiratory Rate (breaths/min)

| | | | | | | | | | |
|---|---|---|---|---|---|---|---|---|---|
| Day 1 | pre-dose | 14.3 | (1.97) | 13.7 | (1.51) | 15.3 | (1.63) | 13.7 | (2.66) |
| | CFB 2 h | 1.3 | (2.42) | 0.7 | (2.07) | 0.7 | (1.63) | 1.0 | (3.95) |
| | CFB 4 h | 2.3 | (0.82) | 1.3 | (3.93) | 0.0 | (1.26) | 1.7 | (2.34) |
| | CFB 6 h | 0.7 | (3.27) | −0.3 | (2.34) | −0.7 | (1.63) | 1.3 | (3.27) |
| | CFB 8 h | 1.3 | (3.72) | −0.7 | (2.07) | −0.3 | (1.97) | 1.0 | (3.29) |
| | CFB 12 h | 1.7 | (1.97) | 1.3 | (2.73) | −0.3 | (2.34) | 2.0 | (2.53) |
| Day 2 | CFB | 0.7 | (2.73) | −1.7 | (1.51) | 0.0 | (2.19) | 2.0 | (1.79) |
| | pre-dose | | | | | | | | |
| | CFB 2 h | −1.0 | (2.10) | −1.3 | (1.63) | −0.7 | (2.42) | 0.3 | (2.66)^{b} |
| Day 3 | CFB | 2.0 | (2.83) | 1.7 | (1.51) | 0.0 | (2.19) | 1.0 | (2.76)^{b} |
| | pre-dose | | | | | | | | |
| | CFB 2 h | 1.3 | (2.42) | 1.3 | (2.42) | 0.3 | (2.34) | 1.3 | (3.01)^{b} |
| Day 4 | CFB | 1.0 | (1.10) | 0.7 | (2.42) | 0.0 | (2.19) | 1.7 | (3.44)^{b} |
| | pre-dose | | | | | | | | |
| | CFB 2 h | 1.0 | (3.29) | 1.0 | (1.67) | −1.0 | (1.67) | 2.7 | (3.01)^{b} |
| Day 5 | CFB | 0.0 | (2.83) | 1.7 | (2.94) | 0.3 | (2.34) | 2.4 | (2.61) |
| | pre-dose | | | | | | | | |
| | CFB 2 h | 0.0 | (2.83) | 1.7 | (3.44) | −0.7 | (1.63) | 0.4 | (2.61) |
| Day 6 | CFB | 0.3 | (3.44) | 3.7 | (1.97) | −1.3 | (3.27) | 0.8 | (4.38) |
| | pre-dose | | | | | | | | |
| | CFB 2 h | 0.3 | (2.34) | 2.7 | (2.73) | −1.0 | (2.10) | 0.8 | (2.68) |
| Day 7 | CFB | 0.0 | (1.26) | 1.3 | (3.72) | −0.3 | (2.34) | 0.4 | (3.29) |
| | pre-dose | | | | | | | | |
| | CFB 2 h | 1.7 | (3.67) | 0.7 | (3.93) | 0.0 | (2.53) | 0.8 | (2.68) |
| Day 8 | CFB | 0.3 | (2.66) | 1.7 | (2.66) | −0.3 | (1.97) | 0.8 | (3.03) |
| | pre-dose | | | | | | | | |
| | CFB 2 h | 0.3 | (2.34) | 2.7 | (2.42) | 0.0 | (3.10) | 0.8 | (3.03) |
| Day 9 | CFB | 0.0 | (2.53) | 2.0 | (1.26) | 0.7 | (1.63) | 1.2 | (3.03) |
| | pre-dose | | | | | | | | |
| | CFB 2 h | −0.3 | (2.94) | 2.3 | (1.51) | −0.7 | (1.03) | 1.2 | (3.03) |
| Day 10 | CFB | 0.7 | (2.42) | 1.0 | (2.10) | 0.7 | (1.63) | 0.8 | (3.63) |
| | pre-dose | | | | | | | | |
| | CFB 2 h | 1.0 | (3.95) | 3.7 | (2.94) | 0.3 | (2.34) | −0.4 | (3.29) |
| | CFB 4 h | 1.3 | (3.27) | 2.0 | (3.79) | 0.5 | (1.76) | 0.8 | (3.63) |
| | CFB 6 h | 1.0 | (4.34) | 2.3 | (3.88) | −0.7 | (1.03) | 2.4 | (3.29) |
| | CFB 8 h | 2.0 | (4.00) | 3.0 | (3.29) | −0.7 | (1.03) | 1.6 | (3.58) |
| | CFB 12 h | 1.3 | (3.27) | 2.3 | (1.97) | −0.7 | (2.07) | 1.6 | (2.61) |
| Day 11 | CFB 24 h | 2.0 | (3.10) | 1.7 | (0.82) | −0.7 | (2.07) | 2.4 | (3.29) |
| Day 12 | CFB 48 h | 1.0 | (2.10) | 1.0 | (2.45) | 1.0 | (2.76) | 2.4 | (3.85) |
| Day 13 | CFB 72 h | 0.0 | (2.83) | 1.7 | (1.51) | −2.0 | (1.79) | 1.6 | (3.58) |

Oxygen Saturation (%)

| | | | | | | | | | |
|---|---|---|---|---|---|---|---|---|---|
| Day 1 | pre-dose | 98.8 | (0.41) | 98.7 | (0.52) | 97.8 | (1.17) | 98.2 | (0.98) |
| | CFB 0.5 h | −0.2 | (0.98) | 0.3 | (0.52) | 1.0 | (0.89) | 0.8 | (0.98) |
| | CFB 1 h | −0.7 | (1.21) | 0.3 | (0.82) | 0.7 | (1.51) | 0.5 | (1.22) |
| | CFB 2 h | −0.8 | (1.17) | 0.2 | (0.41) | 0.7 | (1.03) | 0.2 | (1.17) |
| | CFB 4 h | −0.2 | (0.41) | 0.7 | (0.82) | 0.7 | (1.51) | −0.2 | (1.47) |
| | CFB 6 h | −0.5 | (0.84) | −0.3 | (0.52) | 0.3 | (1.51) | 0.0 | (1.41) |
| | CFB 8 h | −0.3 | (0.52) | 0.0 | (0.63) | 0.5 | (1.22) | 0.2 | (1.33) |
| Day 2 | CFB | −0.2 | (0.98) | 0.3 | (0.52) | 1.0 | (1.41) | 0.2 | (1.17) |
| | pre-dose | | | | | | | | |
| | CFB 2 h | −0.7 | (0.82) | 0.2 | (0.98) | −0.2 | (2.14) | −0.5 | (1.76)^{b} |
| Day 3 | CFB | −0.3 | (0.52) | 0.5 | (0.55) | 0.7 | (1.75) | 0.0 | (1.22) |
| | pre-dose | | | | | | | | |
| | CFB 2 h | −0.5 | (1.22) | 0.2 | (0.41) | 0.2 | (1.72) | −1.0 | (1.22) |
| Day 4 | CFB | −0.5 | (1.05) | 0.5 | (0.55) | 0.2 | (1.72) | 0.2 | (1.17)^{b} |
| | pre-dose | | | | | | | | |
| | CFB 2 h | −0.7 | (1.21) | 0.3 | (0.52) | 0.7 | (2.07) | −0.3 | (1.63)^{b} |
| Day 5 | CFB | −0.3 | (1.03) | 0.7 | (0.52) | 0.0 | (2.00) | −0.4 | (1.67) |
| | pre-dose | | | | | | | | |
| | CFB 2 h | −0.3 | (0.82) | 0.0 | (0.63) | −0.2 | (2.14) | −0.8 | (1.64) |
| Day 6 | CFB | −0.5 | (0.55) | 0.5 | (0.55) | 0.0 | (2.19) | −0.6 | (1.52) |
| | pre-dose | | | | | | | | |
| | CFB 2 h | −0.5 | (0.84) | −0.3 | (0.82) | −0.3 | (2.42) | −1.0 | (1.87) |
| Day 7 | CFB | −0.8 | (0.98) | 0.2 | (0.75) | 0.3 | (1.86) | −0.2 | (1.30) |
| | pre-dose | | | | | | | | |
| Day 8 | CFB | 0.0 | (0.63) | 0.5 | (0.55) | 0.8 | (1.72) | 0.2 | (1.79) |
| | pre-dose | | | | | | | | |
| Day 9 | CFB | −0.2 | (1.17) | 0.5 | (0.55) | 0.5 | (1.52) | 0.2 | (1.48) |
| | pre-dose | | | | | | | | |
| Day 10 | CFB | −1.0 | (1.10) | −0.3 | (0.82) | 0.7 | (1.75) | 0.2 | (1.48) |
| | pre-dose | | | | | | | | |
| | CFB 2 h | −0.7 | (0.82) | 0.2 | (0.75) | 0.2 | (2.40) | −0.6 | (1.52) |
| | CFB 4 h | −0.5 | (1.22) | 0.0 | (0.89) | 0.3 | (2.16) | −0.6 | (1.67) |
| | CFB 6 h | −0.7 | (1.21) | 0.0 | (0.00) | 0.5 | (1.38) | −0.2 | (1.48) |
| | CFB 8 h | −0.5 | (1.22) | −0.2 | (0.75) | 0.7 | (2.07) | −0.4 | (1.14) |

CFB = change from baseline,
SD = standard deviation
^{a}For respiratory rate, N = 5 for this cohort from Day 5 pre-dose onward; for oxygen saturation, N = 5 for this cohort on Day 3 and Day 5 pre-dose onward.
^{b}Mean values include Subject 9018's vital signs during monitoring from Day 2 to Day 4 for an adverse event; however, the subject was not dosed on Day 2.
Baseline is defined as pre-dose on Day 1.

Effects of d-Methadone on Blood Pressure:

The data on blood pressure measurements are shown in the table above. These data show a decrease in blood pressure in subjects treated with d-methadone. While this decrease of systolic and diastolic blood pressure remained in the parameters of safety, it signals regulating effects that are potentially useful for treatment of hypertension and the metabolic syndrome and coronary artery disease, including unstable angina. In fact, the blood pressure lowering effects detailed in this Examples section, and the demonstrated presence of NMDA receptors on extra-neural tissues, including the heart and its conduction system [Gill S S. and Pulido O M. Glutamate Receptors in Peripheral Tissues: Current Knowledge, Future Research and Implications for Toxicology. Toxicologic Pathology 2001: 29 (2) 208-223], suggest that d-methadone may be cardio-protective, both against arrhythmias and ischemic heart disease. Ranolazine, a drug approved for the treatment of angina, inhibits persistent or late inward sodium current in heart muscle in voltage-gated sodium channels, thereby reducing intracellular calcium level; d-methadone has similar regulatory activity on ionic currents, not only on squid neurons but also on chick myoblasts [Horrigan F T and Gilly W F: Methadone block of K⁺current in squid giant fiber lobe neurons. J Gen Physiol. 1996 Feb. 1; 107(2): 243-260], suggesting effects similar to those of ranolazine; furthermore, by regulating NMDAR, d-methadone will also result in decreased intracellular calcium overload. Ranolazine influences Na+K+ currents and while it causes prolongation of the Qtc interval, it appears to be cardio-protective rather than arrhythmogenic [Scirica B M et al., Effect of ranolazine, an antianginal agent with novel electrophysiological properties, on the incidence of arrhythmias in patients with non ST-segment elevation acute coronary syndrome: results from the Metabolic Efficiency with Ranolazine for Less Ischemia in Non ST Elevation ST Elevation Acute Coronary Syndrome Thrombolysis in Myocardial Infarction36 (MERLIN-TIMI 36) randomized controlled trial. Circulation. 2007; 116:1647-1652]. Aside for direct effects on ion currents and NMDA receptors outside of the nervous system, the lowering of blood pressure seen in these subjects might also be mediated by NMDA antagonistic effects at hypothalamic neurons with regulation of the hypothalamic-pituitary axis [Glass M J et al., NMDA Receptor Plasticity in the Hypothalamic Paraventricular Nucleus Contributes to the Elevated Blood Pressure Produced by Angiotensin II. The Journal of Neuroscience, 2015 35(26):9558-9567]. The experimental study by Glass et al., indicates that NMDA receptor plasticity in PVN neurons significantly contributes to the elevated blood pressure mediated by angiotensin II.

Statistical Analysis of Systolic and Diastolic Blood Pressure and O₂ Saturation of the MAD-Study Subjects:

These analyses were performed by means of the GraphPad Prism 5.0 software. The data (means of the subjects of each experimental group) were obtained from the clinical study report “A Phase 1 Study to Investigate the Safety, Tolerability, and Pharmacokinetic Profile of Multiple Ascending Doses of d-Methadone in Healthy Subjects”. A 1-way ANOVA followed by the Dunnett post hoc test was performed to compare the three groups of d-methadone treated subjects with placebo in order to evaluate: (1) The effect of the treatment, irrespective of the day and the time point, on the reduction of systolic and diastolic blood pressure and the increase of O₂ saturation; (2) The effect of the treatment 2 hours after dosing at days 1 to 10; and (3) The effect of the treatment 24 hours after dosing at days 2 to 11.

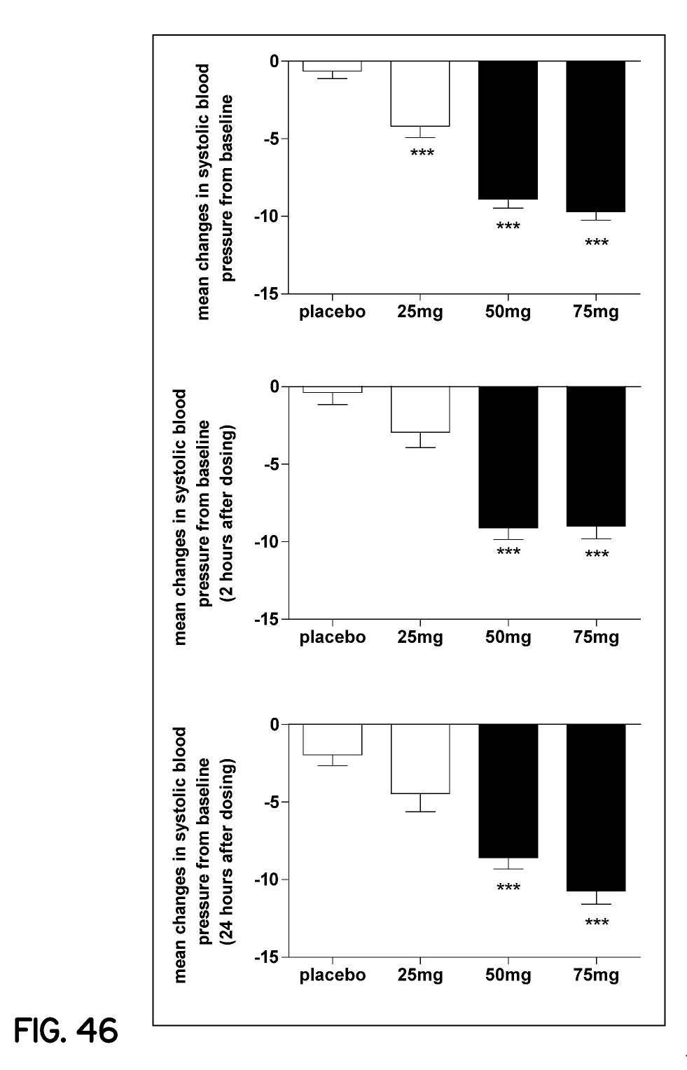

Referring now to FIG. 46, it can be seen that d-methadone treatment significantly decreases systolic blood pressure in the three experimental groups when all the measured time points were considered, whereas only in the 50- and 75-mg groups the mean changes of systolic blood pressure were significantly different from placebo 2 hours and 24 hours after dosing.

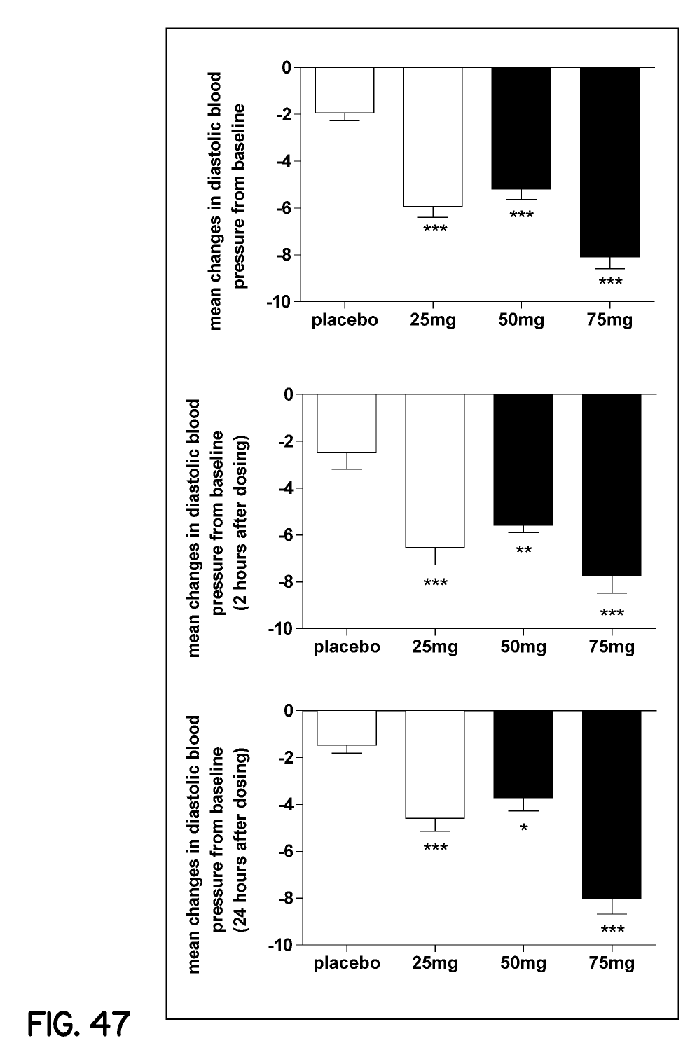

Referring now to FIG. 47, it can be seen that d-methadone treatment significantly decreases diastolic blood pressure in the three experimental groups since the mean changes are significantly different from placebo in the three groups of subjects treated with d-methadone

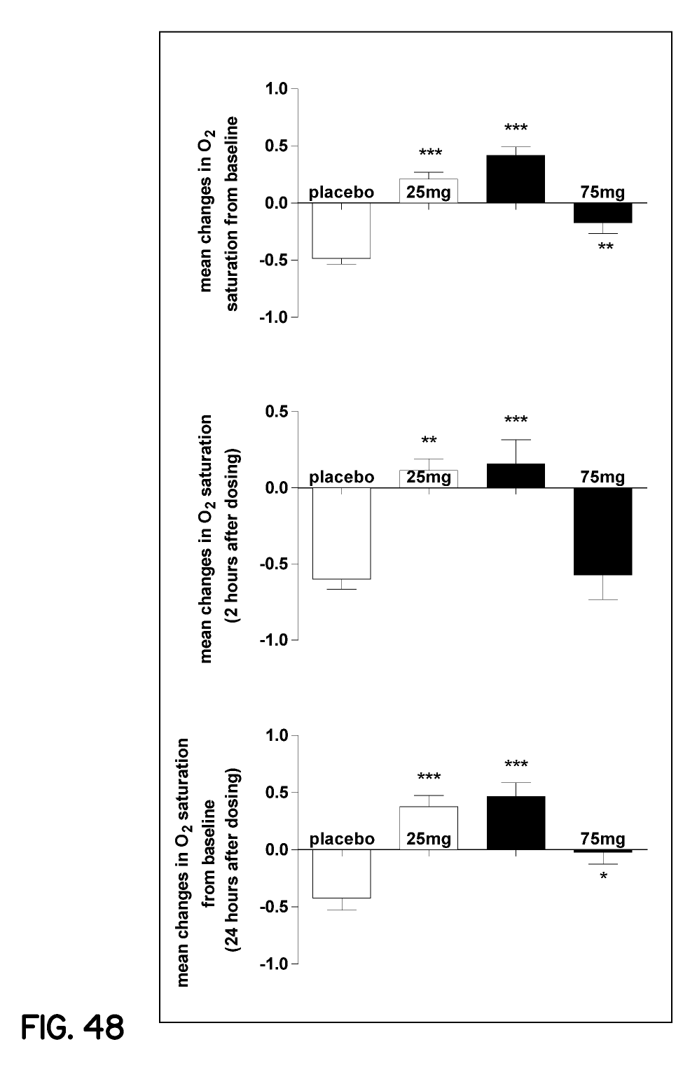

Referring now to FIG. 48, the effect of d-methadone on oxygen saturation can be seen. The mean changes are >0 in the 25- and 50-mg groups (the mean of 02 saturations is therefore increasing in these groups), and the same tendency can be observed in the 75-mg group, where, although the mean change remain <0, a significant difference can be observed with respect to placebo.

Further, in the healthy subjects of the SAD and MAD studies, d-methadone did not cause clinically significant cognitive deficits or psychotomimetic effects (on the Bond-Lader Visual Analog Scale, as will be shown in greater detail in Example 6, below). d-Methadone did not cause symptoms of withdrawal upon abrupt discontinuation after 10 days consecutive of treatment, as tested with the Clinical Opiate Withdrawal Scale (COWS—a test which is well known to those of ordinary skill in the art) pointing away from perceived addictive potential for d-methadone. The lack of significant opioid effects at potentially therapeutic doses and the absence of psychotomimetic effects seen with opioids and other NMDA antagonists (e.g., ketamine and MK-801) and the absence of withdrawal symptoms upon abrupt discontinuation of d-methadone suggest that d-methadone could be used for cognitive improvement. Without the new data provided in this Example (and the other Examples (below), a drug like d-methadone, perceived by those skilled in the art as a drug with probable opioid-like effects and probable psychotomimetic ketamine-like effects, and a drug with addictive potential, would be of little clinical use for improving the cognitive function of patients. The inventors have shown, for the first time, that d-methadone administered to healthy human subjects is devoid of these effects and thus could be successfully used to improve cognitive function in humans.

Cardiac Safety: Effects of d-Methadone on QTc Prolongation, and Treatment-Emergent Adverse Effects (TEAE), MAD Study:

Electrocardiograms (ECGs) were done pre-dose and 2, 4, 6, and 8 hours post-dose from Day 1 to Day 10 and 24 hours post last dose. ECGs were performed after the subjects had been resting in a supine or semi-supine position for at least 5 minutes. The ECG electronically measured and calculated ventricular heart rate and the PR, QRS, QT, and QTc intervals. The Fridericia formula was used for QTc correction.

At the discretion of the investigator, a standard 12-lead ECG with conventional lead placement may have been performed at any time during the study (eg, in the event that potential ischemia or any cardiac abnormality was observed).

Continuous Cardiac Monitoring (Cardiac Telemetry) was performed from pre-dose to at least 8 hours post-dose on Day 1 to Day 10 and included real-time measurements of heart rate and cardiac rhythm.

A Holter monitor was used to collect continuous ECG data. The Holter monitor remained in place with exception for time allowed for personal care and other activities that may have required disconnect from the monitor. The Holter monitor ECG data were sent to iCardiac Technologies for analysis. Continuous Holter recordings were performed on Day 1 through Day 7 and on Day 10 through Day 12. 12-lead ECGs were extracted from the continuous recordings at the following time points (nominal time corresponding to, in all cases), paired with (and preceding) PK blood draws:

Day 1: 45, 30, and 15 minutes pre-dose and 0.5, 1, 2, 4, 6, 8, and 12 hours post-dose
Day 2 to Day 6: 1 hour pre-dose and 2, 4, 6, and 8 hours post-dose
Day 7: 1 hour pre-dose
Day 10: 1 hour pre-dose and 2, 4, 6, 8, and 12 hours post-dose
Day 11: 24 and 36 hours post last dose
Day 12: approximately 48 hours post last dose

The 12-lead Holter and ECG equipment were supplied and supported by iCardiac Technologies. All ECG data were collected using a Global Instrumentation (Manlius, N.Y., USA) M12R ECG continuous 12-lead digital recorder. The continuous 12-lead digital ECG data were stored on SD memory cards. ECGs to be used in the analyses were read centrally by iCardiac Technologies.

The following principles were followed in iCardiac's core laboratory:

(1) ECG analysts were blinded to the subject, visit, and treatment allocation.
(2) Baseline and on-treatment ECGs for a particular subject were over-read on the same lead and were analyzed by the same reader.
(3) The primary analysis lead was lead II. If lead II was not analyzable, then the primary lead of analysis was changed to another lead for the entire subject data set.

Abnormal, not clinically significant, ECG interpretations by the investigator are presented by treatment group and time point in Table 8, below. There were no clinically significant abnormal scheduled ECGs during the study.

TABLE 8

| | | | d-Methadone | | |
|---|---|---|---|---|---|
| | | Placebo (N = 6) | 25 mg (N = 6) | 50 mg (N = 6) | 75 mg (N = 6)^{a} |
| Time Point | | n (%) | | | |
| Day 1 | pre-dose | 0 | 1 (16.7) | 0 | 1 (16.7) |
| | 2 h | 0 | 1 (16.7) | 0 | 1 (16.7) |
| | 4 h | 0 | 1 (16.7) | 1 (16.7) | 2 (33.3) |
| | 6 h | 1 (16.7) | 1 (16.7) | 0 | 1 (16.7) |
| | 8 h | 0 | 1 (16.7) | 0 | 1 (16.7) |
| Day 2 | pre-dose | 1 (16.7) | 1 (16.7) | 1 (16.7) | 2 (33.3) |
| | 2 h | 1 (16.7) | 1 (16.7) | 0 | 1 (16.7) |
| Day 3 | pre-dose | 0 | 2 (33.3) | 3 (50.0) | 1 (16.7) |
| | 2 h | 0 | 1 (16.7) | 3 (50.0) | 2 (40.0) |
| Day 4 | pre-dose | 0 | 1 (16.7) | 2 (33.3) | 2 (33.3) |
| | 2 h | 0 | 1 (16.7) | 1 (16.7) | 1 (16.7) |
| Day 5 | pre-dose | 1 (16.7) | 1 (16.7) | 1 (16.7) | 1 (20.0) |
| | 2 h | 0 | 1 (16.7) | 0 | 1 (20.0) |
| Day 6 | pre-dose | 0 | 1 (16.7) | 0 | 1 (20.0) |
| | 2 h | 2 (33.3) | 2 (33.3) | 0 | 3 (60.0) |
| Day 7 | pre-dose | 1 (16.7) | 1 (16.7) | 0 | 1 (20.0) |
| | 2 h | 1 (16.7) | 2 (33.3) | 0 | 1 (20.0) |
| Day 8 | pre-dose | 1 (16.7) | 1 (16.7) | 0 | 1 (20.0) |
| | 2 h | 1 (16.7) | 1 (16.7) | 0 | 1 (20.0) |
| Day 9 | pre-dose | 1 (16.7) | 1 (16.7) | 0 | 1 (20.0) |
| | 2 h | 2 (33.3) | 1 (16.7) | 0 | 2 (40.0) |
| Day 10 | pre-dose | 1 (16.7) | 1 (16.7) | 0 | 1 (20.0) |
| | 2 h | 1 (16.7) | 1 (16.7) | 0 | 2 (40.0) |
| | 4 h | 1 (16.7) | 1 (16.7) | 0 | 2 (40.0) |
| | 6 h | 1 (16.7) | 0 | 0 | 3 (60.0) |
| | 8 h | 1 (16.7) | 0 | 0 | 2 (40.0) |
| Day 11 | 24 h | 0 | 1 (16.7) | 2 (33.3) | 1 (20.0) |

CFB = change from baseline,
F/U = follow-up,
SD = standard deviation
^{a}For this treatment group, N = 5 for all post-dose time points on Day 3 and from Day 5 pre-dose onward.

There were several ECG-related AEs—all non clinically significant—during the study, as follows:

Subject 9005 experienced ventricular extrasystoles (ie, premature ventricular contractions) on Day 5, approximately 6 hours and 30 minutes after administration of 25 mg d-methadone. This AE was assessed as mild and unrelated to study drug.

Subject 9007 experienced ventricular extrasystoles (ie, premature ventricular contractions with run of bigeminy) on Day 7, approximately 1 hour and 30 minutes after administration of 25 mg d-methadone. This AE was assessed as mild and possibly related to study drug.

Subject 9011 experienced sinus tachycardia on Day 4, 2 hours after dosing with 25 mg d-methadone. This AE was assessed as mild and possibly related to study drug.

Subject 9018 experienced bradycardia on Day 1, 22 hours and 12 minutes following administration of 75 mg d-methadone. This AE was assessed as mild and possibly related to study drug.

Subject 9027 experienced ventricular extrasystoles (ie, premature ventricular contractions) on Day 6, approximately 1 hour and 20 minutes after being dosed with 50 mg d-methadone. This AE was assessed as mild unrelated to study drug. This subject also experienced extrasystoles (ie, bigeminy) on Day 10, 1 hour and 35 minutes after dosing, and ventricular extrasystoles (ie, ventricular ectopy) on Day 10, 23 hours and 15 minutes after dosing. Both AEs on Day 10 were assessed as mild and possibly related to study drug. It should be noted that Subject 9027 had a medical history finding of ongoing ventricular extrasystoles; however, previous assessment by a cardiologist deemed the subject to have stable cardiac status.

Given that QTc prolongation has been a concern with racemic methadone, this ECG abnormality was of particular interest for d-methadone. In this study, a QTcF interval >450 ms in females or >430 ms in males was considered prolonged. Three subjects, all in the 75 mg d-methadone group, had an ECG abnormality of QTcF prolongation as defined above during the study, but none were clinically significant:

Subject 9019 (female) experienced 4 incidences of QTc prolongation, at 4 hours post-dose on Day 6 (455 msec), at 8 hours post-dose on Day 7 (458 msec) and Day 9 (452 msec), and at 6 hours post-dose on Day 10 (452 msec).

Subject 9035 (female) experienced 4 incidences of QTc prolongation, at 2 hours post-dose on Day 6 (454 msec), at 2 hours and 8 hours post-dose on Day 9 (453 msec each), and at 6 hours post-dose on Day 10 (462 msec).

Subject 9036 (male) experienced 1 incidence of QTc prolongation, at 2 hours post-dose on Day 6 (434 msec).

Table 9 below shows a Summary of Abnormal (NCS) Overall Electrocardiogram Interpretation Results (Safety Population):

TABLE 9
Summary of QTcF Interval Changes from Baseline (Safety Population)

| | | | | | | d-Methadone | | | | | | | | | | | |
|---|---|---|---|---|---|---|---|---|---|---|---|---|---|---|---|---|---|
| | | Placebo | | | | 25 mg | | | | 50 mg | | | | 75 mg | | | |
| | | (N = 6) | | | | (N = 6) | | | | (N = 6) | | | | (N = 6)^{a} | | | |
| | | Mean | | Median | | Mean | | Median | | Mean | | Median | | Mean | | Median | |
| Time Point | | (SD) | | (Range) | | (SD) | | (Range) | | (SD) | | (Range) | | (SD) | | (Range) | |
| Day 1 | pre-dose | 401.2 | (18.13) | 403.5 | (369, 424) | 399.0 | (26.81) | 404.0 | (359, 426) | 409.7 | (15.64) | 410.0 | (391, 429) | 412.2 | (12.11) | 409.0 | (400, 435) |
| | CFB 2 h | −4.5 | (9.54) | −1.0 | (−23, 2) | 16.3 | (7.39) | 16.5 | (4, 26) | 3.8 | (10.36) | 4.5 | (−9, 15) | 14.0 | (13.21) | 13.0 | (−5, 32) |
| | CFB 4 h | −7.5 | (7.45) | −5.0 | (−22, −1) | −0.7 | (9.69) | −3.0 | (−12, 16) | −3.3 | (9.93) | −0.5 | (−19, 7) | 11.3 | (10.82) | 15.0 | (−2, 23) |
| | CFB 6 h | −4.7 | (15.24) | −8.0 | (−26, 19) | 3.2 | (12.62) | 1.0 | (−11, 22) | −1.8 | (3.76) | −2.5 | (−6, 5) | 2.2 | (5.46) | 3.5 | (−6, 8) |
| | CFB 8 h | −7.8 | (11.18) | −6.0 | (−28, 5) | −0.2 | (14.27) | 4.5 | (−18, 15) | −2.8 | (4.45) | −2.5 | (−10, 3) | 3.0 | (6.99) | 4.5 | (−6, 10) |
| Day 2 | CFB | 2.0 | (6.54) | 1.0 | (−7, 10) | −5.2 | (13.41) | −8.0 | (−24, 14) | −5.7 | (10.89) | −3.0 | (−19, 9) | 6.8 | (8.59) | 5.0 | (−4, 18) |
| | pre-dose | | | | | | | | | | | | | | | | |
| | CFB 2 h | −3.3 | (15.40) | −1.0 | (−30, 17) | 7.2 | (12.83) | 5.5 | (−8, 24) | 6.0 | (8.00) | 4.5 | (−5, 19) | 12.2 | (14.27)^{b} | 10.5 | (−10, 30)^{b} |
| | CFB 4 h | −9.2 | (6.88) | −7.5 | (−21, −3) | 3.5 | (11.73) | −0.5 | (−8, 18) | −1.8 | (5.49) | −0.5 | (−9, 4) | 8.0 | (11.26)^{b} | 8.5 | (−8, 22)^{b} |
| | CFB 6 h | −4.2 | (12.46) | −1.0 | (−26, 11) | 5.2 | (15.32) | 4.0 | (−12, 25) | 1.0 | (9.96) | −1.0 | (−11, 15) | 6.8 | (9.95)^{b} | 5.5 | (−7, 21)^{b} |
| | CFB 8 h | −5.0 | (10.88) | −3.0 | (−24, 8) | 0.3 | (11.55) | 3.0 | (−16, 16) | −3.8 | (13.41) | −2.0 | (−21, 15) | 7.5 | (7.15)^{b} | 7.0 | (−1, 19)^{b} |
| Day 3 | CFB | −2.5 | (10.05) | −3.0 | (−14, 14) | 1.3 | (17.65) | 3.0 | (−23, 20) | −3.5 | (10.65) | −1.0 | (−21, 9) | 0.5 | (15.42)^{b} | 3.5 | (−27, 18)^{b} |
| | pre-dose | | | | | | | | | | | | | | | | |
| | CFB 2 h | −1.8 | (13.09) | 1.5 | (−23, 13) | 1.5 | (52.11) | 9.5 | (−93, 48) | 9.7 | (9.07) | 13.0 | (−6, 18) | 19.2 | (11.10) | 18.0 | (3, 33) |
| | CFB 4 h | −11.8 | (11.65) | −7.5 | (−32, −1) | 1.7 | (16.50) | 5.0 | (−18, 19) | 1.2 | (10.65) | 2.0 | (−15, 18) | 11.0 | (10.46) | 10.0 | (−2, 25) |
| | CFB 6 h | −10.2 | (13.20) | −7.5 | (−35, 4) | 6.7 | (12.60) | 5.5 | (−7, 21) | 2.2 | (7.78) | 1.5 | (−9, 14) | 14.2 | (9.58) | 14.0 | (1, 28) |
| | CFB 8 h | −6.2 | (8.70) | −6.5 | (−21, 4) | 3.3 | (11.54) | 3.0 | (−8, 17) | −0.8 | (11.81) | 4.0 | (−19, 10) | 16.2 | (11.78) | 12.0 | (4, 35) |
| Day 4 | CFB | −7.0 | (11.05) | −7.5 | (−25, 5) | −2.2 | (12.95) | −1.5 | (−21, 18) | 2.7 | (6.35) | 2.0 | (−6, 10) | 0.7 | (11.91)^{b} | 5.0 | (−23, 9)^{b} |
| | pre-dose | | | | | | | | | | | | | | | | |
| | CFB 2 h | −0.2 | (14.74) | 4.5 | (−28, 11) | 7.2 | (17.97) | 10.5 | (−20, 25) | 4.3 | (11.60) | 7.5 | (−10, 16) | 12.3 | (15.42)^{b} | 18.5 | (−14, 27)^{b} |
| | CFB 4 h | −7.8 | (14.11) | −4.0 | (−35, 5) | 7.5 | (12.14) | 12.5 | (−11, 18) | 2.0 | (9.47) | 1.0 | (−10, 16) | 2.0 | (17.08)^{b} | 6.0 | (−25, 18)^{b} |
| | CFB 6 h | −9.2 | (13.92) | −9.0 | (−30, 11) | 1.2 | (27.48) | 6.0 | (−42, 28) | 4.0 | (8.51) | 4.5 | (−7, 14) | 4.3 | (11.00)^{b} | 6.0 | (−12, 18)^{b} |
| | CFB 8 h | −6.2 | (9.33) | −4.0 | (−22, 6) | 5.5 | (9.79) | 6.0 | (−8, 16) | 5.7 | (10.33) | 6.0 | (−6, 21) | 8.3 | (12.34)^{b} | 10.0 | (−15, 22)^{b} |
| Day 5 | CFB | −5.7 | (9.89) | −7.0 | (−19, 6) | 2.8 | (13.82) | 2.5 | (−18, 21) | −4.7 | (15.00) | −3.0 | (−29, 17) | 7.8 | (8.07) | 4.0 | (0, 17) |
| | pre-dose | | | | | | | | | | | | | | | | |
| | CFB 2 h | −7.5 | (13.90) | −3.5 | (−32, 5) | 13.2 | (18.56) | 7.5 | (−3, 46) | 10.7 | (4.76) | 11.5 | (3, 17) | 20.6 | (9.58) | 15.0 | (12, 32) |
| | CFB 4 h | −5.3 | (11.20) | −3.0 | (−20, 6) | 1.8 | (14.82) | 4.5 | (−16, 17) | 4.2 | (11.51) | 6.0 | (−16, 15) | 12.6 | (8.14) | 10.0 | (3, 25) |
| | CFB 6 h | −5.2 | (14.80) | −3.0 | (−34, 6) | 7.3 | (13.28) | 7.0 | (−7, 25) | 6.7 | (9.63) | 6.5 | (−9, 20) | 15.0 | (7.62) | 10.0 | (9, 26) |
| | CFB 8 h | −5.0 | (11.24) | −2.5 | (−24, 8) | 9.5 | (16.07) | 10.0 | (−10, 29) | 1.0 | (13.28) | −0.5 | (−15, 24) | 14.0 | (3.54) | 13.0 | (11, 20) |
| Day 6 | CFB | −3.5 | (10.71) | −3.5 | (−18, 13) | 6.2 | (14.34) | 5.0 | (−14, 26) | 3.8 | (11.30) | 3.5 | (−11, 16) | 6.8 | (8.90) | 8.0 | (−5, 17) |
| | pre-dose | | | | | | | | | | | | | | | | |
| | CFB 2 h | 1.3 | (10.29) | 3.5 | (−16, 15) | 1.7 | (39.75) | 11.0 | (−73, 33) | 15.0 | (7.69) | 14.5 | (5, 26) | 27.8 | (12.70) | 24.0 | (13, 42) |
| | CFB 4 h | −10.8 | (17.66) | −6.5 | (−43, 6) | 8.8 | (17.06) | 15.0 | (−16, 28) | 8.0 | (10.66) | 9.5 | (−11, 18) | 24.6 | (20.48) | 11.0 | (8, 47) |
| | CFB 6 h | −6.0 | (15.82) | −4.5 | (−35, 12) | 7.0 | (15.23) | 13.5 | (−17, 20) | 12.0 | (11.37) | 12.0 | (−5, 25) | 14.8 | (7.29) | 18.0 | (2, 20) |
| | CFB 8 h | −5.7 | (12.79) | −4.5 | (−26, 10) | 8.2 | (13.91) | 10.5 | (−12, 25) | 6.0 | (6.03) | 6.0 | (−4, 15) | 18.0 | (10.37) | 18.0 | (7, 34) |
| Day 7 | CFB | −0.8 | (8.89) | −4.0 | (−6, 17) | −0.7 | (9.69) | 0.5 | (−13, 10) | 1.5 | (10.84) | 1.5 | (−10, 12) | 11.0 | (7.71) | 10.0 | (3, 23) |
| | pre-dose | | | | | | | | | | | | | | | | |
| | CFB 2 h | −1.8 | (16.96) | 5.0 | (−34, 11) | 1.2 | (33.69) | 7.0 | (−54, 37) | 12.5 | (7.04) | 14.5 | (3, 21) | 11.4 | (5.41) | 9.0 | (5, 18) |
| | CFB 4 h | −10.0 | (13.34) | −10.0 | (−34, 3) | 4.5 | (14.20) | 6.0 | (−18, 24) | 7.3 | (5.39) | 9.0 | (−1, 14) | 14.0 | (7.11) | 13.0 | (7, 24) |
| | CFB 6 h | −6.8 | (14.08) | −2.5 | (−35, 4) | 5.0 | (17.38) | 5.5 | (−19, 27) | 9.8 | (6.18) | 11.5 | (1, 17) | 19.4 | (12.46) | 17.0 | (5, 38) |
| | CFB 8 h | −4.0 | (13.89) | 0.0 | (−31, 7) | 10.3 | (16.67) | 12.5 | (−8, 28) | 12.2 | (9.99) | 16.0 | (−7, 20) | 24.8 | (15.53) | 21.0 | (9, 50) |
| Day 8 | CFB | −1.8 | (10.44) | 2.5 | (−22, 6) | 6.3 | (6.86) | 6.5 | (−3, 18) | 0.0 | (12.30) | 2.0 | (−19, 12) | 4.6 | (5.68) | 5.0 | (−2, 13) |
| | pre-dose | | | | | | | | | | | | | | | | |
| | CFB 2 h | 4.0 | (14.91) | 7.0 | (−25, 16) | 14.8 | (18.17) | 12.0 | (−3, 44) | 13.5 | (8.50) | 13.0 | (4, 29) | 14.0 | (11.92) | 16.0 | (−2, 30) |
| | CFB 4 h | −6.2 | (13.64) | −3.0 | (−32, 6) | 5.0 | (13.78) | 11.5 | (−17, 17) | 8.2 | (7.36) | 5.5 | (2, 21) | 8.6 | (10.90) | 6.0 | (−7, 20) |
| | CFB 6 h | −3.7 | (18.15) | −5.5 | (−34, 17) | 9.5 | (9.48) | 8.5 | (0, 23) | 6.7 | (9.14) | 9.5 | (−5, 17) | 16.4 | (5.98) | 17.0 | (7, 22) |
| | CFB 8 h | −4.8 | (18.55) | 0.5 | (−41, 9) | 11.7 | (15.81) | 11.0 | (−5, 32) | 9.3 | (9.99) | 8.0 | (−1, 23) | 21.0 | (5.61) | 21.0 | (15, 30) |
| Day 9 | CFB | −2.7 | (7.45) | 0.0 | (−17,3) | 1.8 | (10.94) | 0.5 | (−11, 15) | 7.0 | (13.52) | 11.5 | (−11, 23) | 9.0 | (9.82) | 12.0 | (−6, 19) |
| | pre-dose | | | | | | | | | | | | | | | | |
| | CFB 2 h | −0.3 | (11.83) | 4.0 | (−24, 8) | −0.3 | (46.99) | 12.0 | (−92, 41) | 14.5 | (10.03) | 12.0 | (4, 33) | 15.0 | (18.43) | 17.0 | (−9, 39) |
| | CFB 4 h | −5.0 | (13.37) | −5.5 | (−21, 12) | 5.3 | (12.13) | 7.0 | (−13, 18) | 12.0 | (8.58) | 12.0 | (0, 23) | 16.0 | (14.23) | 16.0 | (−2, 34) |
| | CFB 6 h | −7.5 | (14.14) | −3.5 | (−33, 7) | 8.8 | (17.42) | 8.0 | (−15, 29) | 6.3 | (9.46) | 8.0 | (−11, 15) | 16.0 | (8.25) | 15.0 | (6, 27) |
| | CFB 8 h | −7.3 | (16.90) | −3.5 | (−40, 10) | 10.5 | (17.91) | 5.5 | (−6, 38) | 7.7 | (7.28) | 6.5 | (−4, 16) | 22.4 | (17.60) | 11.0 | (7, 44) |
| Day 10 | CFB | −4.0 | (9.82) | −0.5 | (−20, 4) | 1.2 | (12.51) | 1.5 | (−16, 15) | 1.7 | (15.86) | 5.0 | (−20, 17) | 11.2 | (13.44) | 6.0 | (2, 35) |
| | pre-dose | | | | | | | | | | | | | | | | |
| | CFB 2 h | 2.7 | (9.11) | 3.5 | (−10, 14) | 20.2 | (17.01) | 19.0 | (−3, 41) | 14.8 | (12.62) | 15.0 | (−4, 35) | 28.2 | (10.21) | 29.0 | (12, 40) |
| | CFB 4 h | −5.2 | (12.22) | −4.0 | (−27, 8) | 8.0 | (11.40) | 6.5 | (−6, 26) | 9.2 | (10.87) | 11.5 | (−12, 19) | 21.8 | (13.75) | 21.0 | (7, 41) |
| | CFB 6 h | −4.3 | (13.54) | 2.0 | (−30, 5) | 11.3 | (14.87) | 12.0 | (−5, 27) | 7.2 | (7.60) | 8.5 | (−6, 16) | 29.2 | (16.18) | 25.0 | (11, 48) |
| | CFB 8 h | −1.8 | (14.93) | 2.5 | (−31, 11) | 8.0 | (15.99) | 9.0 | (−14, 26) | 10.5 | (16.26) | 9.5 | (−15, 34) | 20.0 | (12.57) | 14.0 | (8, 40) |
| Day 11 | CFB 24 h | −2.0 | (10.35) | −2.0 | (−18, 10) | 2.5 | (20.11) | 4.0 | (−20, 26) | 4.7 | (7.00) | 3.0 | (−3, 14) | 16.0 | (8.80) | 18.0 | (1, 24) |
| F/U 1 | CFB | 0.0 | (13.08) | 5.5 | (−23, 11) | 3.0 | (16.30) | 5.5 | (−16, 20) | 0.0 | (9.38) | 0.5 | (−15, 11) | 6.0 | (6.12) | 6.0 | (−2, 15) |
| F/U 3 | CFB | −1.8 | (9.52) | 0.0 | (−19, 7) | 2.8 | (12.73) | 4.0 | (−17, 16) | −1.7 | (5.65) | −2.5 | (−8, 5) | −3.8 | (8.11) | −8.0 | (−9, 10) |

CFB = change from baseline,
F/U = follow-up,
SD = standard deviation
^{a}For this treatment group, N = 5 for all post-dose time points on Day 3 and from Day 5 pre-dose onward.
^{b}Mean and median values include Subject 9018's electrocardiogram data during monitoring from Day 2 to Day 4 for an adverse event; however, the subject was not dosed on Day 2.
Baseline is defined as pre-dose on Day 1.

For the d-methadone treatment groups, the QTcF interval increased over the duration of the study. On Day 1, the largest mean placebo-corrected CFB values for QTcF (ΔΔQTcF) occurred at 2 hours post-dose: 6.8 msec, 15.2 msec, and 16.0 msec in the 25 mg, 50 mg, and 75 mg d-methadone groups, respectively. On Day 10, these values increased to 12.4 msec (12 hours post-dose), 26.8 msec (2 hours post-dose), and 28.8 msec (8 hours post-dose). 1, 2, and 3 subjects had a CFB value >30 msec in the 25 mg, 50 mg, and 75 mg d-methadone groups, respectively. No subject had a CFB value greater than 60 msec and no subject had a QTcF greater than 480 msec; The maximum QTcF interval observed in the study was 462 ms.

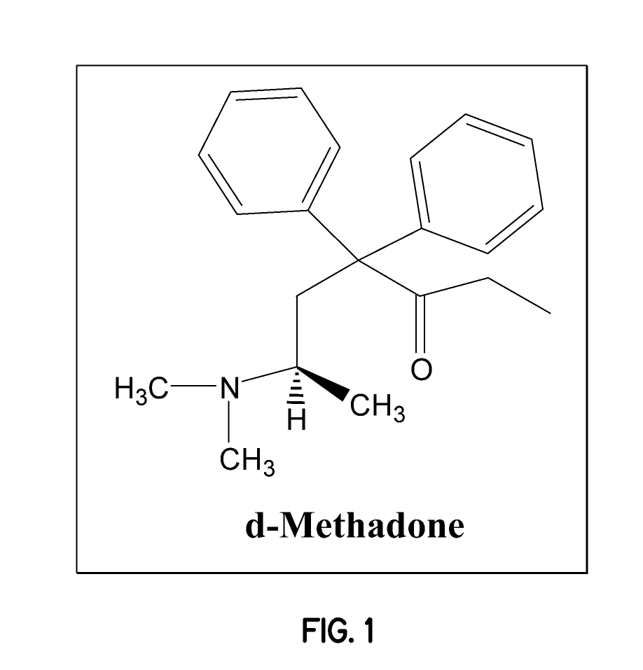

In the exposure-response analysis, the initial investigation of the data indicated a non-linear relationship between ΔΔQTcF and plasma concentrations. A quadratic term was therefore fit and found to be statistically significant, and an investigation was performed into non-linear models. The result of the investigation determined that by using log-transformation on concentration, Conc=log(Conc/C0), the relationship between ΔΔQTcF and the plasma concentration could be accurately modeled. Furthermore, it was noted that the geometric mean Cₘₐₓ of the 50 mg treatment group was higher than that of the 75 mg group and that 3 subjects in the 50 mg group had higher concentrations than in the 75 mg group. An additional sensitivity analysis was therefore performed excluding these 3 subjects from the population. This model provided a better fit to the data. All 3 models confirmed a QTc prolonging effect of d-methadone with a statistically significant slope of the relationship between plasma concentrations and ΔΔQTcF (see FIG. 1 below) for the log-transformation model). The predicted ΔΔQT effect at the observed geometric mean d-methadone plasma concentration for the 2 highest doses (50 mg: 587 ng/mL; 75 mg: 563 ng/mL) varied between 16.0 msec and 21.0 msec, which is notably lower than the observed effect. Extrapolation of the size of the QT effect to patients should therefore be done with caution.

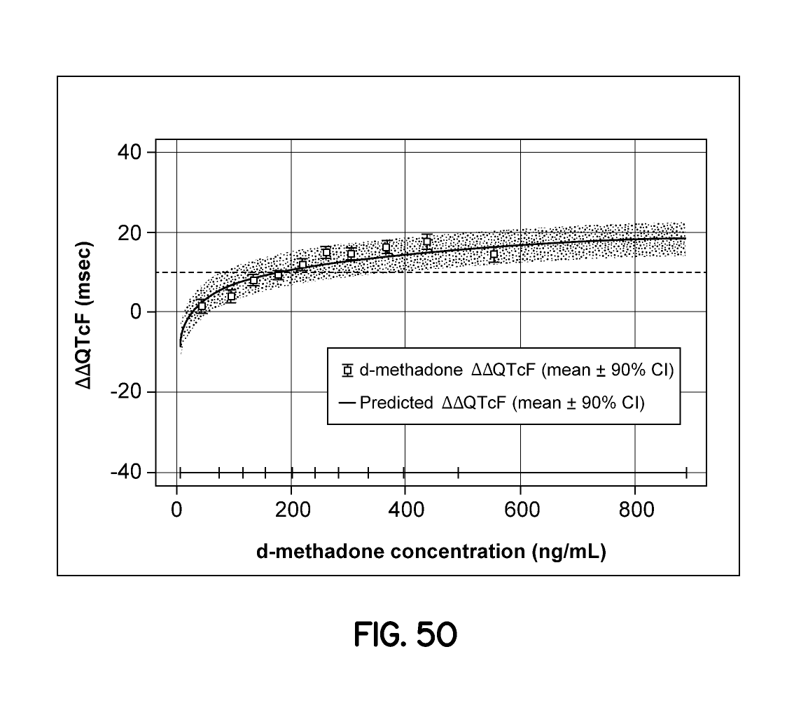

Referring to FIG. 50, one can see model-predicted and observed ΔΔQTcF across deciles of d-methadone plasma concentrations.

To summarize, cardiodynamic ECG analysis in the MAD study showed that the QTcF interval increases in a d-methadone concentration-dependent manner. These increases never reached clinical significance and no subject in the study exhibited pronounced QTcF prolongation defined as change from baseline of >60 msec or absolute QTcF >480 msec.

Cardiac Safety: Effects of d-Methadone on QTc Prolongation, SAD:

Overall ECG interpretation is presented by treatment group and time point in Table 10, below. There were no clinically significant abnormal scheduled ECGs during the study. Overall, the incidence of abnormal ECGs (not clinically significant) was highest in the placebo group (excluding the 100% incidence in the 200 mg d-methadone group of N=1).

TABLE 10
Summary of Overall Electrocardiogram Interpretation Results (Safety Population)

| | | | d-Methadone | | | | | | | |
|---|---|---|---|---|---|---|---|---|---|---|
| | | Placebo | 5 mg | | 20 mg | 60 mg | 100 mg | | 150 mg | 200 mg |
| | | (N = 11) | (N = 6) | | (N = 6) | (N = 6) | (N = 6) | | (N = 6) | (N = 1) |
| Time Point | ECG Result | n (%) | | | | | | | | |
| Pre-dose | Normal | 9 (81.8) | 6 | (100) | 6 (100) | 6 (100) | 6 | (100) | 4 (66.7) | 0 |
| | Abnormal, | 2 (18.2) | 0 | | 0 | 0 | 0 | | 2 (33.3) | 1 (100) |
| | NCS | | | | | | | | | |
| 0.5 h | Normal | 7 (63.6) | 6 | (100) | 5 (83.3) | 5 (83.3) | 5 | (83.3) | 5 (83.3) | 0 |
| | Abnormal, | 4 (36.4) | 0 | | 1 (16.7) | 1 (16.7) | 1 | (16.7) | 1 (16.7) | 1 (100) |
| | NCS | | | | | | | | | |
| 1 h | Normal | 3 (27.3) | 5 | (83.3) | 5 (83.3) | 4 (66.7) | 6 | (100) | 4 (66.7) | 0 |
| | Abnormal, | 8 (72.7) | 1 | (16.7) | 1 (16.7) | 2 (33.3) | 0 | | 2 (33.3) | 1 (100) |
| | NCS | | | | | | | | | |
| 2 h | Normal | 5 (45.5) | 3 | (50.0) | 4 (66.7) | 4 (66.7) | 5 | (83.3) | 4 (66.7) | 0 |
| | Abnormal, | 6 (54.5) | 3 | (50.0) | 2 (33.3) | 2 (33.3) | 1 | (16.7) | 2 (33.3) | 1 (100) |
| | NCS | | | | | | | | | |
| 3 h | Normal | 3 (27.3) | 4 | (66.7) | 4 (66.7) | 5 (83.3) | 3 | (50.0) | 2 (33.3) | 0 |
| | Abnormal, | 8 (72.7) | 2 | (33.3) | 2 (33.3) | 1 (16.7) | 3 | (50.0) | 4 (66.7) | 1 (100) |
| | NCS | | | | | | | | | |
| 5 h | Normal | 5 (45.5) | 4 | (66.7) | 5 (83.3) | 5 (83.3) | 5 | (83.3) | 4 (66.7) | 0 |
| | Abnormal, | 6 (54.5) | 2 | (33.3) | 1 (16.7) | 1 (16.7) | 1 | (16.7) | 2 (33.3) | 1 (100) |
| | NCS | | | | | | | | | |
| 8 h | Normal | 7 (63.6) | 6 | (100) | 5 (83.3) | 4 (66.7) | 5 | (83.3) | 4 (66.7) | 1 (100) |
| | Abnormal, | 4 (36.4) | 0 | | 1 (16.7) | 2 (33.3) | 1 | (16.7) | 2 (33.3) | 0 |
| | NCS | | | | | | | | | |
| 12 h | Normal | 7 (63.6) | 6 | (100) | 5 (83.3) | 3 (50.0) | 6 | (100) | 4 (66.7) | 1 (100) |
| | Abnormal, | 4 (36.4) | 0 | | 1 (16.7) | 3 (50.0) | 0 | | 2 (33.3) | 0 |
| | NCS | | | | | | | | | |
| 24 h | Normal | 9 (81.8) | 5 | (83.3) | 5 (83.3) | 4 (66.7) | 5 | (83.3) | 5 (83.3) | 0 |
| | Abnormal, | 2 (18.2) | 1 | (16.7) | 1 (16.7) | 2 (33.3) | 1 | (16.7) | 1 (16.7) | 1 (100) |
| | NCS | | | | | | | | | |
| 48 h | Normal | 5 (45.5) | 6 | (100) | 4 (66.7) | 4 (66.7) | 5 | (83.3) | 5 (83.3) | 1 (100) |
| | Abnormal, | 6 (54.5) | 0 | | 2 (33.3) | 2 (33.3) | 1 | (16.7) | 1 (16.7) | 0 |
| | NCS | | | | | | | | | |
| 72 h | Normal | 9 (81.8) | 6 | (100) | 5 (83.3) | 5 (83.3) | 6 | (100) | 6 (100) | 1 (100) |
| | Abnormal, | 2 (18.2) | 0 | | 1 (16.7) | 1 (16.7) | 0 | | 0 | 0 |
| | NCS | | | | | | | | | |
| Visit 3 | Normal | 10 (90.9) | 5 | (83.3) | 6 (100) | 5 (83.3) | 6 | (100) | 6 (100) | 1 (100) |

| | | | | | | | | |
|---|---|---|---|---|---|---|---|---|
| Abnormal, | 1 (9.1) | 1 | (16.7) | 0 | 1 (16.7) | 0 | 0 | 0 |
| NCS | | | | | | | | |

NCS = not clinically significant

There were three cardiac-related TEAEs during the study that were observed during telemetry:

First, subject 9005 experienced supraventricular tachycardia approximately 3 hours and 40 minutes following administration of placebo that lasted for less than 1 minute. This TEAE was assessed by the investigator as possibly related to study drug. All scheduled ECGs for this subject were normal.

Second, subject 9036 experienced sinus bradycardia approximately 1 hour and 14 minutes following administration of 60 mg d-methadone. This TEAE lasted for approximately 2 hours and 47 minutes. This TEAE was assessed by the investigator as probably related to study drug. It is noteworthy that this subject had several scheduled ECGs during the study that indicated sinus bradycardia, including at screening and admission, but none were considered clinically significant.

And third, subject 9058 experienced ventricular extrasystoles approximately 3 hours and 39 minutes following administration of placebo that lasted less than 1 minute. This TEAE was assessed by the investigator as possibly related to study drug. This subject had several scheduled ECGs during the study that showed abnormalities, including at screening and admission, but none were considered clinically significant.

All three TEAEs were assessed by the investigator as mild in intensity, and all three subjects recovered without intervention.

A summary of the incidence of QTcF prolongation observed during the study is provided by treatment group and time point in Table 11 below.

TABLE 11
Summary of ECG Abnormal Results: QTcF Prolongation (Safety Population)

| | | | d-Methadone | | | | | |
|---|---|---|---|---|---|---|---|---|
| | Placebo | | 5 mg | 20 mg | 60 mg | 100 mg | 150 mg | 200 mg |
| | (N = 11) | | (N = 6) | (N = 6) | (N = 6) | (N = 6) | (N = 6) | (N = 1) |
| Time Point | n (%) | | | | | | | |
| Pre-dose | 1 | (9.1) | | | | 1 (16.7) | | |
| 0.5 h | | | | | | 1 (16.7) | | |
| 1 h | 2 | (18.2) | 1 (16.7) | | | | | |
| 2 h | | | 2 (33.3) | | | 1 (16.7) | | |
| 3 h | 1 | (9.1) | 1 (16.7) | | | 1 (16.7) | 2 (33.3) | 1 (100) |
| 5 h | 1 | (9.1) | 2 (33.3) | | | 1 (16.7) | 1 (16.7) | |
| 8 h | | | | | | 1 (16.7) | | |
| 12 h | 1 | (9.1) | | | | | 1 (16.7) | |
| 24 h | 1 | (9.1) | | | | 1 (16.7) | | |
| 48 h | | | | | | 1 (16.7) | | |
| Total | 3 | (27.3) | 3 (50.0) | 0 | 0 | 1 (16.7) | 2 (33.3) | 1 (100) |

The QTcF prolongations that occurred in the study are summarized by subject in Table 12 below. All 3 readings and the average are provided for each time point, and pre-dose values are provided as a baseline comparison (prolonged values are in boldface). None of the QTcF prolongations observed during the study were considered clinically significant by the investigator.

TABLE 12
QTcF Prolongations by Subject (Safety Population)

| Treatment | Subject, | | | QTcF (ms)^{a} | | | |
|---|---|---|---|---|---|---|---|
| Group | Sex | Time Point | | Reading 1 | Reading 2 | Reading 3 | Average |
| Placebo | 9016, M | pre-dose | | 429 | 436 | 424 | 430 |
| (N = 11) | | 1 | h | 424 | 432 | 422 | 426 |
| | | 3 | h | 434 | 440 | 429 | 434 |
| | | 5 | h | 427 | 436 | 428 | 430 |
| | | 24 | h | 420 | 440 | 407 | 422 |
| | 9027, M | pre-dose | | 404 | 401 | 416 | 407 |
| | | 1 | h | 407 | 400 | 438 | 415 |
| | 9077, M | pre-dose | | 419 | 415 | 415 | 416 |
| | | 12 | h | 435 | 421 | 420 | 425 |
| 5 mg | 9002, F | pre-dose | | 418 | 428 | 413 | 420 |
| (N = 6) | | 5 | h | 451 | 446 | 439 | 445 |
| | 9003, M | pre-dose | | 413 | 424 | 422 | 420 |
| | | 1 | h | 437 | 411 | 412 | 420 |
| | | 2 | h | 440 | 434 | 421 | 432 |
| | | 3 | h | 424 | 427 | 435 | 429 |
| | | 5 | h | 434 | 435 | 426 | 432 |
| | 9009, M | pre-dose | | 414 | 423 | 426 | 421 |
| | | 2 | h | 413 | 431 | 417 | 420 |
| 100 mg | 9056, M | pre-dose | | 413 | 413 | 436 | 421 |
| (N = 6) | | 0.5 | h | 415 | 424 | 436 | 425 |
| | | 2 | h | 445 | 426 | 446 | 439 |
| | | 3 | h | 453 | 443 | 429 | 442 |
| | | 5 | h | 419 | 448 | 419 | 429 |
| | | 8 | h | 436 | 457 | 444 | 446 |
| | | 24 | h | 432 | 431 | 434 | 432 |
| | | 48 | h | 418 | 433 | 416 | 422 |
| 150 mg | 9088, M | pre-dose | | 416 | 423 | 419 | 419 |
| (N = 6) | | 3 | h | 410 | 434 | 423 | 422 |
| | | 12 | h | 434 | 427 | 429 | 430 |
| | 9099, M | pre-dose | | 400 | 400 | 392 | 397 |
| | | 3 | h | 420 | 431 | 435 | 429 |
| | | 5 | h | 437 | 440 | 431 | 436 |
| 200 mg | 9078, M | pre-dose | | 395 | 403 | 402 | 400 |
| (N = 1) | | 3 | h | 441 | 422 | 416 | 426 |

Prolonged QTc values are shown in bold.

There was 1 female subject who experienced a single QTcF prolongation post-dose, but it was only 1 ms above the threshold of 450 ms. Therefore, the averaged QTcF value for this subject was normal. There were 9 male subjects who experienced at least 1 QTcF prolongation (>430 ms) during the study. However, only 4 of these 9 subjects had an averaged QTcF value that was above the threshold. Subject 9056 experienced the most prolongations during the study and had the maximum QTcF interval observed in the study, at 457 ms. However, the pattern of prolongations for this subject does not appear to be drug-related as prolongations were observed from pre-dose through to 48 hours post-dose.

The overall incidence of QTcF prolongation in the SAD study was low (10 subjects, 23.8%), and no dose-related effects were observed. None of the QTcF prolongations observed were considered clinically significant by the investigator.

These novel data from the MAD and the SAD studies on the cardiac safety of d-methadone—in particular the absence of clinically significant abnormal EKGs—are aligned with the findings by Bart on racemic methadone [Bart G et al., Methadone and the QTc Interval: Paucity of Clinically Significant Factors in a Retrospective Cohort. Journal of Addiction Medicine 2017. 11(6):489-493; Marmor M et al., Coronary artery disease and opioid use. Am J Cardiol. 2004 May 15; 93(10):1295-7] and support further development of d-methadone for the multiplicity of clinical indications outlined in the current application.

### Example 2: d-Methadone Administered Systemically Achieves Levels in the CNS Sufficient to Bind the NMDA Receptor, NET, and SERT, and Potentially Increase BDNF Levels

After establishing (as shown above) that d-methadone administered to humans does not convert to l-methadone and that it is devoid of effects commonly seen with other opioids (e.g., methadone) and side effects seen with other NMDA receptor antagonists (e.g., ketamine) that could interfere with the postulated direct effect of d-methadone on the improvement of cognitive function, the inventors conducted a separate preclinical study in rats to show that d-methadone administered systemically (subcutaneously) achieves levels in the CNS sufficient for the substance to bind to the NMDA receptor, NET, and SERT, and potentially increase BDNF levels and testosterone levels.

Materials and Methods:

Male Sprague Dawley rats (150 g on arrival) from Harlan (Indianapolis, Ind.) were used in the study. Upon receipt, rats were assigned unique identification numbers and were group housed with 3 rats per cage in polycarbonate cages with micro-isolator filter tops. All rats were examined, handled, and weighed prior to initiation of the study to assure adequate health and suitability. Chow and water were provided ad libitum for the duration of the study. Animals were singly-housed for the duration of the study. Test compounds were administered chronically once daily for 15 days. Test compound: d-Methadone (10, 20, and 40 mg/kg; Relmada Therapeutics) was dissolved in saline and administered subcutaneous (S.C.) at a dose volume of 1 ml/kg. Vehicle control: Saline was administered subcutaneous (S.C.) at a dose volume of 1 ml/kg. Plasma and Brain Collection. Plasma and brains were collected from the test compound and vehicle groups. Rats were decapitated and trunk blood was collected into microcentrifuge tubes containing K2EDTA and kept on ice for short term storage. Within 15 minutes the tubes were centrifuged at 1,500 to 2,000×g for 10 to 15 minutes in a refrigerated centrifuge set to maintain 2° C. to 8° C. The plasma was separated from the sample within 20 (±10) minutes after centrifugation and transferred into micro centrifuge tubes and placed on dry ice. Samples were stored in the −80° C. freezer until shipment to 7th wave laboratory. Brains were extracted and frozen on dry ice in polypropylene snap cap vials. All samples were stored in the −80° C. freezer until shipment to 7th wave laboratory.

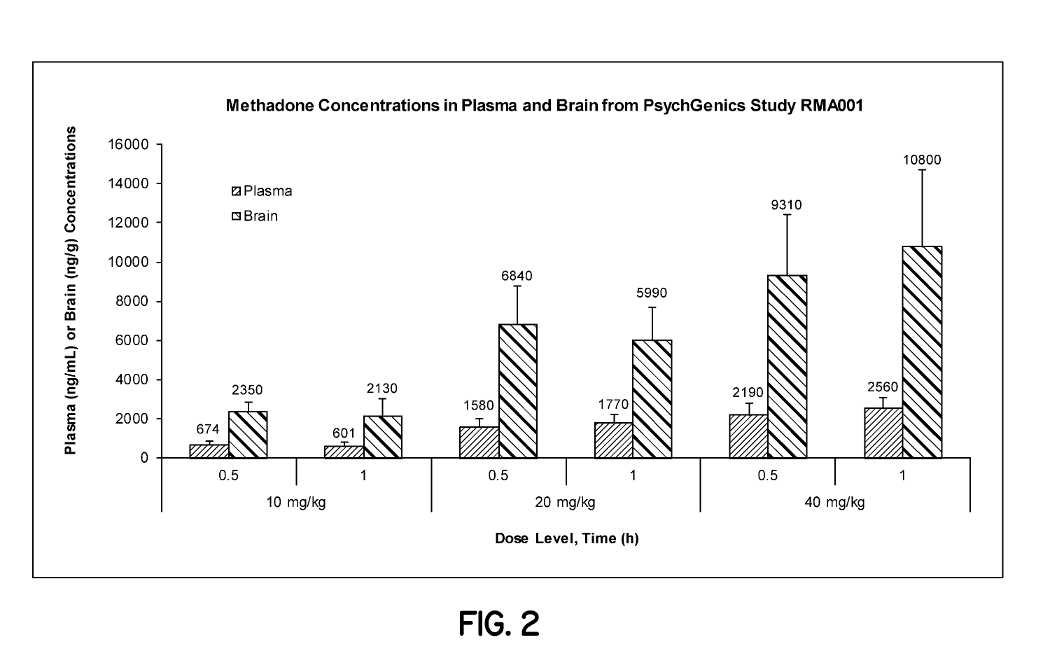

The following data from this study (see Table 13, below, and FIG. 2) show that d-methadone is readily transported across the blood brain barrier and that d-methadone levels are 3-4 fold higher in the brain than in the serum.

TABLE 13

| | | | Plasma Concentration | | | Brain Concentration | | | | | |
|---|---|---|---|---|---|---|---|---|---|---|---|
| | | | (ng/mL) | | | (ng/g) | | | Brain to Plasma Ratio | | |
| Treatment | | | Individual | Group | Group | Individual | Group | Group | Individual | Group | Group |
| Dose Level | Time (h) | Animal ID | Subjects | Mean | SD | Subjects | Mean | SD | Subjects | Mean | SD |
| d-Methadone | 0.5 | RL1711.01 | 685 | 674 | 177 | 2320 | 2350 | 520 | 3.4 | 3.5 | 0.2 |
| 10 mg/kg | | RL1711.02 | 605 | | | 2230 | | | 3.7 | | |
| | | RL1712.02 | 507 | | | 1720 | | | 3.4 | | |
| | | RL1712.03 | 970 | | | 3180 | | | 3.3 | | |
| | | RL1714.02 | 603 | | | 2320 | | | 3.8 | | |
| | 1 | RL1714.03 | 394 | 601 | 208 | 1270 | 2130 | 910 | 3.2 | 3.5 | 0.7 |
| | | RL1718.02 | 370 | | | 1090 | | | 2.9 | | |
| | | RL1720.02 | 738 | | | 3100 | | | 4.2 | | |
| | | RL1721.03 | 832 | | | 2360 | | | 2.8 | | |
| | | RL1722.02 | 672 | | | 2810 | | | 4.2 | | |
| d-Methadone | 0.5 | RL1710.03 | 2170 | 1580 | 440 | 7840 | 6840 | 1960 | 3.6 | 4.4 | 0.6 |
| 20 mg/kg | | RL1714.01 | 1180 | | | 5020 | | | 4.3 | | |
| | | RL1715.03 | 1120 | | | 4620 | | | 4.1 | | |
| | | RL1716.02 | 1760 | | | 9220 | | | 5.2 | | |
| | | RL1717.01 | 1660 | | | 7500 | | | 4.5 | | |
| | 1 | RL1718.03 | 1540 | 1770 | 440 | 5990 | 5990 | 1730 | 3.9 | 3.4 | 0.5 |
| | | RL1721.02 | 2050 | | | 5920 | | | 2.9 | | |
| | | RL1722.01 | 1420 | | | 4010 | | | 2.8 | | |
| | | RL1723.02 | 1420 | | | 5310 | | | 3.7 | | |
| | | RL1727.03 | 2410 | | | 8740 | | | 3.6 | | |
| d-Methadone | 0.5 | RL1710.02 | 1890 | 2190 | 630 | 9450 | 9310 | 3090 | 5.0 | 4.2 | 0.6 |
| 40 mg/kg | | RL1716.01 | 2660 | | | 10600 | | | 4.0 | | |
| | | RL1717.03 | 2210 | | | 8040 | | | 3.6 | | |
| | | RL1718.01 | 1300 | | | 5040 | | | 3.9 | | |
| | | RL1719.02 | 2890 | | | 13400 | | | 4.6 | | |
| | 1 | RL1724.03 | 2830 | 2560 | 520 | 13700 | 10800 | 3900 | 4.8 | 4.1 | 0.8 |
| | | RL1726.03 | 2020 | | | 6460 | | | 3.2 | | |
| | | RL1727.02 | 3150 | | | 14500 | | | 4.6 | | |
| | | RL1729.02 | 2230 | | | 8630 | | | 3.9 | | |
| Vehicle | 0.5 | RL1709.01 | BLQ | BLQ | — | BLQ | BLQ | — | n/a | n/a | — |
| | | RL1713.01 | BLQ | | | BLQ | | | n/a | | |
| | | RL1713.02 | BLQ | | | BLQ | | | n/a | | |
| | | RL1713.03 | BLQ | | | BLQ | | | n/a | | |
| | | RL1715.01 | BLQ | | | BLQ | | | n/a | | |
| | 1 | RL1719.01 | BLQ | BLQ | — | BLQ | BLQ | — | n/a | n/a | — |
| | | RL1725.01 | BLQ | | | BLQ | | | n/a | | |
| | | RL1725.02 | BLQ | | | BLQ | | | n/a | | |
| | | RL1726.01 | BLQ | | | BLQ | | | n/a | | |
| | | RL1730.02 | BLQ | | | BLQ | | | n/a | | |

BLQ = Below the Lower Limit of Quantitation (4.90 ng/mL for plasma; 19.7 ng/g for brain)

The findings shown by this data confirm the potential for d-methadone in the treatment of NS disorders and their manifestations, further suggesting that it might possibly be effective at doses lower than expected based solely on serum pharmacokinetics and thus lowering the possibility of toxicity towards organs outside of the CNS. This higher than expected CNS concentration may also make d-methadone a better candidate than, for example, memantine, for diseases where higher CNS levels of an NMDA receptor antagonist are needed.

### Example 3: The NMDA Antagonistic Effects of d-Methadone are Comparable to Memantine In Vitro

d-Methadone has been previously found to exert NMDAR antagonistic activity by one of the inventors (Gorman, A. L. Elliott K J, Inturrisi C E). The d- and l-isomers of methadone bind to the non-competitive site on the N-methyl-D-aspartate(NMDA) receptor in rat forebrain and spinal cord, Nerurosci Lett 1997: 223:5-8). As has been described above, memantine is an NMDA receptor antagonist approved for moderate to severe Alzheimer's disease (under the trade name Namenda®). Memantine has been found to increase the production of brain-derived neurotrophic factor (BDNF) in rat brain, thus offering one possible explanation for its neuroprotective effects (Marvanova M. et al. The Neuroprotective Agent Memantine InducesBrain-Derived Neurotrophic Factor and trkB Receptor Expression in Rat Brain. Molecular and Cellular Neuroscience 2001; 18, 247-258). And so, the inventors examined the antagonistic effects of d-methadone and memantine on the electrophysiological response of human cloned NMDA NR1/NR2 A and NR1/NR2 B receptors expressed in HEK293 cells.

To do so, this study examined the in vitro effects of ten (10) test articles (shown in Table 14) in the following screen patch assays: (1) NMDA glutamate receptors NR1/NR2A encoded by the human GRIN1 and GRIN2A genes, expressed in HEK293 cells; and (2) NMDA glutamate receptors NR1/NR2B encoded by the human GRIN1 and GRIN2B genes, expressed in HEK293 cells. Loading of the plates in this study are shown in Table 15.

TABLE 14
Test Article Information: Actual concentrations of compounds in experiment.

| | | | Amount | |
|---|---|---|---|---|
| Test | | MW | Received | |
| article # | Test Article ID: | (Salt) | (mg) | NR1/NR2A-B test conc., (μM) |
| 1 | Levorphanol Tartrate | 443.49 | 10 | 0.1, 0.3, 1, 3, 10, 30, 100, 300 |
| 2 | Ketamine HCL | 274.19 | N/A | 0.1, 0.3, 1, 3, 10, 30, 100, 300 |
| 3 | S-Ketamine HCL | 274.19 | 1000 | 0.1, 0.3, 1, 3, 10, 30, 100, 300 |
| 4 | Phencyclidine HCL | 279.85 | N/A | 0.01, 0.03, 0.1, 0.3, 1, 3, 10, 30 |
| 5 | [R, S]-Methadone HCL | 345.91 | 50 | 0.1, 0.3, 1, 3, 10, 30, 100, 300 |
| 6 | [S]-methadone HCL | 345.91 | 10 | 0.1, 0.3, 1, 3, 10, 30, 100, 300 |
| 7 | [R]-methadone - D9 | 318.50 | 20 | 0.1, 0.33, 1.1, 3.3, 10.9, 32.6, 109, 326 |
| 8 | [S]-methadone - D9 | 318.50 | 5 | 0.1, 0.33, 1.1, 3.3, 10.9, 32.6, 109, 326 |
| 9 | [S]-methadone - D10 | 319.51 | 5 | 0.11, 0.32, 1.1, 3.2, 10.8, 32.5, 108, 325 |
| 10 | [S]-methadone - D16 | 325.54 | 5 | 0.11, 0.32, 1.1, 3.2, 10.6, 31.9, 106, 319 |

TABLE 15
Compound plate loading
[Plate load map. Ten compounds, two positive controls, 8 concentrations, four replicates]

| Plate | | | | | | | | | | | | | | |
|---|---|---|---|---|---|---|---|---|---|---|---|---|---|---|
| 1 (2X) | 1 | 2 | 3 | 4 | 5 | 6 | 7 | 8 | 9 | 10 | 11 | 12 | 13 | 14 |
| A | Memantine | | Compound 1 | | Compound 2 | | Compound 3 | | Compound 4 | | Compound 5 | | Compound 6 | |
| B | | | | | | | | | | | | | | |

| C | | | | | | | |
|---|---|---|---|---|---|---|---|
| D | | | | | | | |
| E | | | | | | | |
| F | | | | | | | |
| G | | | | | | | |
| H | | | | | | | |
| I | | | | | | | |
| J | | | | | | | |
| K | | | | | | | |
| L | | | | | | | |
| M | | | | | | | |
| N | | | | | | | |
| O | | | | | | | |
| P | | | | | | | |
| | All wells contain 2X TA concentration | | | | | | |
| A | 10 I.LM | 10 i.i.M | 10 i.i.M | 10 μM | 10 i.i.M | 10 i.i.M | 10 I.LM |
| B | glutamate + | glutamate + | glutamate + | glutamate + | glutamate + | glutamate + | glutamate + |
| C | 50 I.LM | 50 i.i.M | 50 i.i.M | 50 ii.M | 50 i.i.M | 50 I.LM | 50 i.i.M |
| D | glycine | glycine | glycine | glycine | glycine | glycine | glycine |
| E | Memantine | Compound 1 | Compound 2 | Compound 3 | Compound 4 | Compound 5 | Compound 6 |
| F | | | | | | | |
| G | | | | | | | |
| H | | | | | | | |
| I | | | | | | | |
| J | | | | | | | |
| K | | | | | | | |
| L | | | | | | | |
| M | | | | | | | |
| N | | | | | | | |
| O | | | | | | | |
| P | | | | | | | |

All wells contain10 μM glutamate, 50 μM glycine and Ix TA concentration

| Plate | | | | | | | | | | |
|---|---|---|---|---|---|---|---|---|---|---|
| 1 (2X) | 15 | 16 | 17 | 18 | 19 | 20 | 21 | 22 | 23 | 24 |
| A | Compound 7 | | Compound 8 | | Compound 9 | | Compound 10 | | Vehicle | |
| B | | | | | | | | | | |
| C | | | | | | | | | | |
| D | | | | | | | | | | |
| E | | | | | | | | | | |
| F | | | | | | | | | | |
| G | | | | | | | | | | |
| H | | | | | | | | | | |
| I | | | | | | | | | | |
| J | | | | | | | | | | |
| K | | | | | | | | | | |
| L | | | | | | | | | | |
| M | | | | | | | | | | |
| N | | | | | | | | | | |
| O | | | | | | | | | | |
| P | | | | | | | | | | |
| | All wells contain 2X TA concentration | | | | | | | | | |
| A | 10 p.M | | 10 I.LM | | 10 i.i.M | | 10 μM | | 0-100 p.M | |
| B | glutamate + | | glutamate + | | glutamate + | | glutamate + | | glutamate + | |
| C | 50 I.LM | | 50 I.LM | | 50 i.i.M | | 50 μM | | 50 p.M | |
| D | glycine | | glycine | | glycine | | glycine | | glycine | |
| E | Compound 7 | | Compound 8 | | Compound 9 | | Compound 10 | | | |
| F | | | | | | | | | | |
| G | | | | | | | | | | |
| H | | | | | | | | | | |
| I | | | | | | | | | | |
| J | | | | | | | | | | |
| K | | | | | | | | | | |
| L | | | | | | | | | | |
| M | | | | | | | | | | |
| N | | | | | | | | | | |
| O | | | | | | | | | | |
| P | | | | | | | | | | |

All wells contain10 μM glutamate, 50 μM glycine and Ix TA concentration

Materials and Methods

Cloned Test Systems:

Cells used in this study were HEK293 cells (human embryonic kidney cells; Source-Strain: ATCC, Manassas, Va.; Source-Sub Strain: Charles River Corporation, Cleveland, Ohio). Cells were maintained in tissue culture incubators per Charles River standard operating procedure. Stocks were maintained in cryogenic storage. Cells used for electrophysiology were plated in 150-mm plastic culture dishes. Cells were transformed with adenovirus 5 DNA; transfected with ion channel or receptor cDNA.

HEK293 Culture Procedures:

The HEK293 cells were transfected with the appropriate ion channel or receptor cDNA(s) encoding NR1 and NR2A or NR2B. Stable transfectants were selected using the G418 and Zeocin-resistance genes incorporated into the expression plasmid. Selection pressure was maintained with G418 and Zeocin in the culture medium. Cells were cultured in Dulbecco's Modified Eagle Medium/Nutrient Mixture F-12 (D-MEM/F-12) supplemented with 10% fetal bovine serum, 100 U/mL penicillin G sodium, 100 μg/mL streptomycin sulfate, 100 μg/mL Zeocin, 5 μg/mL blasticidin and 500 μg/mL G418.

Test articles effects were evaluated in 8-point concentration-response format (8 replicate wells/concentration). All test and control solutions contained 0.3% DMSO. The test articles formulations were loaded in a 384-well compound plate using an automated liquid handling system (SciClone ALH3000, Caliper LifeScienses)

To verify the sensitivity the assay, the antagonist positive control article (Memantine) was applied at 8 concentrations.

ScreenPatch Procedures (for NR1/NR2A and NR1/NR2B Receptor Antagonist Assays):

As described above, the test systems involved NR1/NR2A and NR1/NR2B ionotropic glutamate receptors expressed in HEK293 cells.

Electrophysiological Procedures: The intracellular solution (mM) used was: 50 mM CsCl, 90 mM CsF, 2 mM MgCl₂, 5 mM EGTA, 10 mM HEPES. It was adjusted to pH 7.2 with CsOH. This solution was prepared in batches and stored refrigerated. In preparation for a recording session, the intracellular solution was loaded into the intracellular compartment of the PPC planar electrode. An extracellular solution, HB-PS (composition in mM) was: NaCl, 137; KCl, 1.0; CaCl₂, 2; HEPES, 10; Glucose, 10. Its pH was adjusted to 7.4 with NaOH (and the solution was refrigerated until use). (Holding potential: −100 mV, potential during antagonist application: −45 mV.) Recording procedure: Extracellular buffer was loaded into the PPC plate wells (11 μL per well). Cell suspension was pipetted into the wells (9 μL per well) of the PPC planar electrode. Whole-cell recording configuration was established via patch perforation with membrane currents recorded by on-board patch clamp amplifiers. Two recording (scans) were performed: (1) during test articles application (for duration of at least 15 seconds) and, (2) agonist (˜EC₈₀ 10 μM L-glutamate) and test article co-application to detect antagonist effects of the test article.

Test Article Administration: The application consisted of the addition of 20 μL of 2× concentrated test article solution during first application. Agonist (10 μM glutamate and 50 μM glycine) mixed with 1× concentrated test article. Addition rate was 10 μL/s (2 second total application time).

The positive control was memantine hydrochloride: 0.1-300 μM glycine (8 concentration dose-response). And the positive control agonist was 0-100 μM L-glutamate (8 concentration dose-response, half log scale).

Data Analysis: Activation was calculated in three ways based on the following measurements: (1) peak current amplitudes, and (2) current amplitude 2 seconds after agonist addition.

Inhibition concentration-response data were fitted to an equation of the form: % Inhibition=% VC+{(% PC−% VC)/[1+([Test]/IC₅₀)^{N}]}, where [Test] was the concentration of test article, IC₅₀ was the concentration of the test article producing half-maximal inhibition, N was the Hill coefficient, % Inhibition was the percentage of ion channel current inhibited at each concentration of the test article. Nonlinear least squares fits were solved with the XLfit add-in for Excel (Microsoft, Redmond, Wash.)

Results

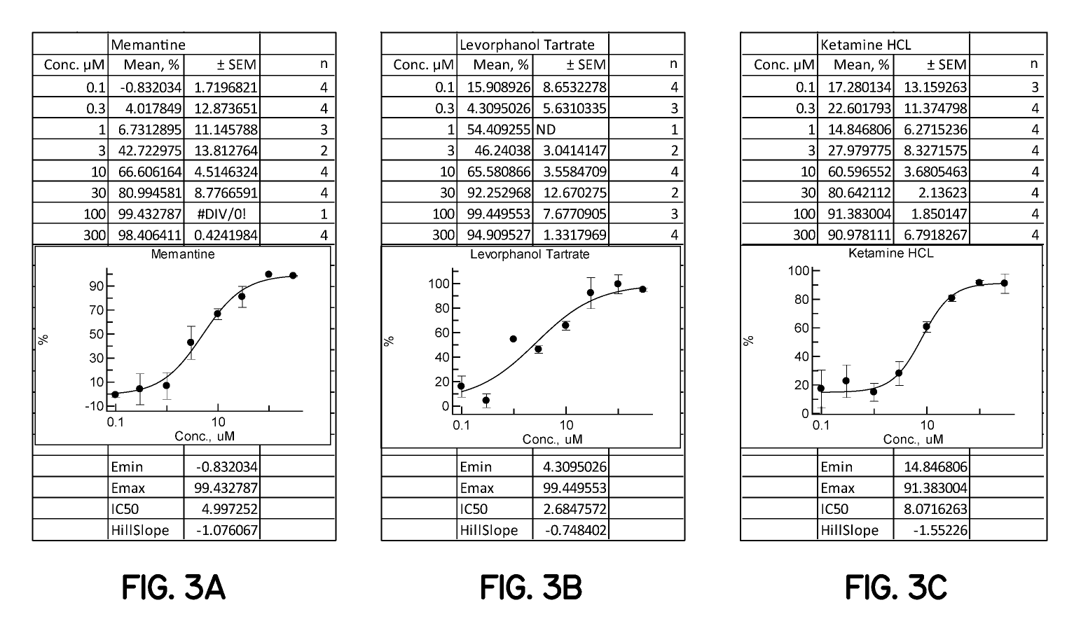

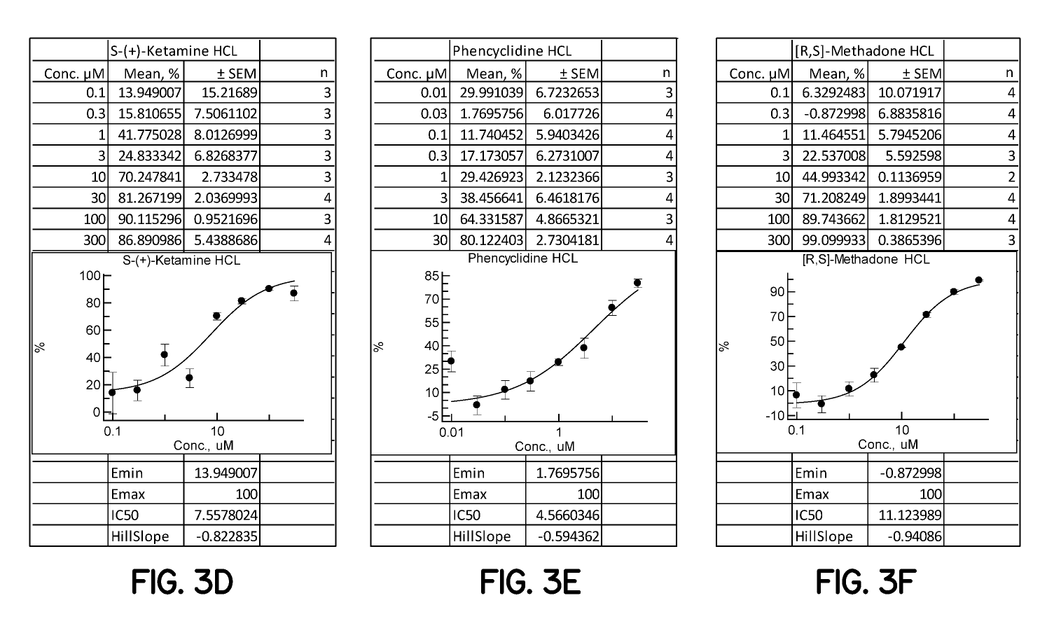

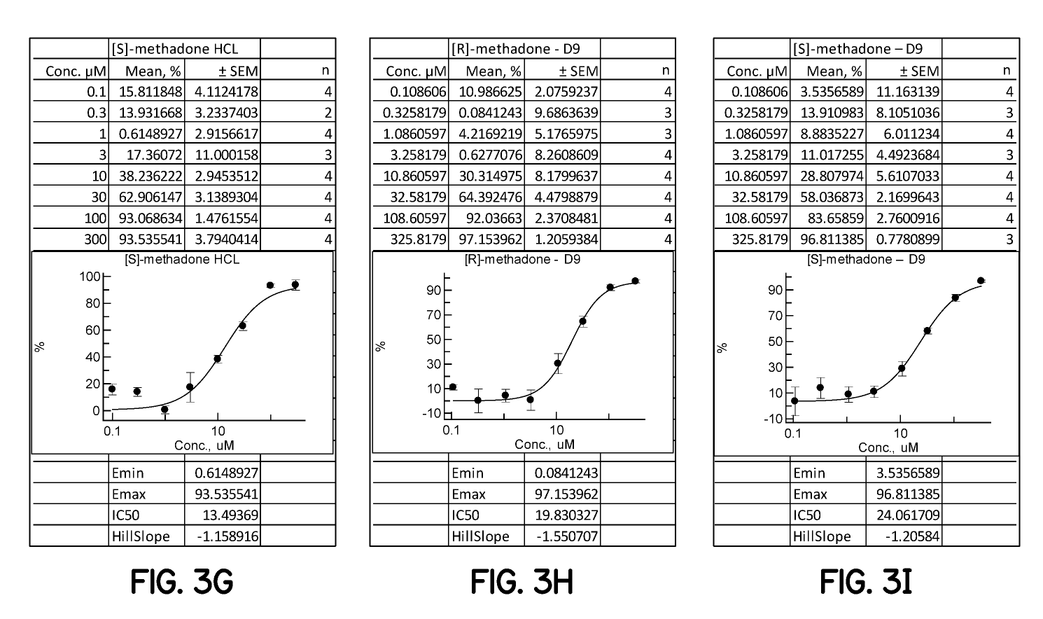

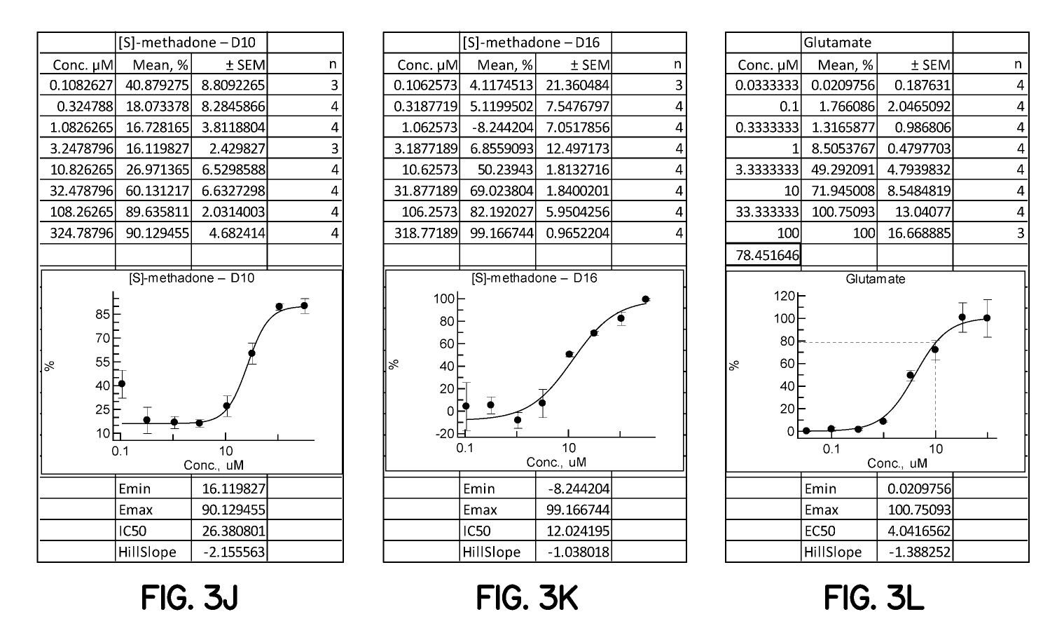

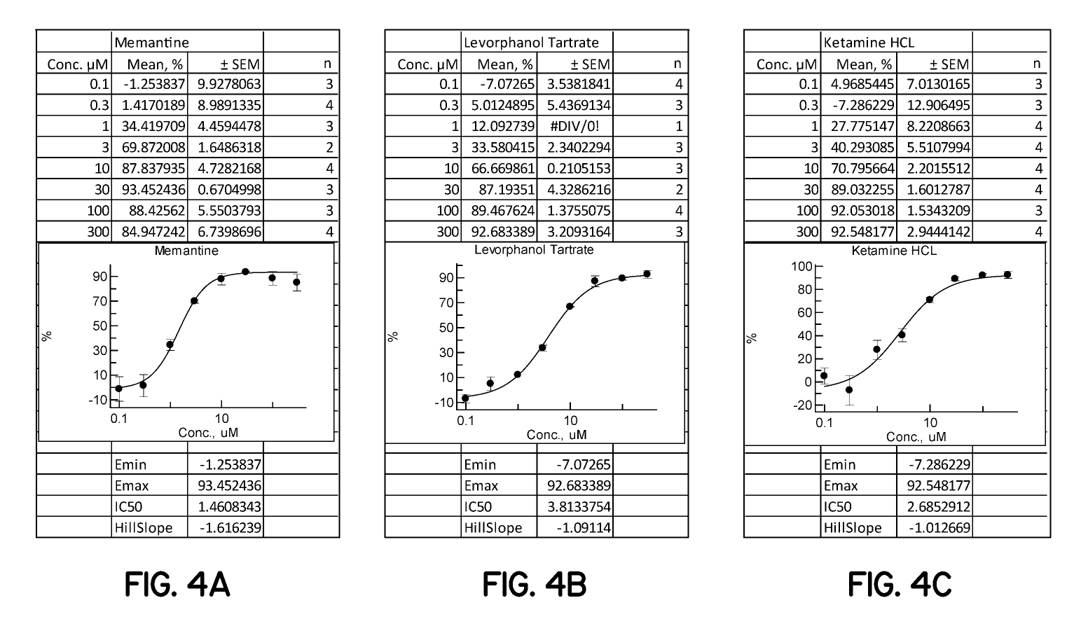

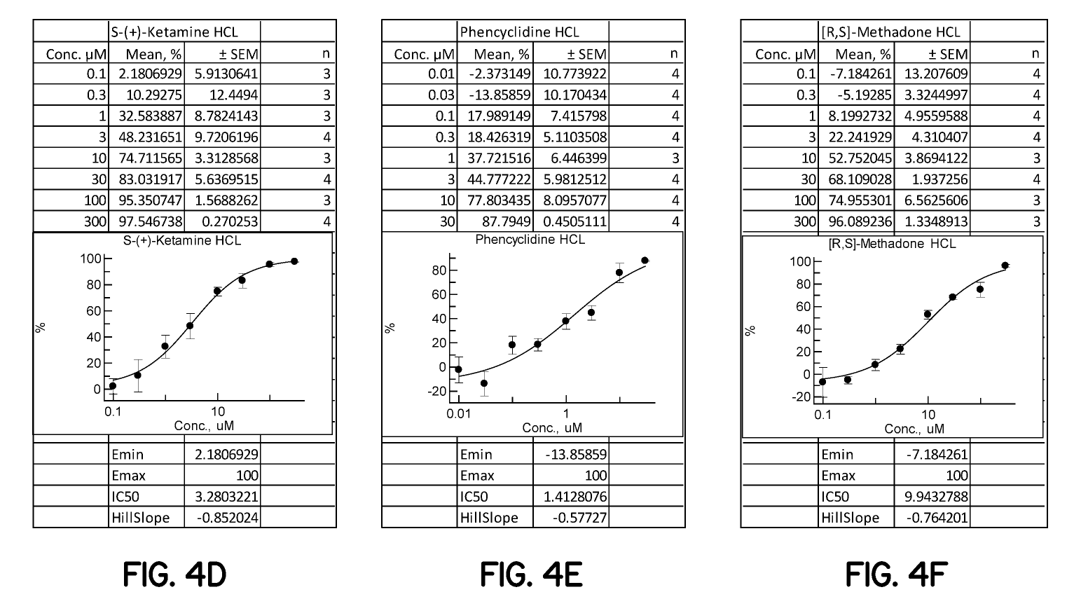

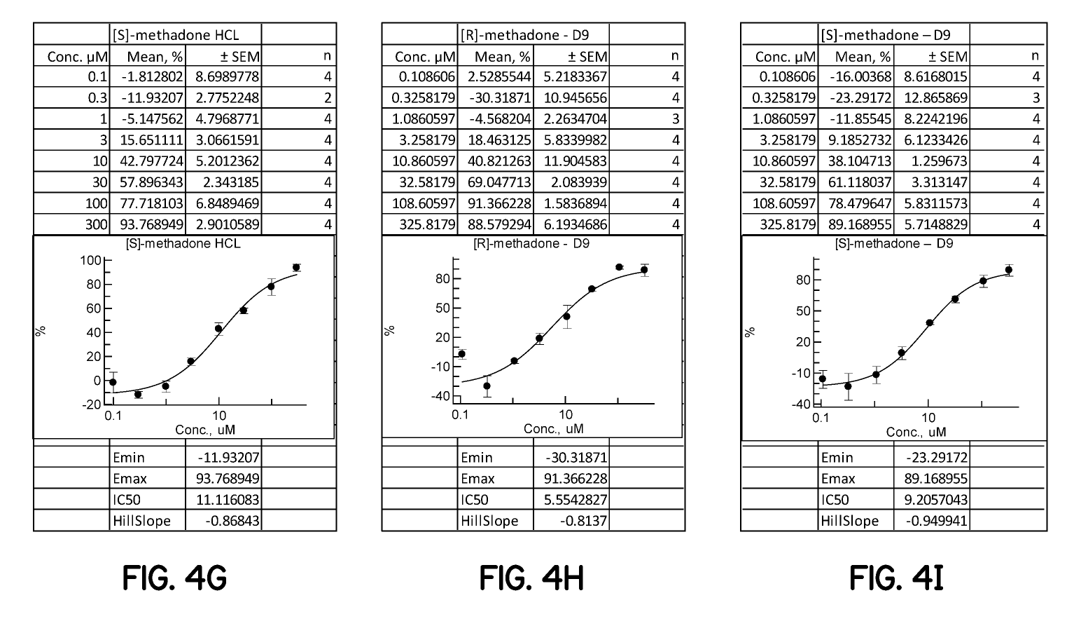

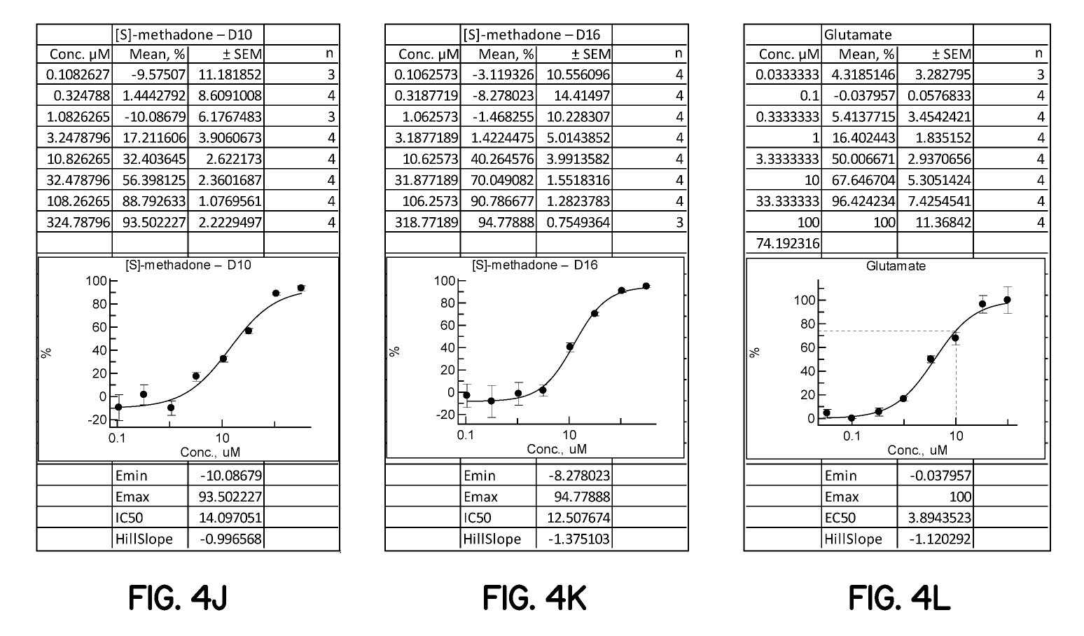

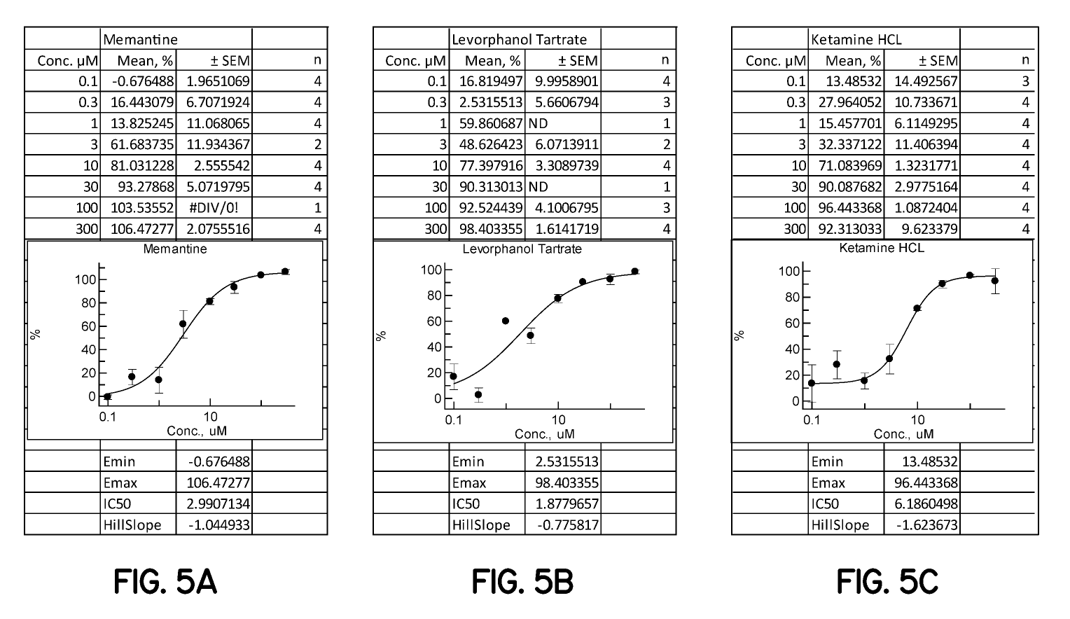

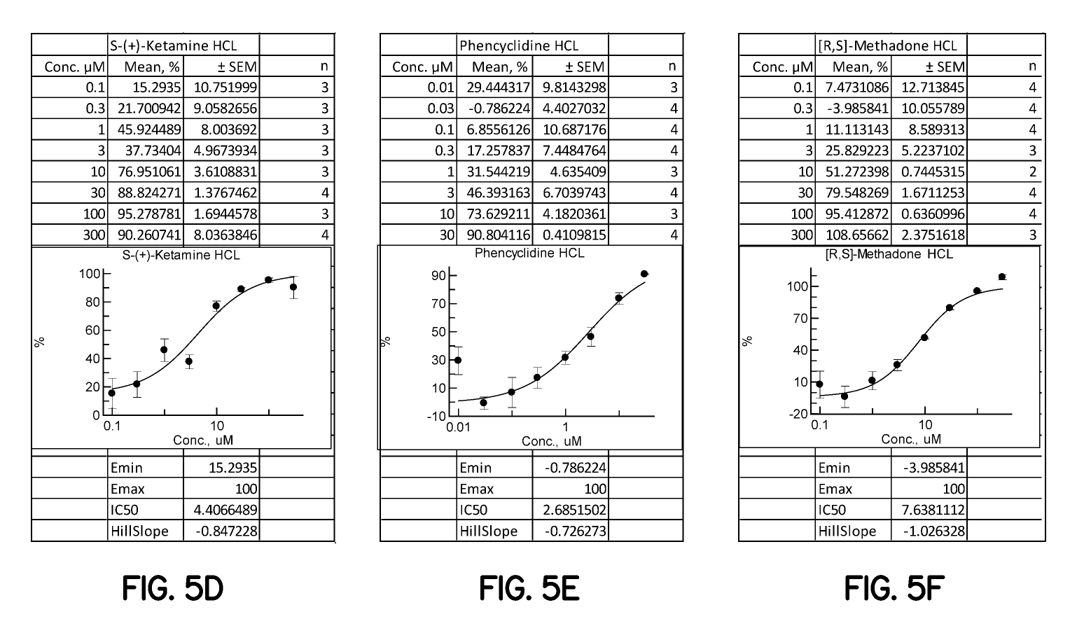

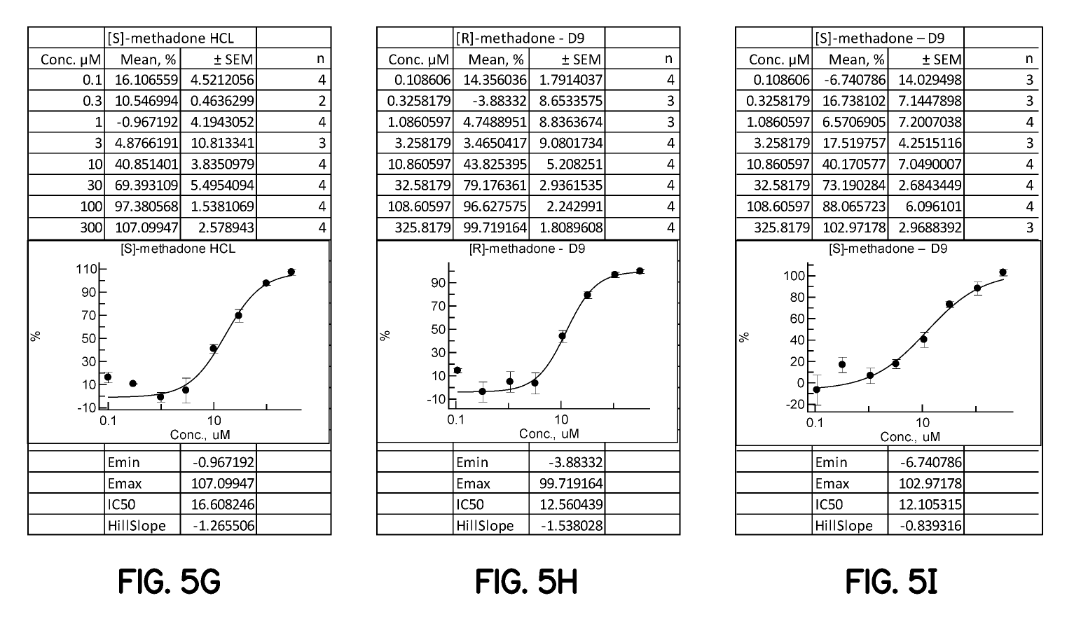

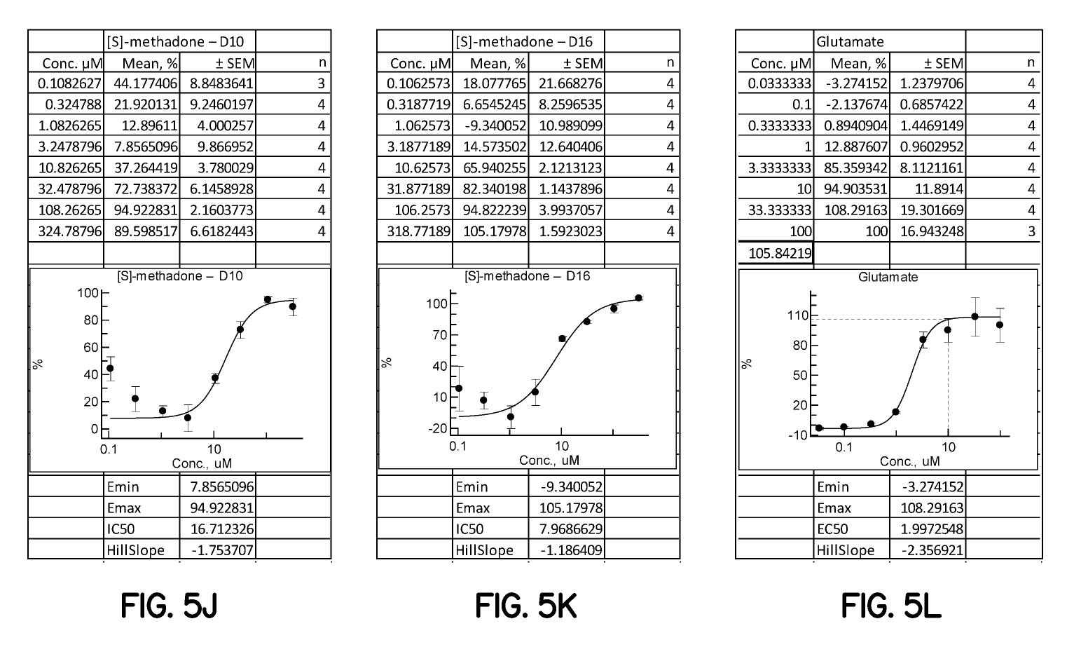

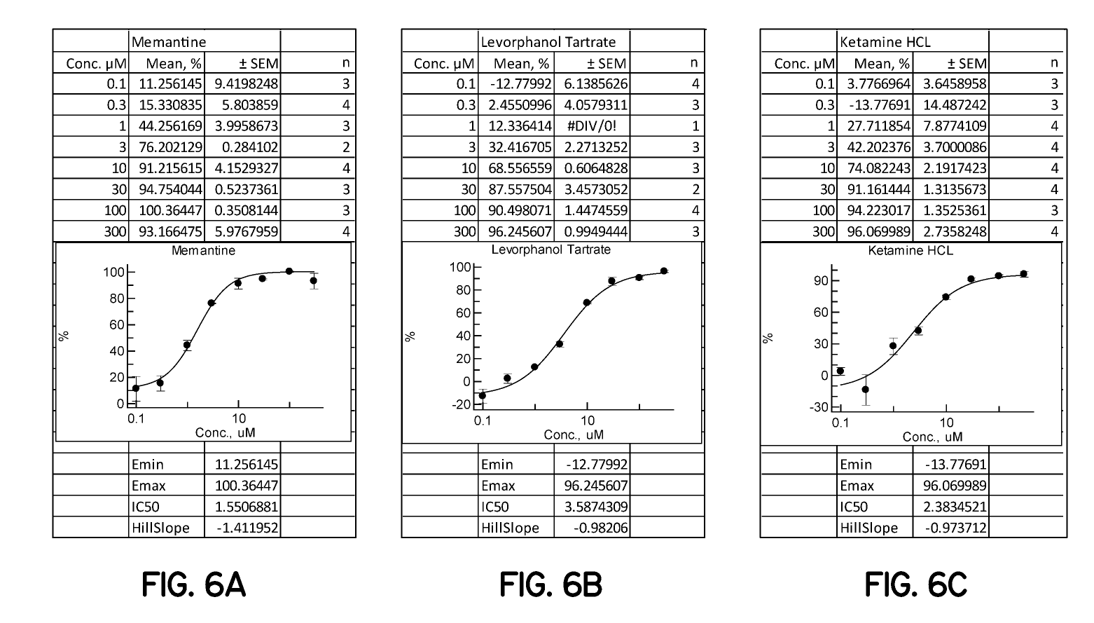

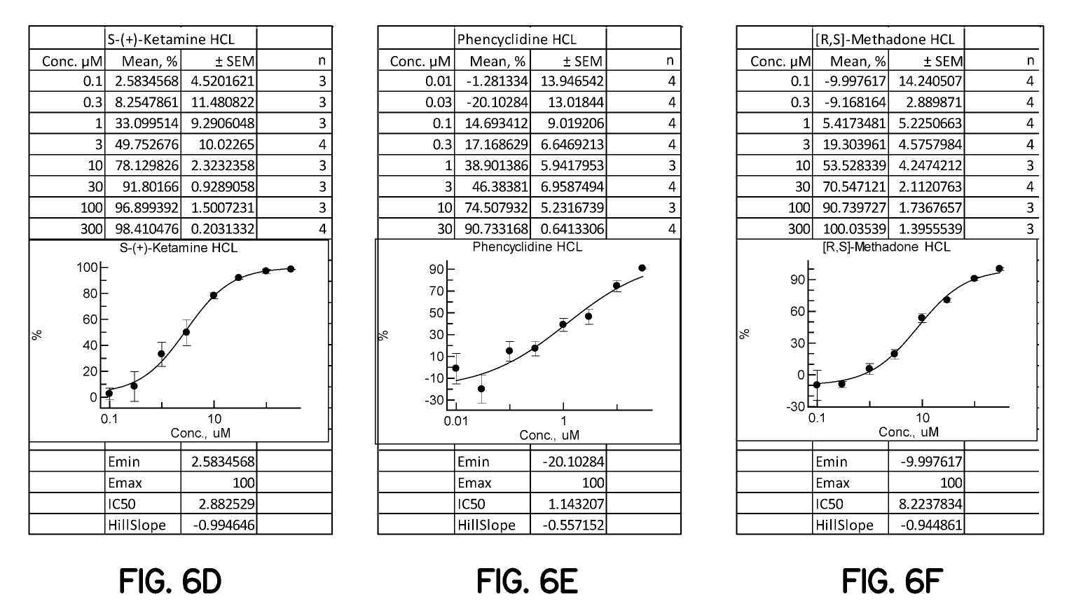

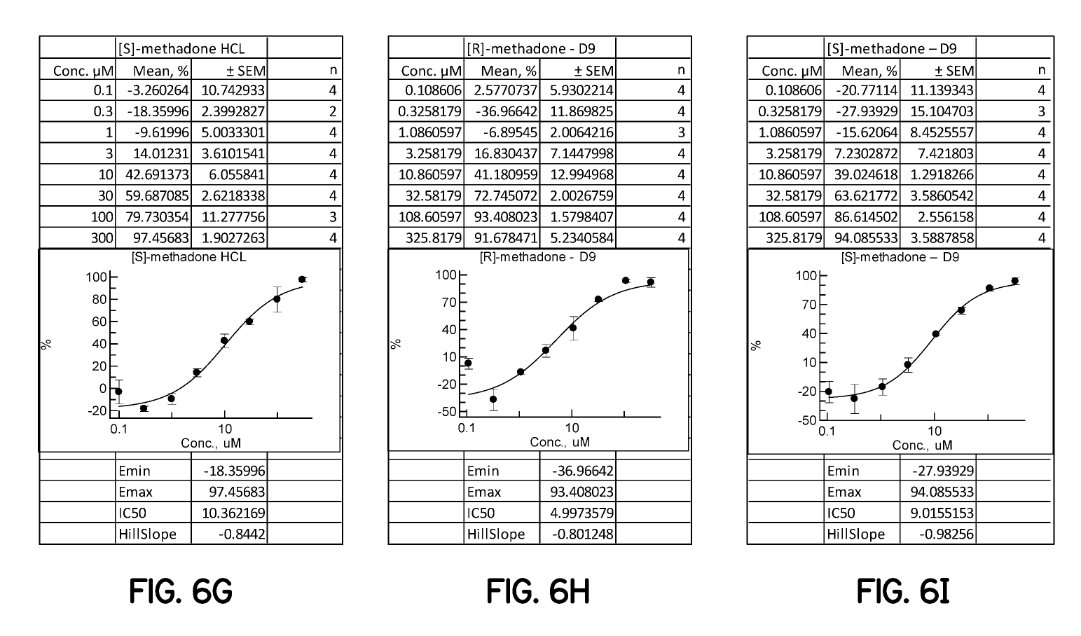

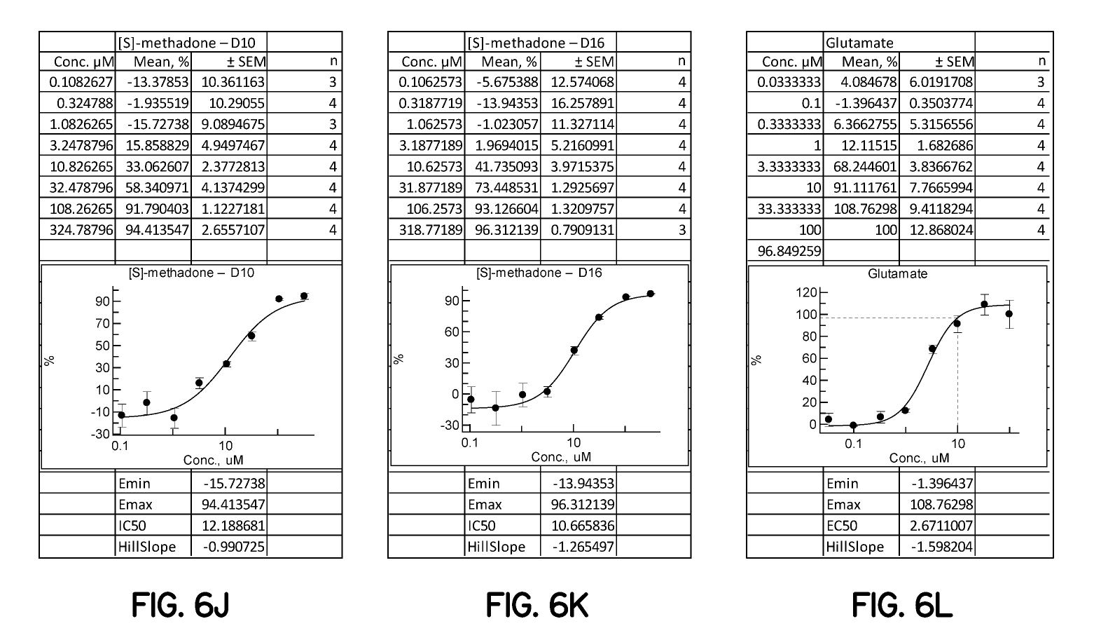

Test articles IC₅₀ and hillslope values for NR1/NR2A and NR1/NR2B are shown in Table 16 and Table 17. Table 16 represents measurements of peak current amplitude and Table 17 represents measurements of steady state current 2 seconds after compounds application. And FIGS. 3A-3L, 4A-4L, 5A-5L, and 6A-6L represent summary data files (numeric information and concentration response curves) for both measurements.

TABLE 16
NR1/NR2A and NR1/NR2B peak current amplitude measurements.
IC₅₀ and Hillslope coefficient for ten TA and positive controls.

| | NR1/NR2A | | NR1/NR2B | |
|---|---|---|---|---|
| Compound ID | EC/IC₅₀ | HillSlope | EC/IC₅₀ | HillSlope |
| Memantine | 5.00 | 1.08 | 1.46 | −1.62 |
| Glutamate 0-100 μM | 4.04* | −1.39 | 3.89* | −1.12 |
| Levorphanol Tartrate | 2.68 | 0.75 | 3.81 | −1.09 |
| Ketamine HCL | 8.07 | 1.55 | 2.69 | −1.01 |
| S-(+)-Ketamine HCL | 7.56 | 0.86 | 3.28 | −0.85 |
| Phencyclidine HCL | 4.57 | 0.60 | 1.41 | −0.58 |
| [R, S]-Methadone HCL | 11.12 | 0.96 | 9.94 | −0.76 |
| [S]-methadone HCL | 13.49 | 1.16 | 11.12 | −0.87 |
| [R]-methadone - D9 | 19.83 | 1.55 | 5.55 | −0.81 |
| [S]-methadone - D9 | 24.06 | 1.21 | 9.21 | −0.95 |
| [S]-methadone - D10 | 26.38 | 2.16 | 14.10 | −1.00 |
| [S]-methadone - D16 | 12.02 | 1.04 | 12.51 | −1.38 |

*EC₅₀ is shown

TABLE 17
NR1/NR2A and NR1/NR2B steady state current amplitude
measurements. IC₅₀ and Hillslope coefficient
for ten TA and positive controls.

| | NR1/NR2A | | NR1/NR2B | |
|---|---|---|---|---|
| Compound ID | EC/IC₅₀ | HillSlope | EC/IC₅₀ | HillSlope |
| Memantine | 2.99 | −1.04 | 1.55 | −1.41 |
| Glutamate 0-100 μM | 2.00* | −2.36 | 2.67* | −1.60 |
| Levorphanol Tartrate | 1.88 | −0.78 | 3.59 | −0.98 |
| Ketamine HCL | 6.19 | −1.62 | 2.38 | −0.97 |
| S-(+)-Ketamine HCL | 4.41 | −0.85 | 2.88 | −0.99 |
| Phencyclidine HCL | 2.69 | −0.73 | 1.14 | −0.56 |
| [R, S]-Methadone HCL | 7.64 | −1.03 | 8.22 | −0.94 |
| [S]-methadone HCL | 16.61 | −1.27 | 10.36 | −0.84 |
| [R]-methadone - D9 | 12.56 | −1.54 | 5.00 | −0.80 |
| [S]-methadone - D9 | 12.11 | −0.84 | 9.02 | −0.98 |
| [S]-methadone - D10 | 16.71 | −1.75 | 12.19 | −0.99 |
| [S]-methadone - D16 | 7.97 | −1.19 | 10.67 | −1.27 |

*EC₅₀ is shown

The results of this study (see Table 18, below) demonstrated approximately equivalent antagonism of peak currents for both compounds in the low μM range.

TABLE 18
(Screen Patch Assay study)

| | NR1/NR2A | NR1/NR2A | NR1/NR2B | NR1/NR2B |
|---|---|---|---|---|
| Compound | IC/IC₅₀ | HillSlope | IC/IC₅₀ | HillSlope |
| memantine | 5.00 | 1.08 | 1.46 | −1.62 |
| d-methadone | 13.49 | 1.16 | 11.12 | −0.87 |

These results suggest that d-methadone could have actions similar to those of memantine on Alzheimer's patients. Furthermore, based on the inventors' findings on cognitive function, d-methadone might be effective for the treatment of mild cognitive impairment and thus d-methadone might offer an improvement over memantine: while memantine is helpful only for moderate or severe dementia, d-methadone was found by the inventors to possibly improve cognitive function in patients with very mild cognitive impairment. Furthermore, d-methadone may also offer alternative options for patients unable to tolerate memantine for various reasons, including renal impairment (d-methadone is excreted by the liver). Another advantage of d-methadone rests in its higher than expected CNS penetration which suggests better efficacy at lower systemic doses.

### Example 4: d-Methadone Increases Serum BDNF in Humans

Methods

Next, in a randomized double blind placebo controlled study of 8 healthy subjects, the inventors tested BDNF levels before and 4 hours after administration of d-methadone (25 mg a day for ten days) [testing for PK and BDNF levels was done pre-treatment and 4 hours after the d-methadone 25 mg dose administration (six patients) or placebo (two patients) on days 2-6 and 10)]. The analysis was performed by means of an ELISA kit—which methods are known to those of ordinary skill in the art. Quantitative determination of BDNF was carried out by standard calibration curves obtained with human recombinant BDNF at concentrations ranging from 0.066 to 16 ng/ml (n=7), processed in exactly the same way as the plasma samples. The calibration curves fitted an allosteric sigmoidal equation (r2≥0.99). Each concentration is the result of three independent determinations. Data are presented as Mean and SD.

Results

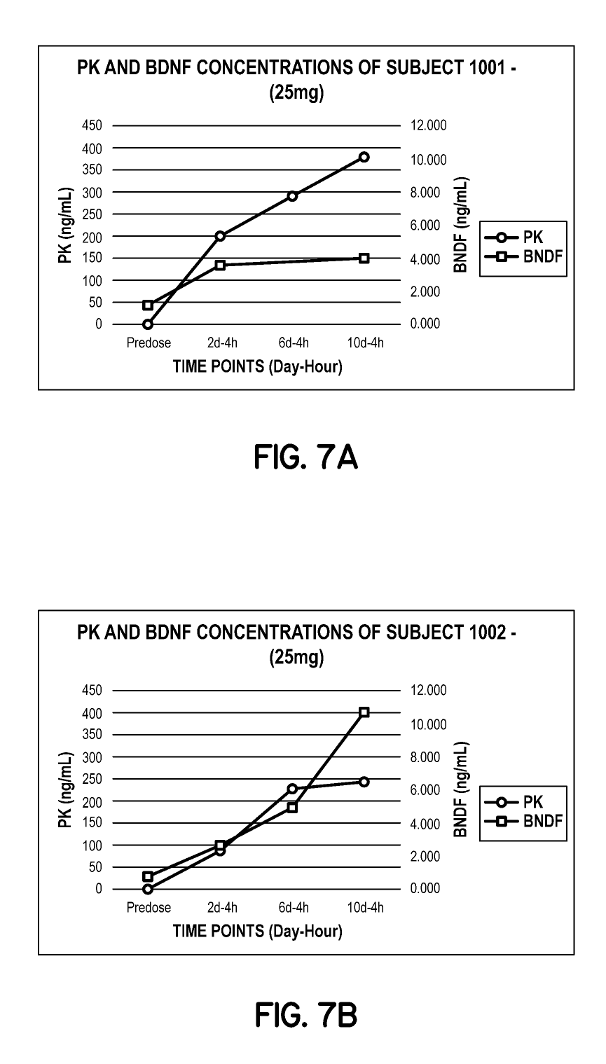

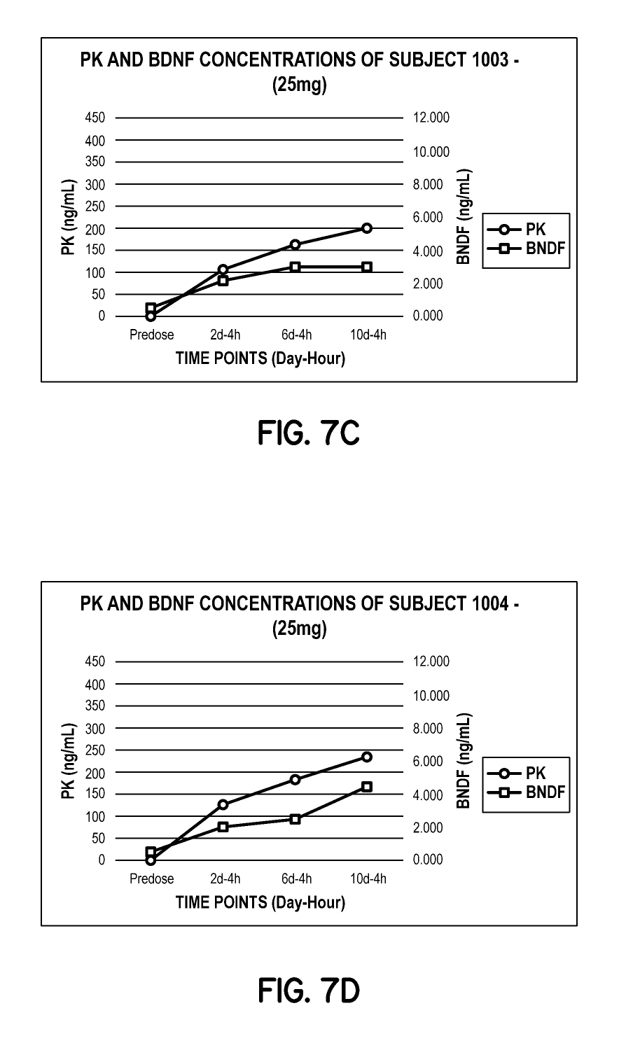

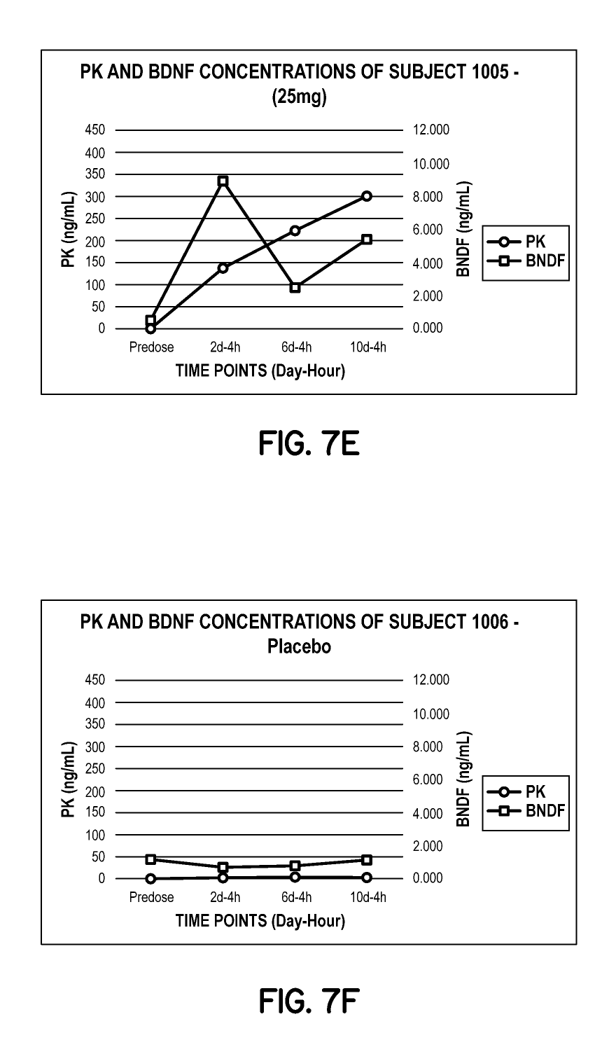

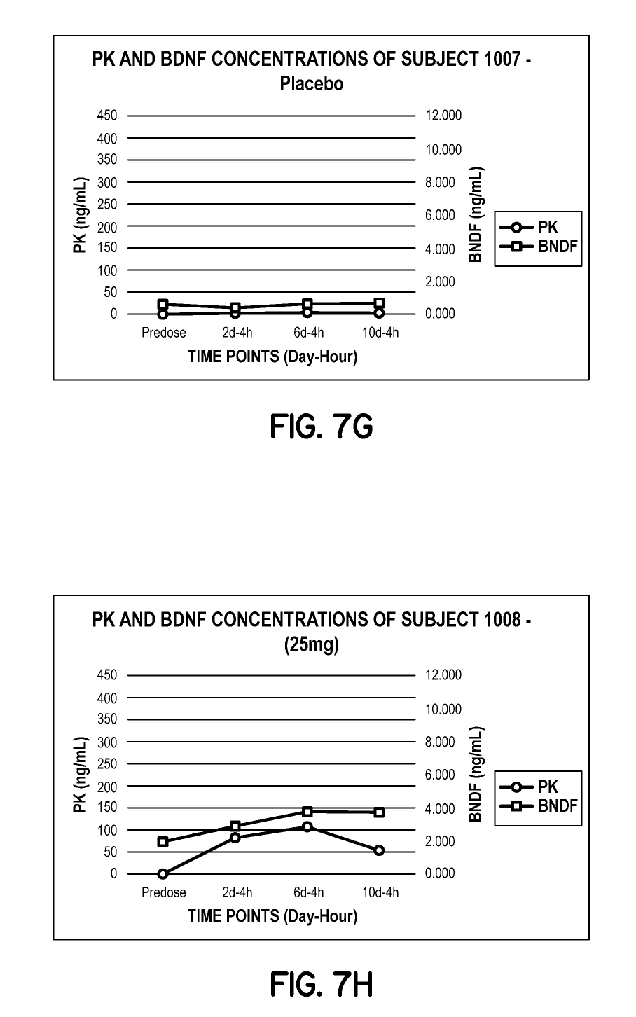

In the d-methadone treatment group, 6 of 6 subjects (100%) showed an increase in BDNF levels post d-methadone treatment compared to BDNF pre-treatment levels, with post-treatment day 10 BDNF serum levels ranging from twice to 17 times the pre-treatment BDNF levels; the smallest increase on day 10 (twice the pre-treatment level) was seen in subject 1008: this subject had the smallest day 10 d-methadone level, Cₘₐₓ and AUC and the longest Tₘₐₓ among all 6 treated subjects, consistent with a lower d-methadone pharmacokinetic disposition with respect to other treated subjects. By contrast, in the placebo subjects (1006 and 1007), where d-methadone levels were 0, the BDNF serum levels either decreased or remained unchanged (see Table 19, below, and FIGS. 7A-7H).

TABLE 19
MAD study 25 mg

| | Time Points | | | | | | | |
|---|---|---|---|---|---|---|---|---|
| | Pre-Treatment | | Day 2 hr 4 | | Day 6 hr 4 | | Day 10 hr 4 | |
| Compound | BDNF | ng/mL | BDNF | ng/L | BDNF | ng/mL | BDNF | ng/mL |
| d-methadone | PK | ng/mL | PK | ng/L | PK | ng/mL | PK | ng/mL |
| 25 mg or | | | | | | | | |
| placebo | | | | | | | | |
| Subject 1001 | BDNF | 1.143 | BDNF 3.603 | | BDNF | n/a | BDNF | 4.004 |
| d-methadone | PK | 0 | PK | 201 | PK | 292 | PK | 381 |
| 25 mg | | | | | | | | |
| Subject 1002 | BDNF 0.612 | | BDNF 2.529 | | BDNF | 4.820 | BDNF | 10.697 |
| d-methadone | PK | 0 | PK | 84.2 | PK | 223 | PK | 240 |
| 25 mg | | | | | | | | |
| Subject 1003 | BDNF | 0.376 | BDNF 2.038 | | BDNF | 2.920 | BDNF | 2.853 |
| d-methadone | PK | 0 | PK | 103 | PK | 160 | PK | 197 |
| 25 mg | | | | | | | | |
| Subject 1004 | BDNF | 0.460 | BDNF 1.862 | | BDNF | 2.348 | BDNF | 4.347 |
| d-methadone | PK | 0 | PK | 121 | PK | 179 | PK | 229 |
| 25 mg | | | | | | | | |
| Subject 1005 | BDNF | 0.497 | BDNF 8.995 | | BDNF | 2.458 | BDNF | 5.459 |
| d-methadone | PK | 0 | PK | 140 | PK | 224 | PK | 304 |
| 25 mg | | | | | | | | |
| Subject 1006 | BDNF | 1.08 | BDNF 0.605 | | BDNF | 0.692 | BDNF | 1.012 |
| PLACEBO | PK | 0 | PK | 0 | PK | 0 | PK | 0 |
| Subject 1007 | BDNF | 0.542 | BDNF 0.319 | | BDNF | 0.578 | BDNF | 0.577 |
| PLACEBO | PK | 0 | PK | 0 | PK | 0 | PK | 0 |
| Subject 1008 | BDNF | 1.922 | BDNF 2.750 | | BDNF | 3.790 | BDNF | 3.733 |
| d-methadone | PK | 0 | PK | 81.4 | PK | 106 | PK | 53 |
| 25 mg | | | | | | | | |

While the significance of these results may be limited by the small number of subjects, the correlation of the BDNF levels and d-methadone levels among 100% of 6 d-methadone treated subjects was strongly statistically significant; when compared to a lack of a similar increase in the two placebo subjects in the same group the results acquired even greater statistical significance (p<0.0001). These results show that d-methadone administered orally at a dose of 25 mg a day to healthy subjects undergoing a potentially stressful event (10-day in-patient clinical trial) significantly up-regulates BDNF serum levels and that this increase correlates with the measured serum d-methadone concentrations (p=0.028 at day 2, p=0.043 at day 6, and p=0.028 at day 10, all vs BDNF serum levels before treatment). The increase in BDNF was present starting on day 2 in all 6 d-methadone treated but not in the placebo treated subjects, and this increase was maintained throughout the whole 10-day study, again only for the d-methadone treated subjects and not the placebo subjects, suggesting a rapid onset and sustained effect of d-methadone on BDNF levels.

Statistical Analysis of Results

The analyses were performed by means of GraphPad Prism 5.0 and SPSS software. And descriptive statistics of BDNF levels (ng/ml) and serum d-methadone (ng/ml) for each time point are reported in Table 20.

TABLE 20
Descriptive Statistics

| | N | Mean | Std. Deviation | Minimum | Maximum |
|---|---|---|---|---|---|
| dMET_D2 | 6 | 121.8 | 44.73 | 81.40 | 201.0 |
| (w/o placebo) | | | | | |
| dMET_D6 | 6 | 197.3 | 63.88 | 106.0 | 292.0 |
| (w/o placebo) | | | | | |
| dMET_D10 | 6 | 234.0 | 110.2 | 53.00 | 381.0 |
| (w/o placebo) | | | | | |
| BDNF_T0 | 8 | .82675 | .528714 | .375 | 1.922 |
| BDNF_D2 | 8 | 2.83763 | 2.712967 | .319 | 8.995 |
| BDNF_D6 | 7 | 2.51514 | 1.538891 | .578 | 4.820 |
| BDNF_D10 | 8 | 4.08525 | 3.141263 | .577 | 10.697 |

Correlation:

The inventors first tested all the data together (plasma levels of BDNF vs PK). Then the inventors tested all data for the treated subjects without the placebo subjects. Then the inventors tested all data for the treated subjects without the baseline data. All the Spearman correlations were significant (p<0.0001). Subsequently, the inventors prepared a data set dividing subjects according to the time points, and analyzed whether BDNF concentration was correlated to PK at D2, 6 and 10. In this case, the correlation was significant at D2 (p=0.040, r=0.73) and D10 (p=0.017, r=0.80) when placebo subjects were considered. Results shown in Table 21 (below).

TABLE 21
Results of Spearman correlation analyses

| Time point | | | |
|---|---|---|---|
| (BDNF vs d-methadone) | N | Spearman r | P value |
| D2 with placebo | 8 | 0.731 | P = 0.04 |
| D2 w/o placebo | 6 | 0.371 | NS |
| D6 with placebo | 7 | 0.559 | NS |
| D6 w/o placebo | 5 | −0.200 | NS |
| D10 with placebo | 8 | 0.802 | P = 0.017 |
| D10 w/o placebo | 6 | 0.543 | NS |

Correlation is significant at the 0.05 level (2-tailed).

Comparison:

The inventors then performed a Wilcoxon Signed Ranks test to compare BDNF concentration at baseline (TO) and at D2, D6 and D10. All the differences were statistically significant. In particular, when considering 8 subjects (treated+placebo): TO-D2 p=0.036, TO-D6 p=0.043, TO-D10 p=0.025; when considering 6 subjects (no placebo): TO-D2 p=0.028, TO-D6 p=0.043, TO-D10 p=0.028. (See Table 22, below.)

TABLE 22
Descriptive Statistics (w/o placebo)

| | N | Mean | Std. Deviation | Minimum | Maximum |
|---|---|---|---|---|---|
| BDNF_T0 | 6 | .83100 | .601393 | .375 | 1.922 |
| BDNF_D2 | 6 | 3.62950 | 2.699290 | 1.862 | 8.995 |
| BDNF_D6 | 5 | 3.26720 | 1.037399 | 2.348 | 4.820 |
| BDNF_D10 | 6 | 5.18217 | 2.831985 | 2.853 | 10.697 |

Test Statistics^{a}

| | BDNF_D2- | BDNF_D6- | BDNF_D10- |
|---|---|---|---|
| | BDNF_T0 | BDNF_T0 | BDNF_T0 |
| Z | −2.201b | −2.023b | −2.201b |
| P value | .028 | .043 | .028 |

^{a}Wilcoxon Signed Ranks Test
bBased on negative ranks

Of note, subjects who were administered 50 mg and 75 mg doses of d-methadone consistently showed an increase in BDNF levels after treatment compared to pre-treatment values but this increase did not reach statistical significance against placebo.

Conclusion:

On the basis of these results, the inventors concluded that the administration of 25 mg d-methadone significantly increases BDNF serum levels in healthy volunteers. Plasma BDNF concentrations are not strongly correlated to the concentrations of the drug measured at the same time point (if placebo subjects are excluded from the data analyzed for correlation, as a rigorous statistical approach would suggest). In these subjects, the modulation of excitatory neuron firing rate by d-methadone's differential actions at the NMDAR subtypes, as shown in table 18 of Example 3 above, may have determined activity-dependent release of BDNF [Kuczewski N et al., Activity-dependent dendritic secretion of brain-derived neurotrophic factor modulates synaptic plasticity. Eur J Neurosci 32:1239-1244]. The administration of d-methadone may reverse the down regulation of BDNF seen in many diseases, including the nervous system disorders, endocrine-metabolic disorders, cardiovascular disorders, age-related disorders, eye disease, skin diseases, or symptoms and manifestations thereof, claimed in this application.

### Example 5: d-Methadone Increases Serum Testosterone Levels in Humans

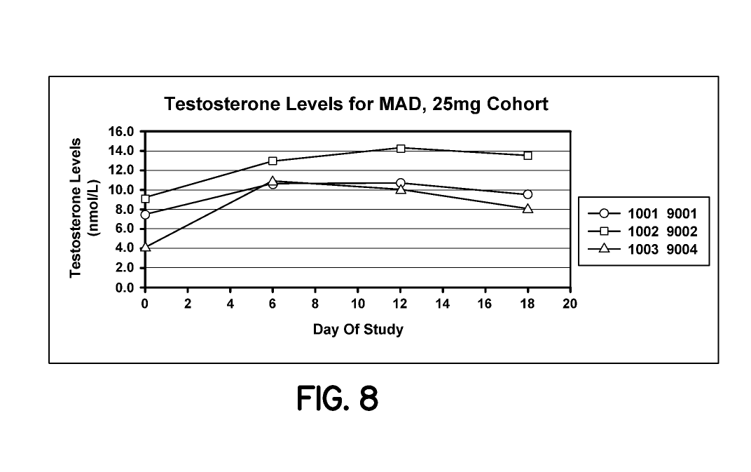

In the same double blind study described above for the BDNF up-regulating effects, d-methadone 25 mg administered once per day for ten days increased testosterone levels in all of the three male subjects tested; furthermore, testosterone serum levels on day 16, 6 days after discontinuation of d-methadone treatment, appeared to trend towards baseline levels—the testosterone levels before d-methadone treatment—corroborating the direct effect of d-methadone on up-regulation of testosterone. The dosing schedule and resulting data is shown below in Table 23 and in FIG. 8. In these same patients, the up-regulation of testosterone correlated with the d-methadone mediated increase in serum BDNF levels described in the section above. The increase in testosterone may be responsible the BDNF increase seen in our male subjects. The increase in BDNF in females may also be hormonally mediated, but hormonal levels were not measured in females.

TABLE 23
Dosing schedule for d-methadone 25 mg: once
a day for 10 days in 3 male subjects

| | | | Testosterone Levels (nmol/L) | | | |
|---|---|---|---|---|---|---|
| | | | normal range: 7.6-31.4 | | | |
| | | | Screening | | | Follow Up |
| | | | (Day −1) | Day 6 | Day 12 | (Day 18 ± 1) |
| Subject | | | −1 | 6 | 12 | 18 |
| 25 mg | 1001 | 9001 | 7.4 | 10.7 | 10.8 | 9.6 |
| 25 mg | 1002 | 9002 | 9.2 | 13.1 | 14.4 | 13.6 |
| 25 mg | 1003 | 9004 | 4.1 | 11.0 | 10.1 | 8.1 |

Statistical Analysis

This analysis were performed by means of the GraphPad Prism 5.0 software.

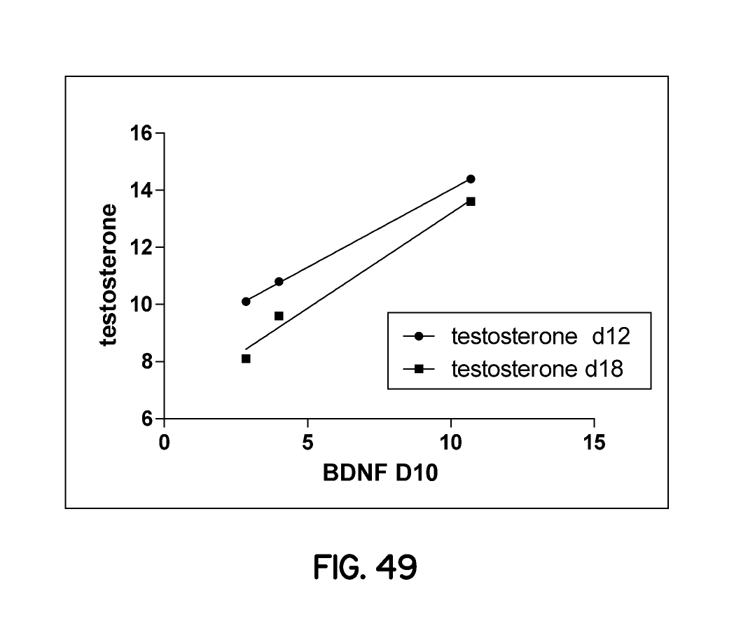

The data (testosterone and BDNF levels of the male 25 mg subject group) were test by a linear regression analysis. As shown in FIG. 49 below and Table 24 below, a r²=0.997 could be observed between testosterone day 12 and BDNF day 10 plasma levels). A Spearman correlation was performed, giving no significant results, due to the limited number of subjects.

TABLE 24
Linear regression analysis results

| | | A | B |
|---|---|---|---|
| rEE | | t 12 | t 18 |
| A | Linear reg. | Y | Y |
| 1 | Best-fit values | | |
| 2 | Slope | 0.5446 ± 0.008713 | 0.6655 ± 0.08746 |
| 3 | Y-intercept when X = 0.0 | 8.580 ± 0.05922 | 6.539 ± 0.5944 |
| 4 | X-intercept when Y = 0.0 | −15.75 | −9.826 |
| 5 | 1/slope | 1.836 | 1.503 |
| 6 | 95% Confidence Intervals | | |
| 7 | Slope | 0.4339 to 0.6553 | −0.4457 to 1.777 |
| 8 | Y-intercept when X = 0.0 | 7.827 to 9.332 | −1.014 to 14.09 |
| 9 | X-intercept when Y = 0.0 | −21.32 to −12.05 | −infinity to 0.6305 |
| 10 | Goodness of Fit | | |
| 11 | r2 | 0.9997 | 0.9830 |
| 12 | Sy.x | 0.05219 | 0.5239 |
| 13 | Is slope significantly non-zero? | | |
| 14 | F | 3908 | 57.91 |
| 15 | DFn, DFd | 1.000, 1.000 | 1.000, 1.000 |
| 16 | P value | 0.0102 | 0.0832 |
| 17 | Deviation from zero? | Significant | Not Significant |
| 18 | Data | | |
| 19 | Number of X values | 3 | 3 |
| 20 | Maximum number of Y replicates | 1 | 1 |
| 21 | Total number of values | 3 | 3 |
| 22 | Number of missing values | 0 | 0 |

The above finding is of importance because those skilled in the art know that opioids, including methadone, have been associated with low testosterone levels. The unexpected finding that d-methadone instead increases testosterone levels provides support for its development for the indications claimed throughout this application and dispels yet another perceived drawback.

### Example 6: d-Methadone Administration to Humans can Result in Improvement of Cognitive Function

When the inventors looked at the pharmacodynamics of d-methadone in healthy volunteers, they were able to confirm the absence of psychotomimetic symptoms even at the higher dosages. The baseline cognitive functions of healthy subjects were in general too high to detect a change in the cognitive domains of the Bond-Lader Visual Analog Scales before and after treatment. However, the SAD 5 mg d-methadone arm of the study compared to the placebo arm (double blind randomized design, six patients in the d-methadone arm and 11 patients in the placebo arm) was found to have improved scores for all of the domains explored by the Bond-Lader Visual Analog Scale pertaining to mental alertness and cognitive functions. The median Tₘₐₓ in the 5 mg d-methadone treatment group was 2.5 hours (range 2-3) and the mean Cₘₐₓ was 53.3 (minimum 29.6, median 48.40 and maximum 83.9). The Bond-Lader VAS scores for each patient were determined 2-3-5 hours post dose (placebo or d-methadone 5 mg).

The results are summarized in Table 25, below, and suggest that there may be a positive cognitive effect from d-methadone at doses as low as 5 mg in healthy subjects: subjects receiving d-methadone 5 mg felt more alert, more clear headed, more quick witted, more attentive, and more proficient. And these findings across the subjects (six subjects receiving one dose of 5 mg d-methadone) were consistent across all of the cognitive domains of the Bond-Lader Visual Analog Scale. In this application, the inventors previously discussed a novel analysis of the data from the study by Moryl et al., (Moryl, N. et al., A phase I study of d-methadone in patients with chronic pain. Journal of Opioid Management 2016: 12:1; 47-55): the inventors were able to discover that patients taking d-methadone experienced improvement in their Modified Mini Mental State scores. These findings taken together suggest that higher doses of d-methadone administered over an extended period of time might instead be helpful for diseases where there is even subtle disruption of normally functioning neural circuits and alteration of normal neural plasticity, and where regulation of select neural pathways and regulation of neural plasticity is required, including the NMDA receptor system and NET system and the up-regulation of BDNF and testosterone levels, and modulation of K⁺, Ca⁺ and Na⁺ currents, all influenced by d-methadone.

TABLE 25
Bond-Lader Visual Analog Scale: mean scores for the
cognitive domains 2-3-5 hours after a single dose
of the study drug (placebo or d-methadone 5 mg)

| Question text | | |
|---|---|---|
| Response anchors | Placebo | d-methadone 5 mg |
| I am feeling drowsy | 33.3 | 19.1 |
| 0: Alert | | |
| 100: Drowsy | | |
| I am feeling clear-headed | 72.5 | 91.9 |
| 0: Muzzy | | |
| 100: Clear-headed | | |
| I am feeling quick witted | 70.2 | 91.8 |
| 0: Mentally slow | | |
| 100: Quick witted | | |
| I am feeling dreamy | 30.7 | 10.5 |
| 0: Attentive | | |
| 100: Dreamy | | |
| I am feeling proficient | 72.4 | 93.6 |
| 0: Incompetent | | |
| 100: Proficient | | |

Because of its clinical effects on cognitive function seen in subjects with metastatic cancer but no known NS impairment (Moryl N et al., A phase I study of d-methadone in patients with chronic pain. Journal of Opioid Management 2016: 12:1; 47-55) uncovered by the inventors and the discovery made by the inventors on cognitive improvement in all tested cognitive domains of the Bond-Lader scale of normal subjects, from a very low, 5 mg, single dose of d-methadone, as described above, aside from its potential therapeutic role in diseases of the NS, d-methadone might also benefit the physiologic global deterioration that occurs with aging. BDNF, a member of neurotrophin growth factor family, physiologically mediates induction of neurogenesis and neuronal differentiation, promotes neuronal growth and survival and maintains synaptic plasticity and neuronal interconnections. BDNF levels have been shown to decrease in tissues with aging [Tapia-Arancibia, L. et al., New insights into brain BDNF function in normal aging and Alzheimer disease. Brain Research Reviews 2008. 59(1):201-20]. Studies using human subjects have found that hippocampal volume decreases with decreasing plasma levels of BDNF [Erickson, K. I. et al., Brain-derived neurotrophic factor is associated with age-related decline in hippocampal volume. The Journal of Neuroscience 2010. 30(15):5368-75].

Thus, a safe, well-tolerated drug, non-addictive and devoid of cognitive opioid-like and psychotomimetic effects, with high CNS penetration and the potential to regulate crucial NS pathways such as the NMDA receptor system and SERT and NET system and potentially increase BDNF and testosterone levels, such as d-methadone, may therefore benefit a large number of patients who presently lack alternatives within the narrow realm of presently approved drugs for CNS disorders and their neurological symptoms and manifestations. And, a drug like d-methadone, shown by the inventors to clinically improve cognitive function in normal subjects and to increase BDNF levels as shown by the inventors, might alleviate or prevent the mild cognitive impairment and other various NS deteriorations that occur during normal or accelerated aging or senescence and that can be reversed or prevented by higher levels of BDNF and or testosterone and by regulating NMDAR activity. As neurons also exert a trophic function and are also essential to maintain muscles, bone, skin and virtually all organs, d-methadone, by preserving neurons from aging through anti-apototic actions mediated by NMDA receptor antagonism with reduced excessive calcium influx in cells (which is pro-apoptotic) and promoting neuronal survival enhancement through BDNF and gonadal steroids including testosterone, holds strong anti-senescence potential in subjects with normal aging and in those with accelerated aging induced by a number of causes including genetic causes (progeria syndromes including Hutchinson-Gilford progeria syndrome (HGPS) and progeroid syndromes and “accelerated aging diseases” (such as Werner syndrome, Cockayne syndrome or xeroderma pigmentosum)) and accelerated aging from external causes such as toxic, traumatic, ischemic, infectious, neoplastic and inflammatory diseases and their treatments, including chemotherapy and radiotherapy (including brain radiotherapy).

The clinical usefulness and applications of novel NMDA receptor antagonists have been limited by their side effects (MK-801, ketamine) or too weak in vivo effects (memantine, amantadine, dextromethorphan). The present inventors have now shown that d-methadone is safe (see Example 1, above) and potentially effective for a multiplicity of clinical indications.

### Example 7: Administration of d-Methadone Results in a Lowering of Blood Glucose in Humans

The inventors also discovered a signal for possible lowering of blood sugar from the administration of d-methadone. In this study, the lowering of blood sugar occurred from a 10 day course of 25 mg d-methadone daily dose in humans: in normo-glycemic healthy volunteers, serum glucose concentration may be lowered on day 10 and day 12 after treatment with d-methadone 25 mg per day for 10 days. The analysis was performed by means of a colorimetric kit. Quantitative determination of glucose was carried out by standard calibration curves constructed with glucose amounts ranging from 0 to 10 nmoles (n=6). The calibration curves showed a linear dependence on glucose amount (r2≥0.992). Data are shown in Table 26 (below).

TABLE 26

| Subject | baseline | Day-10 | Day-12 |
|---|---|---|---|
| 1001 | 5.5 | 5.2 | 5.3 |
| 1002 | 5.3 | 5.3 | 4.5 |
| 1003 | 4.2 | 5.9 | 4.9 |
| 1004 | 5.7 | 5.4 | 4.9 |
| 1005 | 4.6 | 4.9 | 4.1 |
| 1006P | 5.0 | 5.4 | 4.9 |
| 1007P | 4.3 | 5.8 | 4.8 |
| 1008 | 5.1 | 3.2 | 4.9 |

Results

Mean glucose levels increased by +0.95 mmol/I in the placebo group, two patients, 1006 and 1007, on day 10 compared to baseline. Mean glucose levels decreased by −0.08 mmol/I in the six d-methadone treated patients, on day 10 compared to baseline. Mean glucose levels increased by +0.2 mmol/I in the placebo group, two patients, day 12 compared to baseline. And mean glucose levels decreased by −0.43 mmol/I in the six d-methadone treated patients, day 12 compared to baseline.

In this prospective double blind placebo controlled normo-glycemic 8-subject study, a decrease in serum glucose was noted in the treatment group (6 patients) compared to the placebo group (2 patients); the decrease did not appear to be related to d-methadone levels or BDNF levels and persisted for at least 2 days after the cessation of the 10 day d-methadone treatment period.

In this study, normal glucose levels were a requirement for enrollment, thus regression towards the mean should also be considered when looking at the data. Also, as d-methadone may act as a regulator of abnormal (hyperglycemic levels), via NMDA, BDNF and or testosterone regulation or other mechanisms, the results are likely to be more meaningful and reach statistical significance when the study is repeated in a cohort of hyperglycemic patients rather than normo-glycemic subjects.

In summary, the above results signal a possible blood glucose lowering effect of d-methadone. These glucose lowering effects are likely to become more evident when testing is performed in patients with hyperglycemia (diabetes mellitus and metabolic syndrome). While a hypoglycemic effect of high doses of racemic methadone has been previously described [Flory J H et al., Methadone Use and the Risk of Hypoglycemia for Inpatients with Cancer Pain. Journal of pain and symptom management. 2016; 51(1):79-87] this is the first time that the same effect is noted for d-methadone.

### Example 8: Administration of d-Methadone Results in a Dose Dependent Decreased Weight Gain in Rats

In addition to the possible lowering of blood glucose in humans as described above, the inventors also uncovered a signal a dose dependent decreased weight gain from d-methadone administration to rats during an experiment on chronic constrictive nerve injury model of neuropathic pain. Materials and Methods: Male Sprague Dawley rats (150 g on arrival) from Harlan (Indianapolis, Ind.) were used in the study. Upon receipt, rats were assigned unique identification numbers and were group housed with 3 rats per cage in polycarbonate cages with micro-isolator filter tops. All rats were examined, handled, and weighed prior to initiation of the study to assure adequate health and suitability. Chow and water were provided ad libitum for the duration of the study. Animals were singly-housed for the duration of the study. Test compounds were administered chronically once daily for 15 days. Test compound: d-Methadone (10, 20, and 40 mg/kg; Relmada Therapeutics) was dissolved in saline and administered subcutaneous (S.C.) at a dose volume of 1 ml/kg. Vehicle control: Saline was administered subcutaneous (S.C.) at a dose volume of 1 ml/kg. Rats, provided with ad libitum food and water, were administered d-methadone at one of three doses, for 15 days, and their variation in weight from baseline was compared to the weight of rats administered vehicle, as shown in Table 27 below.

TABLE 27

| 10 count per group, 5 groups | Mean baseline weight in grams | Mean weigh on day 15 in grams |
|---|---|---|
| vehicle | 286.6 SD14.5 SE 4.6 | 337.6 SD 18.4 SE 5.8 mean + 51 |
| d-methadone 10 mg/kg | 289.3 SD 18.4 SE 5.8 | 329.9 SD 23.5 SE 7.4 mean + 41 |
| d-methadone 20 mg/kg | 284.4 SD 14.2 SE 4.5 | 320.2 SD 23.2 SE 7.3 mean + 36 |
| d-methadone 40 mg/kg | 286.0 SD 10.3 SE 3.4 | 308.4 SD 9.9 SE 3.3 mean + 22 |

Rats appeared to gain less weight when administered higher doses of d-methadone, suggesting a possible effect on metabolism and or food intake. Data were analyzed by analysis of variance (ANOVA) followed by Fisher LSD post-hoc comparisons. An effect was considered significant if p<0.05. Data are presented as the mean±standard error of the mean (S.E.M.). A significant interaction between treatment and body weights (p<0.001) was observed. All rats gained body weight during the study, however, rats treated with d-Methadone (40 mg/kg) exhibited lower weight gain compared to vehicle-treated animals. And so, the actions of d-methadone as an NMDA antagonist and its potential for increasing BDNF and testosterone levels suggest that d-methadone, which is devoid of the opioid side effects of methadone, could be used to regulate metabolic parameters in patients with altered glucose tolerance such as patients with DM or the metabolic syndrome or overweight and obese patients. Thus, by influencing cognitive function, behavior, and energy balance through its effects on BDNF and testosterone levels, NMDAR, and NET and SERT, d-methadone could therefore be useful for the treatment and prevention of weight gain, obesity, DM and the metabolic syndrome and aging.

### Example 9: d-Methadone Exhibits In Vivo Behavioral Effects Adequate to Exert Clinical Effect and Neuroprotection

The present inventors also performed a forced swim test in rats. While the forced swim test has previously been successfully used to evaluate drugs for a potential for antidepressant effects, the inventors—in this Example—more specifically studied the actual behavioral effects of d-methadone in vivo compared to ketamine.

Ketamine is a well-known NMDA receptor antagonist clinically approved for anesthesia. The clinical usefulness of ketamine, beyond its use as an anesthetic drug, is limited by its psychotomimetic effects. d-Methadone, however, has now been shown by the inventors to be devoid of psychotomimetic effects and other clinically significant opioid side effects at doses that have the potential to improve cognition and other neurological diseases and manifestations (see Example 1, above).

Materials and Methods

Male Sprague Dawley rats (obtained from Envigo; Indianapolis, Ind.) were used in this study. Upon receipt, rats were assigned unique identification numbers (tail marked). Animals were housed 3 per cage in polycarbonate cages with micro-isolator filter tops and acclimated for 7 days. All rats were examined, handled, and weighed prior to the study to assure adequate health and suitability. Rats were maintained on a 12/12 light/dark cycle. The room temperature was maintained between 20° C. and 23° C., with a relative humidity around 50%. Standard rodent chow and water were provided ad libitum for the duration of the study. Animals were randomly assigned across treatment groups with 10 rats per treatment group.

As described above, the compound being tested in this Example was d-methadone. In particular, this Example used d-methadone (obtained from Mallinckrodt, St. Louis, Mo.—lot#1410000367) dissolved in sterile water. In particular, d-Methadone dose formulations were prepared by dissolving a weighed amount of d-methadone in a measured volume of sterile injectable water to achieve concentrations of 10, 20, and 40 mg/mL.

Further, the reference compound for this Example was ketamine (obtained from Patterson Veterinary, Chicago, Ill.—lot# AH013JC) dissolved in saline. Ketamine dose formulations were prepared by dilution of a stock solution of ketamine at 100 mg/mL into the desired dose of 10 mg/mL.

Dose formulations for both d-methadone and ketamine were prepared shortly before use. And, rats were then administered vehicle, ketamine, or d-methadone 24 hours prior to forced swim and locomotor activity tests. Ketamine was administered intraperitoneally (“IP”) at a dose volume of 1 mL/kg. And d-methadone and vehicle were administered subcutaneously (“SC”) at a dose volume of 1 mL/kg.

Forced Swim Procedure:

When rats are forced to swim in a small cylinder from which no escape is possible they readily adopt a characteristic immobile posture and make no further attempts to escape except for small movements needed to prevent them from drowning. The immobility induced by the procedure can be reversed or largely decreased by a wide variety of antidepressants, suggesting that this test is sensitive to antidepressant-like effect. However, since this test will also pick up many false positives (e.g., psychostimulants and antihistaminergics), locomotor activity was also performed to rule out hyperactivity.

All experiments were carried out in ambient temperature under artificial lighting during the light cycle of the rat. Each forced swim chamber was constructed of clear acrylic (height=40 cm; diameter=20.3 cm). All rats were exposed to a swim test (‘habituation’) prior to compound administration. This pre-administration swim test consisted of one 15 min session in individual cylinders containing 23±1° C. water, which was followed 24 h later by the experimental test of 5 min. The water level was 16 cm deep during habituation and 30 cm deep during test. Immobility, climbing, and swimming behaviors were recorded every 5 sec for a total of 60 counts per subject. In the event that an animal was unable to maintain a posture with its nose above water it was removed immediately from the water and therefore eliminated from the study.

Rats were administered vehicle, ketamine, or d-methadone on day 1 (after habituation; 24 hours prior to forced swim testing). The test and the analysis of video files of the test were performed by an observer blind to treatment. Data are represented as a frequency of total behavior over the 5 min trial.

Locomotor Activity Assessment:

Locomotor activity was assessed using the Hamilton Kinder apparatus (commercially available from Kinder Scientific, San Diego, Calif.), which is known to those of ordinary skill in the art. The test chambers were old standard rat cages, different from current housing, (24×45 cm) that fit inside two steel frames (24×46 cm) and are fitted with two-dimensional 4×8 beam grids to monitor horizontal and vertical locomotor activity. Photocell beam interruptions were automatically recorded by a computer system for 60 minutes in 5-minute bins. The analysis was configured to divide the open field of the chamber into a center and a periphery zone. Distance measured from vertical beam breaks.

Rats were brought to the experimental room for at least 1 hour of acclimation to the experimental room prior to the start of testing. A clean cage was used for each rat for testing. Rats were administered vehicle, ketamine, or d-methadone 24 hours prior to locomotor activity testing.

Statistical Analysis:

Data were analyzed by analysis of variance (ANOVA) followed by post-hoc comparisons with Fisher Tests when appropriate (following significant main or interaction effects). An effect was considered significant if p<0.05. Any rats that exhibited individual measures that fell above or below 2 standard deviations from the mean were removed from the analysis.

Results from the Forced Swim Test

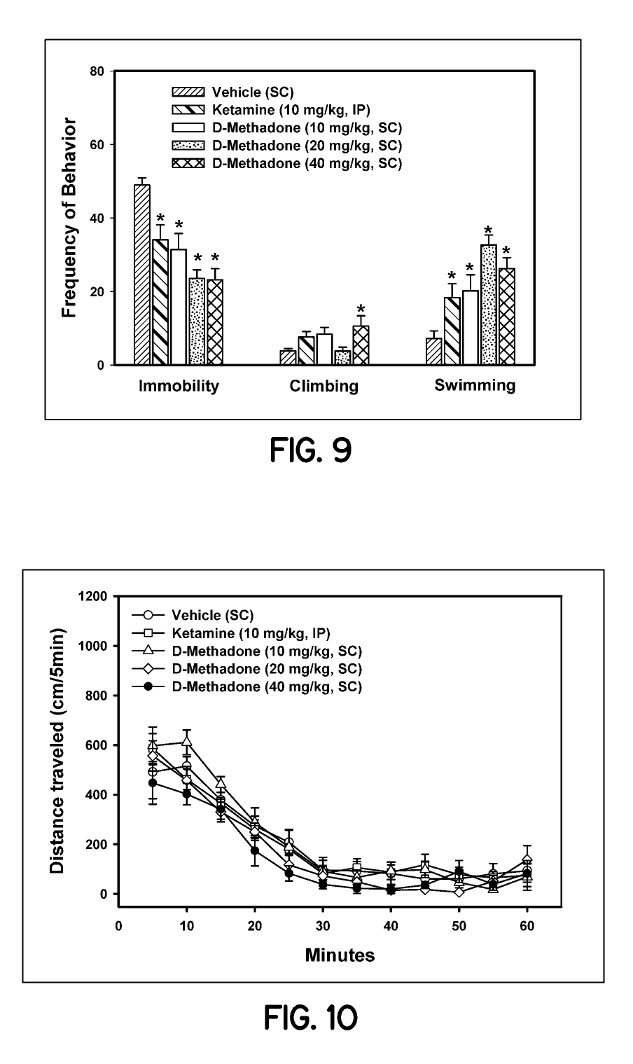

As described above, during the forced swim test procedures, immobility, climbing, and swimming behaviors were recorded every 5 seconds for a total of 60 counts per subject (resulting in a 5 minute trial per subject). Data was represented as a frequency of each behavior during the trial. The effects of ketamine and d-methadone on frequency of immobility, climbing and swimming behavior are shown in FIG. 9 [where data represent the mean±standard error of mean (SEM); *p<0.05 compare to vehicle group].

Immobility:

As can be seen from FIG. 9, d-methadone (10, 20 and 40 mg/kg) and ketamine significantly decreased the frequency of immobility compared to the vehicle-treated animals. The magnitude of effect of d-methadone (20 and 40 mg/kg) was significantly larger than that of ketamine. The statistical data for the forced swim test as it relates to immobility can be seen in Tables 28-30, below.

TABLE 28
ANOVA Table for Immobility

| | DF | Sum of Squares | Mean Square | F-Value | P-Value | Lambda | Power |
|---|---|---|---|---|---|---|---|
| Treatment | 4 | 4064.544 | 1016.136 | 9.318 | <.0001 | 37.273 | 1.000 |
| Residual | 43 | 4689.122 | 109.049 | | | | |

TABLE 29
Means Table for Immobility

| Effect: Treatment | Count | Mean | Std. Dev. | Std. Err. |
|---|---|---|---|---|
| Vehicle | 9 | 49.000 | 5.788 | 1.929 |
| Ketamine (10 mg/kg) | 10 | 34.100 | 12.784 | 4.043 |
| D-Methadone (10 mg/kg) | 10 | 31.400 | 13.898 | 4.395 |
| D-Methadone (20 mg/kg) | 9 | 23.556 | 7.038 | 2.346 |
| D-Methadone (40 mg/kg) | 10 | 23.200 | 9.520 | 3.010 |

TABLE 30
Fisher's PLSD for Immobility

| Effect: Treatment | Mean | Crit. | | |
|---|---|---|---|---|
| Significance Level: 5% | Diff. | Diff. | P-Value | |
| Vehicle, Ketamine (10 mg/kg) | 14.900 | 9.676 | .0034 | S |
| Vehicle, D-Methadone (10 mg/kg) | 17.600 | 9.676 | .0007 | S |
| Vehicle, D-Methadone (20 mg/kg) | 25.444 | 9.928 | <.0001 | S |
| Vehicle, D-Methadone (40 mg/kg) | 25.800 | 9.676 | <.0001 | S |
| Ketamine (10 mg/kg), D-Methadone | 2.700 | 9.418 | .5662 | |
| (10 mg/kg) | | | | |
| Ketamine (10 mg/kg), D-Methadone | 10.544 | 9.676 | .0334 | S |
| (20 mg/kg) | | | | |
| Ketamine (10 mg/kg), D-Methadone | 10.900 | 9.418 | .0243 | S |
| (40 mg/kg) | | | | |
| D-Methadone (10 mg/kg), D-Methadone | 7.844 | 9.676 | .1094 | |
| (20 mg/kg) | | | | |
| D-Methadone (10 mg/kg), D-Methadone | 8.200 | 9.418 | .0862 | |
| (40 mg/kg) | | | | |
| D-Methadone (20 mg/kg), D-Methadone | .356 | 9.676 | .9413 | |
| (40 mg/kg) | | | | |

Climbing:

As can be seen from FIG. 9, d-Methadone (40 mg/kg) significantly increased the frequency of climbing compared to the vehicle-treated animals. The statistical data for the forced swim test as it relates to climbing can be seen in Tables 31-33, below.

TABLE 31
ANOVA Table for Climbing

| | DF | Sum of Squares | Mean Square | F-Value | P-Value | Lambda | Power |
|---|---|---|---|---|---|---|---|
| Treatment | 4 | 339.606 | 84.901 | 2.736 | .0409 | 10.944 | .706 |
| Residual | 43 | 1334.311 | 31.030 | | | | |

TABLE 32
Means Table for Climbing

| Effect: Treatment | Count | Mean | Std. Dev. | Std. Err. |
|---|---|---|---|---|
| Vehicle | 9 | 3.778 | 2.048 | .683 |
| Ketamine (10 mg/kg) | 10 | 7.600 | 4.858 | 1.536 |
| D-Methadone (10 mg/kg) | 10 | 8.400 | 5.816 | 1.839 |
| D-Methadone (20 mg/kg) | 9 | 3.778 | 3.153 | 1.051 |
| D-Methadone (40 mg/kg) | 10 | 10.600 | 8.847 | 2.798 |

TABLE 33
Fisher's PLSD for Climbing

| Effect: Treatment | Mean | Crit. | | |
|---|---|---|---|---|
| Significance Level: 5% | Diff. | Diff. | P-Value | |
| Vehicle, Ketamine (10 mg/kg) | −3.822 | 5.162 | .1426 | |
| Vehicle, D-Methadone (10 mg/kg) | −4.622 | 5.162 | .0779 | |
| Vehicle, D-Methadone (20 mg/kg) | 0.000 | 5.296 | • | |
| Vehicle, D-Methadone (40 mg/kg) | −6.822 | 5.162 | .0108 | S |
| Ketamine (10 mg/kg), D-Methadone | −.800 | 5.024 | .7497 | |
| (10 mg/kg) | | | | |
| Ketamine (10 mg/kg), D-Methadone | 3.822 | 5.162 | .1426 | |
| (20 mg/kg) | | | | |
| Ketamine (10 mg/kg), D-Methadone | −3.000 | 5.024 | .2351 | |
| (40 mg/kg) | | | | |
| D-Methadone (10 mg/kg), D-Methadone | 4.622 | 5.162 | .0779 | |
| (20 mg/kg) | | | | |
| D-Methadone (10 mg/kg), D-Methadone | −2.200 | 5.024 | .3821 | |
| (40 mg/kg) | | | | |
| D-Methadone (20 mg/kg), D-Methadone | −6.822 | 5.162 | .0108 | S |
| (40 mg/kg) | | | | |

Swimming:

As can be seen from FIG. 9, d-methadone (10, 20, and 40 mg/kg) and ketamine significantly increased the frequency of swimming compared to the vehicle-treated animals. Compared to ketamine, rats treated with d-methadone (20 mg/kg) showed increased swimming behavior. The statistical data for the forced swim test as it relates to swimming can be seen in Tables 34-36, below.

TABLE 34
ANOVA Table for Swimming

| | DF | Sum of Squares | Mean Square | F-Value | P-Value | Lambda | Power |
|---|---|---|---|---|---|---|---|
| Treatment | 4 | 3283.061 | 820.765 | 7.556 | .0001 | 30.224 | .996 |
| Residual | 43 | 4670.856 | 108.625 | | | | |

TABLE 35
Means Table for Swimming

| Effect: Treatment | Count | Mean | Std. Dev. | Std. Err. |
|---|---|---|---|---|
| Vehicle | 9 | 7.222 | 6.160 | 2.053 |
| Ketamine (10 mg/kg) | 10 | 18.300 | 12.102 | 3.827 |
| D-Methadone (10 mg/kg) | 10 | 20.200 | 13.774 | 4.356 |
| D-Methadone (20 mg/kg) | 9 | 32.667 | 8.185 | 2.728 |
| D-Methadone (40 mg/kg) | 10 | 26.200 | 9.461 | 2.992 |

TABLE 36
Fisher's PLSD for Swimming

| | Mean | Crit. | | |
|---|---|---|---|---|
| Significance Level: 5% | Diff. | Diff. | P-Value | |
| Vehicle, Ketamine (10 mg/kg) | −11.078 | 9.65 | .0256 | S |
| Vehicle, D-Methadone (10 mg/kg) | −12.978 | 9.65 | .0096 | S |
| Vehicle, D-Methadone (20 mg/kg) | −25.444 | 9.90 | <.000 | S |
| D-Methadone (40 mg/kg) | −18.978 | 9.65 | .0003 | S |
| Ketamine (10 mg/kg), D-Methadone | −1.900 | 9.40 | .6856 | |
| (10 mg/kg) | | | | |
| Ketamine (10 mg/kg), D-Methadone | −14.367 | 9.65 | .0045 | S |
| (20 mg/kg) | | | | |
| Ketamine (10 mg/kg), D-Methadone | −7.900 | 9.40 | .0973 | |
| (40 mg/kg) | | | | |
| D-Methadone (10 mg/kg), D-Methadone | −12.467 | 9.65 | .0126 | S |
| (20 mg/kg) | | | | |
| D-Methadone (10 mg/kg), D-Methadone | −6.000 | 9.40 | .2049 | |
| (40 mg/kg) | | | | |
| D-Methadone (20 mg/kg), D-Methadone | 6.467 | 9.65 | .1840 | |
| (40 mg/kg) | | | | |

Results from Locomotor Activity Assessment

As described above, during the locomotor activity portion of the study, both horizontal locomotor activity (total distance traveled) and vertical locomotor activity (rearing) were examined. The results for each of these types of activities are discussed below.

Total Distance Traveled:

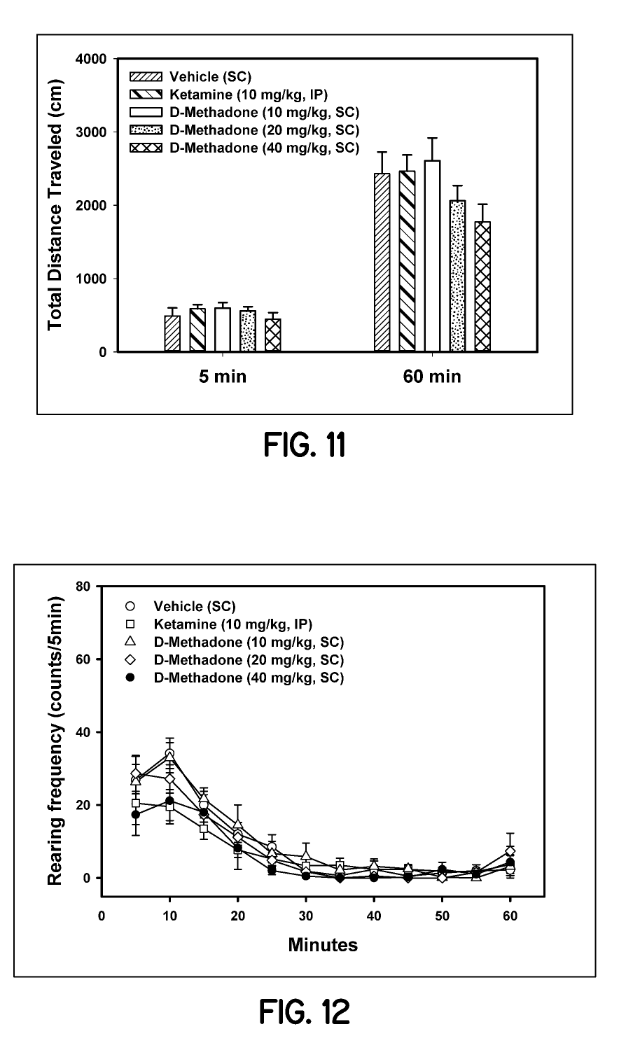

Time course for the effects of ketamine and d-methadone on locomotor activity is shown in FIG. 10 (Data represent mean±SEM). Two way repeated measures ANOVA found no significant treatment effect and no significant treatment×time interaction. Total distance traveled was calculated by summing the data during the 60 minute test and are shown in FIG. 11 (Data represent mean±SEM). One-way ANOVA found no significant effect of ketamine or d-methadone on this measure. In addition, distance traveled during the first 5 minutes of the test which corresponds to the Forced Swim Test time is shown in FIG. 11. One-way ANOVA found no significant treatment effect. The statistical data for locomotor activity for distance traveled can be seen in Tables 37-41, below.

TABLE 37
ANOVA Table for Time

| | DF | Sum of Squares | Mean Square | F-Value | P-Value | Lambda | Power |
|---|---|---|---|---|---|---|---|
| Treatment | 4 | 233651.072 | 58412.768 | 1.740 | .1728 | 6.959 | .448 |
| Subject(Group) | 25 | 839382.917 | 33575.317 | | | | |
| Category for Time | 11 | 10643356.956 | 967577.905 | 104.069 | <.0001 | 1144.755 | 1.000 |
| Category for Time * Treatment | 44 | 349004.461 | 7931.920 | .853 | .7329 | 37.537 | .884 |
| Category for Time * Subject(Group) | 275 | 2556812.417 | 9297.500 | | | | |

TABLE 38
ANOVA Table for min 1-5

| | DF | Sum of Squares | Mean Square | F-Value | P-Value | Lambda | Power |
|---|---|---|---|---|---|---|---|
| Treatment | 4 | 99216.333 | 24804.083 | .644 | .6365 | 2.574 | .179 |
| Residual | 25 | 963579.833 | 38543.193 | | | | |

TABLE 39
Means Table for min 1-5

| Effect: Treatment | Count | Mean | Std. Dev. | Std. Err. |
|---|---|---|---|---|
| vehicle | 6 | 491.333 | 263.595 | 107.612 |
| d-Methadone (10 mg/kg) | 6 | 597.167 | 185.525 | 75.740 |
| d-Methadone (20 mg/kg) | 6 | 556.833 | 149.696 | 61.113 |
| d-Methadone (40 mg/kg) | 6 | 447.500 | 211.237 | 86.237 |
| ketamine (10 mg/kg) | 6 | 586.333 | 147.595 | 60.255 |

TABLE 40
ANOVA Table for Total Distance Traveled

| | DF | Sum of Squares | Mean Square | F-Value | P-Value | Lambda | Power |
|---|---|---|---|---|---|---|---|
| Treatment | 4 | 2803812.867 | 700953.217 | 1.740 | .1728 | 6.959 | .448 |
| Residual | 25 | 10072595.000 | 402903.800 | | | | |

TABLE 41
Means Table for Total Distance Traveled (cm/60 min)

| | Count | Mean | Std. Dev. | Std. Err. |
|---|---|---|---|---|
| vehicle | 6 | 2431.833 | 720.701 | 294.225 |
| d-Methadone (10 mg/kg) | 6 | 2605.500 | 767.001 | 313.127 |
| d-Methadone (20 mg/kg) | 6 | 2060.667 | 511.457 | 208.801 |
| d-Methadone (40 mg/kg) | 6 | 1773.000 | 589.898 | 240.825 |
| ketamine (10 mg/kg) | 6 | 2464.333 | 545.207 | 222.580 |

Rearing:

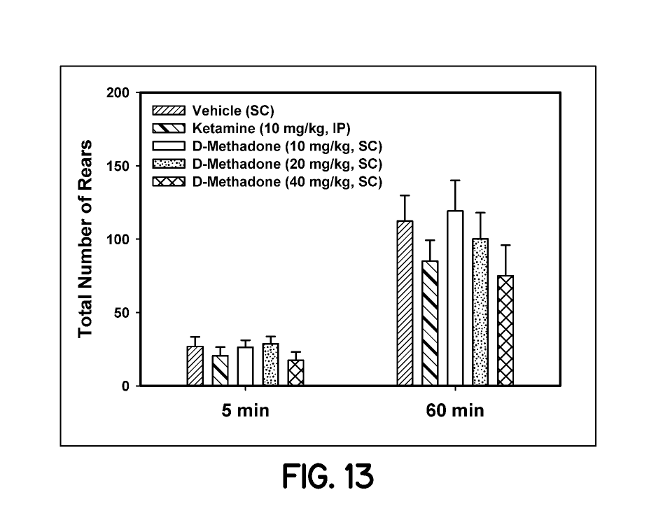

Time course for the effects of ketamine and d-methadone on rearing activity is shown in FIG. 12 (Data represent mean±SEM). Two way repeated measures ANOVA found no significant treatment effect and no significant treatment x time interaction. Total rearing frequency was summed during the 60 minute test and is shown in FIG. 13. One-way ANOVA found no significant effect of ketamine and d-methadone on this measure. In addition rearing during the first 5 minutes of the test which corresponds to the Forced Swim Test time is shown in FIG. 13 (Data represent mean±SEM). One-way ANOVA found no significant treatment effect. The statistical data for locomotor activity for distance traveled can be seen in Tables 42-46, below.

TABLE 42
ANOVA Table for Time

| | DF | Sum of Squares | Mean Square | F-Value | P-Value | Lambda | Power |
|---|---|---|---|---|---|---|---|
| Treatment | 4 | 677.806 | 169.451 | .994 | .4290 | 3.977 | .263 |
| Subject(Group) | 25 | 4261.083 | 170.443 | | | | |
| Category for Time | 11 | 30001.656 | 2727.423 | 59.259 | <.0001 | 651.853 | 1.000 |
| Category for Time * Treatment | 44 | 1866.928 | 42.430 | .922 | .6158 | 40.563 | .915 |
| Category for Time * Subject(Group) | 275 | 12656.917 | 46.025 | | | | |

TABLE 43
ANOVA Table for min 1-5

| | DF | Sum of Squares | Mean Square | F-Value | P-Value | Lambda | Power |
|---|---|---|---|---|---|---|---|
| Treatment | 4 | 551.533 | 137.883 | .730 | .5800 | 2.920 | .199 |
| Residual | 25 | 4722.333 | 188.893 | | | | |

TABLE 44
Means Table for min 1-5

| Effect: Treatment | Count | Mean | Std. Dev. | Std. Err. |
|---|---|---|---|---|
| d-Methadone (10 mg/kg) | 6 | 26.333 | 11.776 | 4.807 |
| d-Methadone (20 mg/kg) | 6 | 28.667 | 12.111 | 4.944 |
| d-Methadone (40 mg/kg) | 6 | 17.333 | 13.952 | 5.696 |
| ketamine (10 mg/kg) | 6 | 20.500 | 14.391 | 5.875 |
| Vehicle | 6 | 26.833 | 16.043 | 6.549 |

TABLE 45
ANOVA Table for Total Rearing

| | DF | Sum of Squares | Mean Square | F-Value | P-Value | Lambda | Power |
|---|---|---|---|---|---|---|---|
| Treatment | 4 | 8133.667 | 2033.417 | .994 | .4290 | 3.977 | .263 |
| Residual | 25 | 51133.000 | 2045.320 | | | | |

TABLE 46
Means Table for Total Rearing

| Effect: Treatment | Count | Mean | Std. Dev. | Std. Err. |
|---|---|---|---|---|
| d-Methadone (10 mg/kg) | 6 | 119.167 | 51.199 | 20.902 |
| d-Methadone (20 mg/kg) | 6 | 100.167 | 43.815 | 17.887 |
| d-Methadone (40 mg/kg) | 6 | 75.000 | 51.268 | 20.930 |
| ketamine (10 mg/kg) | 6 | 85.000 | 35.060 | 14.313 |
| Vehicle | 6 | 112.333 | 42.754 | 17.454 |

Conclusions

The study described in this Example evaluated the behavioral effects of d-methadone (10, 20, and 40 mg/kg) following a single administration, 24 hours prior to test. Regarding the forced swim test: At all doses tested, d-methadone significantly decreased immobility of the rats compared to the vehicle, suggesting NMDA mediated behavioral effects. In addition, the effect of d-methadone (20 and 40 mg/kg) on immobility was larger than the effect seen with ketamine (10 mg/kg). Further, d-methadone (40 mg/kg) significantly increased the frequency of climbing compared to the vehicle-treated animals. d-Methadone (10, 20, and 40 mg/kg) and ketamine significantly increased the frequency of swimming compared to the vehicle-treated animals. Compared to ketamine, rats treated with d-methadone (20 mg/kg) showed increased swimming behavior. It should be noted that the effects of d-methadone (10, 20, and 40 mg/kg) in the forced swim test were not confounded by any changes in the locomotor activity of the rats. Taken together the results of this forced swim test in rats suggest that d-methadone has in vivo behavioral effects that are comparable to or stronger than those seen with ketamine, and that are adequate to exert clinical effects likely related to actions on the NMDAR, NET, SERT systems, and modulation of neurotrophins and or testosterone in humans.

As d-methadone showed no evidence of psychotomimetic effects or other limiting side effects at potentially therapeutic doses (example 1), the results of the forced swim rat test suggest that d-methadone potentially has clinically useful in vivo NMDAR antagonistic effects which may be indicated for an array of neurological diseases and symptoms where regulation of the NMDARs, excitotoxicity, BDNF, testosterone and neuronal plasticity modulation are implicated.

### Example 10: The Female Urine Sniffing Test (FUST) and Novelty-Suppressed Feeding Test (NSFT) Demonstrate that d-Methadone Exhibits In Vivo Behavioral Effects Adequate to Exert Clinical Effects and Neuroprotection

While FUST is sensitive to the acute effects of antidepressants and NSFT is sensitive to the acute administration of anxiolytics and chronic antidepressant treatment, they also both depend on memory and learning and therefore the results discussed above may also suggest an effect of d-methadone on memory and learning, independent of effects on mood or anxiety.

The objective of the study of this Example was to examine the influence of d-methadone, which has NMDA competitive antagonist properties, on rat behaviors, compared to the NMDA receptor antagonist Ketamine.

Behavioral Testing:

Initial studies examined the influence of d-methadone or ketamine on behavior in the FUST and the NSFT. The female urine sniffing test (FUST) was designed to monitor reward-seeking activity in rodents sensitive to acute administration of antidepressants. The novelty-suppressed feeding test (NSFT) measures a rodent's aversion to eating in a novel environment. This test assesses the latency of an animal to approach and eat a familiar food in an aversive environment. The test is sensitive to acute administration of anxiolytics and chronic antidepressant treatment but insensitive to acute antidepressants.

FUST was conducted according to the published procedures (which are known to those of ordinary skill in the art). Rats were habituated for 60 min to a cotton-tipped applicator dipped in tap water placed in their home cage. For the test, rats are first exposed to a cotton tip dipped in tap water for 5 min, and 45 min later exposed to another cotton tip infused with fresh female urine. Male behavior was video recorded and total time spent sniffing the cotton-tipped applicator is determined. For NSFT, rats were food deprived for 24 hr and then placed in an open field with food pellets in the center; latency to eat is recorded in seconds. As a control, food consumption in the home cage is quantified.

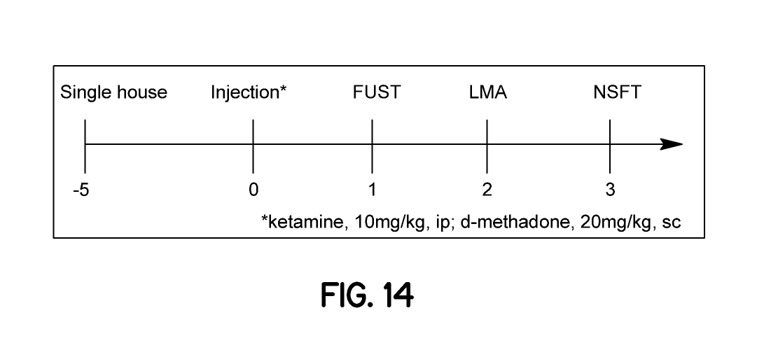

Drug administration: Rats were administered vehicle, ketamine (10 mg/kg, ip), or d-methadone (20 mg/kg, sc). Behavior in the FUST was conducted 24 hr and NSFT 72 hr after dosing (the general schedule for administration is shown in FIG. 14).

Results

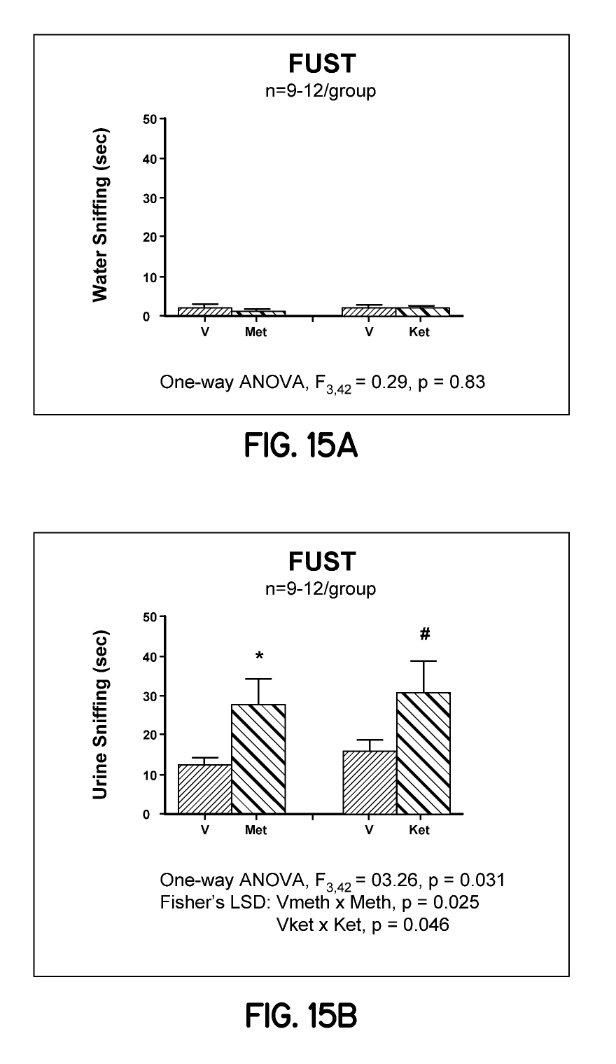

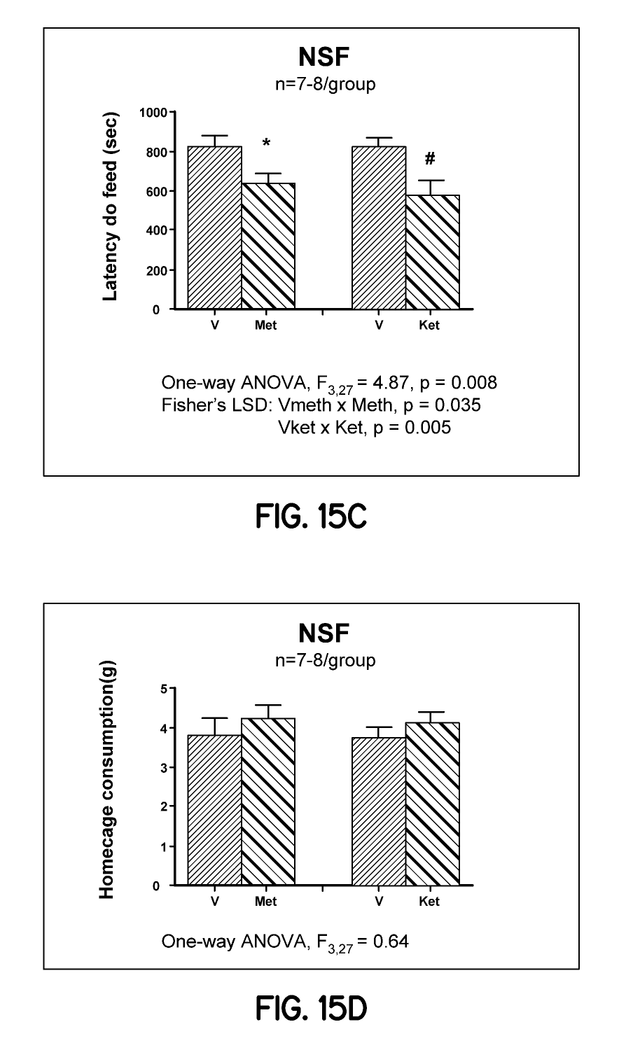

The results of the FUST are shown in FIGS. 15A and 15B, and demonstrate that administration of ketamine increases the time male rats spent engaged in sniffing female urine compared to vehicle group (FIG. 15B). Similarly, a single dose of d-methadone increased the time spent sniffing female urine compared to vehicle. In contrast, ketamine or d-methadone had no effect on time sniffing water, demonstrating that the effect of drug treatment was specific to the rewarding effects of female urine (FIG. 15A). Thus, both compounds resulted in statistically significant changes in rodent behavior, suggesting a d-methadone effect in humans compatible with acute and chronic antidepressant actions, anxiolytic actions and possibly an improvement in memory and learning, independent of mood or anxiety.

The results of the NSFT are shown in FIGS. 15C and 15D, and demonstrate that a single dose of ketamine significantly decreases the latency to eat in a novel open field. Similarly, a single dose of d-methadone also significantly decreased the latency to enter and eat in the novel feed. In contrast, neither ketamine or methadone influenced latency to feed in the home cage. These findings demonstrate that ketamine and d-methadone produce rapid antidepressant-like actions in the NSFT, effects that are only observed after chronic administration of an SSRI antidepressant. Thus, both compounds resulted in statistically significant changes in rodent behavior, suggesting a d-methadone effect in humans compatible with acute and chronic antidepressant actions, anxiolytic actions and possibly an improvement in memory and learning, independent of mood or anxiety. As d-methadone showed no evidence of psychotomimetic effects or other limiting side effects at potentially therapeutic doses (example 1), the results of the FUST and the NSFT suggest that d-methadone potentially has clinically useful in vivo NMDAR antagonistic effects which may be indicated for an array of neurological diseases and symptoms where regulation of the NMDARs, excitotoxicity, BDNF, testosterone and neuronal plasticity modulation are implicated.

### Example 11: d-Methadone Inhibits Both N E and Serotonin Re-Uptake

Inhibitory activity of d-methadone on norepinepherine and serotonin uptake was reported by Codd et. al. (1995), and confirmed and extended with two new in vitro studies (Study 1 and Study 2) presented by the inventors in this Example.

In summary, the present in vitro testing results revealed that (S)-methadone hydrochloride (d-methadone) showed significant inhibition (in the range of the test standards) of the uptake of serotonin by the serotonin transporter (SERT or 5-HT) and norepinephrine uptake by the norepinephrine transporter (NET). Both the SERT and the NET are the target of many antidepressant medications and these transporters are implicated in many psychiatric and neurological conditions.

Study 1

The purpose of this study was to test 7 compounds in binding assays and in enzyme and uptake assays. In particular, 7 compounds [oxymorphone hydrochloride monohydrate, (S)-methadone hydrochloride, (R)-methadone hydrochloride, tapentadol hydrochloride, and three deuterated d-methadone compounds referred to herein as d-methadone “D9,” “D10,” and “D16” ] were tested at 1.0E-05 M. The formula for each of D9, D10, and D16 are as follows:

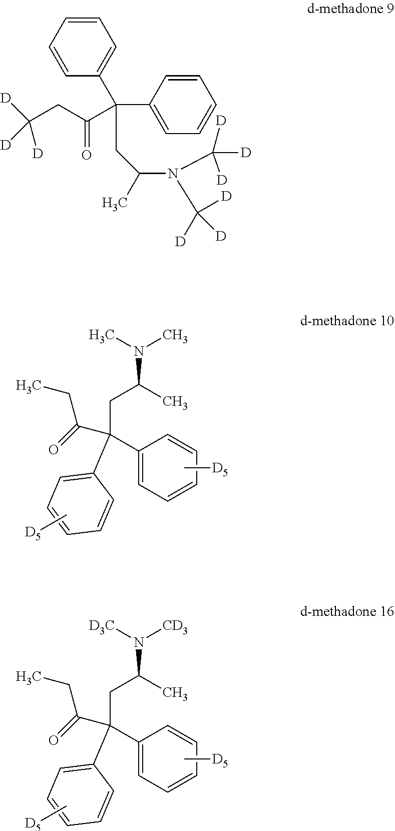

Compound binding was calculated as a % inhibition of the binding of a radioactively labeled ligand specific for each target. And, compound enzyme inhibition effect was calculated as a % inhibition of control enzyme activity.

Results showing an inhibition or stimulation higher than 50% were considered to represent significant effects of the test compounds. And, such effects were observed here and are listed in the following Tables 47-53.

TABLE 47
Oxymorphone Hydrochloride Monohydrate

| Assay | 1.0E−05 M |
|---|---|
| δ (DOP) (h) (agonist radioligand) | 96.8% |
| κ (KOP) (agonist radioligand) | 98.4% |
| μ (MOP) (h) (agonist radioligand) | 99.6% |

TABLE 48
(S)-Methadone hydrochloride

| Assay | 1.0E−05 M |
|---|---|
| δ (DOP) (h) (agonist radioligand) | 59.6% |
| κ (KOP) (agonist radioligand) | 86.5% |
| μ (MOP) (h) (agonist radioligand) | 99.8% |
| norepinephrine uptake | 61.6% |
| 5-HT uptake | 91% |

TABLE 49
(R)-Methadone hydrochloride

| Assay | 1.0E−05 M |
|---|---|
| δ (DOP) (h) (agonist radioligand) | 92.2% |
| κ (KOP) (agonist radioligand) | 91.4% |
| μ (MOP) (h) (agonist radioligand) | 99.3% |
| norepinephrine uptake | 90.3% |
| 5-HT uptake | 101.4% |

TABLE 50
Tapentadol Hydrochloride

| Assay | 1.0E−05 M |
|---|---|
| norepinephrine uptake | 94.1% |
| 5-HT uptake | 89.1% |

TABLE 51
Compound D-Methadone-D9

| Assay | 1.0E−05 M |
|---|---|
| κ (KOP) (agonist radioligand) | 84.2% |
| μ (MOP) (h) (agonist radioligand) | 97.5% |
| norepinephrine uptake | 61.4% |
| 5-HT uptake | 90.5% |

TABLE 52
Compound D-Methadone-D10

| Assay | 1.0E−05 M |
|---|---|
| κ (KOP) (agonist radioligand) | 86.7% |
| μ (MOP) (h) (agonist radioligand) | 97.6% |
| norepinephrine uptake | 70.2% |
| 5-HT uptake | 98% |

TABLE 53
Compound D-Methadone-D16

| Assay | 1.0E−05 M |
|---|---|
| κ (KOP) (agonist radioligand) | 84.5% |
| μ (MOP) (h) (agonist radioligand) | 96.8% |
| norepinephrine uptake | 71.8% |
| 5-HT uptake | 95.2% |

Compounds: The experiments of this study included both test compounds (shown in Table 54, below), and reference compounds. The test compounds were manufactured by Relmada Therapeutics (New York, N.Y.).

TABLE 54
Test Compounds

| | | Reference | Batch | | | | Received | Stock | |
|---|---|---|---|---|---|---|---|---|---|
| Client Compound ID | Compound ID | Number | Number | FW | MW | Purity | Form | solution | Flag |
| Oxymorphone | 100025153-1 | LGCFOR0673.00 | 16035 | 355.81 | — | 97.7 | Powder | 1.E−02 M | — |
| Hydrochloride | | | | | | | | DMSO | |
| Monohydrate | | | | | | | | | |
| (S)-Methadone | 100025153-2 | — | 6-JGC-41-1 | 345.91 | — | 98.0 | Powder | 1.E−02 M | — |
| hydrochloride | | | | | | | | MeOH | |
| (R)-Methadone | 100025153-3 | — | 6-JGC-51-1 | 345.91 | — | 98.0 | Powder | 1.E−02 M | — |
| hydrochloride | | | | | | | | MeOH | |
| Tapentadol | 100025153-4 | — | 1-NSR-55-3 | 257.8 | — | 98.0 | Powder | 1.E−02 M | — |
| Hydrochloride | | | | | | | | MeOH | |
| D-Methadone-D9 | 100025153-5 | CSPF-284 | FC05111502 | 318.5 | — | 99.5 | Powder | 1.E−02 M | — |
| | | | | | | | | DMSO | |
| D-Methadone-D10 | 100025153-6 | CSQ-21303RB | FC05141501 | 319.51 | — | 99.8 | Powder | 1.E−02 M | — |
| | | | | | | | | DMSO | |
| D-Methadone-D16 | 100025153-7 | CSQ-21465RB | FC05141502 | 325.54 | — | 99.9 | Powder | 1.E−02 M | — |
| | | | | | | | | DMSO | |

FW: Formula Weight
— MW: Molecular Weight

Reference Compounds:

In each experiment and if applicable, the respective reference compound was tested concurrently with the test compounds, and the data were compared with historical values determined at Eurofins Cerep (Celle I′Evescault, France). The experiment was accepted in accordance with Eurofins validation Standard Operating Procedure.

Materials and Methods

Experimental Conditions:

The experimental conditions and protocols are summarized in Tables 55 and 56, below. Table 55 is particular to conditions and protocols for the binding assays. And Table 56 is particular to conditions and protocols for the enzyme and uptake assays. Minor variations to the experimental protocol described in those Tables may have occurred during the testing, however, they have no impact on the quality of the results obtained.

TABLE 55
In Vitro Pharmacology: Binding Assays

| | | | | | | | | |
|---|---|---|---|---|---|---|---|---|
| | | | | | Non | | Detection | |
| Assay | Source | Ligand | Conc. | Kd | Specific | Incubation | Method | Bibl. |

Receptors

| δ (DOP) (h) | human | [³H]DADLE | 0.5 | nM | 0.73 | nM | naltrexone | 120 | min | Scintillation | 501 |
|---|---|---|---|---|---|---|---|---|---|---|---|
| (agonist | recombinant | | | | | | (10 μM) | RT | | counting | |
| radioligand) | (CHO cells) | | | | | | | | | | |
| κ (KOP) | rat | [³H]U | 1 | nM | 2 | nM | naloxone | 60 | min | Scintillation | 771 |
| (agonist | recombinant | 69593 | | | | | (10 μM) | RT | | counting | |
| radioligand) | (CHO cells) | | | | | | | | | | |
| μ (MOP) (h) | human | [³H]DAMGO | 0.5 | nM | 0.35 | nM | naloxone | 120 | min | Scintillation | 260 |

| | | | | |
|---|---|---|---|---|
| (agonist | recombinant | (10 μM) | RT | counting |
| radioligand) | (HEK-293 | | | |
| | cells) | | | |

Ion channels

| | | | | | | | | | | | |
|---|---|---|---|---|---|---|---|---|---|---|---|
| NMDA | rat cerebral | [³H]CGP | 5 | nM | 23 | nM | L-glutamate | 60 | min | Scintillation | 221 |
| (antagonist | cortex | 39653 | | | | | (100 μM) | 4° | C. | counting | |
| radioligand) | | | | | | | | | | | |

TABLE 56
In Vitro Pharmacology: Enzyme and Uptake Assays

| | | | | | | |
|---|---|---|---|---|---|---|
| | | Substrate/ | | | | |
| | | Stimulus/ | | Measured | Detection | |
| Assay | Source | Tracer | Incubation | Component | Method | Bibl. |

Transporters

| | | | | | | | |
|---|---|---|---|---|---|---|---|
| norepinephrine | rat hypothalamus | [³H]NE | 20 | min | [³H]NE | Scintillation | 184 |
| uptake | synaptosomes | (0.2 μCi/ml) | 37 | ° C. | incorporation into | counting | |
| | | | | | synaptosomes | | |
| 5-HT uptake | rat brain | [³H]5-HT | 15 | min | [³H]5-HT | Scintillation | 184 |
| | synaptosomes | (0.2 μCi/ml) | 37 | ° C. | incorporation into | counting | |
| | | | | | synaptosomes | | |

Results

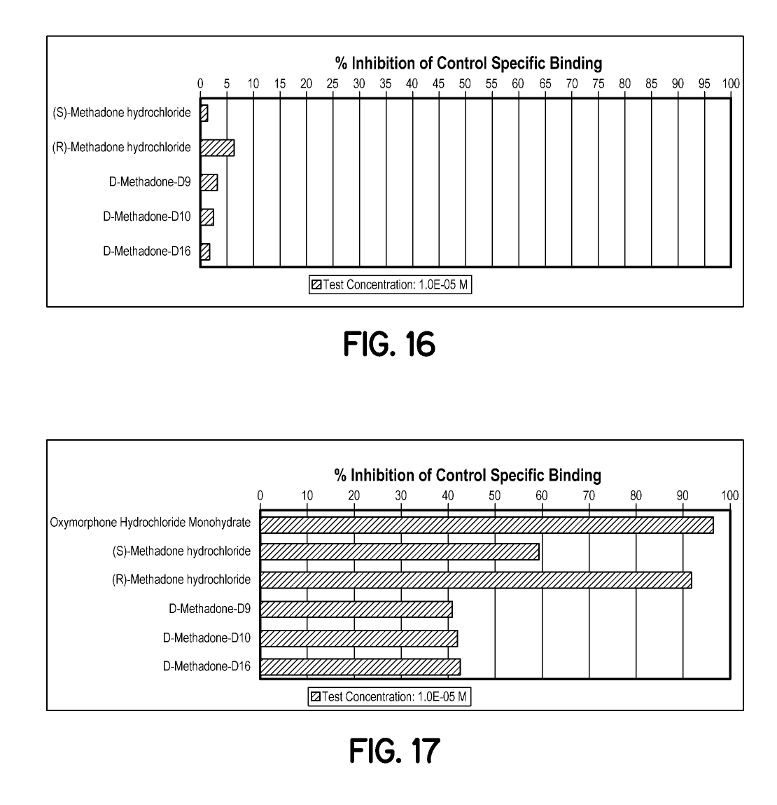

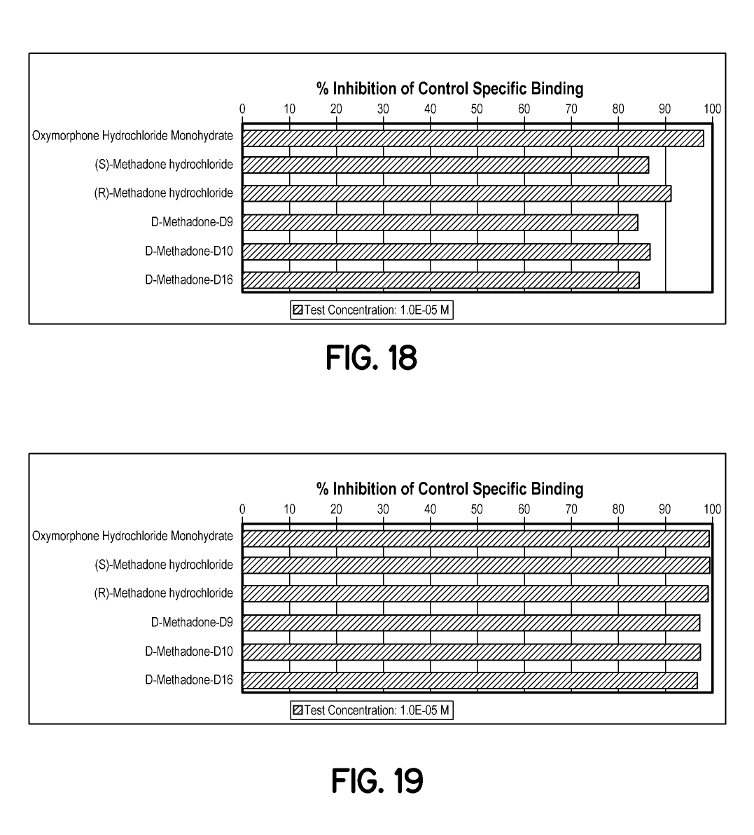

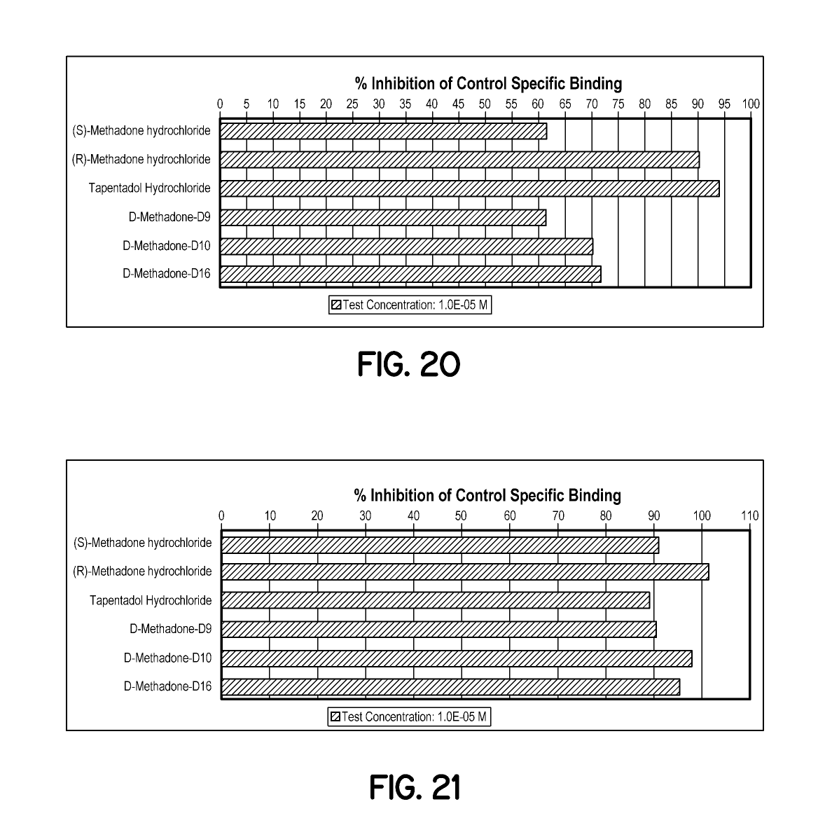

Results of the assays of this Study 1 of the Example are shown in Tables 57-60, below, and in FIGS. 16-21. Tables 57 and 58 show the results of in vitro pharmacology binding assays for test compounds and reference compounds, respectively. And FIGS. 16-19 show results for the binding assays for test compounds. Tables 59 and 60 show the results of in vitro pharmacology enzyme and uptake assays for test compounds and reference compounds, respectively. And FIGS. 20 and 21 show results for enzyme and uptake assays for test compounds.

TABLE 57
Test Compound Results for In Vitro Pharmacology: Binding Assays

| | | Test | % Inhibition of Control Specific Binding | | |
|---|---|---|---|---|---|
| Compound I.D. | Client Compound I.D. | Concentration | 1^{st} | 2^{nd} | Mean |

NMDA (antagonist radioligand)

| | | | | | |
|---|---|---|---|---|---|
| 100025153-2 | (S)-Methadone hydrochloride | 1.0E−05 M | −0.5 | 3.1 | 1.3 |
| 100025153-3 | (R)-Methadone hydrochloride | 1.0E−05 M | 2.4 | 10.3 | 6.4 |
| 100025153-5 | D-Methadone-D9 | 1.0E−05 M | 2.5 | 3.6 | 3.0 |
| 100025153-6 | D-Methadone-D10 | 1.0E−05 M | −1.3 | 6.2 | 2.4 |
| 100025153-7 | D-Methadone-D16 | 1.0E−05 M | −2.4 | 5.9 | 1.8 |

δ (DOP) (h) (agonist radioligand)

| | | | | | |
|---|---|---|---|---|---|
| 100025153-1 | Oxymorphone Hydrochloride | 1.0E−05 M | 96.5 | 97.1 | 96.8 |
| | Monohydrate | | | | |
| 100025153-2 | (S)-Methadone hydrochloride | 1.0E−05 M | 60.4 | 58.7 | 59.6 |
| 100025153-3 | (R)-Methadone hydrochloride | 1.0E−05 M | 93.1 | 91.3 | 92.2 |
| 100025153-5 | D-Methadone-D9 | 1.0E−05 M | 39.6 | 42.8 | 41.2 |
| 100025153-6 | D-Methadone-D10 | 1.0E−05 M | 42.3 | 42.1 | 42.2 |
| 100025153-7 | D-Methadone-D16 | 1.0E−05 M | 40.5 | 45.0 | 42.7 |

κ (KOP) (agonist radioligand)

| | | | | | |
|---|---|---|---|---|---|
| 100025153-1 | Oxymorphone Hydrochloride | 1.0E−05 M | 97.7 | 99.0 | 98.4 |
| | Monohydrate | | | | |
| 100025153-2 | (S)-Methadone hydrochloride | 1.0E−05 M | 89.5 | 83.4 | 86.5 |
| 100025153-3 | (R)-Methadone hydrochloride | 1.0E−05 M | 90.5 | 92.4 | 91.4 |
| 100025153-5 | D-Methadone-D9 | 1.0E−05 M | 83.1 | 85.4 | 84.2 |
| 100025153-6 | D-Methadone-D10 | 1.0E−05 M | 86.1 | 87.4 | 86.7 |
| 100025153-7 | D-Methadone-D16 | 1.0E−05 M | 82.8 | 86.2 | 84.5 |

μ (MOP) (h) (agonist radioligand)

| | | | | | |
|---|---|---|---|---|---|
| 100025153-1 | Oxymorphone Hydrochloride | 1.0E−05 M | 98.6 | 100.6 | 99.6 |
| | Monohydrate | | | | |
| 100025153-2 | (S)-Methadone hydrochloride | 1.0E−05 M | 99.8 | 99.8 | 99.8 |
| 100025153-3 | (R)-Methadone hydrochloride | 1.0E−05 M | 100.3 | 98.4 | 99.3 |
| 100025153-5 | D-Methadone-D9 | 1.0E−05 M | 97.2 | 97.8 | 97.5 |
| 100025153-6 | D-Methadone-D10 | 1.0E−05 M | 98.9 | 96.3 | 97.6 |
| 100025153-7 | D-Methadone-D16 | 1.0E−05 M | 96.5 | 97.2 | 96.8 |

TABLE 58
Reference Compound Results for In
Vitro Pharmacology: Binding Assays

| | | | |
|---|---|---|---|
| Compound I.D. | IC₅₀ (M) | Kᵢ (M) | nH |

NMDA (antagonist radioligand)

| | | | |
|---|---|---|---|
| CGS 19755 | 1.3E−06 M | 1.1E−06 M | 1.2 |

δ (DOP) (h) (agonist radioligand)

| | | | |
|---|---|---|---|
| DPDPE | 3.6E−09 M | 2.2E−09 M | 1.0 |

κ (KOP) (agonist radioligand)

| | | | |
|---|---|---|---|
| U 50488 | 1.6E−09 M | 1.1E−09 M | 1.5 |

μ (MOP) (h) (agonist radioligand)

| | | | |
|---|---|---|---|
| DAMGO | 4.1E−10 M | 1.7E−10 M | 0.9 |
| DAMGO | 4.2E−10 M | 1.7E−10 M | 0.8 |

TABLE 59
Test Compound Results for In Vitro Pharmacology, Enzyme and Update Assays

| | | Test | % Inhibition of Control Values | | |
|---|---|---|---|---|---|
| Compound I.D. | Client Compound I.D. | Concentration | 1^{st} | 2^{nd} | Mean |

norepinephrine uptake

| | | | | | |
|---|---|---|---|---|---|
| 100025153-2 | (S)-Methadone hydrochloride | 1.0E−05 M | 70.3 | 53.0 | 61.6 |
| 100025153-3 | (R)-Methadone hydrochloride | 1.0E−05 M | 96.5 | 84.1 | 90.3 |
| 100025153-4 | Tapentadol Hydrochloride | 1.0E−05 M | 103.4 | 84.8 | 94.1 |
| 100025153-5 | D-Methadone-D9 | 1.0E−05 M | 69.7 | 53.0 | 61.4 |
| 100025153-6 | D-Methadone-D10 | 1.0E−05 M | 73.4 | 66.9 | 70.2 |
| 100025153-7 | D-Methadone-D16 | 1.0E−05 M | 82.9 | 60.7 | 71.8 |

5-HT uptake

| | | | | | |
|---|---|---|---|---|---|
| 100025153-2 | (S)-Methadone hydrochloride | 1.0E−05 M | 92.7 | 89.3 | 91.0 |
| 100025153-3 | (R)-Methadone hydrochloride | 1.0E−05 M | 103.1 | 99.6 | 101.4 |
| 100025153-4 | TapentadolHydrochloride | 1.0E−05 M | 91.1 | 87.2 | 89.1 |

TABLE 60
Reference Compound Results for In Vitro
Pharmacology, Enzyme and Update Assays

| | | |
|---|---|---|
| Compound I.D. | IC₅₀ (M) | nH |

norepinephrine uptake

| | | |
|---|---|---|
| Protriptyline | 3.6E−09 M | n/a |

5-HT uptake

| | | |
|---|---|---|
| Imipramine | 4.0E−08 M | n/a |
| Imipramine | 4.0E−08 M | n/a |

Results showing an inhibition (or stimulation for assays run in basal conditions) higher than 50% are considered to represent significant effects of the test compounds. 50% is the most common cut-off value for further investigation (determination of IC₅₀ or EC₅₀ values from concentration-response curves) that we would recommend.

Results showing an inhibition (or stimulation) between 25% and 50% are indicative of weak to moderate effects (in most assays, they should be confirmed by further testing as they are within a range where more inter-experimental variability can occur).

Results showing an inhibition (or stimulation) lower than 25% are not considered significant and mostly attributable to variability of the signal around the control level.

Low to moderate negative values have no real meaning and are attributable to variability of the signal around the control level. High negative values (>50%) that are sometimes obtained with high concentrations of test compounds are generally attributable to non-specific effects of the test compounds in the assays. On rare occasion they could suggest an allosteric effect of the test compound.

Analysis and Expression of Results (In Vitro Pharmacology: Binding Assays):

The results are expressed as a percent of control specific binding

$$\frac{measured specific binding}{control specific binding} * 100$$

and as a percent inhibition of control specific binding

$$100 - ( \frac{measured specific binding}{control specific binding} * 100 )$$

obtained in the presence of the test compounds.

The IC₅₀ values (concentration causing a half-maximal inhibition of control specific binding) and Hill coefficients (nH) were determined by non-linear regression analysis of the competition curves generated with mean replicate values using Hill equation curve fitting

$$Y = D + [ \frac{A - D}{1 + {( C \text{/} {C}_{50} )}^{nH}} ]$$

where Y=specific binding, A=left asymptote of the curve, D=right asymptote of the curve, C=compound concentration, C₅₀=IC₅₀, and nH=slope factor. This analysis was performed using software developed at Cerep (Hill software) and validated by comparison with data generated by the commercial software SigmaPlot® 4.0 for Windows® (© 1997 by SPSS Inc.).

The inhibition constants (Kᵢ) were calculated using the Cheng Prusoff equation

$${K}_{i} = \frac{{IC}_{50}}{( 1 + L \text{/} {K}_{D} )}$$

where L=concentration of radioligand in the assay, and K_{D}=affinity of the radioligand for the receptor. A scatchard plot is used to determine the K_{D}.

Analysis and Expression of Results (In Vitro Pharmacology: Enzyme and Uptake Assays):

The results are expressed as a percent of control specific activity

$$\frac{measured specific binding}{control specific binding} * 100$$

and as a percent inhibition of control specific activity

$$100 - ( \frac{measured specific binding}{control specific binding} * 100 )$$

obtained in the presence of the test compounds.

The IC₅₀ values (concentration causing a half-maximal inhibition of control specific activity), EC₅₀ values (concentration producing a half-maximal increase in control basal activity), and Hill coefficients (nH) were determined by non-linear regression analysis of the inhibition/concentration-response curves generated with mean replicate values using Hill equation curve fitting

$$Y = D + [ \frac{A - D}{1 + {( C \text{/} {C}_{50} )}^{nH}} ]$$

where Y=specific activity, A=left asymptote of the curve, D=right asymptote of the curve, C=compound concentration, C₅₀=IC₅₀ or EC₅₀, and nH=slope factor.

This analysis was performed using software developed at Cerep (Hill software) and validated by comparison with data generated by the commercial software SigmaPlot® 4.0 for Windows® (© 1997 by SPSS Inc.).

Study 2

The purpose of this study was to test 7 compounds in binding assays and in enzyme and uptake assays. In particular, 7 compounds [oxymorphone hydrochloride monohydrate, (S)-methadone hydrochloride, (R)-methadone hydrochloride, tapentadol hydrochloride, D9, D10, and D6] were tested at several concentrations for IC₅₀ or EC₅₀ determination. Compound binding was calculated as a % inhibition of the binding of a radioactively labeled ligand specific for each target. And, compound enzyme inhibition effect was calculated as a % inhibition of control enzyme activity.

Results showing an inhibition or stimulation higher than 50% are considered to represent significant effects of the test compounds. And, such effects were observed here and are listed in the following Tables 61-67. Only the calculable IC₅₀ and EC₅₀ are reported below.

TABLE 61
Oxymorphone Hydrochloride Monohydrate

| | | | | | |
|---|---|---|---|---|---|
| Assay | IC₅₀ | Kᵢ | K_{B} | EC₅₀ | nH |

| | | | |
|---|---|---|---|
| δ (DOP) (h) (agonist | 2.5E−07 M | 1.5E−07 M | 0.8 |
| radioligand) | | | |
| κ (KOP) (agonist | 5.5E−08 M | 3.7E−08 M | 0.8 |
| radioligand) | | | |
| μ (MOP) (h) (agonist | 5.1E−10 M | 2.1E−10 M | 0.9 |
| radioligand) | | | |

TABLE 62
Tapentadol Hydrochloride

| | | | | | |
|---|---|---|---|---|---|
| Assay | IC₅₀ | Kᵢ | K_{B} | EC₅₀ | nH |

| | |
|---|---|
| 5-HT uptake | 8.5E−07 M |
| norepinephrine uptake | 2.6E−07 M |

TABLE 63
(S)-Methadone Hydrochloride

| Assay | IC₅₀ | Kᵢ | K_{B} | EC₅₀ | nH |
|---|---|---|---|---|---|
| 5-HT uptake | 1.2E−07 M | | | | |
| δ (DOP) (h) (agonist | 8.1E−06 M | 4.8E−06 M | | | 1 |
| radioligand) | | | | | |
| κ (KOP) (agonist | 1.3E−06 M | 8.5E−07 M | | | 1.2 |
| radioligand) | | | | | |
| μ (MOP) (h) (agonist | 2.5E−08 M | 1.0E−08 M | | | 0.8 |
| radioligand) | | | | | |
| norepinephrine uptake | 8.2E−06 M | | | | |

TABLE 64
(R)-Methadone Hydrochloride

| Assay | IC₅₀ | Kᵢ | K_{B} | EC₅₀ | nH |
|---|---|---|---|---|---|
| 5-HT uptake | 3.3E−08 M | | | | |
| δ (DOP) (h) (agonist | 1.2E−06 M | 7.2E−07 M | | | 0.6 |
| radioligand) | | | | | |
| κ (KOP) (agonist | 3.9E−07 M | 2.6E−07 M | | | 0.6 |
| radioligand) | | | | | |
| μ (MOP) (h) (agonist | 4.2E−09 M | 1.7E−09 M | | | 1.1 |
| radioligand) | | | | | |
| norepinephrine uptake | 1.2E−06 M | | | | |

TABLE 65
Compound D-Methadone-D9

| Assay | IC₅₀ | Kᵢ | K_{B} | EC₅₀ | nH |
|---|---|---|---|---|---|
| 5-HT uptake | 1.1E−06 M | | | | |
| δ (DOP) (h) (agonist | 1.6E−05 M | 9.5E−06 M | | | 0.8 |
| radioligand) | | | | | |
| κ (KOP) (agonist | 8.6E−07 M | 5.7E−07 M | | | 0.6 |
| radioligand) | | | | | |
| μ (MOP) (h) (agonist | 2.3E−08 M | 9.5E−09 M | | | 0.8 |
| radioligand) | | | | | |
| norepinephrine uptake | 7.2E−06 M | | | | |
| PCP (antagonist | 4.2E−06 M | 2.4E−06 M | | | 0.8 |
| radioligand) | | | | | |

TABLE 66
Compound D-Methadone-D10

| Assay | IC₅₀ | Kᵢ | K_{B} | EC₅₀ | nH |
|---|---|---|---|---|---|
| 5-HT uptake | 4.0E−07 M | | | | |
| δ (DOP) (h) (agonist | 1.3E−05 M | 8.0E−06 M | | | 0.9 |
| radioligand) | | | | | |
| κ (KOP) (agonist | 1.4E−06 M | 9.4E−07 M | | | 0.8 |
| radioligand) | | | | | |
| μ (MOP) (h) (agonist | 3.6E−08 M | 1.5E−08 M | | | 0.8 |
| radioligand) | | | | | |
| norepinephrine uptake | 6.5E−06 M | | | | |
| PCP (antagonist | 2.8E−06 M | 1.6E−06 M | | | 0.8 |
| radioligand) | | | | | |

TABLE 67
Compound D-Methadone-D16

| Assay | IC₅₀ | Kᵢ | K_{B} | EC₅₀ | nH |
|---|---|---|---|---|---|
| 5-HT uptake | 4.7E−07 M | | | | |
| δ (DOP) (h) (agonist | 1.6E−05 M | 9.2E−06 M | | | 0.8 |
| radioligand) | | | | | |
| κ (KOP) (agonist | 1.5E−06 M | 1.0E−06 M | | | 0.9 |
| radioligand) | | | | | |
| μ (MOP) (h) (agonist | 4.2E−08 M | 1.7E−08 M | | | 1.3 |
| radioligand) | | | | | |
| norepinephrine uptake | 6.7E−06 M | | | | |
| PCP (antagonist | 5.3E−06 M | 3.0E−06 M | | | 1.2 |
| radioligand) | | | | | |

Compounds: The experiments of this study included both test compounds (shown in Table 68, below), and reference compounds. The test compounds were manufactured by Relmada Therapeutics (New York, N.Y.).

TABLE 68
Test Compounds

| | | Reference | Batch | | | | Received | Stock | |
|---|---|---|---|---|---|---|---|---|---|
| Client Compound ID | Compound ID | Number | Number | FW | MW | Purity | Form | solution | Flag |
| Oxymorphone | 100025989-1 | LGCFOR0673.00 | 16035 | 355.81 | — | 97.7 | Powder | 1.E−02 M | — |
| Hydrochloride | | | | | | | | DMSO | |
| Monohydrate | | | | | | | | | |
| Tapentadol | 100025989-2 | — | 1-NSR-55-3 | 257.8 | — | 98.0 | Powder | 1.E−02 M | — |
| Hydrochloride | | | | | | | | MeOH | |
| (S)-Methadone | 100025989-3 | — | 6-JGC-41-1 | 345.91 | — | 98.0 | Powder | 1.E−02 M | — |
| hydrochloride | | | | | | | | MeOH | |
| (R)-Methadone | 100025989-4 | — | 6-JGC-51-1 | 345.91 | — | 98.0 | Powder | 1.E−02 M | — |
| hydrochloride | | | | | | | | MeOH | |
| D-Methadone-D9 | 100025989-5 | CSPF-284 | FC05111502 | 318.5 | — | 99.5 | Powder | 1.E−02 M | — |
| | | | | | | | | DMSO | |
| D-Methadone-D10 | 100025989-6 | CSQ-21303RB | FC05141501 | 319.51 | — | 99.8 | Powder | 1.E−02 M | — |
| | | | | | | | | DMSO | |
| D-Methadone-D16 | 100025989-7 | CSQ-21465RB | FC05141502 | 325.54 | — | 99.9 | Powder | 1.E−02 M | — |
| | | | | | | | | DMSO | |

FW: Formula Weight
— MW: Molecular Weight

Reference Compounds: In each experiment and if applicable, the respective reference compound was tested concurrently with the test compounds, and the data were compared with historical values determined at Eurofins Cerep (Celle I′Evescault, France). The experiment was accepted in accordance with Eurofins validation Standard Operating Procedure.

Materials and Methods

Experimental Conditions:

The experimental conditions and protocols are summarized in Tables 69 and 70, below. Table 69 is particular to conditions and protocols for the binding assays. And Table 70 is particular to conditions and protocols for the enzyme and uptake assays. Minor variations to the experimental protocol described below may have occurred during the testing, they have no impact on the quality of the results obtained.

TABLE 69
In Vitro Pharmacology: Binding Assays

| | | | | | | | | |
|---|---|---|---|---|---|---|---|---|
| | | | | | Non | | Detection | |
| Assay | Source | Ligand | Conc. | Kd | Specific | Incubation | Method | Bibl. |

Receptors

| δ (DOP) (h) | human | [³H]DADLE | 0.5 | nM | 0.73 | nM | naltrexone | 120 | min | Scintillation | 501 |
|---|---|---|---|---|---|---|---|---|---|---|---|
| (agonist | recombinant | | | | | | (10 μM) | RT | | counting | |
| radioligand) | (CHO cells) | | | | | | | | | | |
| κ (KOP) | rat | [³H]U | 1 | nM | 2 | nM | naloxone | 60 | min | Scintillation | 771 |
| (agonist | recombinant | 69593 | | | | | (10 μM) | RT | | counting | |
| radioligand) | (CHO cells) | | | | | | | | | | |
| μ (MOP) (h) | human | [³H]DAMGO | 0.5 | nM | 0.35 | nM | naloxone | 120 | min | Scintillation | 260 |

| | | | | |
|---|---|---|---|---|
| (agonist | recombinant | (10 μM) | RT | counting |
| radioligand) | (HEK-293 | | | |
| | cells) | | | |

Ion channels

| | | | | | | | | | | | |
|---|---|---|---|---|---|---|---|---|---|---|---|
| PCP | rat cerebral | [³H]TCP | 10 | nM | 13 | nM | MK 801 | 120 | min | Scintillation | 257 |

| | | | | | |
|---|---|---|---|---|---|
| (antagonist | cortex | (10 μM) | 37° | C. | counting |
| radioligand) | | | | | |

TABLE 70
In Vitro Pharmacology: Enzyme and Uptake Assays

| | | | | | | |
|---|---|---|---|---|---|---|
| | | Substrate/ | | | | |
| | | Stimulus/ | | Measured | Detection | |
| Assay | Source | Tracer | Incubation | Component | Method | Bibl. |

Transporters

| | | | | | | | |
|---|---|---|---|---|---|---|---|
| norepinephrine | rat hypothalamus | [³H]NE | 20 | min | [³H]NE | Scintillation | 184 |
| uptake | synaptosomes | (0.2 μCi/ml) | 37° | C. | incorporation into | counting | |
| | | | | | synaptosomes | | |
| 5-HT uptake | rat brain | [³H]5-HT | 15 | min | [³H]5-HT | Scintillation | 184 |
| | synaptosomes | (0.2 μCi/ml) | 37° | C. | incorporation into | counting | |
| | | | | | synaptosomes | | |

Results

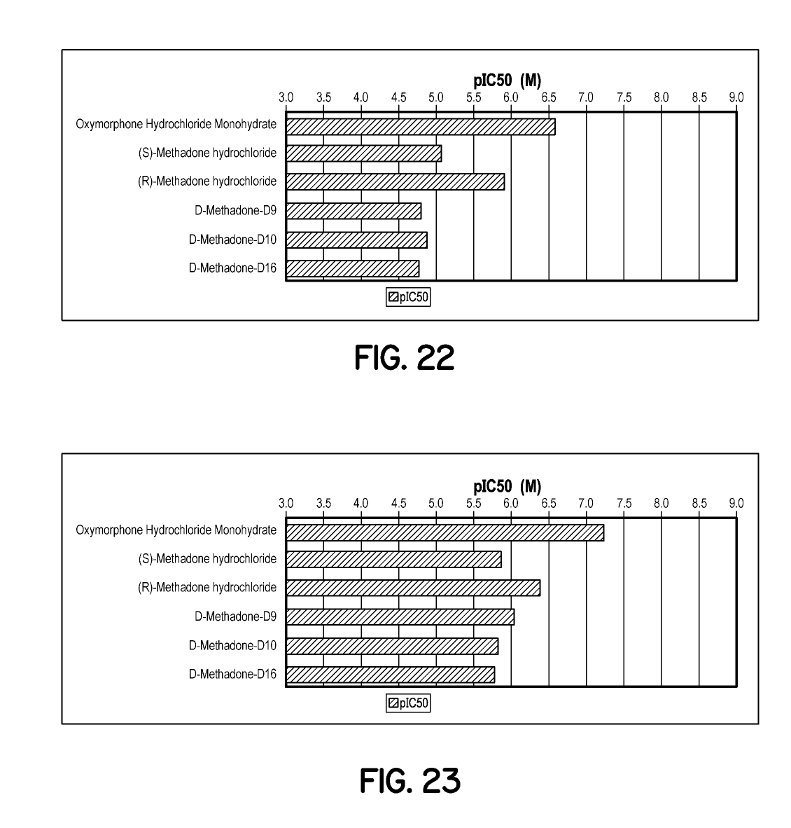

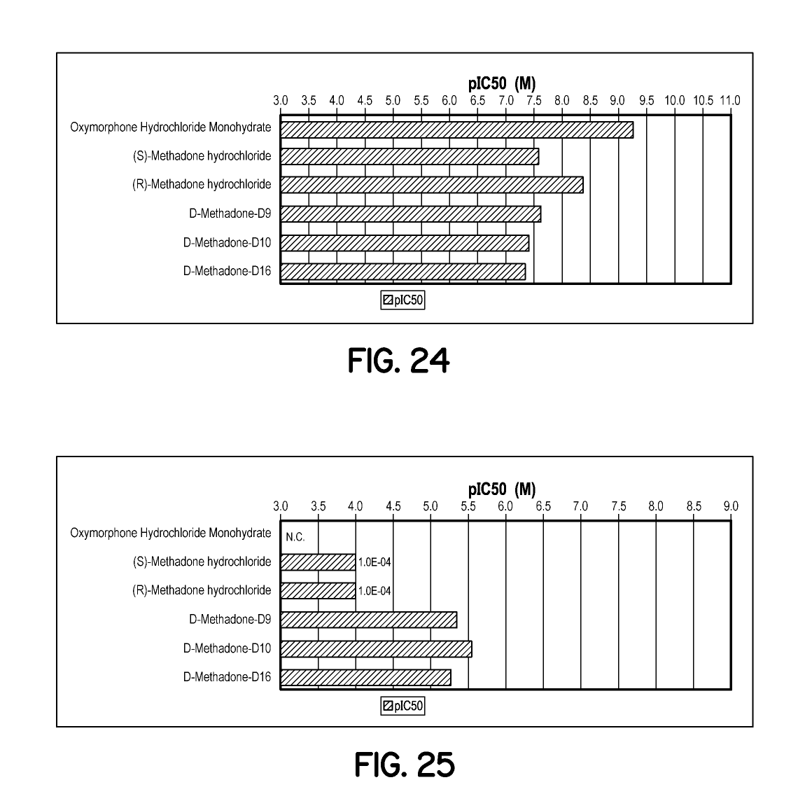

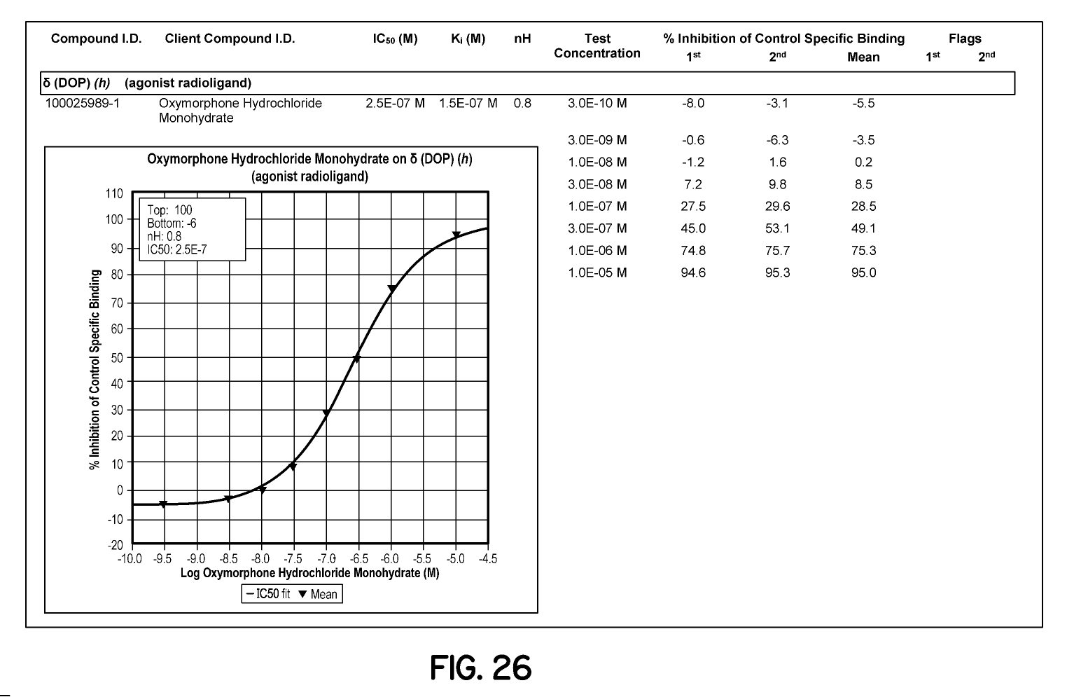

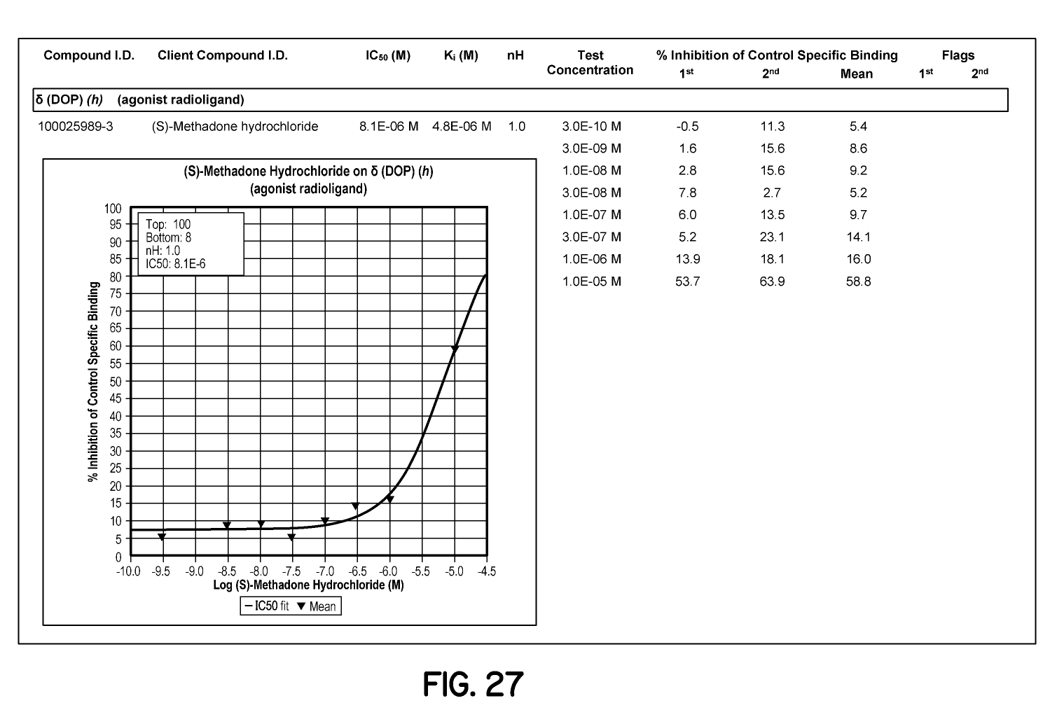

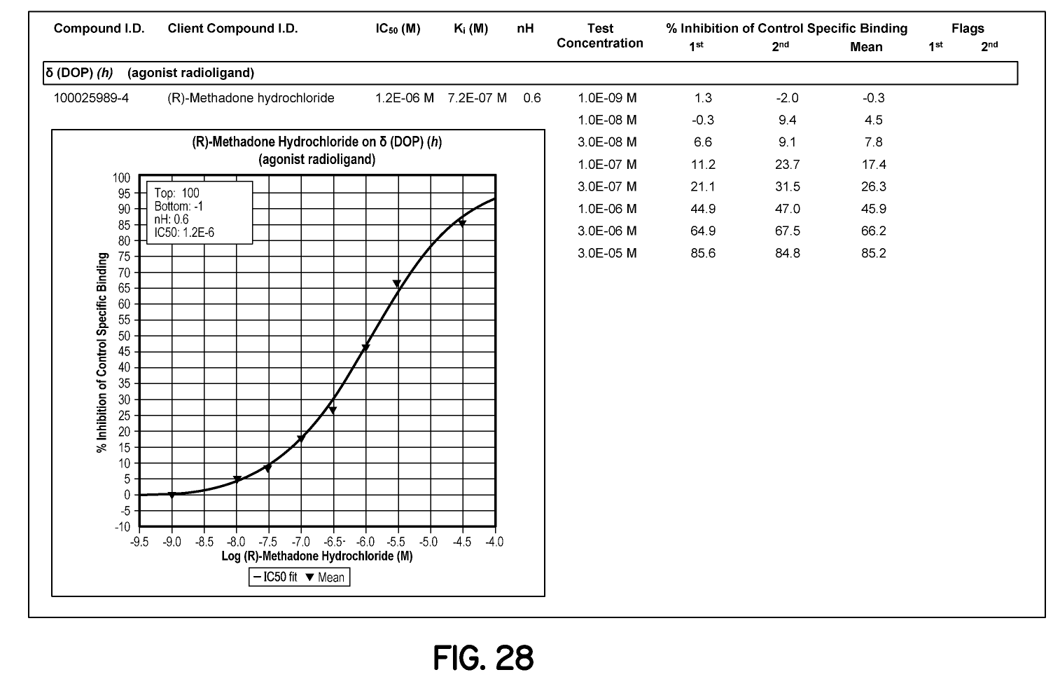

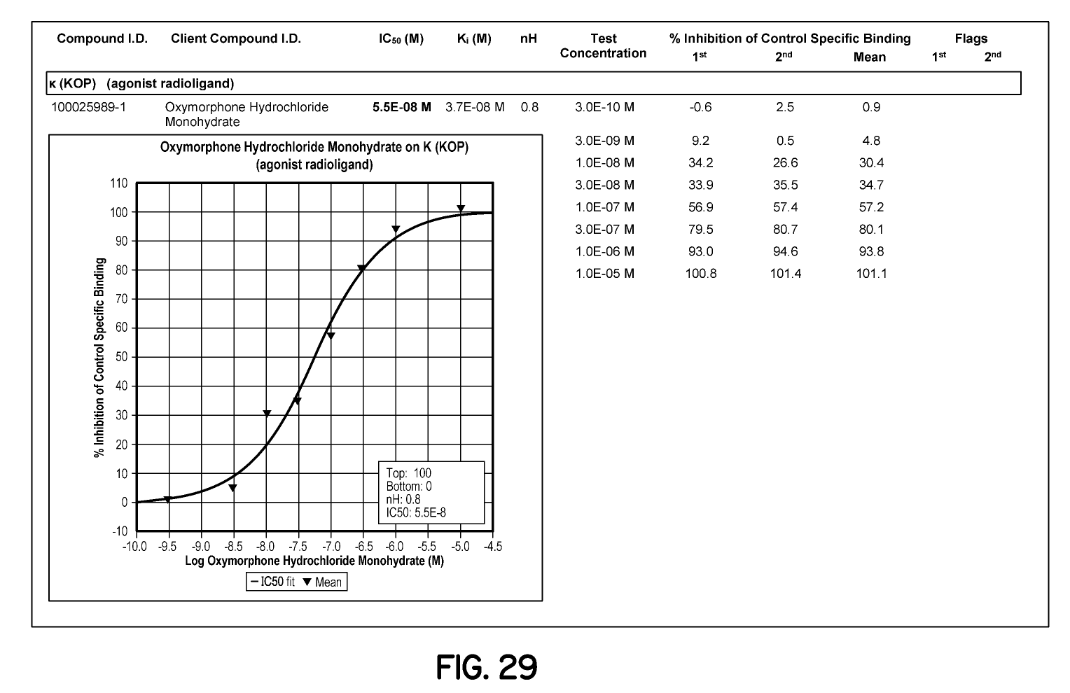

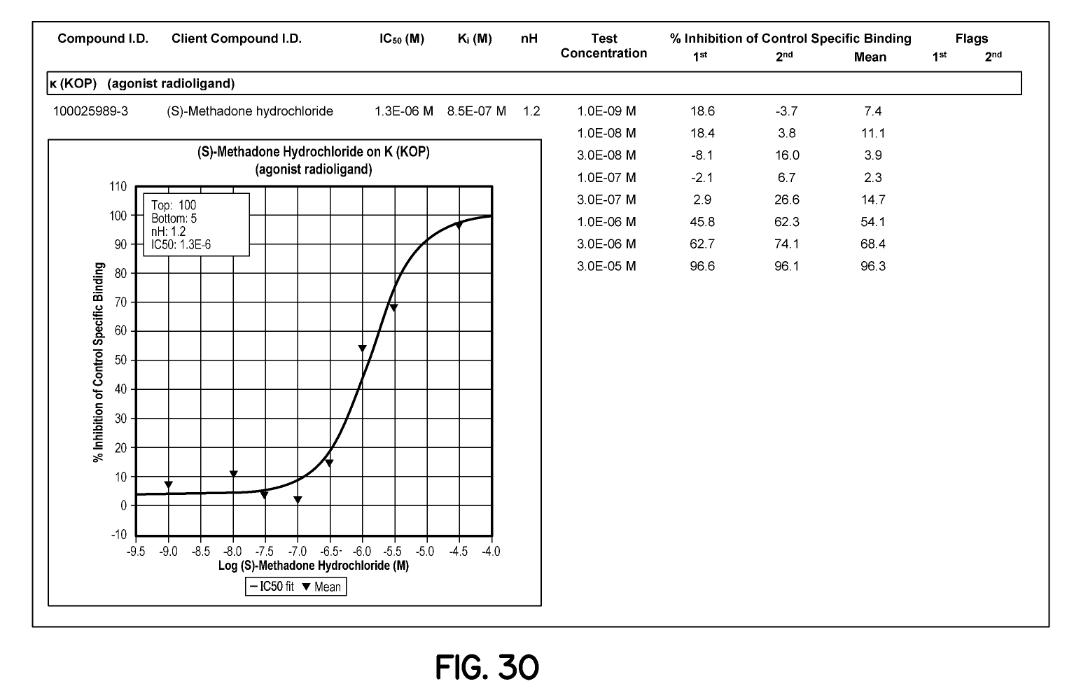

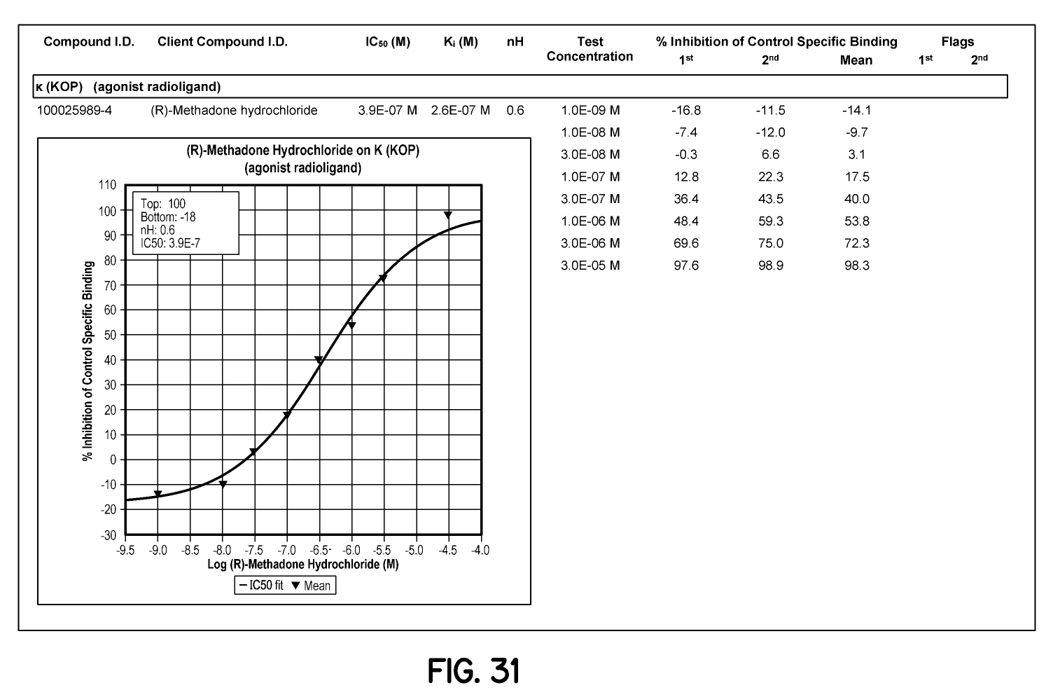

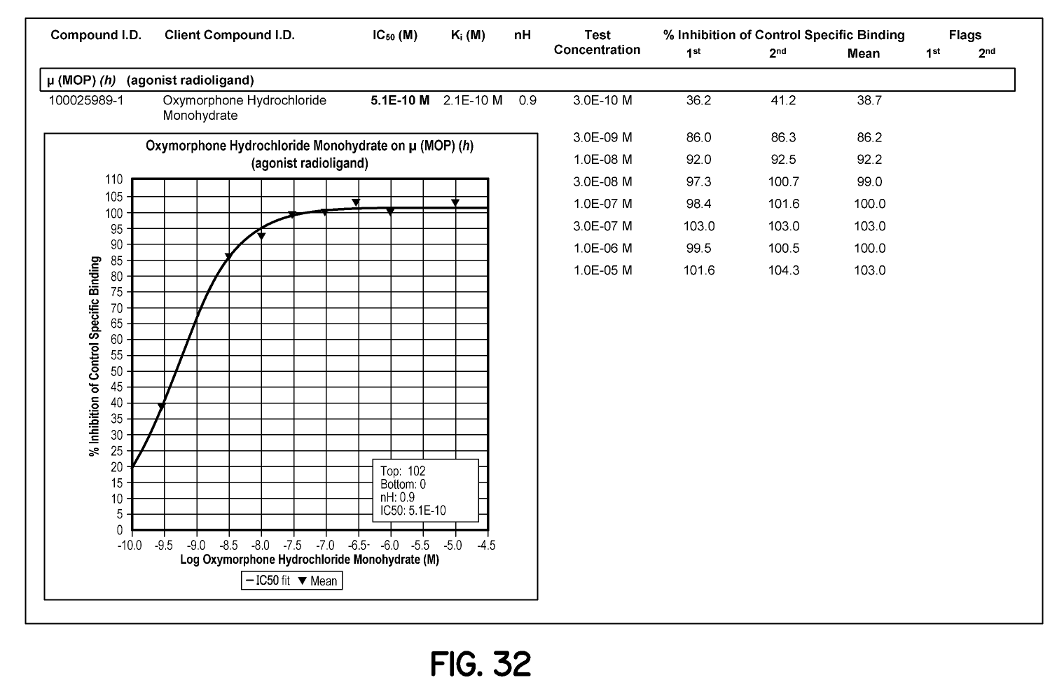

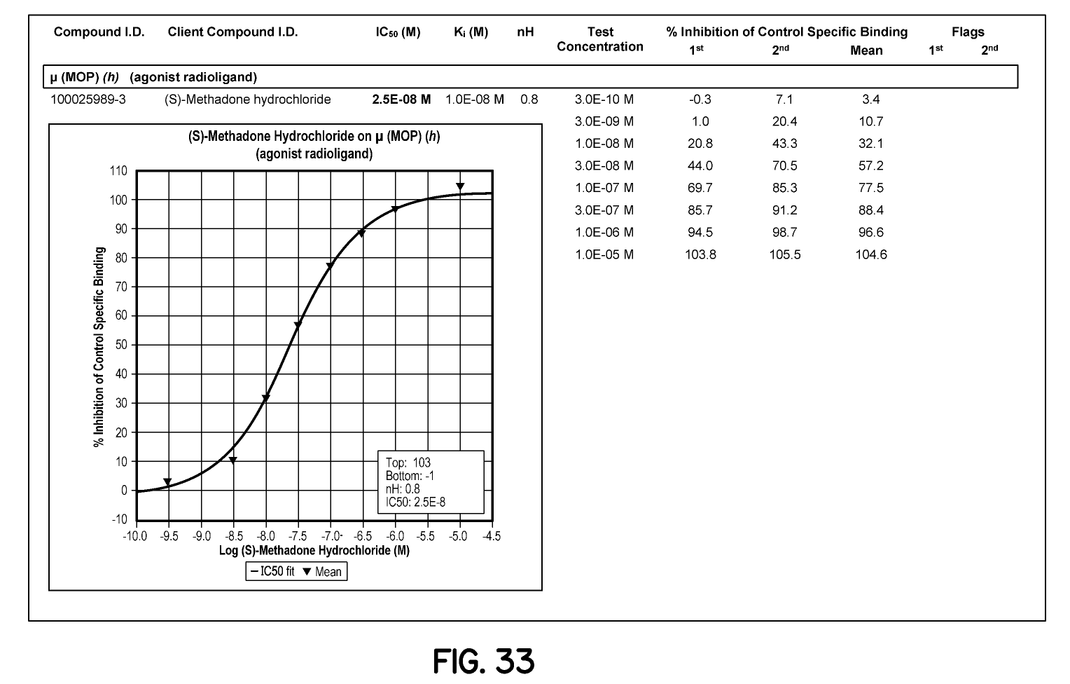

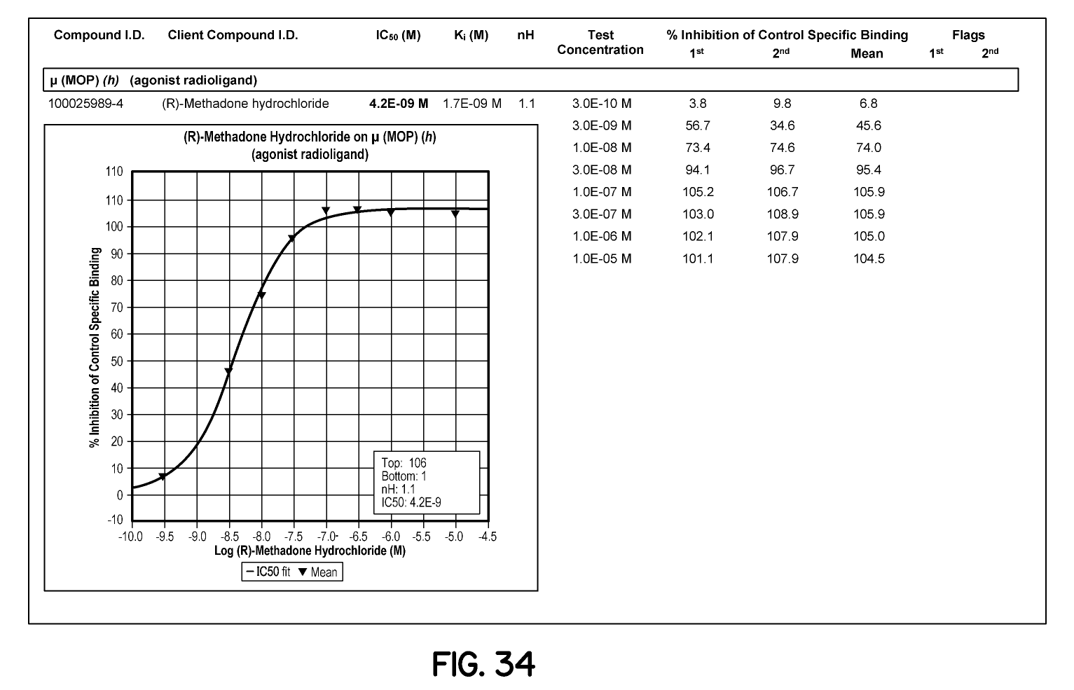

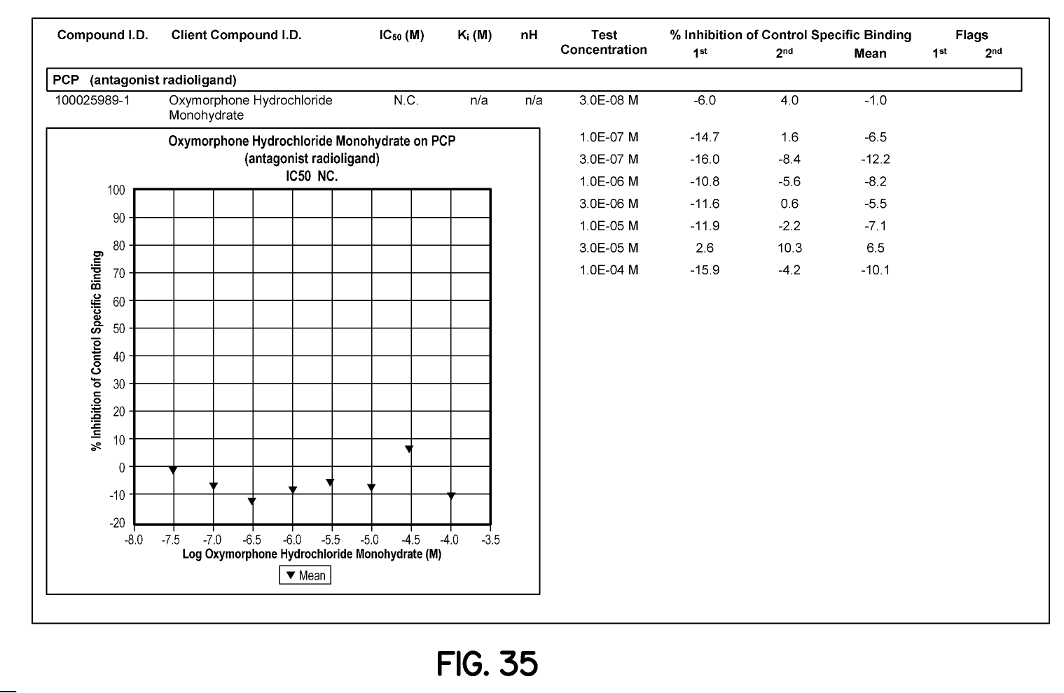

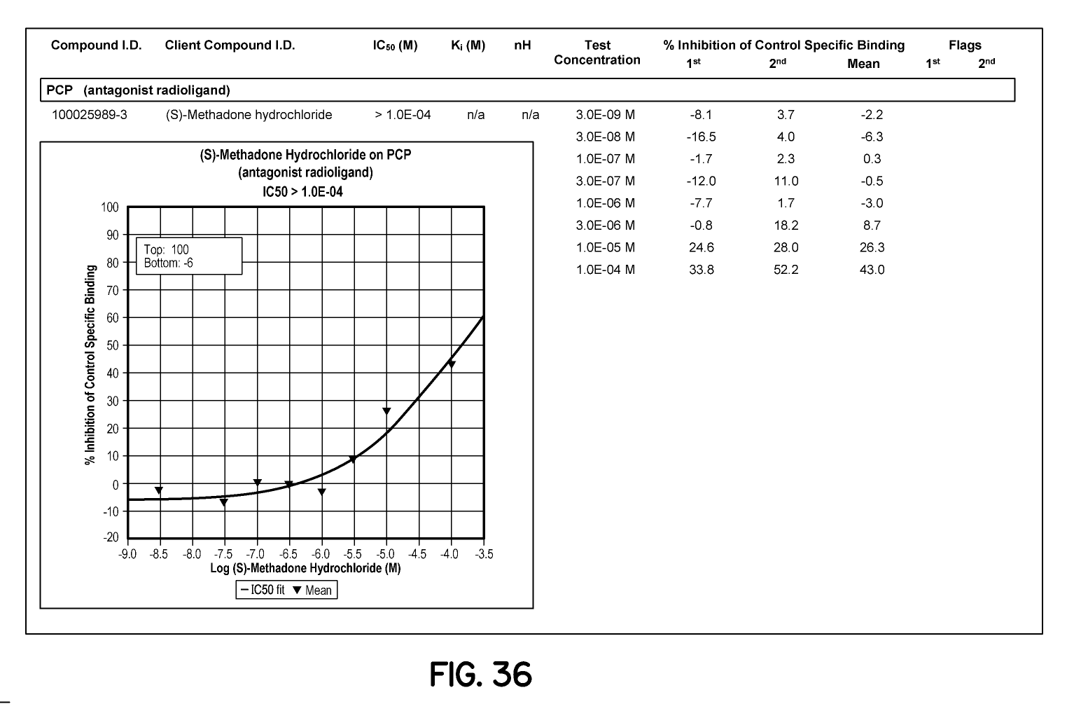

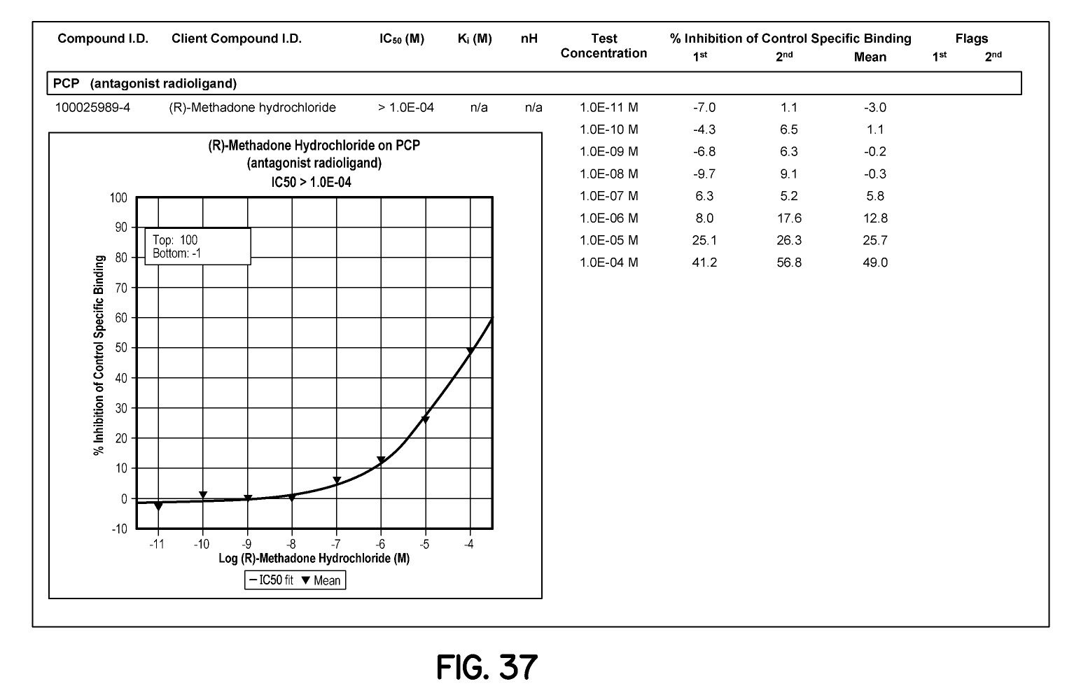

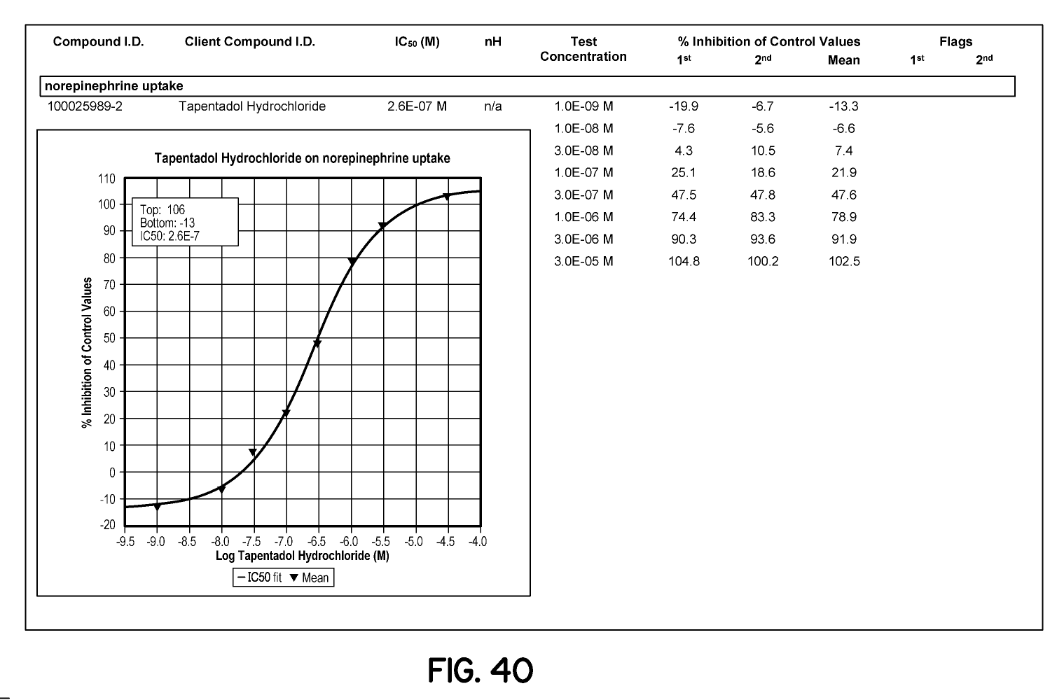

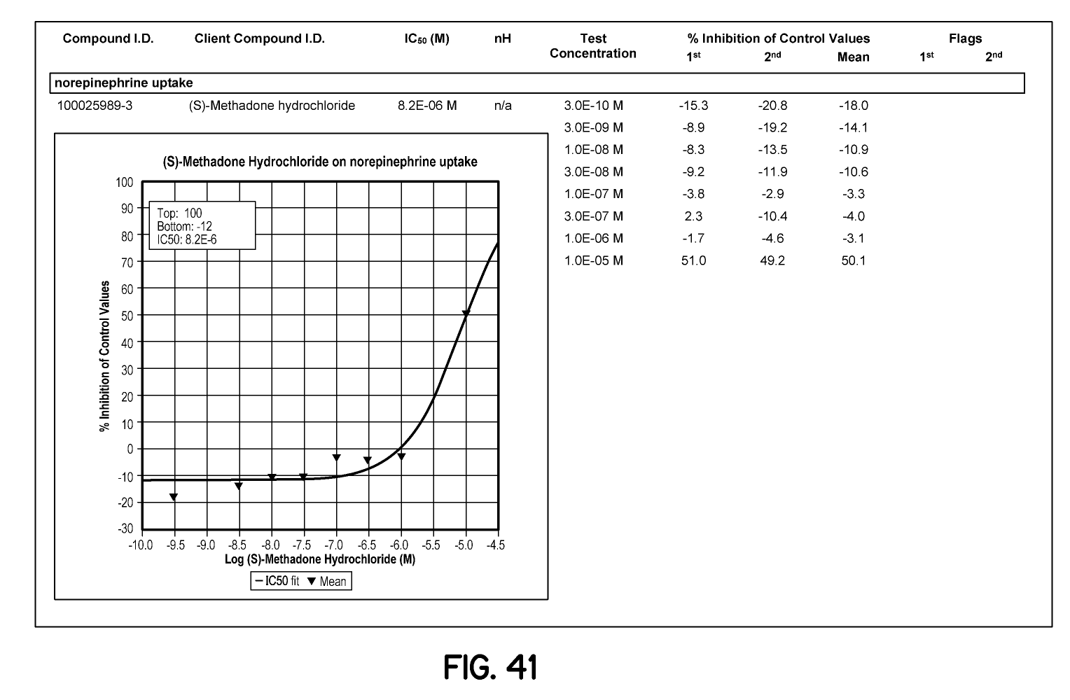

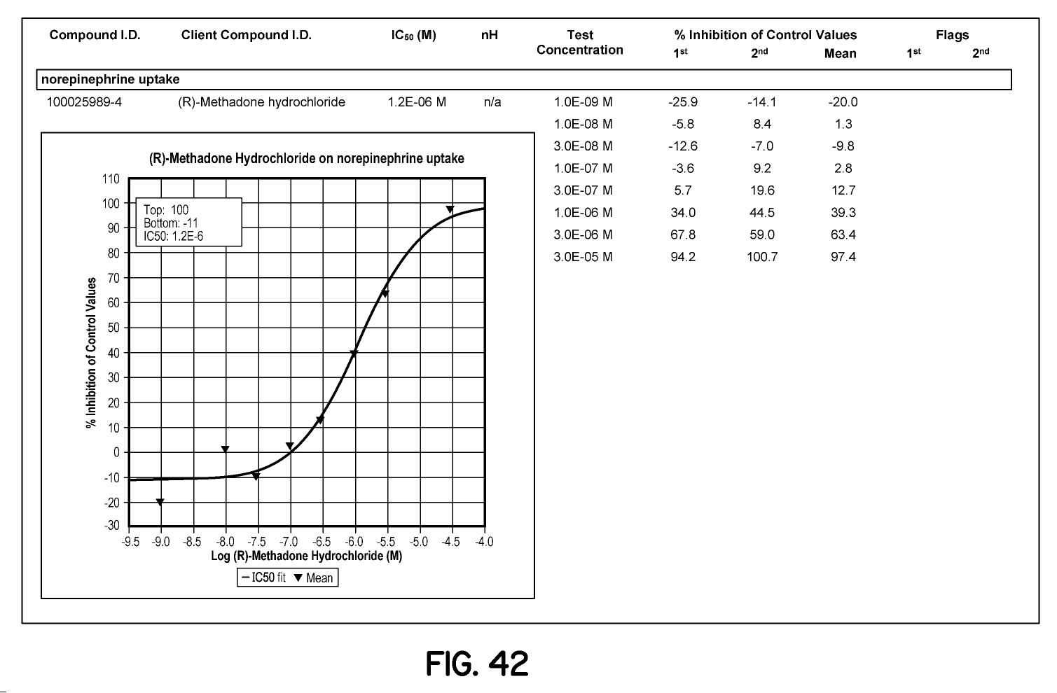

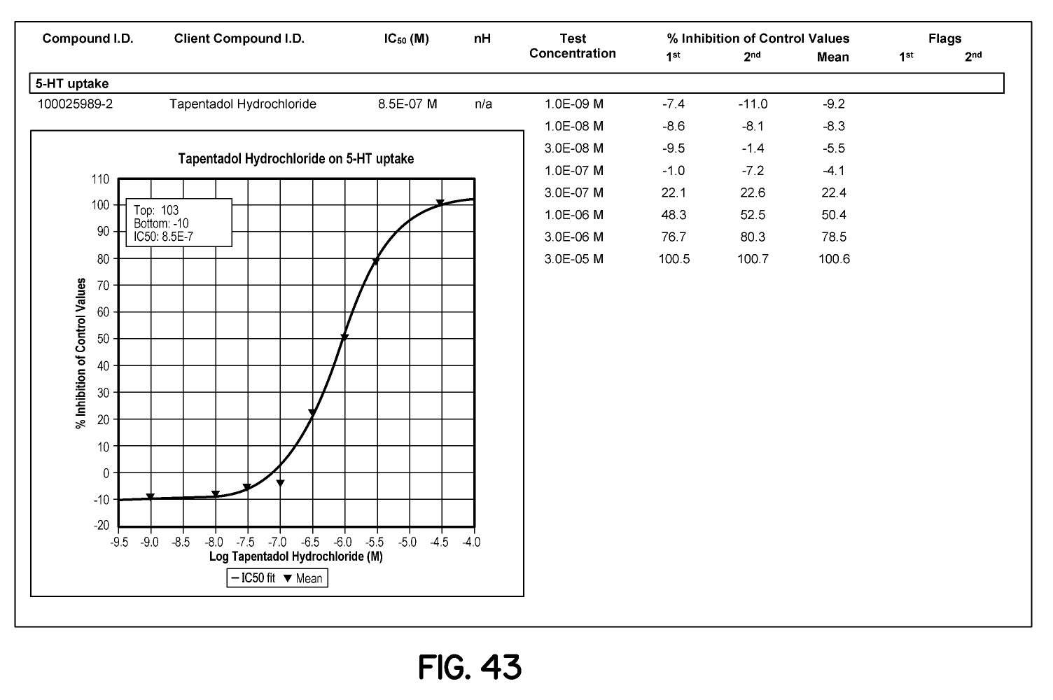

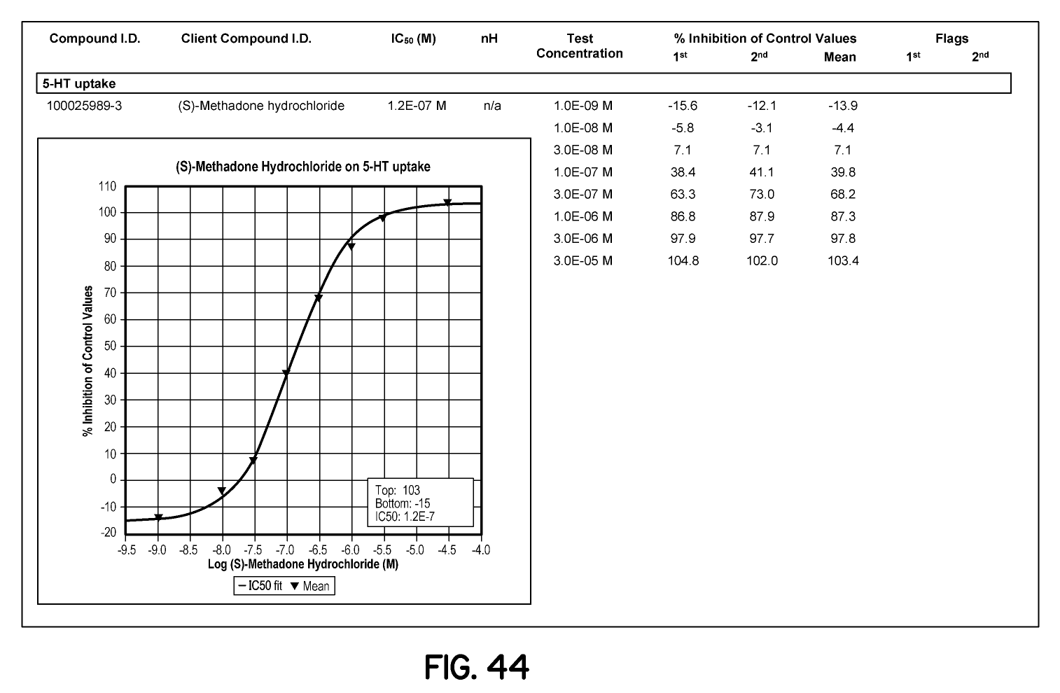

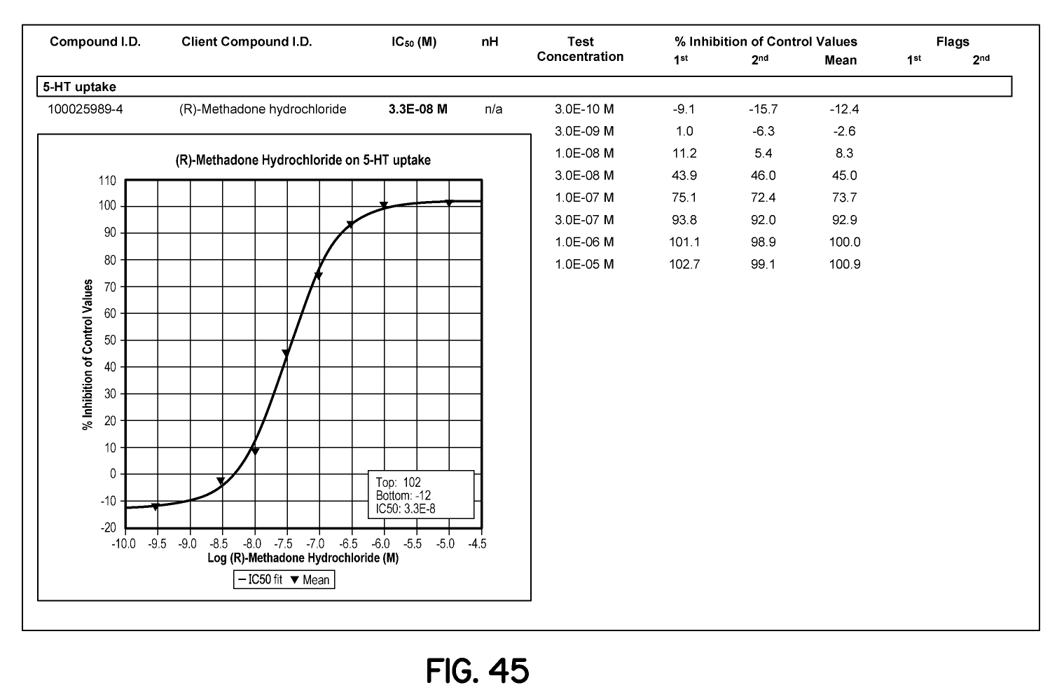

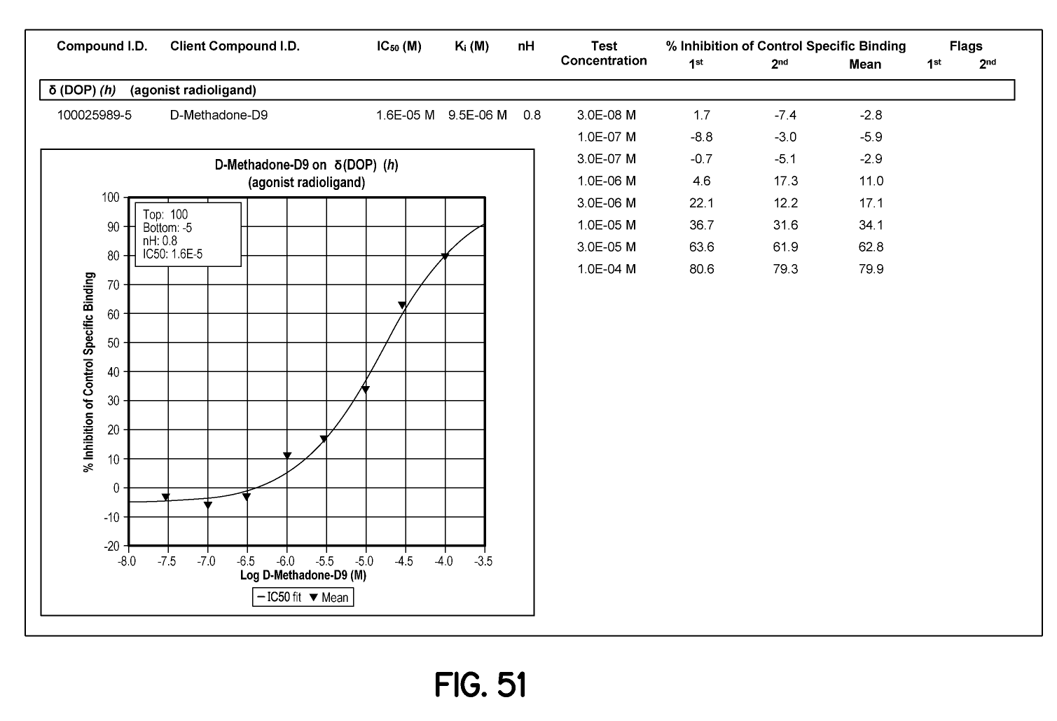

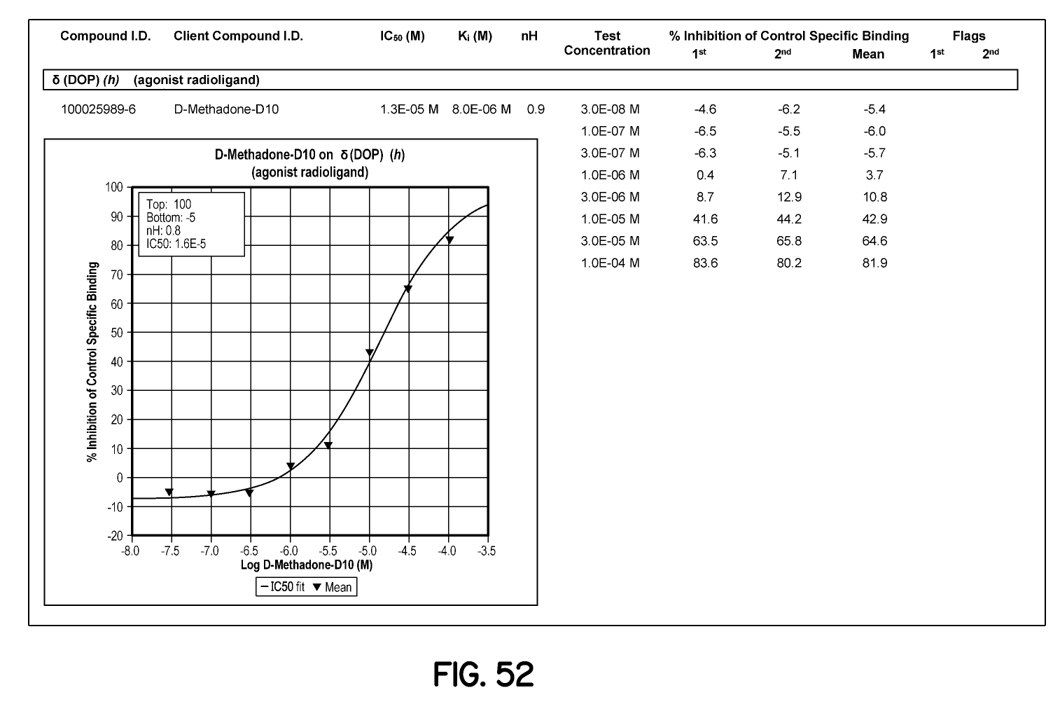

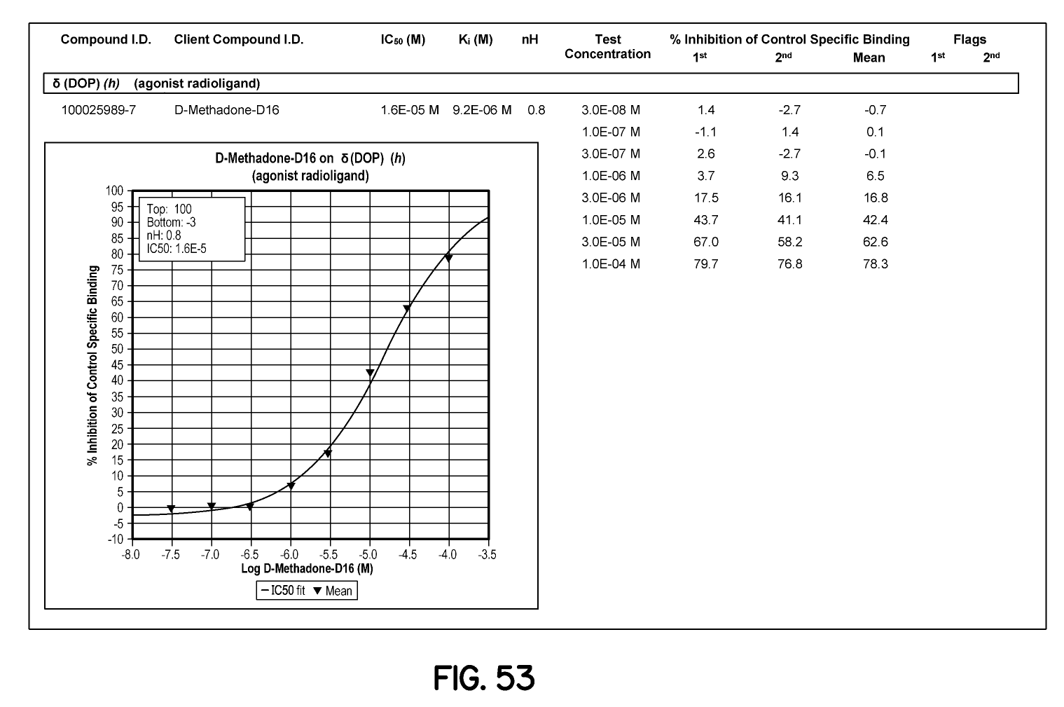

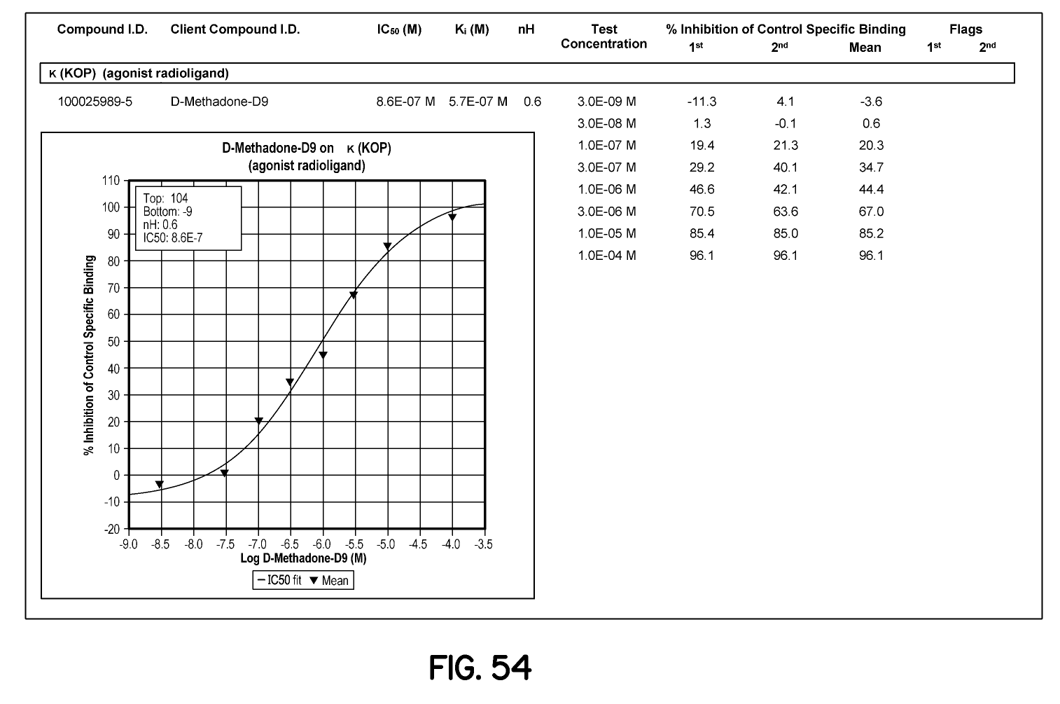

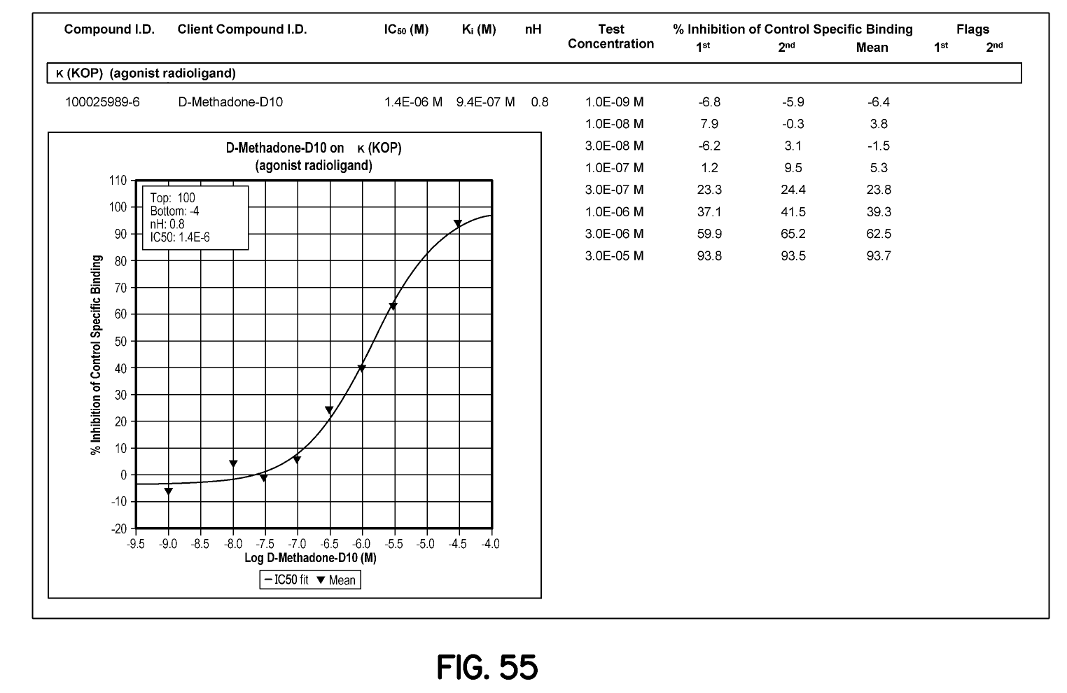

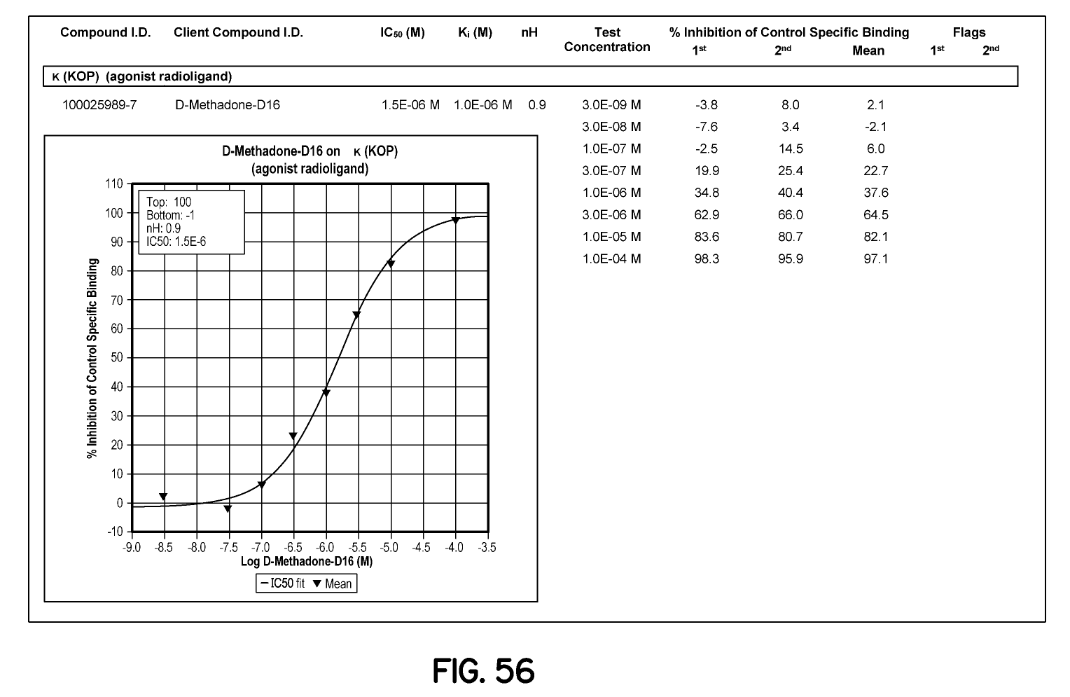

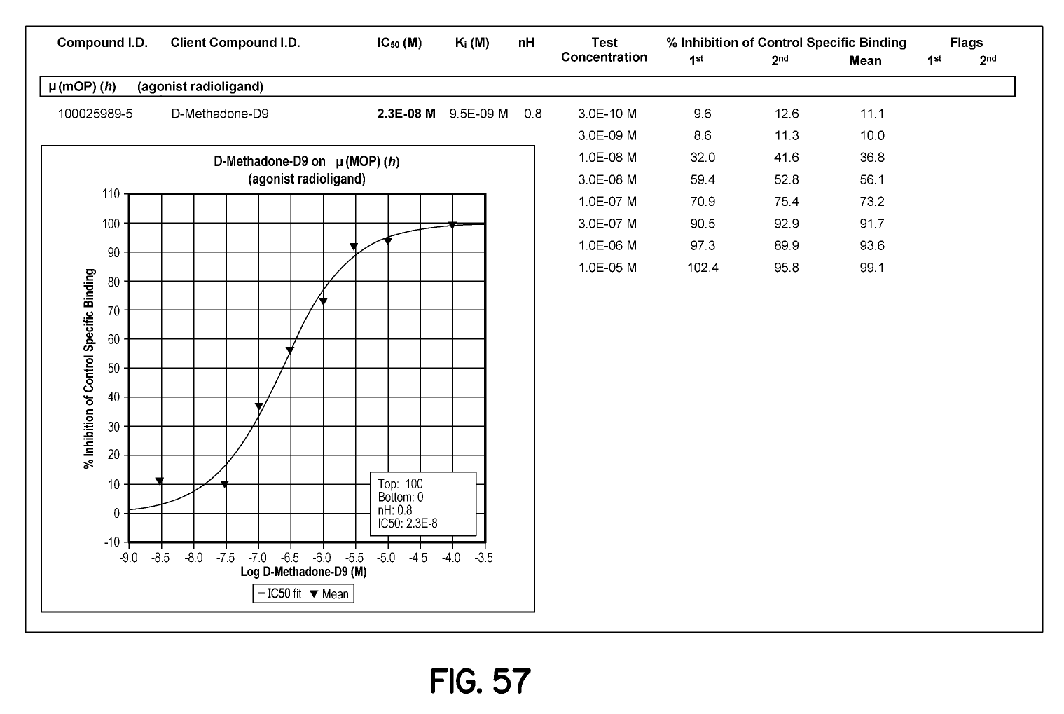

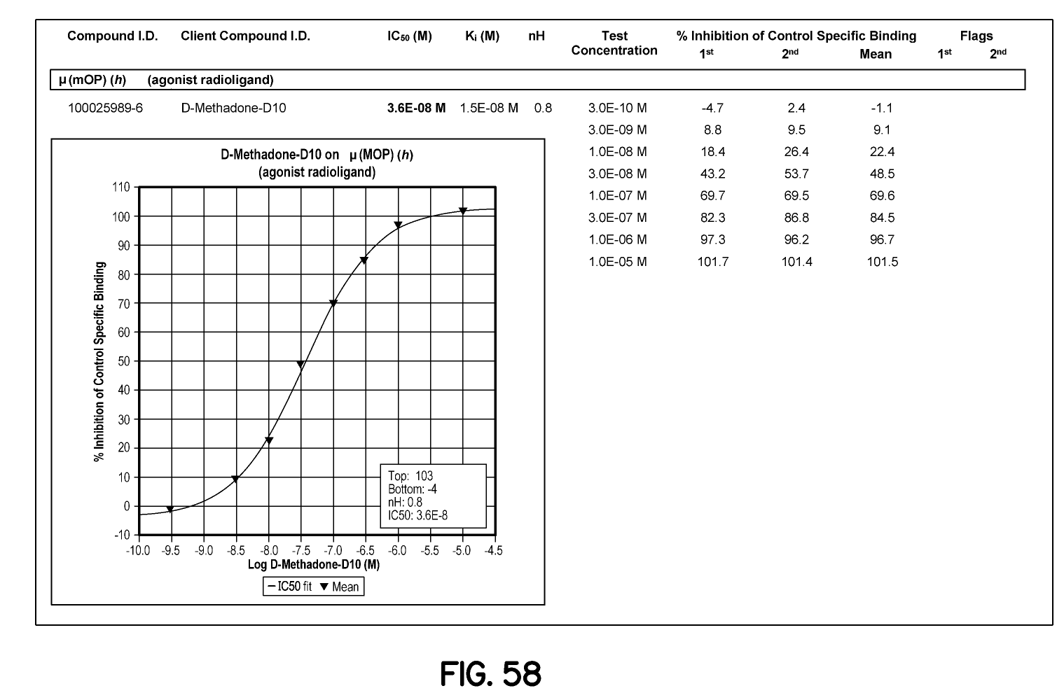

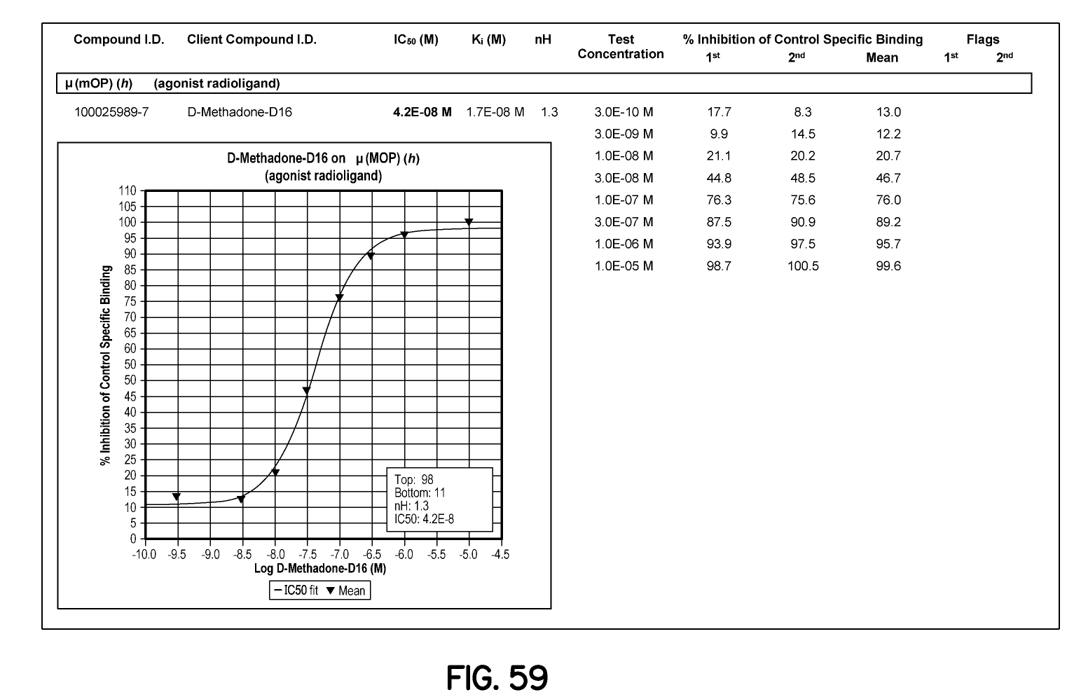

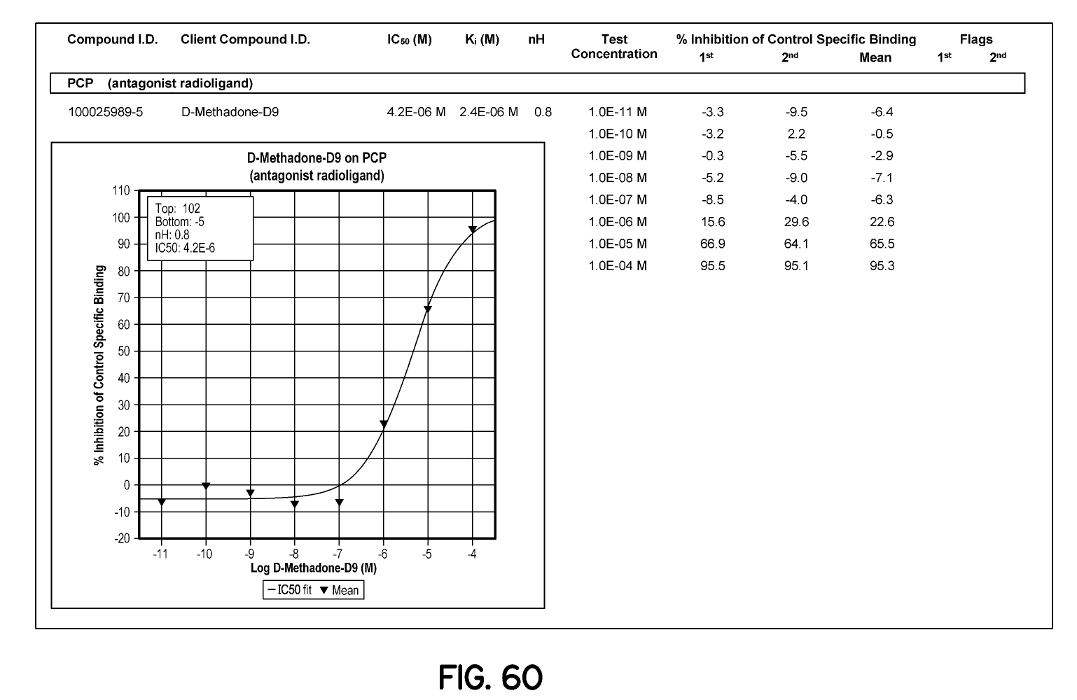

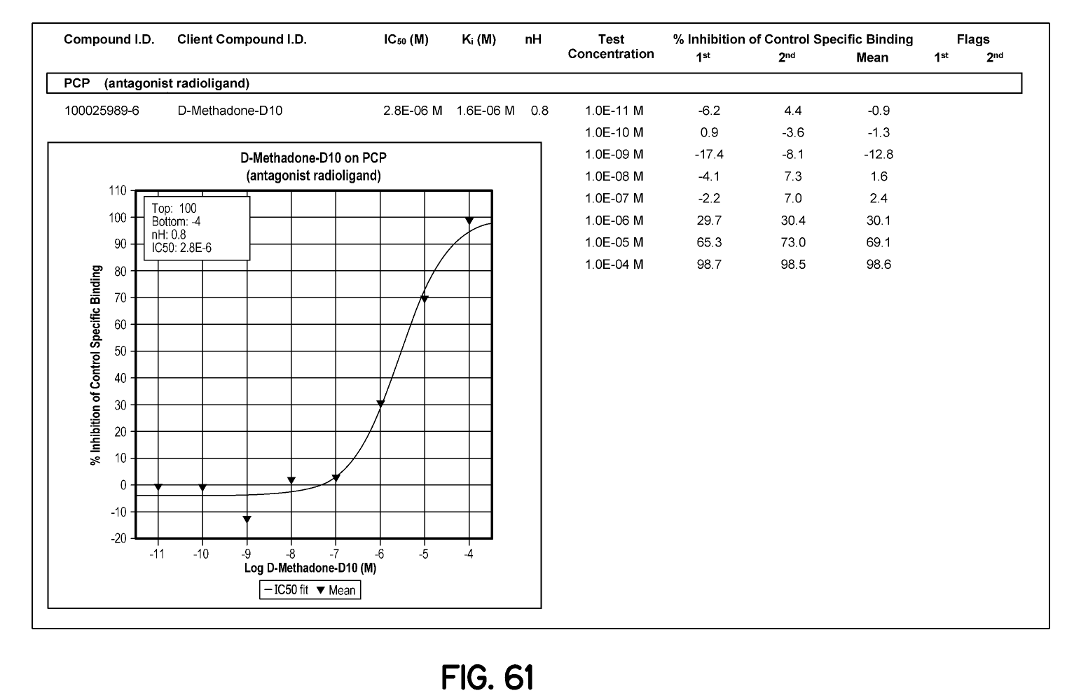

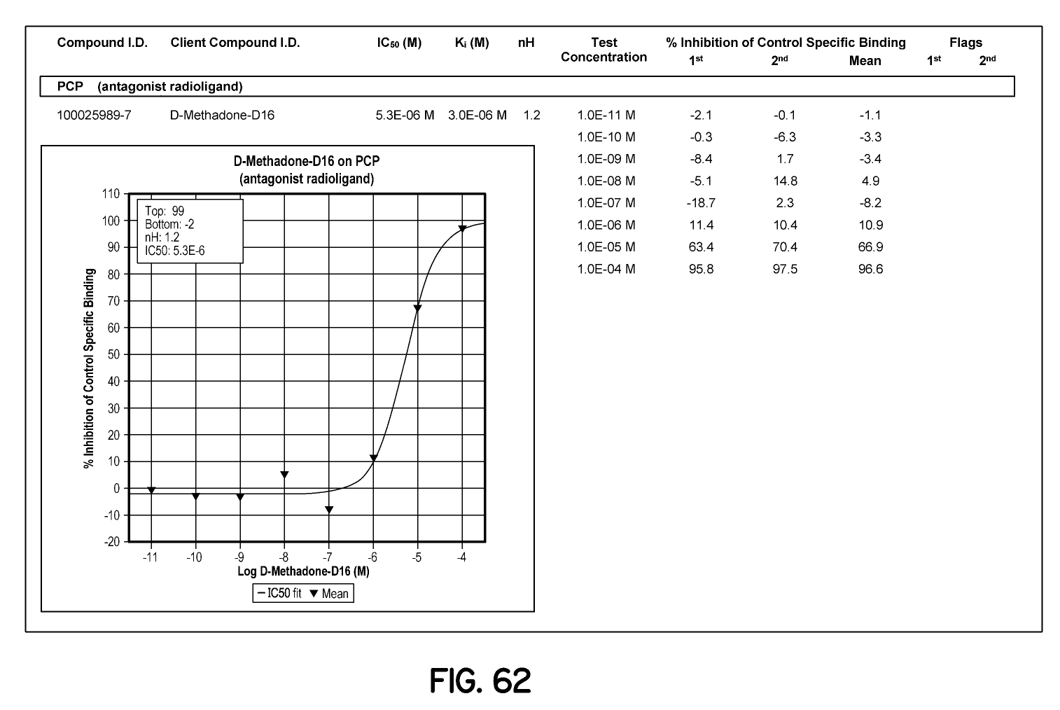

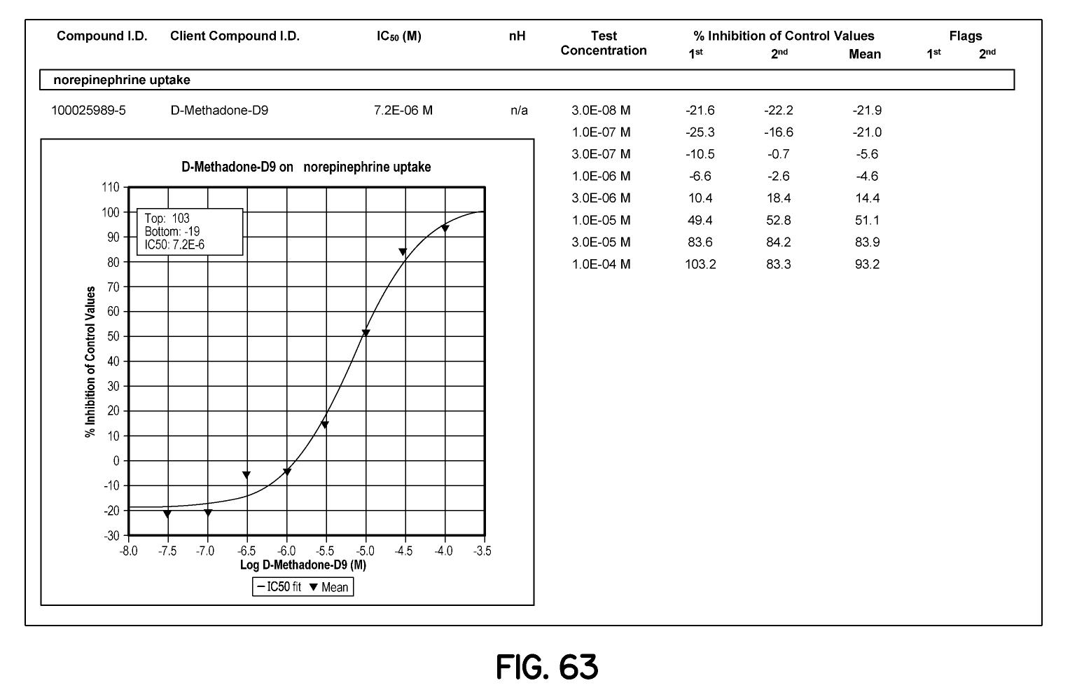

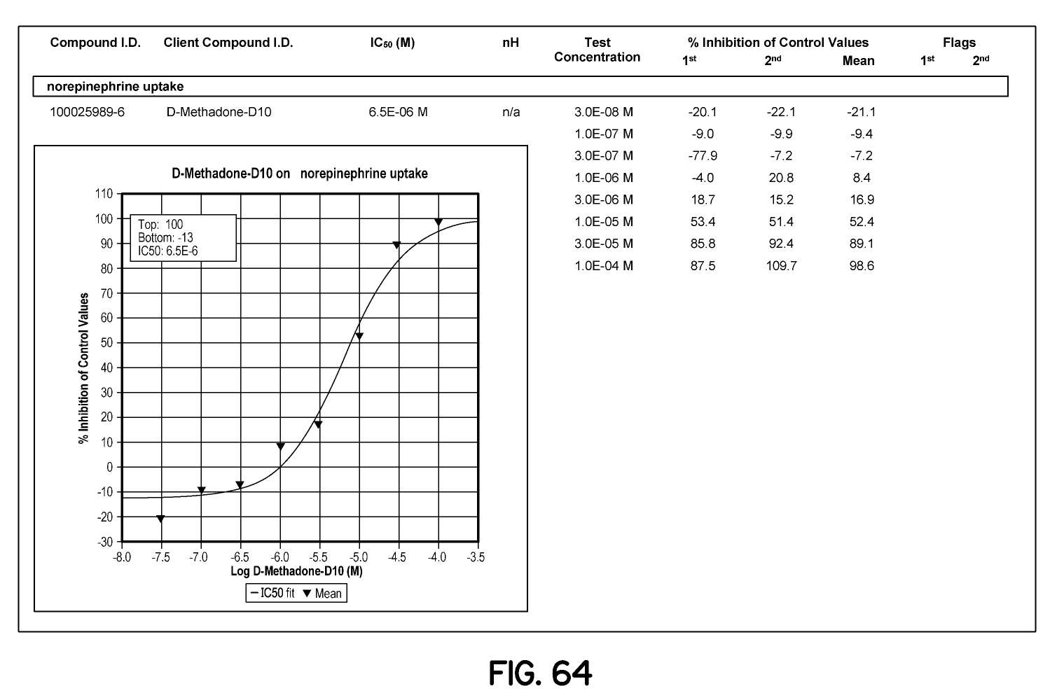

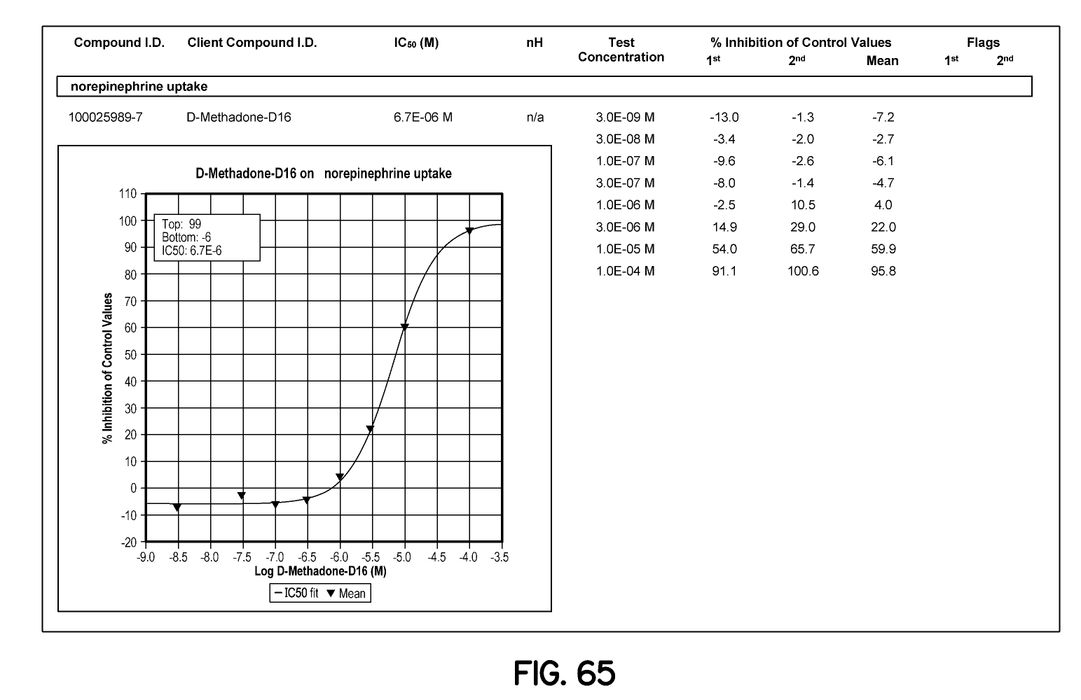

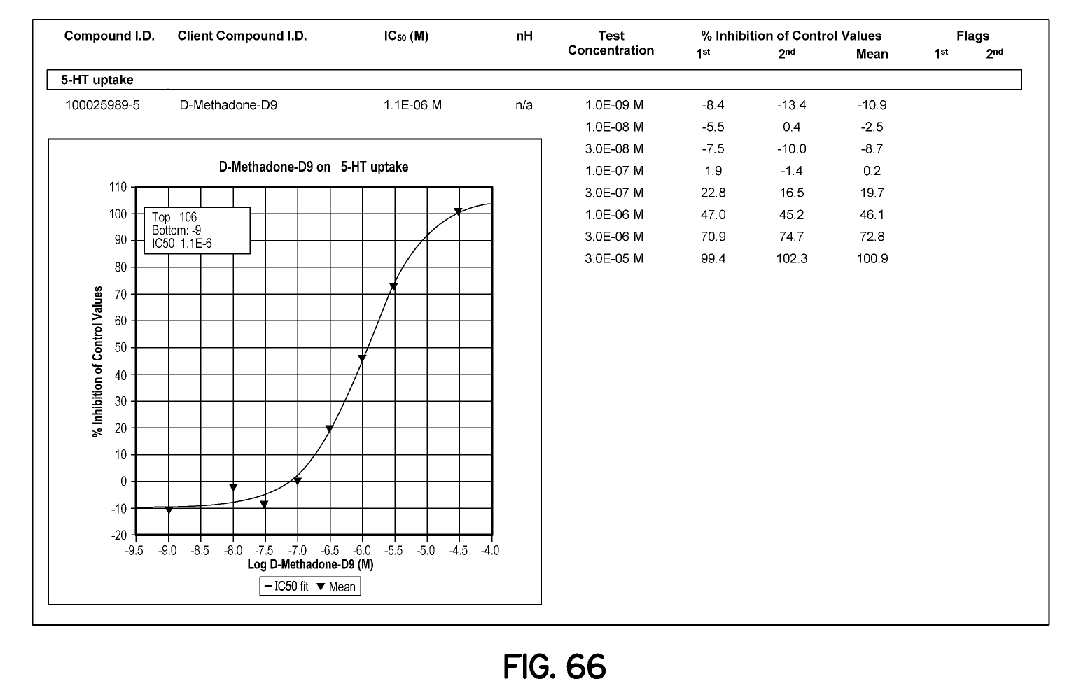

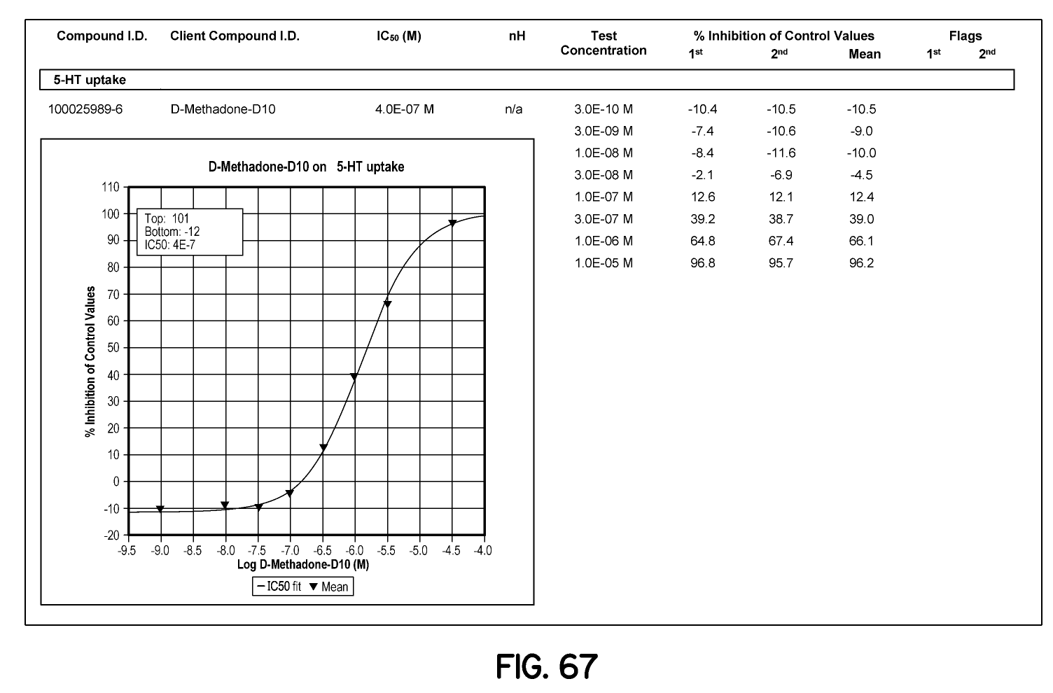

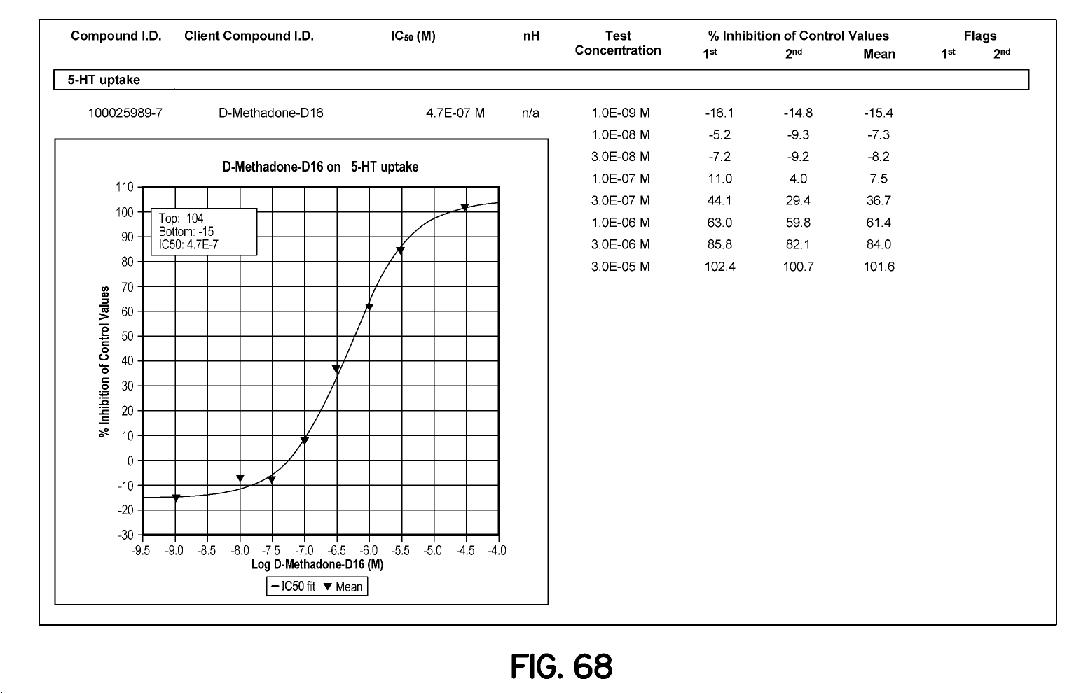

Results of the assays of this Study 2 of the Example are shown in FIGS. 22-45 and 51-68, and in Tables 71 and 72 (below).

In Vitro Pharmacology: Binding Assays (IC₅₀ Determination: Test Compound Results:

Results of the determination of IC₅₀ in test compounds in the in vitro pharmacology binding assays are shown in FIGS. 22-37 and 51-62.

TABLE 71
IC₅₀ Determination: Reference Compound Results

| | | | |
|---|---|---|---|
| Compound I.D. | IC₅₀ (M) | Kᵢ (M) | nH |

δ (DOP) (h) (agonist radioligand)

| | | | |
|---|---|---|---|
| DPDPE | 3.9E−09 M | 2.3E−09 M | 1.1 |
| DPDPE | 2.9E−09 M | 1.7E−09 M | 0.9 |
| DPDPE | 3.8E−09 M | 2.2E−09 M | 0.9 |

κ (KOP) (agonist radioligand)

| | | | |
|---|---|---|---|
| U 50488 | 1.4E−09 M | 9.0E−10 M | 1.5 |
| U 50488 | 1.4E−09 M | 9.6E−10 M | 1.3 |
| U 50488 | 1.1E−09 M | 7.5E−10 M | 1.0 |

μ (MOP) (h) (agonist radioligand)

| | | | |
|---|---|---|---|
| DAMGO | 4.9E−10 M | 2.0E−10 M | 0.7 |
| DAMGO | 3.4E−10 M | 1.4E−10 M | 0.7 |
| DAMGO | 9.4E−10 M | 3.9E−10 M | 1.0 |
| DAMGO | 1.0E−09 M | 4.3E−10 M | 0.6 |

PCP (antagonist radioligand)

| | | | |
|---|---|---|---|
| MK 801 | 4.9E−09 M | 2.8E−09 M | 0.9 |
| MK 801 | 9.8E−09 M | 5.5E−09 M | 1.1 |

In Vitro Pharmacology: Enzyme and Uptake Assays (IC₅₀ Determination: Test Compound Results):

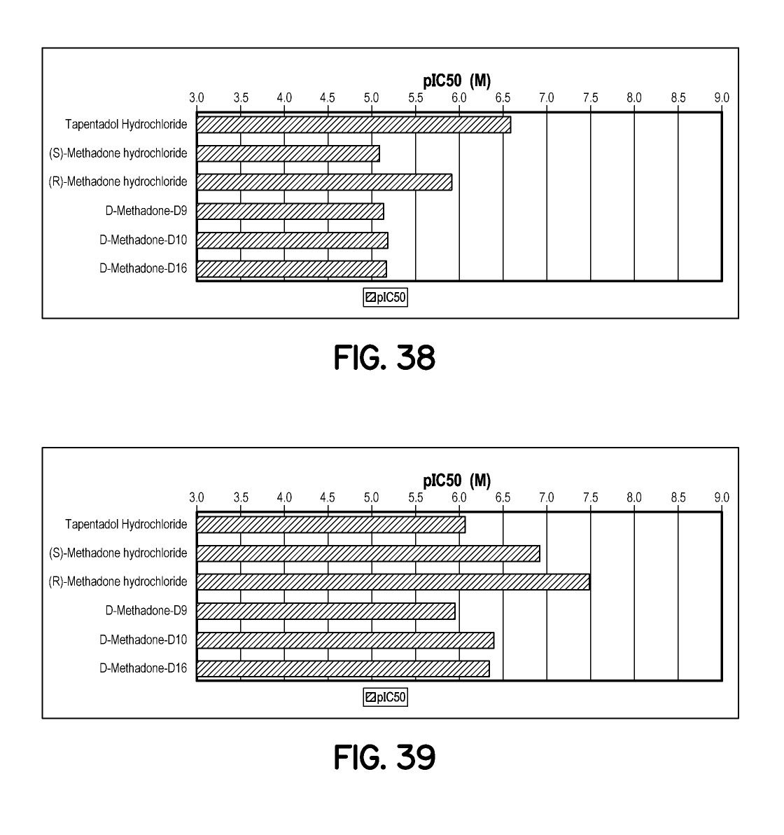

Results of the determination of IC₅₀ in test compounds in the in vitro pharmacology and uptake assays are shown in FIGS. 38-45 and 63-68.

TABLE 72
IC₅₀ Determination: Reference Compound Results

| | | |
|---|---|---|
| Compound I.D. | IC₅₀ (M) | nH |

norepinephrine uptake

| | | |
|---|---|---|
| protriptyline | 1.7E−09 M | n/a |

5-HT uptake

| | | |
|---|---|---|
| imipramine | 4.1E−08 M | n/a |

Results showing an inhibition (or stimulation for assays run in basal conditions) higher than 50% are considered to represent significant effects of the test compounds. 50% is the most common cut-off value for further investigation (determination of IC₅₀ or EC₅₀ values from concentration-response curves) that we would recommend.

Results showing an inhibition (or stimulation) between 25% and 50% are indicative of weak to moderate effects (in most assays, they should be confirmed by further testing as they are within a range where more inter-experimental variability can occur).

Results showing an inhibition (or stimulation) lower than 25% are not considered significant and mostly attributable to variability of the signal around the control level.

Low to moderate negative values have no real meaning and are attributable to variability of the signal around the control level. High negative values (≥50%) that are sometimes obtained with high concentrations of test compounds are generally attributable to non-specific effects of the test compounds in the assays. On rare occasion they could suggest an allosteric effect of the test compound.

Analysis and Expression of Results (In Vitro Pharmacology: Binding Assays): The results are expressed as a percent of control specific binding

$$\frac{measured specific binding}{control specific binding} * 100$$

and as a percent inhibition of control specific binding

$$100 - ( \frac{measured specific binding}{control specific binding} * 100 )$$

obtained in the presence of the test compounds.

The IC₅₀ values (concentration causing a half-maximal inhibition of control specific binding) and Hill coefficients (nH) were determined by non-linear regression analysis of the competition curves generated with mean replicate values using Hill equation curve fitting

$$Y = D + [ \frac{A - D}{1 + {( C \text{/} {C}_{50} )}^{nH}} ]$$

where Y=specific binding, A=left asymptote of the curve, D=right asymptote of the curve, C=compound concentration, C₅₀=IC₅₀, and nH=slope factor. This analysis was performed using software developed at Cerep (Hill software) and validated by comparison with data generated by the commercial software SigmaPlot® 4.0 for Windows® (© 1997 by SPSS Inc.).

The inhibition constants (Kᵢ) were calculated using the Cheng Prusoff equation

$${K}_{i} = \frac{{IC}_{50}}{( 1 + L \text{/} {K}_{D} )}$$

where L=concentration of radioligand in the assay, and K_{D}=affinity of the radioligand for the receptor. A scatchard plot is used to determine the K_{D}.

Analysis and Expression of Results (In Vitro Pharmacology: Enzyme and Uptake Assays):

The results are expressed as a percent of control specific activity

$$\frac{measured specific binding}{control specific binding} * 100$$

and as a percent inhibition of control specific activity

$$100 - ( \frac{measured specific binding}{control specific binding} * 100 )$$

obtained in the presence of the test compounds.

The IC₅₀ values (concentration causing a half-maximal inhibition of control specific activity), EC₅₀ values (concentration producing a half-maximal increase in control basal activity), and Hill coefficients (nH) were determined by non-linear regression analysis of the inhibition/concentration-response curves generated with mean replicate values using Hill equation curve fitting

$$Y = D + [ \frac{A - D}{1 + {( C \text{/} {C}_{50} )}^{nH}} ]$$

where Y=specific activity, A=left asymptote of the curve, D=right asymptote of the curve, C=compound concentration, C₅₀=IC₅₀ or EC₅₀, and nH=slope factor.

This analysis was performed using software developed at Cerep (Hill software) and validated by comparison with data generated by the commercial software SigmaPlot® 4.0 for Windows® (© 1997 by SPSS Inc.).

Deuterated and Tritium d-Methadone and d-Methadone Analogues

As presented throughout this application, the experimental and clinical evidence presented, analyzed and interpreted by the inventors supports the use of d-methadone for many clinical indications. One of the experimental studies analyzed by the inventors suggests that deuterium incorporation increases the NMDA antagonistic affinity of d-methadone. It is unknown if this change in antagonistic activity at the NMDA receptor after deuteration of d-methadone will be reproducible in different studies and if it will potentially modify the clinical effects of d-methadone. However, because changes in NMDAR antagonistic activity may change the clinical effects of d-methadone, the inventors plan to investigate the structural features of deuterium methadone that resulted in the higher antagonist affinity, incorporate these features into d-methadone and d-methadone analogue, and further evaluate deuterated d-methadone and deuterated d-methadone analogues for the same clinical indications proposed for d-methadone. Examples of deuterated d-methadone showing increased NMDA affinity are presented herein. Examples of deuterated d-methadone analogue compounds include: (−)-[Acetyl-2H3]α-acetylmethadol hydrochloride; and (−)-[2,2,3-2H3]α-Acetylmethadol hydrochloride. While tritium (hydrogen-3) reacts with other substances in a manner similar to hydrogen, the difference in their masses sometimes causes differences in chemical properties of the compounds. Examples of tritium d-methadone analogue compounds with possibly clinically useful NMDA blocking activity include: (−)-[1,2-3H]α-Acetylnormethadol hydrochloride; (−)-[1,1,1,2,2,3-2H6]α-Acetylmethadol hydrochloride; (−)-[1,2-3H2]α-Acetylmethadol [see DRUG SUPPLY PROGRAM CATALOG 25TH EDITION MAY 2016 (The National Institute on Drug Abuse (NIDA) Drug Supply Program (DSP)].

As described above, available drugs for the treatment of NS disorders and their neurological symptoms and manifestations are few and often have side effects that limit their use. Additional therapeutic strategies are needed for eye diseases, endocrine-metabolic diseases and the control of blood pressure. Based on the scientific work described throughout this application, including in the Examples section, and the clinical experience of the inventors, d-methadone is expected to be well tolerated by the majority of patients with these disorders and has the potential to act as a modulator of neurotransmission and neuronal plasticity in circumscribed areas of altered function, rather than acting on all cells. Specifically, d-methadone is expected to exert its regulatory functions in circumscribed regions where the NMDA system is chronically and pathologically up-regulated, and/or where the NET and SERT systems are down-regulated, or where BDNF or testosterone levels are inadequate, without significantly influencing normally functioning cells. And so, d-methadone will possibly: (1) be effective and well tolerated for various NS disorders (such as early Alzheimer's disease); (2) be more effective and better tolerated than memantine for various NS disorders (such as moderate and severe Alzheimer's disease); (3) offer an alternative to patients unable to tolerate memantine because of renal impairment or other reasons; (4) be better tolerated than available drugs, including stimulants, for ADHD and other disorders of the cognitive function, of learning and of memory; (5) be effective and better tolerated than methadone for restless leg syndrome, epilepsy, fibromyalgia, migraine and other headaches and peripheral neuropathy of different etiology; (6) offer a therapeutic option for CNS diseases and symptoms for which there are very few or no options available; and (7) be effective for eye diseases and symptoms, endocrine-metabolic diseases and the control of blood pressure

The embodiments of the present invention recited herein are intended to be merely exemplary and those skilled in the art will be able to make numerous variations and modifications to it without departing from the spirit of the present invention. Notwithstanding the above, certain variations and modifications, while producing less than optimal results, may still produce satisfactory results. All such variations and modifications are intended to be within the scope of the present invention as defined by the claims appended hereto.

What is claimed is:

1. A method for treating or preventing nervous system disorders, endocrine-metabolic disorders, cardiovascular disorders, age-related disorders, eye diseases, skin diseases, or symptoms and manifestations thereof, or for improving cognitive function, the method comprising:
administering to a subject a substance chosen from d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, 2-ethylidene-1,5-dimethyl-3,3-diphenylpyrrolidine (“EDDP”), 2-ethyl-5-methyl-3,3-diphenylpyrroline (“EMDP”), d-isomethadone, normethadone, N-methyl-methadone, N-methyl-d-methadone, N-methyl-l-methadone, l-moramide, pharmaceutically acceptable salts thereof, and mixtures thereof;
wherein the substance is isolated from its enantiomer or synthesized de novo; and
wherein the administering of the substance occurs under conditions effective for the substance to:
(a) regulate the levels of brain-derived neurotrophic factor (BDNF) or testosterone in the subject,
(b) bind to an NMDA receptor, a NET, or a SERT of the subject or
(c) modulate K⁺, Ca²⁺, or Na⁺ currents of cells of the subject.

2. The method of claim 1, wherein the substance is d-methadone.

3. The method of claim 2, wherein the administering of d-methadone is performed orally, buccally, sublingualy, rectally, vaginally, nasally, via aerosol, transdermally, parenterally, epidurally, intrathecally, intra-auricularly, intraocularly, or topically including eye drops and other ophthalmic formulations, including iontophoresis and dermatologic formulations.

4. The method of claim 2, further comprising administering a second substance to the subject in combination with the administering of d-methadone.

5. The method of claim 4, wherein the second substance in combination with d-methadone is chosen from: NMDA channel blockers chosen from memantine, dextromethorphan, and amantadine; ketamine; (±)-5-(Aminocarbonyl)-10,11-dihydro-5H-dibenzo[a,d]cyclohepten-5,10-imine hydrochloride (ADCI HCl); CGS 19755 (Selfotel); glycine/NMDA receptor antagonists chosen from 7-Chloro-4-hydroxy-3-(-3-phenoxyphenyl)-2(1H)quinoline (L 701,324); (+)-((R)-3-Amino-1-hydroxypyrrolidin-2-one [(+)-(R)-HA-966]; (±)-3-Amino-1-hydroxypyrrolidin-2-one [(±)-HA-966]; cholinesterase inhibitors; mood stabilizers; anti-psychotics; clozapine; CNS stimulants; amphetamines; anti-depressants; anxiolytics; lithium; magnesium; zinc; glutamine; glutamate; aspartame; aspartate; analgesics; opioidergic drugs; opioid antagonistschosen from naltrexone, nalmefene, naloxone, 1-naltrexol, dextronaltrexone, Nociceptin Opioid Receptor (NOP) antagonists, and selective k-opioid receptor antagonists; nicotine receptor agonists and nicotine; tauroursodeoxycholic acid (TUDCA); other bile acids, obethicolic acid, idebenone, phenylbutyric acid (PBA), other aromatic fatty acids, calcium-channel blockers, nitric oxide synthase inhibitors, levodopa, bromocriptine, other anti-Parkinson drugs, riluzole, edavarone, antiepileptic drugs, prostaglandins, beta-blockers, alpha-adrenergic agonist, carbonic anhydrase inhibitors, parasympathomimetics, epinephrine, hyperosmotic agents, hypoglycemic agents, antihypertensive agents, anti-ischemic agents, anti-obesity drugs, corticosteroids, immunosuppressants, and non steroidal anti-inflammatory drugs.

6. The method of claim 1, wherein the subject is a mammal.

7. The method of claim 5, wherein the mammal is a human.

8. The method of claim 4, wherein the administering of the second substance and the d-methadone is performed orally, buccally, sublingualy, rectally, vaginally, nasally, via aereosol, trans-dermally, parenterally, epidurally, intrathecally, intra-auricularly, intraocularly, including implanted depot formulations, or topically, including eye drops and other ophthalmic formulations, including iontophoresis and dermatologic formulations.

9. The method of claim 2, further comprising: administering at least one of the following compounds in combination with the administering of d-methadone: methadone, l-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, isomethadone, l-isomethadone, d-isomethadone, normethadone and N-methyl-methadone, N-methyl-d-methadone, N-methyl-l-methadone, phenaxodone, l-phenaxodone, d-phenaxodone; diampromide, l-diampromide and d-diampromide; moramide, d-moramide and l-moramide, levopropoxyphene.

10. The method of claim 2, wherein the d-methadone is in the form of a pharmaceutically acceptable salt.

11. The method of claim 2, wherein the d-methadone is administered intravenously

12. The method of claim 2, wherein the d-methadone is delivered at a total daily dosage of about 0.01 mg to about 5,000 mg.

13. The method of claim 1, wherein the nervous system disorder is chosen from Alzheimer's disease; presenile dementia; senile dementia; vascular dementia; Lewy body dementia; cognitive impairment; Parkinson's disease; Parkinsonian related disorders; disorders associated with accumulation of beta amyloid protein; disorders associated with accumulation or disruption of tau protein and its metabolites, frontal variant, primary progressive aphasias, semantic dementia, progressive non fluent aphasia, corticobasal degeneration, supranuclear palsy; epilepsy; NS trauma; NS infections; NS inflammation, cytopathology from toxins; stroke; multiple sclerosis; Huntington's disease; mitochondrial disorders; Leigh syndrome; LHON; Fragile X syndrome; Angelman syndrome; hereditary ataxias; neuro-otological and eye movement disorders; neurodegenerative diseases of the retina; amyotrophic lateral sclerosis; tardive dyskinesias; hyperkinetic disorders; attention deficit hyperactivity disorder; attention deficit disorders; restless leg syndrome; Tourette's syndrome; schizophrenia; autism spectrum disorders; tuberous sclerosis; Rett syndrome; cerebral palsy; disorders of the reward system; binge eating disorder; trichotillomania; dermotillomania; nail biting; migraine; fibromyalgia; and peripheral neuropathy of any etiology.

14. The method of claim 1, wherein the symptom or manifestation of nervous system disorders is chosen from a decline, impairment, or abnormality in cognitive abilities chosen from executive function, attention, cognitive speed, memory, language functions, orientation in space and time, praxis, ability to perform actions, ability to recognize faces or objects, concentration, and alertness; abnormal movements chosen from akathisia, bradykinesia, tics, myoclonus, dyskinesias, dystonias, tremors, and restless leg syndrome; parasomnias; insomnia; disturbed sleep pattern; psychosis; delirium; agitation; headache; motor weakness; spasticity; impaired physical endurance; sensory impairment; dysesthesias; dysautonomia; ataxia; impairment of balance or coordination; tinnitus; neuro-otological and eye movement impairments; neurological symptoms and manifestations of alcohol withdrawal, chosen from delirium, headache, tremors, and hallucinations; impaired social skills, hyperventilation; apnea; hand wringing; scoliosis; microcephaly; and self injurious behavior chosen from trichotillomania, dermotillomania, nail biting; and itching.

15. The method of claim 1, wherein the endocrine-metabolic disorder is chosen from the metabolic syndrome, obesity, hyperglycemia, type 2 diabetes mellitus, high blood pressure, coronary artery disease chosen from myocardial infarction, angina, and unstable angina, nonalcoholic fatty liver disease (NAFLD), nonalcoholic steatohepatitis (NASH), hypogonadism, testosterone insufficiency, hypothalamic-pituitary axis disorders, BDNF insufficiency chosen fromWAGR syndrome, 11p deletion, and 11p inversion, Prader-Willi syndrome, Smith-Magenis syndrome, and ROHHAD syndrome.

16. The method of claim 1, wherein the disorder associated with physiologic or accelerated aging and its symptoms and manifestations is chosen from cognitive impairments, sarcopenia, osteoporosis, sexual dysfunction, impaired physical endurance, sensory impairment, and impairment of hearing, smell, taste, balance or vision.

17. The method of claim 1, wherein the eye disease or symptom is chosen from optic nerve diseases, retinal diseases, vitreal diseases, corneal diseases, glaucoma, dry eye syndrome, and midriasis.

18. The method of claim 1, wherein the skin disease or symptom is chosen from psoriasis, eczema, vitiligo, and skin inflammation from multiple causes chosen from autoimmune diseases and physical causes or radiation therapy; and self injurious behavior chosen from trichotillomania, dermotillomania, nail biting; and itching.

19. The method of claim 13, 14, 15, 16, 17, or 18, wherein the substance is d-methadone, and wherein the method further comprises administering naltrexone in combination with the d-methadone.

20. The method of claim 19, wherein the combination of d-methadone and naltrexone is further administered to treat or prevent one or more of cough; pain; neuropathic pain; alcohol withdrawal; psychiatric disorders chosen from depression, anxiety, pseudobulbar affect, fatigue, and obsessive compulsive disorder; self-injurious behaviors chosen from trichotillomania, dermotillomania, and nail biting; depersonalization disorder; addiction to prescription drugs, illicit drugs, or alcohol; and behavioral addictions.

21. The method of claim 13, 14, 15, 16, 17, or 18, wherein the substance is d-methadone, and wherein the method further comprises administering a second substance in combination with d-methadone, wherein the second substance is chosen from magnesium, magnesium threonate, zinc, and pharmaceutically acceptable salts thereof.

22. The method of claim 21, wherein the combination of d-methadone and said second substance is further administered to treat one or more of cough; pain; neuropathic pain; alcohol withdrawal; addiction to prescription drugs, illicit drugs, or alcohol; and behavioral addictions.

23. A method for treating or preventing a condition including nervous system disorders, endocrine-metabolic disorders, cardiovascular disorders, age-related disorders, eye diseases, skin diseases, or symptoms and manifestations thereof, or for improving cognitive function, the method comprising:
administering naltrexone to a subject in combination with at least one substance chosen from methadone, l-methadone, d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, EDDP, EMDP, isomethadone, l-isomethadone, d-isomethadone, normethadone, N-methyl-methadone, N-methyl-d-methadone, N-methyl-l-methadone, phenaxodone, l-phenaxodone, d-phenaxodone; diampromide, l-diampromide, d-diampromide, moramide, d-moramide, l-moramide, racemorphan-like drugs, dextromethorphan, racemorphan, dextrorphan, 3-methoxymorphinan, 3-hydroxymorphinan, levorphanol, levallorphan, buprenorphine, tramadol, meperidine, pethidine, normeperidine, norpethidine, propoxyphene, norpropoxyphene, dextropropoxyphene, levopropoxyphene, fentanyl, norfentanyl, morphine, oxycodone, hydromorphone, and metabolites thereof;
wherein the substance is isolated from its enantiomer or synthesized de novo; and
wherein the administering of the substance occurs under conditions effective for the substance to:
(a) regulate the levels of brain-derived neurotrophic factor (BDNF) or testosterone in the subject,
(b) bind to an NMDA receptor, a NET, or a SERT of the subject or
(c) modulate K⁺, Ca²⁺, or Na⁺ currents of cells of the subject.

24. A method for treating or preventing a condition including nervous system disorders, endocrine-metabolic disorders, cardiovascular disorders, age-related disorders, eye diseases, skin diseases, or symptoms and manifestations thereof, or for improving cognitive function, the method comprising:
administering to a subject a substance chosen from d-isomethadone, l-moramide, levopropoxyphene, metabolites thereof, and combinations thereof;
wherein the substance is isolated from its enantiomer or synthesized de novo; and
wherein the administering of the substance occurs under conditions effective for the substance to:
(a) regulate the levels of brain-derived neurotrophic factor (BDNF) or testosterone in the subject,
(b) bind to an NMDA receptor, a NET, or a SERT of the subject or
(c) modulate K⁺, Ca²⁺, or Na⁺ currents of cells of the subject.

25. A method for treating or preventing nervous system disorders, endocrine-metabolic disorders, cardiovascular disorders, age-related disorders, eye diseases, skin diseases, or symptoms and manifestations thereof, or for improving cognitive function, the method comprising:
administering to a subject a substance chosen from deuterated or tritium analogues of: d-methadone, beta-d-methadol, alpha-l-methadol, beta-l-methadol, alpha-d-methadol, acetylmethadol, d-alpha-acetylmethadol, l-alpha-acetylmethadol, beta-d-acetylmethadol, beta-l-acetylmethadol, d-alpha-normethadol, l-alpha normethadol, noracetylmethadol, dinoracetylmethadol, methadol, normethadol, dinormethadol, 2-ethylidene-1,5-dimethyl-3,3-diphenylpyrrolidine (“EDDP”), 2-ethyl-5-methyl-3,3-diphenylpyrroline (“EMDP”), d-isomethadone, normethadone, N-methyl-methadone, N-methyl-d-methadone, N-methyl-l-methadone, l-moramide, and levopropoxyphene;
wherein the substance is isolated from its enantiomer or synthesized de novo; and
wherein the administering of the substance occurs under conditions effective for the substance to:
(a) regulate the levels of brain-derived neurotrophic factor (BDNF) or testosterone in the subject,
(b) bind to an NMDA receptor, a NET, or a SERT of the subject or
(c) modulate K⁺, Ca²⁺, or Na⁺ currents of cells of the subject.

26. The method of claim 25, further comprising administering d-methadone in combination with said substance.
